(19) United States
(12) Patent Application Publication
(10) Pub. No.: US 2007/0167421 A1
(43) Pub. Date: Jul. 19, 2007

(54) Pyrimidine derivatives and use thereof as agricultural and horticultural fungicides

(76) Inventors: Mashito Ito, Ibaraki (JP); Mamoru Hatazawa, Ibaraki (JP); Yasuo Araki, Tochigi (JP); Tetsuya Inuta, Tochigi (JP)

Correspondence Address: BAYER CROPSCIENCE LP;Patent Department, 100 BAYER ROAD, PITTSBURGH, PA, 15205-9741, US

(21) Appl. No.: 10/589,113

(22) PCT Filed: Feb. 11, 2005
(86) PCT No.: PCT/EP05/01383

§ 371 Date: Nov. 14, 2006

(30) Foreign Application Priority Data
Feb. 19, 2004 (JP) 2004-043405

(51) Int. Cl. A01N 43/54; C07D 403/02

(52) U.S. Cl. 514/183; 514/256; 514/269; 514/235.5; 514/252.14; 514/252.18; 544/122; 544/295; 544/317; 544/324

(57) ABSTRACT

This invention relates to an agrohorticultural fungicide containing benzylpyrimidine derivatives represented by the formula 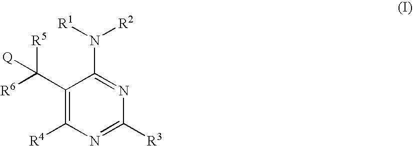
wherein R¹, R², R³, R⁴, R⁵, and R⁶ are as defined in the disclosure.

The present invention relates to the use of benzylpyrimidine derivatives as agricultural and horticultural fungicides, to novel benzylpyrimidine derivatives and to a process for their preparation.

It has been already known that some kinds of pyrimidine derivatives show an action as fungicides (cf. for example, German Patent Specification No. 4029649, PCT International Laid-open Pamphlet WO 02/74753, PCT International Laid-open Pamphlet WO 03/43993, European Patent Specification No. 4034762, European Patent Specification No. 407899, Japanese Laid-open Patent Publication No. 283246/1996).

It has been also known that some kinds of pyrimidine derivatives have various physiological activities (cf. for example, PCT International Laid-open Pamphlet WO 92/18498: Enhancement of anti-tumor activities, PCT International Laid-open Pamphlet WO 99/19305: Action to central nervous system, PCT International Laid-open Pamphlet WO 00/61562: Action to nervous system, Swiss Patent Specification No. 479591: Pharmacological action).

Further, in the field of organic chemistry, various pyrimidine derivatives have been synthesized and reported (cf. for example, Journal of Organic Chemistry, Vol. 65, p. 9261-9264 (2000), Armyanskii Khimicheskii Zhurnal, Vol. 22, No. 5, p. 401-405 (1969), Armyanskii Khimicheskii Zhurnal, Vol. 23, No. 5, p. 462-468 (1970), Armyanskii Khimicheskii Zhurnal, Vol. 24, No. 1, p. 45-50 (1971), Armyanskii Khimicheskii Zhurnal, Vol. 24, No. 8, p. 721-726 (1971),).

It has now been found that a group of benzylpyrimidine derivatives of the following formula (I) have fungicidal activities; 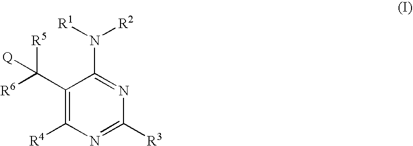
wherein
R¹ and R² form, together with the nitrogen atom to which they are bonded, a 3 to 10-membered heterocyclic group that may be optionally substituted, and may contain further one to three hetero atoms selected from the group consisting of nitrogen atom, oxygen atom and S(O)ₙ, besides the nitrogen atom to which R¹ and R² are bonded,
n represents 0, 1 or 2,
R³ represents hydrogen, halogen, cyano, hydroxy, amino, azido, alkyl, haloalkyl, alkoxyalkyl, cycloalkyl, alkenyl, alkynyl, alkoxy, haloalkoxy, alkenyloxy, haloalkenyloxy, alkylthio, alkenylthio, haloalkenylthio, alkylsulfinyl, alkylsulfonyl, phenoxy that may be optionally substituted, benzyloxy that may be optionally substituted, phenyl that may be optionally substituted, phenylalkyl that may be optionally substituted, phenoxyalkyl that may be optionally substituted, or 5 to 10-membered heterocyclic group that contains one to four hetero atoms selected from the group consisting of nitrogen atom, oxygen atom and sulfur atom and may be optionally substituted with a group selected from the group consisting of halogen, alkyl and haloalkyl, or
R³ represents a group selected from the group consisting of the following groups A-H and J-M 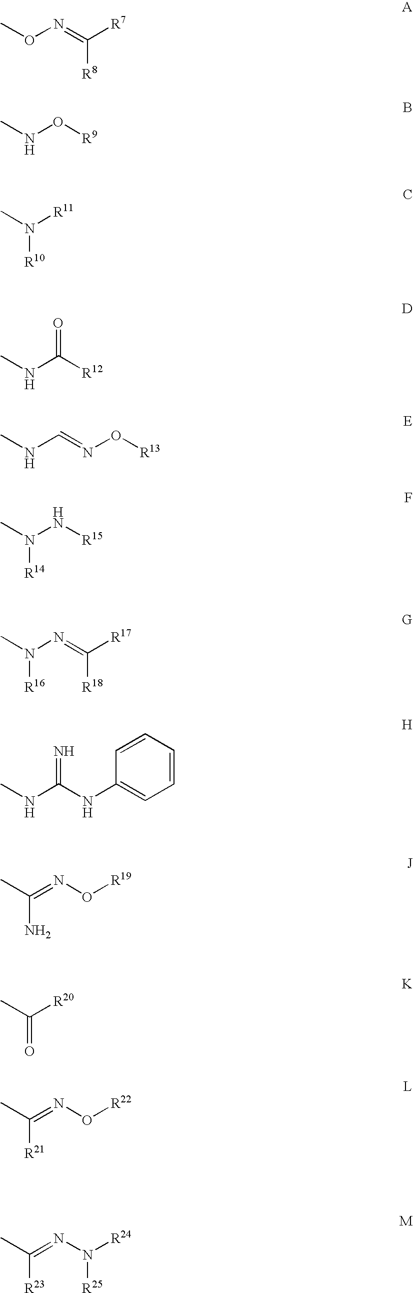
in which
R⁷ represents hydrogen atom, alkyl or haloalkyl, and
R⁸ represents alkyl, phenyl, alkoxy or cyano, or
R⁷ and R⁸ form, together with the carbon atom to which they are bonded, cycloalkylidene,
R⁹ represents alkyl, haloalkenyl or benzyl,
R¹⁰ represents hydrogen atom or alkyl,
R¹¹ represents alkyl, alkoxyalkyl, dialkylaminoalkyl, phenyl, benzyl or cyano,
R¹² represents alkyl or phenyl,
R¹³ represents alkyl or benzyl,
R¹⁴ represents hydrogen atom or alkyl,
R¹⁵ represents hydrogen atom, haloalkyl or phenyl,
R¹⁶ represents hydrogen atom or alkyl,
R¹⁷ represents hydrogen atom, alkyl or haloalkyl,
R¹⁸ represents alkyl or phenyl,
R¹⁹ represents hydrogen atom or alkyl,
R²⁰ represents alkyl,
R²¹ represents alkyl,
R²² represents alkyl, alkenyl, haloalkenyl, alkoxyalkyl, phenoxyalkyl or alkoxycarbonylalkyl,
R²³ represents alkyl,
R²⁴ represents hydrogen atom or alkyl,
R²⁵ represents alkyl or phenyl,
R²⁴ and R²⁵ form, together with the nitrogen atom to which they are bonded, a 5 to 8-membered saturated-monoheterocyclic group that may be optionally substituted, and may contain further one or two hetero atoms selected from the group consisting of nitrogen atom, oxygen atom and S(O)ₙ, besides the nitrogen atom to which R²⁴ and R²⁵ are bonded,
R⁴ represents hydrogen atom, halogen, cyano, alkyl, haloalkyl, alkenyl, alkynyl, alkoxy, haloalkoxy, alkylthio, haloalkylthio, alkylsulfinyl, alkylsulfonyl or group 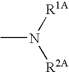
R⁵ and R⁶ each independently represents hydrogen atom, halogen, alkyl, haloalkyl, or phenyl that may be optionally substituted, and
Q represents aryl that may be optionally substituted or a 5 or 6-membered heterocyclic group that contains one hetero atom selected from the group consisting of nitrogen atom, oxygen atom and sulfur atom and may be optionally substituted.

The benzylpyrimidine derivatives of the following formula (IA) being included in the aforementioned formula (I), according to the present invention are novel compounds that have not been described in the existing publications.

The formula 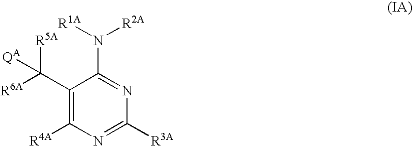
wherein
R^{1A} and R^{2A} form, together with the nitrogen atom to which they are bonded, a
3 to 10-membered heterocyclic group that may be optionally substituted, and may contain further one to three hetero atoms selected from the group consisting of nitrogen atom, oxygen atom and S(O)ₘ, besides the nitrogen atom to which R^{1A} and R^{2A} are bonded,
m represents 0, 1 or 2,
R^{3A} represents hydrogen, halogen, cyano, hydroxy, amino, azido, alkyl, haloalkyl, alkoxyalkyl, cycloalkyl, alkenyl, alkynyl, alkoxy, haloalkoxy, alkenyloxy, haloalkenyloxy, alkylthio, alkenylthio, haloalkenylthio, alkylsulfinyl, alkylsulfonyl, phenoxy that may be optionally substituted, benzyloxy that may be optionally substituted, phenyl that may be optionally substituted, phenylalkyl that may be optionally substituted, phenoxyalkyl that may be optionally substituted, or 5 to 10-membered heterocyclic group that contains one to four hetero atoms selected from the group consisting of nitrogen atom, oxygen atom and sulfur atom and may be optionally substituted with a group selected from the group consisting of halogen, alkyl and haloalkyl, or
R^{3A} represents a group selected from the group consisting of the following groups A-H and J-M 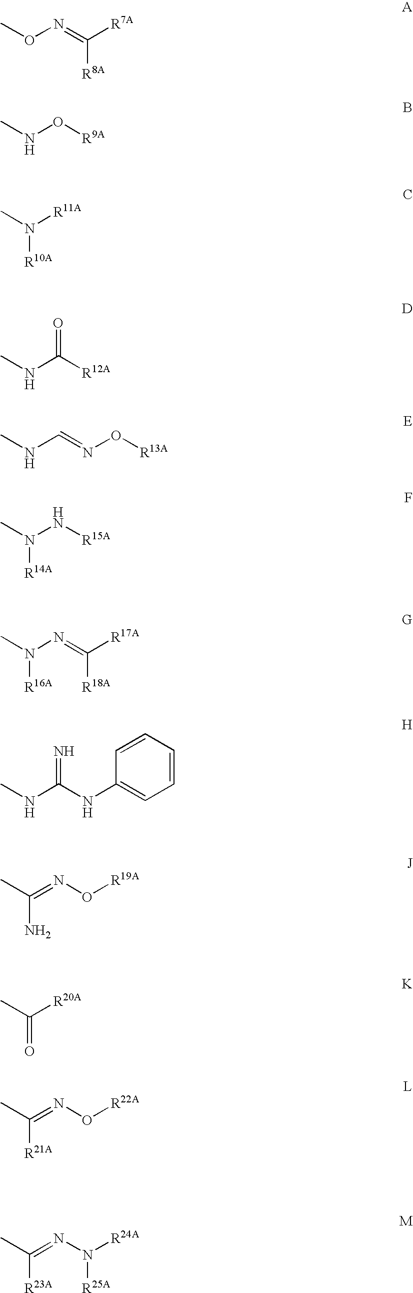
in which
R^{7A} represents hydrogen atom, alkyl or haloalkyl, and
R^{8A} represents alkyl, phenyl, alkoxy or cyano, or
R^{7A} and R^{8A} form, together with the carbon atom to which they are bonded, cycloalkylidene,
R^{9A} represents alkyl, haloalkenyl or benzyl,
R^{10A} represents hydrogen atom or alkyl,
R^{11A} represents alkyl, alkoxyalkyl, dialkylaminoalkyl, phenyl, benzyl or cyano,
R^{12A} represents alkyl or phenyl,
R^{13A} represents alkyl or benzyl,
R^{14A} represents hydrogen atom or alkyl,
R^{15A} represents hydrogen atom, haloalkyl or phenyl,
R^{16A} represents hydrogen atom or alkyl,
R^{17A} represents hydrogen atom, alkyl or haloalkyl,
R^{18A} represents alkyl or phenyl,
R^{19A} represents hydrogen atom or alkyl,
R^{20A} represents alkyl,
R^{21A} represents alkyl,
R^{22A} represents alkyl, alkenyl, haloalkenyl, alkoxyalkyl, phenoxyalkyl or alkoxycarbonylalkyl,
R^{23A} represents alkyl,
R^{24A} represents hydrogen atom or alkyl,
R^{25A} represents alkyl or phenyl,
R^{24A} and R^{25A} form, together with the nitrogen atom to which they are bonded, a 5 to 8-membered saturated-monoheterocyclic group that may be optionally substituted, and may contain further one or two hetero atoms selected from the group consisting of nitrogen atom, oxygen atom and S(O)ₙ, besides the nitrogen atom to which R^{24A} and R^{25A} are bonded,
R^{4A} represents hydrogen atom, halogen, cyano, alkyl, haloalkyl, alkenyl, alkynyl, alkoxy, haloalkoxy, alkylthio, haloalkylthio, alkylsulfinyl, alkylsulfonyl or group 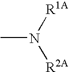
R^{5A} and R^{6A} each independently represents hydrogen atom, halogen, alkyl, haloalkyl, or phenyl that may be optionally substituted, and
Q^{A} represents aryl that may be optionally substituted or a 5 or 6-membered heterocyclic group that contains one hetero atom selected from the group consisting of nitrogen atom, oxygen atom and sulfur atom and may be optionally substituted,
provided that, the following cases (T-1)-(T-6) are excluded:
(T-1) the case in which group 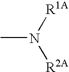
represents 1-indolyl, 1-pyrrolyl, 1-imidazolyl, 3-oxopiperidino or 4-oxopiperidino, R^{3A} represents hydrogen atom, R^{4A} represents hydrogen atom, and Q^{A} represents 1-naphthyl or phenyl group that may be optionally substituted by one or two groups selected from the group consisting of chloro, bromo, methyl, ethyl and trifluoromethyl,
(T-2) the case in which group 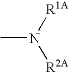
represents 3-oxopiperidino, 4-oxopiperidino, 4-hydroxypiperidino, 4-carbamoylpiperidino, 4-methylpiperazino, 4-ethylpiperazino, 4-(2-hydroxyethyl)piperazino or morpholino, R^{3A} represents amino, R^{4A} represents hydrogen atom, and Q^{A} represents 3-pyridyl or phenyl group that may be optionally substituted by one to three groups selected from the group consisting of fluoro, chloro, bromo, methyl, ethyl, isopropyl, trifluoromethyl, hydroxy, methoxy and 4-chlorobenzyloxy,
(T-3) the case in which group 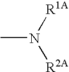
represents piperidino, 4-hydroxypiperidino, 4-methylpiperazino, morpholino, 6,7-dimethoxy-1,2,3,4-tetrahydroisoquinolin-2-yl or 6,7-dimethoxy-1-(3,4-dimethoxybenzyl)-1,2,3,4-tetrahydroisoquinolin-2-yl, R^{3A} represents chloro, dimethylamino, anilino, 2-(2-hydroxyethoxy)ethylamino, piperidino, 4-hydroxypiperidino, 4-carbamoylpiperidino, 4-methylpiperazino or morpholino,
R^{4A} represents hydrogen atom, and Q^{A} represents phenyl group that may be optionally substituted by one or two groups selected from the group consisting of methyl and methoxy,
(T-4) the case in which group 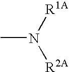
represents 1-pyrrolidinyl, piperidino, morpholino or 1-pyrrolyl, R^{3A} represents methyl or methoxymethyl, R^{4A} represents chloro, and Q^{A} represents phenyl or 1-naphthyl,
(T-5) the case in which group 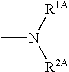
represents 1-azilidinyl, piperidino or morpholino, R^{3A} represents methylthio, R^{4A} represents chloro, and Q^{A} represents phenyl group substituted by methoxy, ethoxy, n-propoxy, iso-propoxy, n-butoxy, iso-butoxy or allyloxy,
(T-6) the case in which group 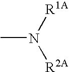
represents 1-azilidinyl, R^{3A} represents hydrogen atom or amino, R^{4A} represents chloro, and Q^{A} represents phenyl group substituted by methoxy, ethoxy or allyloxy.

The compound of the formula (IA) can be obtained by a process in which

a) In case that R^{3A} represents hydrogen, alkyl, haloalkyl, alkoxyalkyl, cycloalkyl, alkenyl, alkylthio, alkenylthio, haloalkenylthio, phenyl that may be optionally substituted, phenylalkyl that may be optionally substituted, phenoxyalkyl that may be optionally substituted, or 5 to 10-membered heterocyclic group that contains one to four hetero atoms selected from the group consisting of nitrogen atom, oxygen atom and sulfur atom and may be optionally substituted by a group selected from the group consisting of halogen, alkyl and haloalkyl, and R^{4A} represents hydrogen atom, halogen, alkyl, haloalkyl or alkenyl:
compounds of the formula (II) 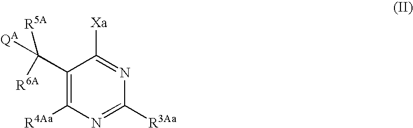
wherein
Xa represents halogen, preferably chloro or bromo,
R^{3Aa} represents hydrogen, alkyl, haloalkyl, alkoxyalkyl, cycloalkyl, alkenyl, alkylthio, alkenylthio, haloalkenylthio, phenyl that may be optionally substituted, phenylalkyl that may be optionally substituted, phenoxyalkyl that may be optionally substituted, or 5 to 10-membered heterocyclic group that contains one to four hetero atoms selected from the group consisting of nitrogen atom, oxygen atom and sulfur atom and may be optionally substituted by a group selected from the group consisting of halogen, alkyl and haloalkyl, and
R^{4Aa} represents hydrogen atom, halogen, alkyl, haloalkyl or alkenyl,
R^{5A}, R^{6A} and Q^{A} have the same definition as aforementioned, are reacted with compounds of the formula (III) 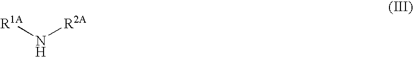
wherein
R^{1A} and R^{2A} have the same definition as aforementioned,
in the presence of innert solvents, and if appropriate, in the presence of an acid binder,
or
b) in case that R^{3A} represents alkylsulfinyl or alkylsulfonyl and R^{4A} represents hydrogen atom, halogen, cyano, alkyl, haloalkyl, alkenyl, alkynyl, alkoxy, haloalkoxy or group 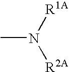
or
R^{3A} represents hydrogen, alkyl, haloalkyl, alkoxyalkyl, cycloalkyl, alkenyl, phenyl that may be optionally substituted, phenylalkyl that may be optionally substituted, phenoxyalkyl that may be optionally substituted, or 5 to 10-membered heterocyclic group that contains one to four hetero atoms selected from the group consisting of nitrogen atom, oxygen atom and sulfur atom and may be optionally substituted by a group selected from the group consisting of halogen, alkyl and haloalkyl, and R^{4A} represents alkylsulfinyl or alkylsulfonyl:
compounds of the formula (IAb) 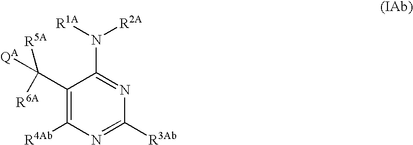
wherein
R^{3Ab} represents alkylthio, and R^{4Ab} represents hydrogen atom, halogen, cyano, alkyl, haloalkyl, alkenyl, alkynyl, alkoxy, haloalkoxy or group 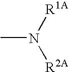
or
R^{3Ab} represents hydrogen, alkyl, haloalkyl, alkoxyalkyl, cycloalkyl, alkenyl, phenyl that may be optionally substituted, phenylalkyl that may be optionally substituted, phenoxyalkyl that may be optionally substituted, or 5 to 10-membered heterocyclic group that contains one to four hetero atoms selected from the group consisting of nitrogen atom, oxygen atom and sulfur atom and may be optionally substituted by a group selected from the group consisting of halogen, alkyl and haloalkyl, and R^{4Ab} represents allylthio,
R^{1A}, R^{2A}, R^{5A}, R^{6A} and Q^{A} have the same definition as aforementioned,
are reacted with an oxidizing agent in the presence of innert solvents,
or,
c) in case that R^{3A} represents cyano, hydroxy, azido, alkynyl, alkoxy, haloalkoxy, alkenyloxy, haloalkenyloxy, alkylthio, alkenylthio, haloalkenylthio, phenoxy that may be optionally substituted, benzyloxy that may be optionally substituted, or 5 to 10-membered heterocyclic group that contains one to four hetero atoms selected from the group consisting of nitrogen atom, oxygen atom and sulfur atom and may be optionally substituted by a group selected from the group consisting of halogen, alkyl and haloalkyl, or represents the aforementioned group A, group B, group C, group F, group G or group H, and
R^{4A} represents hydrogen atom, halogen, alkyl, haloalkyl, alkenyl, alkynyl, alkoxy, haloalkoxy, cyano or group 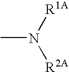
compounds of the formula (IAc) 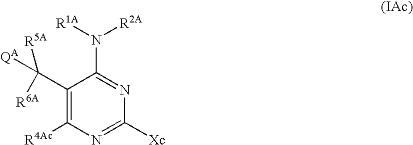
   wherein
   Xc represents halogen, preferably chloro, bromo or iodo, or methylsulfonyl,
   R^{4Ac} represents hydrogen atom, halogen, alkyl, haloalkyl, alkenyl, alkynyl, alkoxy, haloalkoxy, cyano or group 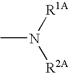
   R^{1A}, R^{2A}, R^{5A}, R^{6A} and Q^{A} have the same definition as aforementioned,
   are reacted with compounds of the formula (IV)
   Y—R^{3Ac} (IV)
   wherein
   Y represents hydrogen, sodium, potassium, copper, trimethylsilyl or tetraalkylammonium,
   R^{3Ac} represents cyano, hydroxy, azido, alkynyl, alkoxy, haloalkoxy, alkenyloxy, haloalkenyloxy, alkylthio, alkenylthio, haloalkenylthio, phenoxy that may be optionally substituted, benzyloxy that may be optionally substituted, or 5 to 10-membered heterocyclic group that contains one to four hetero atoms selected from the group consisting of nitrogen atom, oxygen atom and sulfur atom and may be optionally substituted by a group selected from the group consisting of halogen, alkyl and haloalkyl, or represents the aforementioned group A, group B, group C, group F, group G or group H,
   in the presence of innert solvents, and if appropriate, in the presence of an acid binder, and if appropriate, in the presence of a catalyst,
   or
   d) In case that R^{3A} represents hydrogen, alkyl, haloalkyl, alkoxyalkyl, cycloalkyl, alkenyl, alkylthio, alkenylthio, haloalkenylthio, phenyl that may be optionally substituted, phenylalkyl that may be optionally substituted, phenoxyalkyl that may be optionally substituted, or 5 to 10-membered heterocyclic group that contains one to four hetero atoms selected from the group consisting of nitrogen atom, oxygen atom and sulfur atom and may be optionally substituted by a group selected from the group consisting of halogen, alkyl and haloalkyl, and
   R^{4A} represents cyano, alkynyl, alkoxy, haloalkoxy, alkylthio, haloalkylthio or group 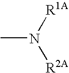
compounds of the formula (IAd) 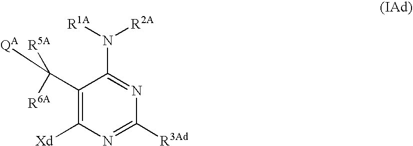
   wherein
   Xd represents halogen, preferably chloro, bromo or iodo, or methylsulfonyl,
   R^{3Ad} represents hydrogen, alkyl, haloalkyl, alkoxyalkyl, cycloalkyl, alkenyl, alkylthio, alkenylthio, haloalkenylthio, phenyl that may be optionally substituted, phenylalkyl that may be optionally substituted, phenoxyalkyl that may be optionally substituted, or 5 to 10-membered heterocyclic group that contains one to four hetero atoms selected from the group consisting of nitrogen atom, oxygen atom and sulfur atom and may be optionally substituted by a group selected from the group consisting of halogen, alkyl and haloalkyl,
   R^{1A}, R^{2A}, R^{5A}, R^{6A} and Q^{A} have the same definition as aforementioned,
   are reacted with compounds of the formula (V)
   Y—R^{4Ad} (V)
   wherein
   Y represents hydrogen, sodium, potassium, copper, trimethylsilyl or tetraalkylammonium,
   R^{4Ad} represents cyano, alkynyl, alkoxy, haloalkoxy, alkylthio, haloalkylthio, or group 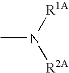
   in the presence of innert solvents, and if appropriate, in the presence of an acid binder, and if appropriate, in the presence of a catalyst,
   or
   e) In case that R^{3A} represents hydrogen, alkyl, haloalkyl, alkoxyalkyl, cycloalkyl, alkylthio, phenyl that may be optionally substituted, phenylalkyl that may be optionally substituted, phenoxyalkyl that may be optionally substituted, or 5 to 10-membered heterocyclic group that contains one to four hetero atoms selected from the group consisting of nitrogen atom, oxygen atom and sulfur atom and may be optionally substituted by a group selected from the group consisting of halogen, alkyl and haloalkyl, and
   R^{4A} represents hydrogen: compounds of the formula (IAe) 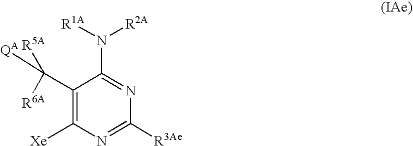
   wherein
   Xe represents halogen, preferably chloro, bromo or iodo,
   R^{3Ae} represents hydrogen, alkyl, haloalkyl, alkoxyalkyl, cycloalkyl, alkylthio, phenyl that may be optionally substituted, phenylalkyl that may be optionally substituted, phenoxyalkyl that may be optionally substituted, or 5 to 10-membered heterocyclic group that contains one to four hetero atoms selected from the group consisting of nitrogen atom, oxygen atom and sulfur atom and may be optionally substituted by a group selected from the group consisting of halogen, alkyl and haloalkyl,
   R^{1A}, R^{2A}, R^{5A}, R^{6A} and Q^{A} have the same definition as aforementioned,
   are hydrogenated in the presence of innert solvents, and if appropriate, in the presence of a catalyst, and if appropriate, in the presence of an acid binder,
   or
   f) In case that R^{3A} represents hydrogen, halogen, cyano, hydroxy, amino, azido, alkyl, haloalkyl, alkoxyalkyl, cycloalkyl, alkynyl, alkoxy, haloalkoxy, alkylthio, haloalkylthio, alkylsulfinyl, alkylsulfonyl, phenoxy that may be optionally substituted, benzyloxy that may be optionally substituted, phenyl that may be optionally substituted, phenylalkyl that may be optionally substituted, phenoxyalkyl that may be optionally substituted, or 5 to 10-membered heterocyclic group that contains one to four hetero atoms selected from the group consisting of nitrogen atom, oxygen atom and sulfur atom and may be optionally substituted by a group selected from the group consisting of halogen, alkyl and haloalkyl, or the aforementioned groups A-H or groups J-M,
   R^{4A} represents hydrogen atom, halogen, cyano, alkyl, haloalkyl, alkynyl, alkoxy, haloalkoxy, alkylthio, haloalkylthio, alkylsulfinyl, alkylsulfonyl, or group 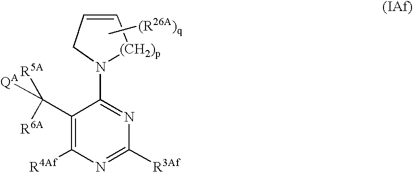
compounds of the formula (IAf) 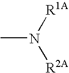
   wherein
   R^{3Af} represents hydrogen, halogen, cyano, hydroxy, amino, azido, alkyl, haloalkyl, alkoxyalkyl, cycloalkyl, alkynyl, alkoxy, haloalkoxy, alkylthio, haloalkylthio, alkylsulfinyl, alkylsulfonyl, phenoxy that may be optionally substituted, benzyloxy that may be optionally substituted, phenyl that may be optionally substituted, phenylalkyl that may be optionally substituted, phenoxyalkyl that may be optionally substituted, or 5 to 10-membered heterocyclic group that contains one to four hetero atoms selected from the group consisting of nitrogen atom, oxygen atom and sulfur atom and may be optionally substituted by a group selected from the group consisting of halogen, alkyl and haloalkyl, or the aforementioned groups A-H or groups J-M,
   R^{4Af} represents hydrogen atom, halogen, cyano, alkyl, haloalkyl, alkynyl, alkoxy, haloalkoxy, alkylthio, haloalkylthio, alkylsulfinyl, alkylsulfonyl, or group 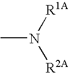
   R^{5A}, R^{6A} and Q^{A} have the same definition as aforementioned,
   R^{26A} represents alkyl, p-represents 1 or 2, q represents 0, 1 or 2,
   are reacted with difluorocarbene derived from sodium chlorodifluoroacetate or with dichlorocarbene derived from chloroform, in the presence of innert solvents, and if appropriate, in the presence of a phasetransfer catalyst,
   or
   g) In case that R^{3A} represents amino:
compounds of the formula (IAg) 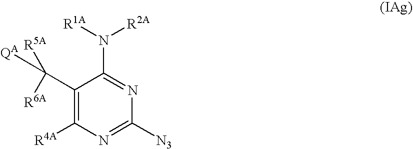
   wherein
   R^{1A}, R^{2A}, R^{4A}, R^{5A}, R^{6A} and Q^{A} have the same definition as aforementioned,
   are hydrogenated or reacted with metal hydride in the presence of innert solvents, and if appropriate, in the presence of a catalyst,
   or
   h) In case that R^{3A} represents halogen:
   First Step:
   compounds of the formula (IAh) 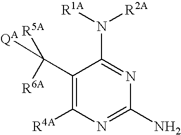
   wherein
   R^{1A}, R^{2A}, R^{4A}, R^{5A}, R^{6A} and Q^{A} have the same definition as aforementioned,
   are reacted with nitrite ester or nitrous acid in the presence of innert solvents, and if appropriate, in the presence of acid catalyst to form a diazonium salt,
   Second Step:

The diazonium salts obtained in the above-mentioned first step is reacted according to Sandmeyer process or Gattermann process in the presence of copper halide, potassium halide or copper powder,

in the presence innert solvents, and if appropriate, in the presence of acid catalyst,

or

i) In case that R^{3A} represents the aforementioned group E:

First Step:

compounds of the aforementioned formula (IAh) are reacted with dimethylformamide dimethylacetal in the presence of innert solvents,

Second Step:

compounds of the formula (VI), obtained in the above-mentioned first step, 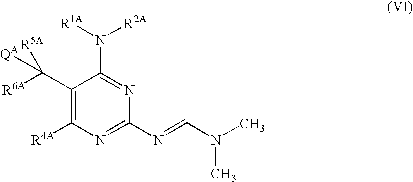
wherein
R^{1A}, R^{2A}, R^{4A}, R^{5A}, R^{6A} and Q^{A} have the same definition as aforementioned,
are reacted with compounds of the formula (VII) 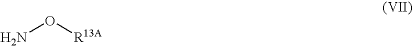
wherein
R^{13A} has the same definition as aforementioned,
in the presence of innert solvents, and if appropriate, in the presence of an acid binder, and if appropriate, in the presence of an acid catalyst,
or
j) In case that R^{3A} represents the aforementioned group D:
compounds of the formula (IAh) are reacted with compounds of the formula (VIII) 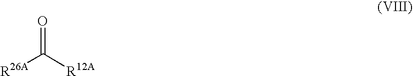
wherein
R^{26A} represents chloro or group 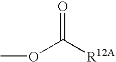
wherein
R^{12A} has the same definition as aforementioned,
in the presence of innert solvents, and if appropriate, in the presence of an acid binder,
or
k) In case that R^{3A} represents the aforementioned group K, and
R^{4A} represents hydrogen atom, halogen, alkyl, haloalkyl, alkenyl, alkynyl, alkoxy, haloalkoxy, allylthio, haloalkylthio, alkylsulfinyl, alkylsulfonyl, or group 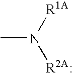
compounds of the formula (IAk) 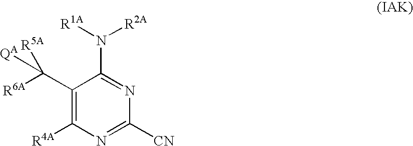
wherein
R^{4A} represents hydrogen atom, halogen, alkyl, haloalkyl, alkenyl, alkynyl, alkoxy, haloalkoxy, alkylthio, haloalkylthio, alkylsulfinyl, alkylsulfonyl, or group 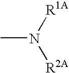
and
R^{1A}, R^{2A}, R^{5A}, R^{6A} and Q^{A} have the same definition as aforementioned,
are reacted with compounds of the formula (IX)
R^{20A}—Mg—Xk (IX)
wherein
Xk represents halogen, preferably chloro, bromo or iodo,
R^{20A} has the same definition as aforementioned,
in the presence of innert solvents,
or
1) In case that R^{3A} represents the aforementioned group L or group M, and
R^{4A} represents hydrogen atom, halogen, alkyl, haloalkyl, alkenyl, alkynyl, alkoxy, haloalkoxy, allylthio, haloalkylthio, alkylsulfinyl, alkylsulfonyl, or group 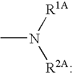
compounds of the formula (IAl)
wherein
R^{27A} represents alkyl, 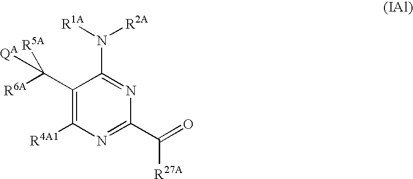
R^{4Al} represents hydrogen atom, halogen, alkyl, haloalkyl, alkenyl, alkynyl, alkoxy, haloalkoxy, alkylthio, haloalkylthio, alkylsulfinyl, alkylsulfonyl, or group 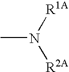
and
R^{1A}, R^{2A}, R^{5A}, R^{6A} and Q^{A} have the same definition as aforementioned,
are reacted with compounds of the formula (X)
H₂N—R^{28A} (X)
wherein
R^{28A} represents group
—O—R^{22A}
or group 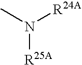
wherein
R^{22A}, R^{24A}, and R^{25A} have the same definition as aforementioned,

In the presence of innert solvents, and if appropriate, in the presence of acid

binder, and if appropriate, in the presence of acid catalyst,

or 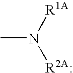
m) In case that R^{3A} represents the aforementioned group J, and
compounds of the formula (IAk) are reacted with compounds of the formula (XI)
H₂NO—R^{19A} (XI)
wherein
R^{19A} has the same definition as aforementioned,

In the presence of innert solvents, and if appropriate, in the presence of acidbinder, and if appropriate, in the presence of acid catalyst.

Active component compounds of the formula (I) of the present invention show a strong plant disease controlling action, in particular against phytopathogenic fungi.

In the present specification,

“Halogen” represents fluoro, chloro, bromo or iodo, preferably represents fluoro, chloro or bromo.

“Alkyl” can be straight-chain or branched-chain and there can be mentioned, for example, C₁₋₆alkyl, specifically methyl, ethyl, n- or iso-propyl, n-, iso-, sec- or tert-butyl, n- or neo-pentyl, n-hexyl etc.

“Cycloalkyl”: there can be mentioned, for example, cyclopropyl, cyclobutyl, cyclopentyl, cyclohexyl, cycloheptyl, etc.

“Cycloalkylidene”: there can be mentioned, for example, cyclopentylidene, cyclohexylidene, cycloheptylidene, cyclooctylidene, etc.

“Alkenyl” can be straight-chain or branched-chain and there can be mentioned, for example, C₂₋₇alkenyl, specifically vinyl, allyl, isopropenyl, 1-propenyl, 1-butenyl, 2-butenyl, 3-butenyl, 1-methyl-1-propenyl, 2-methyl-1-propenyl, 1-pentenyl, 2-pentenyl, 1-hexenyl, 2-hexenyl, 1-heptenyl, 2-heptenyl, etc.

“Alkynyl” can be straight-chain or branched-chain and there can be mentioned, for example, C₂₋₇alkynyl, specifically ethynyl, 1-propynyl, 2-propynyl, 1-butynyl 2-butynyl, 3-butynyl, 1-pentynyl, 2-pentynyl, 1-hexynyl, 2-hexynyl, 1-heptynyl, 2-heptynyl, etc.

“Alkoxy” represents an alkyl-O-group, whose alkyl part has the above-mentioned meaning and can be, for example, C₁₋₆alkoxy, and there can be specifically mentioned methoxy, ethoxy, n- or iso-propoxy, n-, iso-, sec- or tert-butoxy, n-pentyloxy, n-hexyloxy, etc.

“Alkenyloxy” represents an alkenyl-O-group, whose alkenyl part has the above-mentioned meaning and there can be mentioned, for example, allyloxy, 2-butenyloxy, 3-butenyloxy, 2-methyl-4-pentenyloxy, etc.

“Alkylthio” represents an alkyl-5-group, whose alkyl part has the above-mentioned meaning and can be, for example, C₁₋₆alkylthio, and there can be specifically mentioned methylthio, ethylthio, n- or iso-propylthio, n-, iso-, sec- or tert-butylthio, n-pentylthio, n-hexylthio, etc.

“Alkenylthio” represents an alkenyl-5-group, whose alkenyl part has the above-mentioned meaning and there can be mentioned, for example, allylthio, 2-butenylthio, 3-butenylthio, etc.

“Alkylsulfinyl” represents an alkyl-S(O)-group, whose alkyl part has the above-mentioned meaning and can be, for example, C₁₋₆alkylsulfinyl, and there can be specifically mentioned, for example, methylsulfinyl, ethylsulfinyl, n- or iso-propylsulfinyl, n-, iso-, sec- or tert-butylsulfinyl, n-pentylsulfinyl, n-hexylsulfinyl, etc.

“Alkylsulfonyl” represents an alkyl-SO₂-group, whose alkyl part has the above-mentioned meaning and can be, for example, C₁₋₆alkylsulfonyl, and there can be specifically mentioned, for example, methylsulfonyl, ethylsulfonyl, n- or iso-propylsulfonyl, n-, iso-, sec- or tert-butylsulfonyl, n-pentylsulfonyl, n-hexylsulfonyl, etc.

“Alkylcarbonyl”: there can be mentioned, for example, methylcarbonyl (acetyl), ethylcarbonyl (propionyl), etc.

“Alkylcarbonylamino”: there can be mentioned, for example, methylcarbonylamino, ethylcarbonylamino, etc.

“Alkoxycarbonyl”: there can be mentioned, for example, methoxycarbonyl, ethoxycarbonyl, etc.

“Haloalkyl” represents a straight-chain or branched-chain alkyl, at least one of whose hydrogen is substituted by halogen and there can be mentioned, for example, C₁₋₆alkyl substituted by one to six fluoro, chloro and/or bromo, and as specific examples there can be mentioned fluoromethyl, chloromethyl, dichloromethyl, bromomethyl, difluoromethyl, trifluoromethyl, chlorodifluoromethyl, dichloromethyl, trichloromethyl, 2,2,2-trifluoroethyl, 2-chloro-1,1,2-trifluoroethyl, 3-fluoropropyl, 3-chloropropyl, 2,2,3,3,3-pentafluoropropyl, 1,2,2,3,3,3-hexafluoropropyl, etc.

“Haloalkylene”: there can be mentioned, for example, difluoromethylene, dichloromethylene, etc.

Haloalkyl part in “haloalkoxy”, “haloalkylthio”, “haloalkylcarbonyl” and “haloalkylcarbonylamino” can be of the same definition as the aforementioned “haloalkyl” and specifically as “haloalkoxy” there can be mentioned, for example, difluoromethoxy, trifluoromethoxy, chlorodifluoromethoxy, dichloromethoxy, 2-fluoroethoxy, 2-chloroethoxy, 2,2,2-trifluoroethoxy, 3-chloropropoxy, etc., as “haloalkylthio” there can be mentioned, for example, difluoromethylthio,

trifluoromethylthio, 2,2,2-trifluoroethylthio, 3-fluoropropylthio, etc., as “haloalkylcarbonyl” there can be mentioned, for example, trifluoromethylcarbonyl, trichloromethylcarbonyl, 1,1,2,2-tetrafluoroethylcarbonyl, perfluoroethylcarbonyl, perfluoroheptylcarbonyl, etc. and as “haloalkylcarbonylamino” there can be mentioned, for example, trifluoromethylcarbonylamino, etc.

“Haloalkenyl” represents a straight-chain or branched-chain alkenyl, at least one of whose hydrogen is substituted with halogen and there can be mentioned, for example, 2-chloro-2-propenyl, 3-chloro-2-propenyl, 3,3-dichloro-2-propenyl, 3-chloro-4,4,4-trifluoro-2-butenyl, etc.

Haloalkenyl part in “haloalkenyloxy” and “haloalkenylthio” can be of the same definition as the aforementioned “haloalkenyl” and specifically as “haloalkenyloxy” there can be mentioned, for example, 2-chloro-2-propenyloxy, 3-chloro-2-propenyloxy, 3,3-dichloro-2-propenyloxy, 3-chloro-4,4,4-trifluoro-2-butenyloxy, etc., and as “haloalkenylthio” there can be mentioned, for example, 2-chloro-2-propenylthio, 3-chloro-2-propenylthio, 3,3-dichloro-2-propenylthio, 3-chloro-4,4,4-trifluoro-2-butenylthio, etc.

“Phenylalkyl”: there can be mentioned, for example, benzyl, 1-phenylethyl, phenethyl, 1-phenylpropyl, 2-phenylpropyl, 3-phenylpropyl, etc.

“Phenoxyalkyl”: there can be mentioned, for example, phenoxymethyl, 1-phenoxyethyl, 2-phenoxyethyl, 1-phenoxypropyl, 2-phenoxypropyl, 3-phenoxypropyl, etc.

“Alkoxyalkyl”: there can be mentioned, for example, methoxymethyl, 2-methoxyethyl, 1-methoxyethyl, 3-methoxypropyl, ethoxymethyl, 2-ethoxyethyl, etc.

“Dialkylaminoalkyl”: there can be mentioned, for example, dimethylaminomethyl, 2-dimethylaminoethyl, 1-dimethylaminoethyl, 3-dimethylaminopropyl, diethylaminomethyl, 2-diethylaminoethyl, etc.

“Alkoxycarbonylalkyl”: there can be mentioned, for example, methoxycarbonylmethyl, ethoxycarbonylmethyl, (n- or iso-) propyloxycarbonylmethyl, (n-, iso-, sec.- or tert-)butyloxycarbonylmethyl, 2-methoxycarbonylethyl, 3-methoxycarbonylpropyl, etc.

“Hydroxyalkyl”: there can be mentioned, for example, hydroxymethyl, 2-hydroxyethyl, etc.

“Anilinoalkyl”: there can be mentioned, for example, anilinomethyl, 2-anilinoethyl, etc.

“Aryl”: there can be mentioned, for example, phenyl, 1-naphthyl, 2-naphthyl, etc.

The heterocyclic group in “R¹ and R² form, together with the nitrogen atom to which they are bonded, a 3 to 10-membered heterocyclic group that may contain further one to three hetero atoms selected from the group consisting of nitrogen atom, oxygen atom and S(O)ₙ besides the nitrogen atom to which R¹ and R² are bonded” and “5 to 10-membered heterocyclic group that contains one to four hetero atoms selected from the group consisting of nitrogen atom, oxygen atom and sulfur atom”, defined in the group 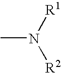
and
the heterocyclic group in “R²⁴ and R²⁵ form, together with the nitrogen atom to which they are bonded, a 5 to 8-membered, saturated, monocyclic, heterocyclic group that may contain further hetero one or two atoms selected from the group consisting of nitrogen atom, oxygen atom and S(O)ₙ, besides the nitrogen atom to which R²⁴ and R²⁵ are bonded”, defined in the group 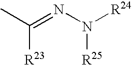
includes saturated heterocyclic group, unsaturated heterocyclic group and aromatic heterocyclic group.

Thus, as “saturated heterocyclic group” there can be mentioned monovalent group derived from, for example, aziridine, azetidine, pyrrolidine, piperidine, piperazine, morpholine, thiomorpholine, thiomorpholine-1,1-dioxide, perhydroazepine, perhydroazocine, perhydro-1,2-diazepine, perhydro-1,2,5-oxadiazepine, perhydroindole, perhydroquinoline, perhydroisoquinoline, etc.

“Unsaturated heterocyclic group”: there can be mentioned monovalent group derived from, for example, 3-pyrroline, 2-pyrazoline, thiazolidine, 2,3-dihydroindole, 1,2,3,3a,4,7,7a-heptahydroisoindole, 1,2,3,6-tetrahydropyridine, 1,4,5,6-tetrahydropiridazine, etc.

“Aromatic heterocyclic group”: there can be mentioned monovalent group derived from, for example, pyrrole, furan, thiophene, pyrazole, imidazole, thiazole, pyridine, pyridazine, pyrimidine, pyrazine, 1,2,3-triazole, 1,2,4-triazole, tetrazole, 1H-indazole, quinoline, isoquinoline, etc.

In the plant pest controlling active compounds of the aforementioned formula (I), preferably there can be mentioned the compounds in which

R¹ and R² form, together with the nitrogen atom to which they are bonded, a heterocyclic group which is a monovalent group derived from a heterocycle selected from aziridine, azetidine, pyrrolidine, 3-pyrroline, piperidine, perhydroazepine, perhydroazocine, perhydro-1,2-diazepine, perhydro-1,2,5-oxadiazepine, 2-pyrazoline, thiazolidine, perhydroindole, 1,2,3,3a,4,7,7a-heptahydroisoindole, 1,2,3,6-tetrahydropyridine, perhydroquinoline, perhydroisoquinoline, 1,4,5,6-tetrahydropyridazine, morpholine, thiomorpholine, thiomorpholine-1,1-dioxide, piperazine, pyrrole, pyrazole, imidazole, 1,2,3-triazole, 1,2,4-triazole, tetrazole or 1H-indazole and may be optionally substituted by one to three groups selected from the group consisting of fluoro, bromo, C₁₋₄alkyl, C₁₋₄haloalkyl, C₁₋₄alkoxy, C₁₋₄alkylthio, benzylthio, hydroxyC₁₋₄alkyl, C₁₋₄alkoxyC₁₋₄alkyl, anilinoC₁₋₄alkyl, C₁₋₄haloalkylene, C₁₋₄alkoxy-carbonyl, benzyloxycarbonyl, C₁₋₄alkyl-carbonyl, C₁₋₇haloalkyl-carbonyl, phenyl, benzyl, pyridyl, hydroxy, oxo, cyano, carboxy, carbamoyl, C₁₋₄alkoxy-carbonylC₁₋₄alkyl, C₁₋₄alkyl-carbonylamino and C₁₋₄haloalkyl-carbonylamino,

R³ represents hydrogen, chloro, bromo, cyano, hydroxy, amino, azido, C₁₋₆alkyl, C₁₋₆haloalkyl, C₁₋₆alkoxyC₁₋₆alkyl, C₃₋₇cycloalkyl, C₂₋₇alkenyl, C₂₋₇alkynyl, C₁₋₆alkoxy, C₁₋₆haloalkoxy, C₂₋₇alkenyloxy, C₂₋₇haloalkenyloxy, C₁₋₆alkylthio, C₂₋₇alkenylthio, C₂₋₇haloalkenylthio, C₁₋₆alkylsulfinyl, C₁₋₆alkylsulfonyl, phenoxy, benzyloxy, phenyl that may be optionally substituted by one or two groups selected from the group consisting of chloro, C₁₋₆alkyl, C₁₋₆alkoxy and C₁₋₆haloalkyl, phenylC₁₋₄alkyl that may be optionally chloro-substituted, or phenoxyC₁₋₄alkyl that may be optionally chloro-substituted, or

R³ represents a heterocyclic group which is a monovalent group derived from a heterocycle selected from pyrrolidine, piperidine, morpholine, thiomorpholine, piperazine, thiophene, thiazole, pyridine, quinoline, isoquinoline, pyrazine, pyridazine, pyrimidine, imidazole, pyrazole, tetrazole, 1,2,4-triazole and 2,3-dihydroindole, and may be optionally substituted by a group selected from the group consisting of chloro, bromo, C₁₋₆alkyl and C₁₋₆haloalkyl, or
R³ represents a group selected from the group consisting of the following groups A-H and J-M 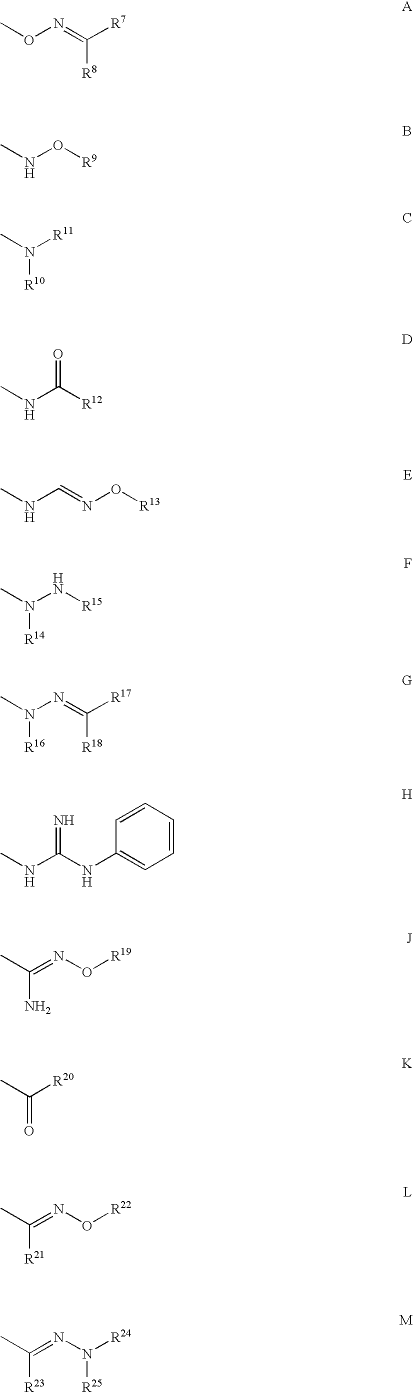
in which
R⁷ represents hydrogen atom, C₁₋₆alkyl or C₁₋₆haloalkyl,
R⁸ represents C₁₋₆alkyl, phenyl, C₁₋₆alkoxy or cyano,
R⁷ and R⁸ form, together with the carbon atom to which they are bonded, C₅₋₈cycloalkylidene,
R⁹ represents C₁₋₆alkyl, C₂₋₇haloalkenyl or benzyl,
R¹⁰ represents hydrogen atom or C₁₋₆alkyl,
R¹¹ represents C₁₋₆alkyl, C₁₋₆alkoxyC₁₋₆alkyl, di(C₁₋₆alkyl)aminoC₁₋₆alkyl,
phenyl, benzyl or cyano,
R¹² represents C₁₋₆alkyl or phenyl,
R¹³ represents C₁₋₆alkyl or benzyl,
R¹⁴ represents hydrogen atom or C₁₋₆alkyl,
R¹⁵ represents hydrogen atom, C₁₋₆haloalkyl or phenyl,
R¹⁶ represents hydrogen atom or C₁₋₆alkyl,
R¹⁷ represents hydrogen atom, C₁₋₆alkyl or C₁₋₆haloalkyl,
R¹⁸ represents C₁₋₆alkyl or phenyl,
R¹⁹ represents hydrogen atom or C₁₋₆alkyl,
R²⁰ represents C₁₋₆alkyl,
R²¹ represents C₁₋₆alkyl,
R²² represents C₁₋₆alkyl, C₂₋₇alkenyl, C₂₋₇haloalkenyl, C₁₋₆alkoxyC₁₋₆alkyl, phenoxyC₁₋₆alkyl or C₁₋₆alkoxycarbonylC₁₋₆alkyl,
R²³ represents C₁₋₆alkyl,
R²⁴ represents hydrogen atom or C₁₋₆alkyl,
R²⁵ represents C₁₋₆alkyl or phenyl,
R²⁴ and R²⁵ form, together with the nitrogen atom to which they are bonded, a saturated-monocyclic, heterocyclic group which is a monovalent group derived from a monoheterocycle selected from the group consisting of pyrrolidine, piperidine, morpholine and piperazine and may be optionally substituted by C₁₋₄alkyl,
R⁴ represents hydrogen atom, fluoro, chloro, cyano, C₁₋₆alkyl,
C₁₋₆haloalkyl, C₂₋₇alkenyl, C₂₋₇alkynyl, C₁₋₆alkoxy, C₁₋₆haloalkoxy, C₁₋₆alkylthio,
C₁₋₆haloalkylthio, C₁₋₆alkylsulfinyl, C₁₋₆alkylsulfonyl or pyrazolyl that may be optionally C₁₋₆alkyl-substituted or C₁₋₆haloalkyl-substituted,
R⁵ and R⁶ each independently represents hydrogen atom, fluoro, C₁₋₄alkyl, C₁₋₄haloalkyl or phenyl, and
Q represents naphthyl, phenyl that may be optionally substituted, pyridyl that may be optionally substituted, thienyl that may be optionally substituted, or furyl that may be optionally substituted, wherein substituents to phenyl, pyridyl, thienyl and furyl are one to five groups selected from the group consisting of fluoro, chloro, C₁₋₄alkyl, C₁₋₄haloalkyl, C₁₋₄alkoxy, C₁₋₄haloalkoxy, cyano, nitro, amino and phenyl.

In the plant pest controlling active compounds of the aforementioned formula (I), particularly preferably there can be mentioned the compounds in which

R¹ and R² form, together with the nitrogen atom to which they are bonded, a heterocyclic group which is a monovalent group derived from a heterocycle selected from the group consisting of aziridine, azetidine, pyrrolidine, 3-pyrroline, piperidine, perhydroazepine, perhydroazocine, perhydro-1,2-diazepine, perhydro-1,2,5-oxadiazepine, 2-pyrazoline, thiazolidine, perhydroindole, 1,2,3,3a,4,7,7a-heptahydroisoindole, 1,2,3,6-tetrahydropyridine, perhydroquinoline, perhydroisoquinoline, 1,4,5,6-tetrahydropyridazine, morpholine, thiomorpholine, thiomorpholine-1,1-dioxide, piperazine, pyrrole, pyrazole, imidazole, 1,2,3-triazole, 1,2,4-triazole, tetrazole and 1H-indazole and may be optionally substituted with 1-3 groups selected from the group consisting of fluoro, bromo, methyl, ethyl, n-propyl, fluoromethyl, trifluoromethyl, 2,2,2-trifluoroethyl, methoxy, methylthio, benzylthio, hydroxymethyl, 2-hydroxyethyl, methoxymethyl, anilinomethyl, difluoromethylene, dichloromethylene, methoxycarbonyl, ethoxycarbonyl, benzyloxycarbonyl, acetyl, trifluoromethylcarbonyl, trichloromethylcarbonyl, 1,1,2,2-tetrafluoroethylcarbonyl, perfluoroethylcarbonyl, perfluoroheptylcarbonyl, phenyl, benzyl, 2-pyridyl, hydroxy, oxo, cyano, carboxy, carbamoyl, ethoxycarbonylmethyl, methylcarbonylamino and trifluoromethylcarbonylamino,

R³ represents hydrogen, chloro, cyano, hydroxy, amino, azido, methyl, ethyl, iso-propyl, tert-butyl, trifluoromethyl, methoxymethyl, cyclopropyl, allyl, ethynyl, 1-propynyl, methoxy, ethoxy, n-propyloxy, n-butyloxy, 2,2,2-trifluoroethyloxy, allyloxy, 2-methyl-4-pentenyloxy, 3-chloro-4,4,4-trifluoro-2-butenyloxy, methylthio, ethylthio, n- or iso-propylthio, n-, sec- or tert-butylthio, allylthio, 3,3-dichloroallylthio, methylsulfinyl, methylsulfonyl, phenoxy, benzyloxy, phenyl that may be optionally substituted with 1-2 groups selected from the group consisting of chloro, methyl, methoxy and trifluoromethyl, benzyl that may be optionally chloro-substituted, or phenoxymethyl that may be optionally chloro-substituted, or

R³ represents a heterocyclic group which is a monovalent group derived from a heterocycle selected from the group consisting of pyrrolidine, piperidine, morpholine, thiomorpholine, piperazine, thiophene, thiazole, pyridine, quinoline, isoquinoline, pyrazine, pyridazine, pyrimidine, imidazole, pyrazole, tetrazole, 1,2,4-triazole and 2,3-dihydroindole, and may be optionally substituted with a group selected from the group consisting of chloro, bromo, methyl and trifluoromethyl, or
R³ represents a group selected from the group consisting of the following groups A-H and J-M 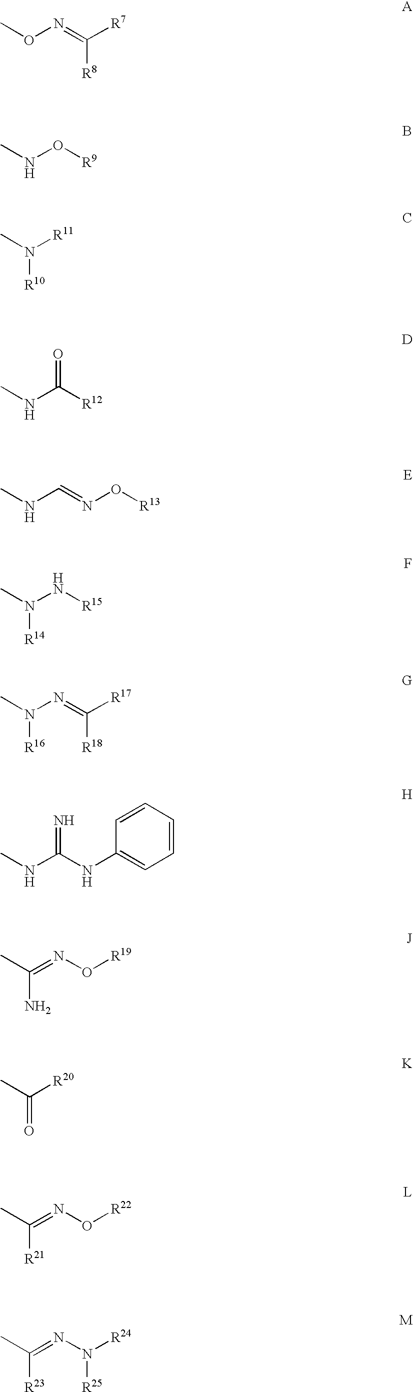
in which
R⁷ represents hydrogen atom, methyl or trifluoromethyl,
R⁸ represents methyl, iso- or tert-butyl, neo-pentyl, phenyl, ethoxy or cyano, or
R⁷ and R⁸ form, together with the carbon atom to which they are bonded, cyclopentylidene or cyclohexylidene,
R⁹ represents methyl, 3,3-dichloroallyl or benzyl,
R¹⁰ represents hydrogen atom, methyl or ethyl,
R¹¹ represents methyl, ethyl, iso-propyl, methoxyethyl, dimethylaminoethyl, phenyl, benzyl or cyano,
R¹² represents methyl or phenyl,
R¹³ represents methyl or benzyl,
R¹⁴ represents hydrogen atom or methyl,
R¹⁵ represents hydrogen atom, 2,2,2-trifluoroethyl or phenyl,
R¹⁶ represents hydrogen atom or methyl,
R¹⁷ represents hydrogen atom, methyl or trifluoromethyl,
R¹⁸ represents methyl or phenyl,
R¹⁹ represents hydrogen atom or methyl,
R²⁰ represents methyl, ethyl, n- or iso-propyl,
R²¹ represents methyl or ethyl,
R²² represents methyl, ethyl, n-propyl, n- or tert-butyl, allyl, 2-chloro-2-propenyl, 3-chloro-2-propenyl, 3,3-dichloro-2-propenyl, 2-methoxyethyl, 2-phenoxypropyl or tert-butoxycarbonylmethyl,
R²³ represents methyl,
R²⁴ represents hydrogen atom or methyl,
R²⁵ represents iso-propyl or phenyl,
R²⁴ and R²⁵ form, together with the nitrogen atom to which they are bonded, a saturated-monoheterocyclic group which is a monovalent group derived from a monoheterocycle selected from the group consisting of pyrrolidine, piperidine, morpholine and piperazine and may be optionally substituted with methyl,
R⁴ represents hydrogen atom, chloro, cyano, methyl, trifluoromethyl, allyl, ethynyl, 1-propynyl, methoxy, 2,2,2-trifluoroethoxy, methylthio, C₁₋₆haloalkylthio, methylsulfinyl, methylsulfonyl or pyrazolyl that may be optionally methyl-substituted or trifluoromethyl-substituted,
R⁵ and R⁶ each independently represents hydrogen atom, fluoro, methyl, ethyl, iso-propyl, trifluoromethyl or phenyl, and
Q represents naphthyl, phenyl that may be optionally substituted, pyridyl that may be optionally substituted, thienyl that may be optionally substituted, or furyl that may be optionally substituted, wherein substituents to phenyl, pyridyl, thienyl and furyl are 1-5 groups selected from the group consisting of fluoro, chloro, methyl, tert-butyl, trifluoromethyl, methoxy, trifluoromethoxy, cyano, nitro, amino and phenyl,

Similarly, in the compounds of the aforementioned formula (IA), there can be mentioned the compounds in which R^{1A}, R^{2A}, R^{3A}, R^{4A}, R^{5A}, R^{6A}, R^{7A}, R^{8A}, R^{9A}, R^{10A}, R^{11A}, R^{12A}, R^{13A}, R^{14A}, R^{15A}, R^{16A}, R^{17A}, R^{18A}, R^{19A}, R^{20A}, R^{21A}, R^{22A}, R^{23A}, R^{24A}, R^{25A} and Q^{A} each has the same definition as the definition of R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, R²², R²³, R²⁴, R²⁵ and Q mentioned in the definition of the preferable compounds of the aforementioned formula (I), respectively,

provided that, the following cases (T-1)-(T-6) are excluded:

(T-1) the case in which group 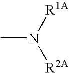
represents 1-pyrrolyl, 1-imidazolyl, 3-oxopiperidino or 4-oxopiperidino, R^{3A} represents hydrogen atom, R^{4A} represents hydrogen atom, and Q^{A} represents 1-naphthyl or phenyl group that may be optionally substituted by one or two groups selected from the group consisting of chloro, methyl, ethyl and trifluoromethyl,
(T-2) the case in which group 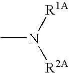
represents 3-oxopiperidino, 4-oxopiperidino, 4-hydroxypiperidino, 4-carbamoylpiperidino, 4-methylpiperazino, 4-ethylpiperazino, 4-(2-hydroxyethyl)piperazino or morpholino, R^{3A} represents amino, R^{4A} represents hydrogen atom, and Q^{A} represents 3-pyridyl or phenyl group that may be optionally substituted by one to three groups selected from the group consisting of fluoro, chloro, methyl, ethyl, isopropyl, trifluoromethyl and methoxy,
(T-3) the case in which group 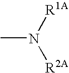
represents piperidino, 4-hydroxypiperidino, 4-methylpiperazino or morpholino, R^{3A} represents chloro, dimethylamino, anilino, piperidino, 4-methylpiperazino or morpholino, R^{4A} represents hydrogen atom, and Q^{A} represents phenyl group that may be optionally substituted by one or two groups selected from the group consisting of methyl and methoxy,
(T-4) the case in which group 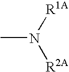
represents 1-pyrrolidinyl, piperidino, morpholino or 1-pyrrolyl, R^{3A} represents methyl or methoxymethyl, R^{4A} represents chloro, and Q^{A} represents phenyl or 1-naphthyl,
(T-5) the case in which group 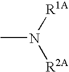
represents 1-azilidinyl, piperidino or morpholino, R^{3A} represents methylthio, R^{4A} represents chloro, and Q^{A} represents phenyl group substituted by methoxy, ethoxy, n-propoxy, iso-propoxy, n-butoxy or iso-butoxy,
(T-6) the case in which group 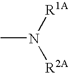
represents 1-azilidinyl, R^{3A} represents hydrogen atom or amino, R^{4A} represents chloro, and Q^{A} represents phenyl group substituted by methoxy or ethoxy,
as preferable.

Moreover, in the compounds of the aforementioned formula (IA), the compounds in which R^{1A}, R^{2A}, R^{3A}, R^{4A}, R^{5A}, R^{6A}, R^{7A}, R^{8A}, R^{9A}, R^{10A}, R^{11A}, R^{12A}, R^{13A}, R^{14A}, R^{15A}, R^{16A}, R^{17A}, R^{18A}, R^{19A}, R^{20A}, R^{21A}, R^{22A}, R^{23A}, R^{24A}, R^{25A} and Q^{A} each has the same definition as the definition of R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, R²², R²³, R²⁴, R²⁵ and Q mentioned in the definition of the particularly preferable compounds of the aforementioned formula (I), respectively,

provided that, the following cases (T-1)-(T-6) are excluded:

(T-1) the case in which group 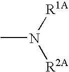
represents 1-pyrrolyl, 1-imidazolyl, 3-oxopiperidino or 4-oxopiperidino, R^{3A} represents hydrogen atom, R^{4A} represents hydrogen atom, and Q^{A} represents 1-naphthyl or phenyl group that may be optionally substituted by one or two groups selected from the group consisting of chloro, methyl and trifluoromethyl,
(T-2) the case in which group 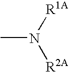
represents 3-oxopiperidino, 4-oxopiperidino, 4-hydroxypiperidino, 4-carbamoylpiperidino, 4-methylpiperazino, 4-ethylpiperazino, 4-(2-hydroxyethyl)piperazino or morpholino, R^{3A} represents amino, R^{4A} represents hydrogen atom, and Q^{A} represents 3-pyridyl or phenyl group that may be optionally substituted by one to three groups selected from the group consisting of fluoro, chloro, methyl, trifluoromethyl and methoxy,
(T-3) the case in which group 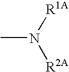
represents piperidino, 4-hydroxypiperidino, 4-methylpiperazino or morpholino, R^{3A} represents chloro, dimethylamino, anilino, piperidino, 4-methylpiperazino or morpholino, R^{4A} represents hydrogen atom, and Q^{A} represents phenyl group that may be optionally substituted by one or two groups selected from the group consisting of methyl and methoxy,
(T-4) the case in which group 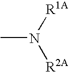
represents 1-pyrrolidinyl, piperidino, morpholino or 1-pyrrolyl, R^{3A} represents methyl
or methoxymethyl, R^{4A} represents chloro, and Q^{A} represents phenyl or 1-naphthyl,
(T-5) the case in which group 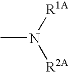
represents 1-azilidinyl, piperidino or morpholino, R^{3A} represents methylthio, R^{4A} represents chloro, and Q^{A} represents phenyl group substituted with methoxy,
(T-6) the case in which group 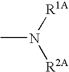
represents 1-azilidinyl, R^{3A} represents hydrogen atom or amino, R^{4A} represents chloro, and Q^{A} represents phenyl group substituted with methoxy,
are particularly preferable.

The aforementioned preparation process (a) can be illustrated by the following reaction scheme in case that, for example, 5-benzyl-4,6-dichloropirimidine and pyrrolidine are used as starting materials.

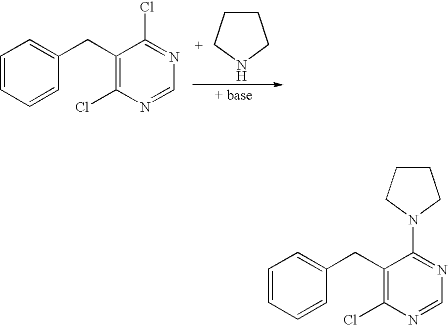

The aforementioned preparation process (b) can be illustrated by the following reaction scheme in case that, for example, 5-benzyl-4-chloro-2-methylthio-6-pyrrolidin-1-yl-pirimidine is used as starting material and, for example, m-chloroperbenzoic acid, as oxidizing agent.

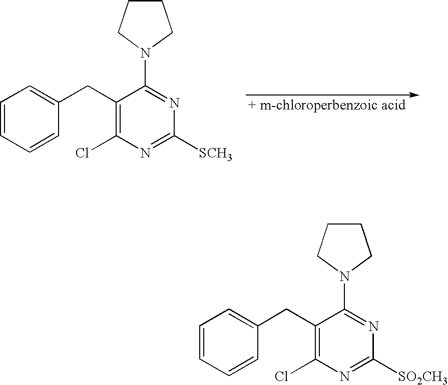

The aforementioned preparation process (c) can be illustrated by the following reaction scheme in case that, for example, 5-benzyl-4-chloro-2-methanesulfonyl-6-pyrrolidin-1-yl-pirimidine and pyrazole are used as starting materials.

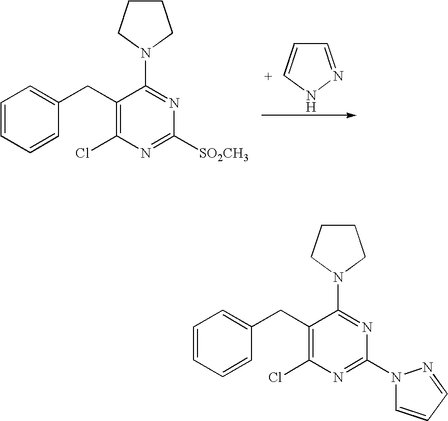

The aforementioned preparation process (d) can be illustrated by the following reaction scheme in case that, for example, 4-chloro-5-(3-fluorobenzyl)-6-pyrrolidin-1-yl-pirimidine and sodium methoxide are used as starting materials.

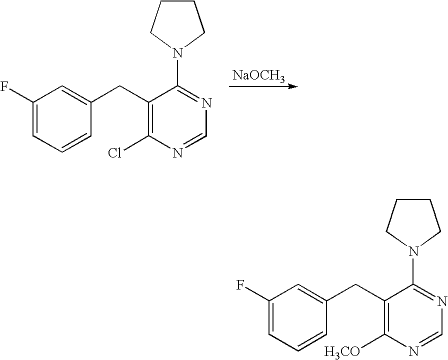

The aforementioned preparation process (e) can be illustrated by the following reaction scheme in case that a starting material, for example, 4-chloro-5-(3-fluorobenzyl)-6-pyrrolidin-1-yl-pirimidine is catalytically hydrogenated.

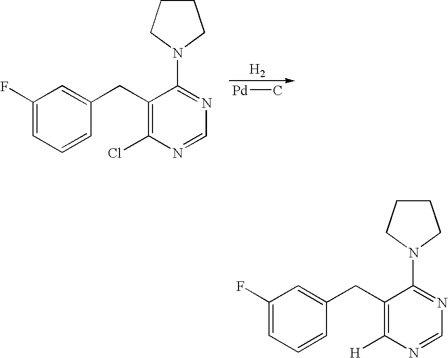

The aforementioned preparation process (f) can be illustrated by the following reaction scheme in case that, for example, 5-benzyl-4-chloro-6-(2,5-dihydropyrrol-1-yl)pyrimidine and sodium chlorodifluoroacetate are used as starting materials.

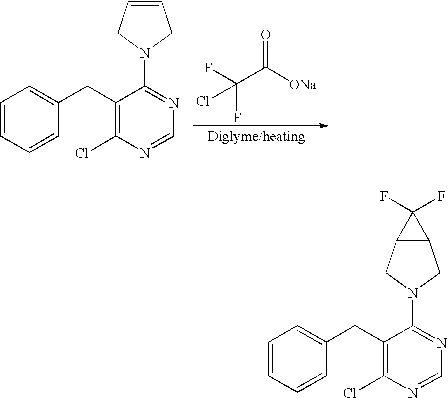

The aforementioned preparation process (g) can be illustrated by the following reaction scheme in case that, for example, 2-azido-5-benzyl-4-chloro-6-(pyrrolidin-1-yl)pyrimidine and sodium borohydride are used as starting materials.

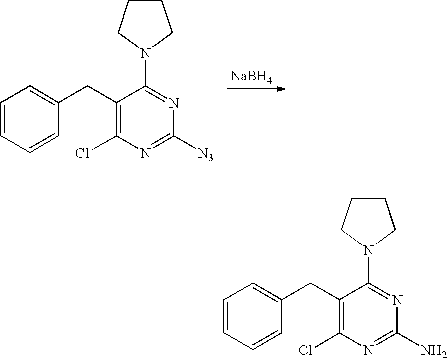

The aforementioned preparation process (h) can be illustrated by the following reaction scheme in case that, for example, 5-benzyl-4-chloro-6-(pyrrolidin-1-yl)pyrimidin-2-ylamine and tert-butyl nitrite and copper (II) chloride are used as starting materials (Sandmeyer process).

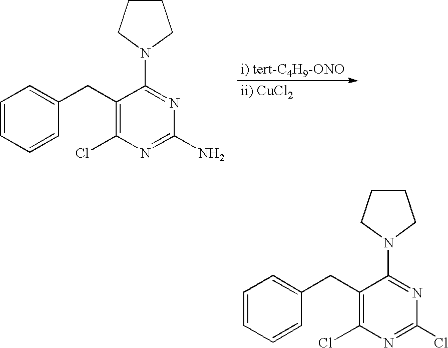

The aforementioned preparation process (i) can be illustrated by the following reaction scheme in case that, for example, 5-benzyl-4-chloro-6-(pyrrolidin-1-yl)pyrimidin-2-ylamine and dimethylformamide dimethylacetal and O-methylhydroxylammonium chloride are used as starting materials.

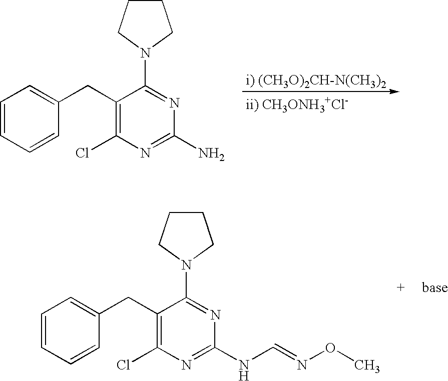

The aforementioned preparation process (j) can be illustrated by the following reaction scheme in case that, for example, 5-benzyl-4-chloro-6-(pyrrolidin-1-yl)pyrimidin-2-ylamine and acetic anhydride are used as starting materials.

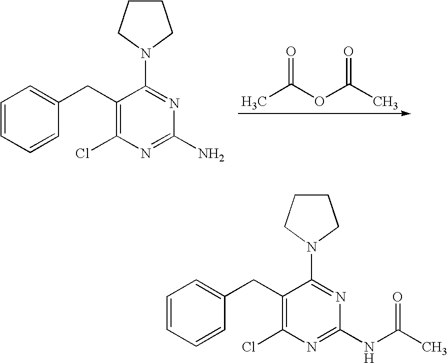

The aforementioned preparation process (k) can be illustrated by the following reaction scheme in case that, for example, 5-benzyl-4-chloro-6-(pyrrolidin-1-yl)pyrimidine-2-carbonitrile and methyl magnesium bromide are used as starting materials.

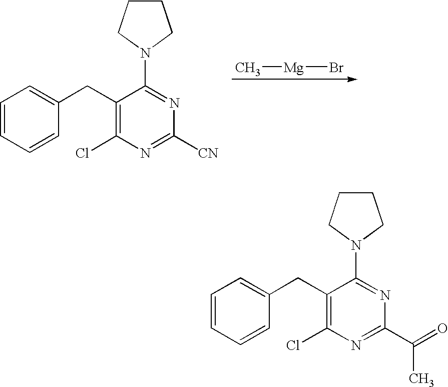

The aforementioned preparation process (l) can be illustrated by the following reaction scheme in case that, for example, 1-(5-benzyl-4-chloro-6-(pyrrolidin-1-yl)pyrimidin-2-yl)ethanone and O-ethylhydroxylammonium chloride are used as starting materials.

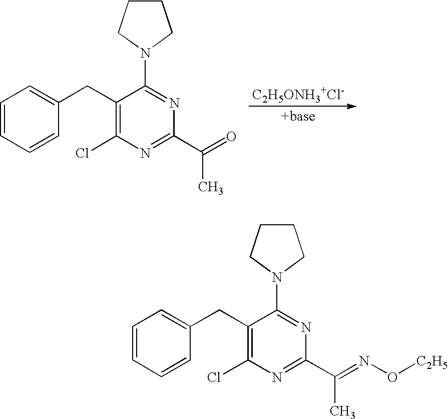

The aforementioned preparation process (m) can be illustrated by the following reaction scheme in case that, for example, 5-benzyl-4-chloro-6-(pyrrolidin-1-yl)pyrimidine-2-carbonitrile and hydroxylammonium chloride are
used as starting materials.

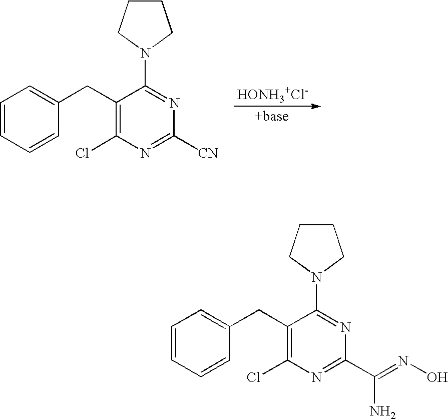

The compounds of the formula (II), starting materials in the above-mentioned preparation process (a), which are partly novel compounds and are not described in the existing literatures, can be easily prepared, for example, by reacting a compound represented by the formula 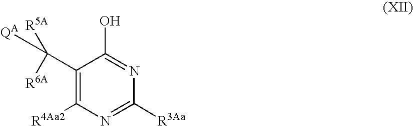
wherein
R^{4Aa2} represents hydrogen atom, hydroxy, alkyl, haloalkyl or alkenyl,
R^{3Aa}, R^{5A}, R^{6A} and Q^{A} have the same definition as aforementioned,
with a halogenating agent, for example, phosphorus oxychloride, phosphorus oxybromide, etc. according to the process described in Journal of Heterocyclic Chemistry, Vol. 29, p. 1369-1370 (1992); Journal of Organic Chemistry, Vol. 32, No. 2, p. 1591-1596 (1967), etc.

The compounds of the above-mentioned formula (XII), which are partly novel compounds and are not described in the existing literatures, can be easily prepared, for example, by reacting a compound represented by the formula 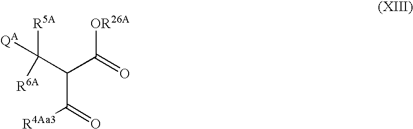
wherein
R^{4Aa3} represents hydrogen atom, alkyl, haloalkyl, alkenyl or C₁₋₄alkoxy,
R^{26A} represents C₁₋₄alkyl,
R^{5A}, R^{6A} and QA have the same definition as aforementioned,
with a compound represented by the formula 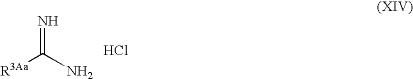
wherein
R^{3Aa} has the same definition as aforementioned,
according to the process described in, for example, Journal of the American Chemical Society, Vol. 77, p. 745-749 (1955); Journal of the American Chemical Society, Vol. 69, p. 2941-2942 (1938), etc.

The above-mentioned formula (XIII), which is also partly novel compounds that are not described in the existing literatures, can be easily prepared, for example, by reacting a compound represented by the formula 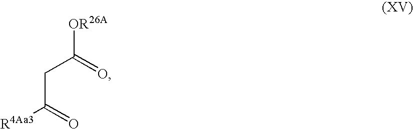
wherein
R^{4Aa3} and R^{26A} have the same definition as aforementioned,
with a compound represented by the formula 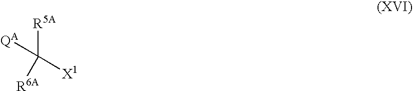
wherein
X¹ represents halogen, preferably chloro, bromo or iodo,
R^{5A}, R^{6A} and Q^{A} have the same definition as aforementioned,
according to the process described in, for example, Japanese Laid-open Patent Publication No. 228500/1999 etc.

The compounds of the above-mentioned formulae (XV) and (XVI) are per se known compounds.

The above-mentioned formula (XIV), which is partly novel compounds that are not described in the existing literatures, can be easily prepared, for example, from a compound represented by the formula
R^{3Aa}—CN (XVII)
wherein
R^{3Aa} has the same definition as aforementioned,
by treating according to the process described in Journal of Organic Chemistry, Vol. 26, p. 412-418 (1961); Journal of Organic Chemistry, Vol. 34, p. 292-296 (1969); Chemical Reviews Washington, D. C., Vol. 35, p. 351-425 (1944), etc.

The compounds of the above-mentioned formula (XVII) are per se known compounds.

The compounds of the formula (III), starting materials in the above-mentioned preparation process (a), are per se known compounds.

As specific examples for the compounds of the formula (II), used as the starting materials in the above-mentioned preparation process (a), can be mentioned as follows:
5-benzyl-4,6-dichloropyrimidine
5-benzyl-4,6-dichloro-2-methylpyrimidine
4,6-dichloro-5-(3-fluorobenzyl)pyrimidine
4,6-dichloro-5-(3-chlorobenzyl)pyrimidine
4,6-dichloro-5-(2,6-difluorobenzyl)pyrimidine
4,6-dichloro-5-(3,5-difluorobenzyl)pyrimidine
5-benzyl-4-chloro-6-methylpyrimidine
5-benzyl-4,6-dichloro-2-methylthiopyrimidine
5-benzyl-4,6-dichloro-2-(pyridin-2-yl)pyrimidine
5-benzyl-4,6-dichloro-2-(pyridin-3-yl)pyrimidine
5-benzyl-4,6-dichloro-2-(pyridin-4-yl)pyrimidine
5-benzyl-4,6-dichloro-2-(pyrazin-2-yl)pyrimidine, and so on.

As specific examples for the compounds of the formula (XII), used as starting materials in the preparation of the compounds of the aforementioned formula (II), the following can be mentioned:
5-benzylpyrimidin-4,6-diol,
5-(3-fluorobenzyl)pyrimidin-4,6-diol,
5-(3-chlorobenzyl)pyrimidin-4,6-diol,
5-benzyl-2-(pyridin-2-yl)pyrimidin-4,6-diol,
5-benzyl-2-methylpyrimidin-4,6-diol,
5-benzyl-2-methylthiopyrimidin-4,6-diol, and so on.

As specific examples for the compounds of the formula (XIII), used as starting materials in the preparation of the compounds of the aforementioned formula (XII), the following can be mentioned:
diethyl benzylmalonate,
ethyl 2-benzylacetoacetate
diethyl 2-(3-fluorobenzyl)malonate,
diethyl 2-(3-chlorobenzyl)malonate, and so on.

As specific examples for the compounds of the formula (XIV), used as starting materials in the preparation of the compounds of the aforementioned formula (XII), the following can be mentioned:
formamidine hydrochloride,
acetamidine hydrochloride,
tert-butylcarbamidine hydrochloride,
trifluoroacetamidine,
cyclopropylcarbamidine hydrochloride,
benzamidine hydrochloride,
2-(4-chlorophenoxy)-acetamidine hydrochloride,
pyrrolidinoformamidine hydrobromide,
morpholinoformamidine hydrobromide,
2-amidinothiophene hydrochloride,
3-amidinopyridine hydrochloride,
2-methylthiazole-4-carboxyamidine hydrochloride, and so on.

As specific examples for the compounds of the formula (XV), used as the starting materials in the preparation of the compounds of the aforementioned formula (XIII), the following can be mentioned:
diethyl malonate
methyl actoacetate
ethyl butyrylacetate
ethyl 4,4,4-trifluoroacetoacetate
methyl 3-oxo-6-octenoate, and so on.

As specific examples for the compounds of the formula (XVI), used as the starting materials in the preparation of the compounds of the aforementioned formula (XII), the following can be mentioned:
benzyl bromide,
1-phenylethyl bromide,
3-methylbenzyl bromide,
2-nitrobenzyl bromide,
3-fluorobenzyl bromide,
3-chlorobenzyl bromide,
3-(bromomethyl)benzonitrile,
4-tert-butylbenzyl bromide,
4-(trifluoromethyl)benzyl bromide,
2-(bromomethyl)naphthalene,
3-chloro-2-(chloromethyl)-5-(trifluoromethyl)pyridine,
2-chloro-5-(chloromethyl)pyridine,
2-chloro-5-(chloromethyl)thiophene,
2-(bromomethyl)-5-nitrofuran, and so on.

As specific examples for the compounds of the formula (XVII, used as the starting materials in the preparation of the compounds of the aforementioned formula (XIV), the following can be mentioned:
benzonitrile,
2-cyanopyridine,
2-quinolinecarbonitrile,
1-isoquinolinecarbonitrile,
3-isoquinolinecarbonitrile,
cyanopyrazine, and so on.

As specific examples for the compounds of the formula (III), used as

the starting materials in the above-mentioned preparation process (a), the following can be mentioned:

2-methylazilidine
azetidine,
pyrrolidine,
2-pyrrolidone,
2-methylpyrrolidine,
3-pyrroline,
thiazolidine,
pyrrole,
2-pyrazoline,
pyrazole,
imidazole,
1H-1,2,3-triazole,
1H-1,2,4-triazole,
1H-tetrazole,
indoline,
piperidine,
4-methylpiperidine,
morpholine,
thiomorpholine,
piperazine,
hexamethyleneimine,
heptamethyleneimine,
octahydroindole, and so on.

The compounds of the formula (IAb), used as the starting materials in the above-mentioned preparation process (b), can be prepared by the aforementioned preparation processes (a), (d), (e) or (f) and as their specific examples the following can be mentioned:
5-benzyl-4-chloro-2-methylthio-6-(pyrrolidin-1-yl)pyrimidine,
5-benzyl-4-chloro-2-methylthio-6-(piperidin-1-yl)pyrimidine,
5-benzyl-4-chloro-6-(4-methylpiperidin-1-yl)-2-methylthiopyrimidine,
4-(5-benzyl-6-chloro-2-methylthiopyrimidin-4-yl)morpholine,
2-allylthio-5-benzyl-4-chloro-6-(pyrrolidin-1-yl)pyrimidine,
5-benzyl-4-chloro-2-(3,3-dichloroallylthio)-6-(pyrrolidin-1-yl)pyrimidine, and so on.

As oxidizing agents used in the above-mentioned preparation process (b), there can be mentioned, for example, m-chloroperbenzoic acid, hydrogen peroxide, and so on.

The compounds of the formula (IAc), used as the starting materials in the above-mentioned preparation process (c), are compounds that can be prepared by the aforementioned preparation processes (b) or (h) and as their specific examples the following can be mentioned:
5-benzyl-4-chloro-2-methylsulfonyl-6-(pyrrolidin-1-yl)pyrimidine,
5-benzyl-4-chloro-2-methylsulfonyl-6-(piperidin-1-yl)pyrimidine,
5-benzyl-4-chloro-2-methylsulfonyl-6-(4-methylpiperidin-1-yl)pyrimidine,
4-(5-benzyl-6-chloro-2-methylsulfonylpyrimidin-4-yl)morpholine,
5-benzyl-2,4-dichloro-6-(pyrrolidin-1-yl)pyrimidine, and so on.

The compounds of the formula (IV), used as the starting materials in the above-mentioned preparation process (c), are per se known compounds and can be prepared according to the process described in, for example, Bulletin of the Chemical Society of Japan, Vol. 64, p. 2948-2953 (1991); Journal of Organic Chemistry, Vol. 31, p. 677-681 (1966); Journal of the American Chemical Society, Vol. 75, p. 4053-4054 (1953), etc. As their specific examples the following can be mentioned:

sodium cyanide, copper cyanide, tetrabutylammonium cyanide, sodium azide, 1-hexyne, ethynyltrimethylsilane, sodium methoxide, 2,2,2-trifluoroethanol, allyl alcohol, 3-chloro-4,4,4-trifluoro-2-buten-1-ol, sodium thiomethoxide, phenol, benzyl alcohol, pyrrolidine, pyrazole, imidazole, 1,2,4-triazole, cyclopentane oxime, 2-(hydroxyimino)propanenitrile, O-benzylhydroxylamine, aniline, hydrazine hydrate, N-methyl-N-(1-phenylethylidene)hydrazine, N-phenylguanidine, and so on.

The compounds of the formula (IAd), used as the starting materials in the above-mentioned preparation process (d), can be prepared by the aforementioned preparation processes (a) or (f) and as their specific examples the following can be mentioned:
4-chloro-5-(3-fluorobenzyl)-6-(pyrrolidin-1-yl)pyrimidine,
5-benzyl-4-chloro-2-(pyrazol-1-yl)-6-(pyrrolidin-1-yl)pyrimidine,
5-benzyl-4-chloro-6-(piperidin-1-yl)-2-(pyridin-2-yl)pyrimidine,
3-(5-benzyl-6-chloropyrimidin-4-yl)-6,6-difluoro-3-azabicyclo[3,1,0]hexane, and so on.

The compounds of the formula (V), used as the starting materials in the above-mentioned preparation process (d) are per se known compounds and as their specific examples the following can be mentioned:

sodium cyanide, potassium cyanide, copper (I) cyanide, sodium methoxide, 2,2,2-trifluoroethanol, sodium thiomethoxide, 2,2,2-trifluoroethanethiol, 1-hexyne, pyrazole, imidazole, 1,2,4-triazole, and so on.

The compounds of the formula (IAe), used as the starting materials in the above-mentioned preparation process (e), are compounds that can be prepared by the above-mentioned preparation processes (a) or (f) and as their specific examples the following can be mentioned:
4-chloro-5-(3-fluorobenzyl)-6-pyrrolidin-1-yl)pyrimidine,
5-benzyl-4-chloro-2-(pyrazol-1-yl)-6-(pyrrolidin-1-yl)pyrimidine,
5-benzyl-4-chloro-6-(piperidin-1-yl)-2-(pyridin-2-yl)pyrimidine,
3-(5-benzyl-6-chloropyrimidin-4-yl)-6,6-difluoro-3-azabicyclo[3,1,0]hexane, and so on.

As catalyst used in the above-mentioned preparation process (e), there can be mentioned, for example, palladium-carbon and so on.

The compounds of the formula (IAf), used as the starting materials in the above-mentioned preparation process (f), can be prepared by the aforementioned preparation processes (a), (c) or (d) and as their specific examples the following can be mentioned:
5-benzyl-4-chloro-6-(2,5-dihydropyrrol-1-yl)pyrimidine,
5-benzyl-4-(2,5-dihydropyrrol-1-yl)-6-methoxypyrimidine,
4-chloro-6-(3,6-dihydro-2H-pyridin-1-yl)-5-(3-fluorobenzyl)-2-(1,2,4-triazol-1-yl)pyrimidine, and so on.

The compounds of the formula (IAg), use as the starting materials in the above-mentioned preparation process (g), can be prepared by the aforementioned preparation process (c) and as their specific examples the following can be mentioned:
2-azido-4-chloro-5-(3-chlorobenzyl)-6-(pyrrolidin-1-yl)pyrimidine,
2-azido-5-(6-chloropyridin-3-ylmethyl)-4-(pyrrolidin-1-yl)pyrimidine,
2-azido-4-chloro-6-(2,5-dihydropyrrol-1-yl)-5-(naphthalen-2-ylmethyl)pyrimidine and so on.

As catalyst used in the above-mentioned preparation process (g), there can be mentioned, for example, palladium-carbon and so on.

As metal hydrides used in the above-mentioned preparation process (g), there can be mentioned, for example, sodium borohydride, lithium aluminium hydride, and so on.

The compounds of the formula (IAh), used as the starting materials in the first step of the above-mentioned preparation process (h), the first step of the above-mentioned preparation process (i) and the above-mentioned preparation process (j) can be prepared by the aforementioned preparation processes (c) or (g) and as their specific examples the following can be mentioned:
4-chloro-6-(pyrrolidin-1-yl)-5-(3,4,5-trifluorobenzyl)pyrimidin-2-ylamine,
5-benzyl-4-chloro-6-(pyrrolidin-1-yl)pyrimidin-2-ylamine,
5-benzyl-4-chloro-6-(4,5-dihydropyrazol-1-yl)pyrimidin-2-ylamine, and so on.

As nitrite esters used in the first step of the above-mentioned preparation process (h), there can be mentioned, for example, tert-butyl nitrite etc., and nitrous acid can be formed on the spot, for example, by exposing sodium nitrite to an acidic condition.

As copper halides or potassium halides used in the second step of the above-mentioned preparation process (h), there can be mentioned, for example, copper (I) chloride, copper (II) chloride, copper (I) bromide, copper (II) bromide, potassium iodide, and so on.

As specific examples of the compounds of the formula (VI), use as the starting materials in the second step of the above-mentioned preparation process (i), the following can be mentioned:
N′-(5-benzyl-4-chloro-6-(pyrrolidin-1-yl)pyrimidin-2-yl)-N,N-dimethylformamidine,
N′-(4-chloro-6-(piperidin-1-yl)-5-(pyridin-2-ylmethyl)pyrimidin-2-yl)-N,N-dimethyl formamidine,
N′-(4-chloro-5-(5-nitrofuran-2-ylmethyl)-6-(pyrrolidin-1-yl)pyrimidin-2-yl)-N,N-dimethylformamidine, and so on.

The compounds of the formula (VII), used as the starting materials in the above-mentioned preparation process (i) are per se known compounds and as their specific examples the following can be mentioned:
O-methylhydroxylamine,
O-ethylhydroxylamine,
O-isopropylhydroxylamine,
O-benzylhydroxylamine, and so on.

The compounds of the formula (VIII), used as the starting materials in the above-mentioned preparation process (j) are per se known compounds and as their specific examples the following can be mentioned:

acetic anhydride, propionic anhydride, acetyl chloride, n-butyryl chloride, benzoyl chloride, and so on.

The compounds of the formula (IAk), used as the starting materials in the above-mentioned preparation process (k) and the above-mentioned preparation process (m) can be prepared by the aforementioned preparation processes (c) or (d) and as their specific examples the following can be mentioned:
5-benzyl-4-chloro-6-(pyrrolidin-1-yl)pyrimidine-2-carbonitrile,
5-(3-fluorobenzyl)-4-(4-methylpiperadin-1-yl)pyrimidine-2-carbonitrile,
4-(2,3-dihydroindol-1-yl)-5-(3-fluoro-4-trifluoromethylbenzyl)pyrimidine-2-carbonitrile, and so on.

The compounds of the formula (IX), used as the starting materials in the above-mentioned preparation process (k) are per se known compounds and can be also prepared according to the process described in, for example, Journal of the American Chemical Society, Vol. 94, p. 5421-5434 (1972) etc. As their specific examples the following can be mentioned:
methyl magnesium bromide,
isopropyl magnesium bromide,
pentyl magnesium bromide, and so on.

The compounds of the formula (IAl), used as the starting materials in the above-mentioned preparation process (l) can be prepared by the aforementioned preparation process (k) and as their specific examples the following can be mentioned:
1-(5-benzyl-4-chloro-6-(pyrrolidin-1-yl)pyrimidin-2-yl)ethanone,
1-(5-benzyl-4-methyl-6-(pyrrolidin-1-yl)pyrimidin-2-yl)ethanone,
1-(5-benzyl-4-methoxy-6-(piperidin-1-yl)pyrimidin-2-yl)propan-1-one, and so on.

The compounds of the formula (X), used as the starting materials in the above-mentioned preparation process (l) are per se known compounds and as their specific examples the following can be mentioned:
O-ethylhydroxylamine,
O-(3-chloroallyl)hydroxylamine,
O-(2-methoxyethyl)hydroxylamine,
phenylhydrazine,
1-methyl-1-phenylhydrazine, and so on.

The compounds of the formula (XI), used as the starting materials in the above-mentioned preparation process (m) are per se known compounds and as their specific examples the following can be mentioned:
hydroxylamine,
O-methylhydroxylamine,
O-ethylhydroxylamine, and so on.

The compounds of the formula (IAc), Xc of which represents iodo, used as the starting materials in the above-mentioned preparation process (c), can be easily prepared from compounds, Xc of which is chloro, according to the process described in, for example, Journal of Heterocyclic Chemistry, Vol. 23, p. 1079-1084 (1986); Journal of the Chemical Society, (c), p. 1204-1209 (1967), etc. and the compounds of the formula (IAd), Xd of which represents iodo, starting materials in the above-mentioned preparation process (d), can be easily prepared from compounds, Xd of which is chloro, according to the similar process,

The reaction of the above-mentioned preparation process (a) can be conducted in an appropriate diluent. As examples of the diluent usable in that case there can be mentioned aliphatic, alicyclic and aromatic hydrocarbons (may be optionally chlorinated), for example, pentane, hexane, cyclohexane, petroleum ether, ligroine, benzene, toluene, xylene, dichloromethane, chloroform, carbon tetrachloride, 1,2-dichloroethane, chlorobenzene, dichlorobenzene, etc.; ethers, for example, ethyl ether, methyl ethyl ether, isopropyl ether, butyl ether, dioxane, dimethoxyethane (DME), tetrahydrofuran (THF), diethylene glycol dimethyl ether (DGM), etc.; ketones, for example, acetone, methyl ethyl ketone (MEK), methyl isopropyl ketone, methyl isobutyl ketone (MIBK), etc.; nitriles, for example, acetonitrile, propionitrile, acrylonitrile, etc.; esters, for example, ethyl acetate, amyl acetate, etc.; acid amides, for example, dimethylformamide (DMF), dimethylacetamide (DMA), N-methylpyrrolidone, 1,3-dimethyl-2-imidazolidinone, hexamethylphosphoric triamide (HMPA), etc.; sulfones, sulfoxides, for example, dimethyl sulfoxide (DMSO), sulfolane, etc.; bases, for example, pyridine etc.

The preparation process (a) can be conducted in the presence of an acid binder, and as said acid binder there can be mentioned, for example, as inorganic bases, hydrides, hydroxides, carbonates and bicarbonates, etc. of alkali metals and alkaline earth metals, for example, sodium hydride, lithium hydride, sodium hydrogen carbonate, potassium hydrogen carbonate, sodium carbonate, potassium carbonate, lithium hydroxide, sodium hydroxide, potassium hydroxide, calcium hydroxide, etc.; inorganic alkali metal amides, for example, lithium amide, sodium amide, potassium amide, etc.; as organic bases, alcoholates, tertiary amines, dialkylaminoanilines and pyridines, for example, triethylamine, 1,1,4,4-tetramethylethylenediamine (TMEDA), N,N-dimethylaniline, N,N-diethylaniline, pyridine, 4-dimethylaminopyridine (DMAP), 1,4-diazabicyclo[2,2,2]octane (DABCO) and 1,8-diazabicyclo[5,4,0]undec-7-ene (DBU), etc.; organic amine hydrochlorides, for example, pyridine hydrochloride, triethylamine hydrochloride, etc.; amine sulfonates, for example, pyridine p-toluenesulfonate, triethylamine p-toluenesulfonate, etc.

The preparation process (a) can be conducted in a substantially wide range of temperature. There can be applied temperatures generally of about −78 to about 180° C., preferably about −20 to about 120° C. Although said reaction is conducted desirably under normal pressure, it can be conducted optionally under elevated pressure or under reduced pressure.

In conducting the preparation process (a), the aimed compound can be obtained, for example, by reacting 1.1 to 8.0 moles of a compound of the formula (III) to 1 mole of a compound of the formula (II) in a diluent, for example, tetrahydrofuran, in the presence of triethylamine.

The reaction of the above-mentioned preparation process (b) can be conducted in an appropriate diluent. As examples of the diluent usable in that case there can be mentioned water; aliphatic, alicyclic and aromatic hydrocarbons (may be optionally chlorinated), for example, pentane, hexane, cyclohexane, petroleum ether, ligroine, benzene, toluene, xylene, dichloromethane, chloroform, carbon tetrachloride, 1,2-dichloroethane, chlorobenzene, dichlorobenzene, etc.; alcohols, for example, methanol, ethanol, isopropanol, butanol, ethylene glycol, etc.; esters, for example, ethyl acetate, amyl acetate, etc.; carboxylic acids, for example, acetic acid etc.

The preparation process (b) can be conducted in the presence of a catalyst and as example of said catalyst there can be mentioned, for example, tungstates etc.

The preparation process (b) can be conducted in a substantially wide range of temperature. There can be applied temperatures generally of about −78 to about 180° C., preferably about −20 to about 120° C. Although said reaction is conducted desirably under normal pressure, it can be conducted optionally under elevated pressure or under reduced pressure

In conducting the preparation process (b), the aimed compound can be obtained, for example, by reacting 2.0 to 2.4 moles of m-chloroperbenzoic acid (MCPBA) to 1 mole of a compound of the formula (IAb) in a diluent, for example,

dichloromethane.

The reaction of the above-mentioned preparation process (c) can be conducted in an appropriate diluent. As examples of the diluent usable in that case there can be mentioned aliphatic, alicyclic and aromatic hydrocarbons (may be optionally chlorinated), for example, pentane, hexane, cyclohexane, petroleum ether, ligroine, benzene, toluene, xylene, dichloromethane, chloroform, carbon tetrachloride, 1,2-dichloroethane, chlorobenzene, dichlorobenzene, etc.; ethers, for example, ethyl ether, methyl ethyl ether, isopropyl ether, butyl ether, dioxane, dimethoxyethane (DME), tetrahydrofuran (THF), diethylene glycol dimethyl ether (DGM), etc.; ketones, for example, acetone, methyl ethyl ketone (MEK), methyl isopropyl ketone, methyl isobutyl ketone (MIBK), etc.; nitriles, for example, acetonitrile, propionitrile, acrylonitrile, etc.; alcohols, for example, methanol, ethanol, isopropanol, butanol, ethylene glycol, etc.; acid amides, for example, dimethylformamide (DMF), dimethylacetamide (DMA), N-methylpyrrolidone, 1,3-dimethyl-2-imidazolidinone, hexamethylphosphoric triamide (HMPA), etc.; sulfones, sulfoxides, for example, dimethyl sulfoxide (DMSO), sulfolane, etc.; bases, for example, pyridine etc.

The preparation process (c) can be conducted in the presence of an acid binder, and as said acid binder there can be mentioned, for example, as inorganic bases, hydrides, hydroxides, carbonates and bicarbonates, etc. of alkali metals and alkaline earth metals, for example, sodium hydride, lithium hydride, sodium hydrogen carbonate, potassium hydrogen carbonate, sodium carbonate, potassium carbonate, lithium hydroxide, sodium hydroxide, potassium hydroxide, calcium hydroxide, etc.; inorganic alkali metal amides, for example, lithium amide, sodium amide, potassium amide, etc.; as organic bases, alcoholates, tertiary amines, dialkylaminoanilines and pyridines, for example, triethylamine, 1,1,4,4-tetramethylethylenediamine (TMEDA), N,N-dimethylaniline, N,N-diethylaniline, pyridine, 4-dimethylaminopyridine (DMAP), 1,4-diazabicyclo[2,2,2]octane (DABCO) and 1,8-diazabicyclo[5,4,0]undec-7-ene (DBU), etc.; organic lithium compounds, for example, methyl lithium, n-butyl lithium, sec-butyl lithium, tertbutyl lithium, phenyl lithium, dimethyl copper lithium, lithium diisopropyl amide, lithium cyclohexyl isopropyl amide, lithium dicyclohexyl amide, n-butyl lithium.DABCO, n-butyl lithium.DBU, n-butyl lithium.TMEDA, etc.; organic amine hydrochlorides, for example, pyridine hydrochloride, triethylamine hydrochloride, etc.; amine sulfonates, for example, pyridine p-toluenesulfonate, triethylamine p-toluenesulfonate, etc.

The preparation process (c) can be conducted in the presence of a catalyst and as example of said catalyst there can be mentioned, for example, palladium catalysts such as dichlorobis(triphenylphosphine) palladium, etc., metal catalysts such as copper (I) iodide etc.

The preparation process (c) can be conducted in a substantially wide range of temperature. There can be applied temperatures generally of about −78 to about 180° C., preferably about 0 to about 150° C. Although said reaction is conducted desirably under normal pressure, it can be conducted optionally under elevated pressure or under reduced pressure.

In conducting the preparation process (c), the aimed compound can be obtained, for example, by reacting 1.5 to 2.5 moles of a compound of the formula (IV) to 1 mole of a compound of the formula (IAc) in a diluent, for example, DMF, in the presence of potassium carbonate.

The reaction of the above-mentioned preparation process (d) can be conducted in an appropriate diluent. As examples of the diluent usable in that case there can be mentioned water; aliphatic, alicyclic and aromatic hydrocarbons (may be optionally chlorinated), for example, pentane, hexane, cyclohexane, petroleum ether, ligroine, benzene, toluene, xylene, dichloromethane, chloroform, carbon tetrachloride, 1,2-dichloroethane, chlorobenzene, dichlorobenzene, etc.; ethers, for example, ethyl ether, methyl ethyl ether, isopropyl ether, butyl ether, dioxane, dimethoxyethane (DME), tetrahydrofuran (THF), diethylene glycol dimethyl ether (DGM), etc.; ketones, for example, acetone, methyl ethyl ketone (MEK), methyl isopropyl ketone, methyl isobutyl ketone (MIBK), etc.; nitriles, for example, acetonitrile, propionitrile, acrylonitrile, etc.; alcohols, for example, methanol, ethanol, isopropanol, butanol, ethylene glycol, etc.; esters, for example, ethyl acetate, amyl acetate, etc.; acid amides, for example, dimethylformamide (DMF), dimethylacetamide (DMA), N-methylpyrrolidone, 1,3-dimethyl-2-imidazolidinone, hexamethylphosphoric triamide (HMPA), etc.; sulfones, sulfoxides, for example, dimethyl sulfoxide (DMSO), sulfolane, etc.; bases, for example, pyridine etc.

The preparation process (d) can be conducted in the presence of an acid binder, and as said acid binder there can be mentioned, for example, as inorganic bases, hydrides, hydroxides, carbonates and bicarbonates, etc. of alkali metals and alkaline earth metals, for example, sodium hydride, lithium hydride, sodium hydrogen carbonate, potassium hydrogen carbonate, sodium carbonate, potassium carbonate, lithium hydroxide, sodium hydroxide, potassium hydroxide, calcium hydroxide, etc.; inorganic alkali metal amides, for example, lithium amide, sodium amide, potassium amide, etc.; as organic bases, alcoholates, tertiary amines, dialkylaminoanilines and pyridines, for example, triethylamine, 1,1,4,4-tetramethylethylenediamine (TMEDA), N,N-dimethylaniline, N,N-diethylaniline, pyridine, 4-dimethylaminopyridine (DMAP), 1,4-diazabicyclo[2,2,2]octane (DABCO) and 1,8-diazabicyclo[5,4,0]undec-7-ene (DBU), etc.; organic lithium compounds, for example, methyl lithium, n-butyl lithium, sec-butyl lithium, tertbutyl lithium, phenyl lithium, dimethyl copper lithium, lithium diisopropyl amide, lithium cyclohexyl isopropyl amide, lithium dicyclohexyl amide, n-butyl lithium.DABCO, n-butyl lithium.DBU, n-butyl lithium.TMEDA, etc.; organic amine hydrochlorides, for example, pyridine hydrochloride, triethylamine hydrochloride, etc.; amine sulfonates, for example, pyridine p-toluenesulfonate, triethylamine p-toluenesulfonate, etc.

The preparation process (d) can be conducted in the presence of a catalyst and as example of said catalyst there can be mentioned, for example, palladium catalysts such as dichlorobis(triphenylphosphine) palladium etc. and metal catalysts such as copper (I) iodide etc.

The preparation process (d) can be conducted in a substantially wide range of temperature. There can be applied temperatures generally of about −78 to about 180° C., preferably about −20 to about 120° C. Although said reaction is conducted desirably under normal pressure, it can be conducted optionally under elevated pressure or under reduced pressure.

In conducting the preparation process (d), the aimed compound can be obtained, for example, by reacting 1.5 to 2.5 moles of a compound of the formula (V) to 1 mole of a compound of the formula (IAd) in a diluent, for example, THF, in the presence of triethylamine.

The reaction of the above-mentioned preparation process (e) can be conducted in an appropriate diluent. As examples of the diluent usable in that case there can be mentioned water; aromatic hydrocarbons, for example, benzene, toluene, xylene, etc.; alcohols, for example, methanol, ethanol, isopropanol, butanol, ethylene glycol, etc.; esters, for example, ethyl acetate, amyl acetate, etc.; carboxylic acids, for example, acetic acid etc.

The preparation process (e) can be conducted in the presence of a catalyst and as said catalyst there can be mentioned, for example, palladium carbon etc.

The preparation process (e) can be conducted in the presence of an acid binder, and as said acid binder there can be mentioned, for example, as inorganic bases, hydrides, hydroxides, carbonates and bicarbonates, etc. of alkali metals and alkaline earth metals, for example, sodium hydride, lithium hydride, sodium hydrogen carbonate, potassium hydrogen carbonate, sodium carbonate, potassium carbonate, lithium hydroxide, sodium hydroxide, potassium hydroxide, calcium hydroxide, etc.

The preparation process (e) can be conducted in a substantially wide range of temperature. There can be applied temperatures generally of about −40 to about 180° C., preferably about 0 to about 140° C. Although said reaction is conducted desirably under normal pressure, it can be conducted optionally under elevated pressure or under reduced pressure.

In conducting the preparation process (e), the aimed compound can be obtained, for example, by reacting a catalytic amount of palladium carbon to 1 mole of a compound of the formula (IAe) in a diluent, for example, toluene-ethanol, in the presence of aqueous solution of sodium carbonate and in hydrogen atmosphere.

The reaction of the above-mentioned preparation process (f) can be conducted in an appropriate diluent. As examples of the diluent usable in that case there can be mentioned water; aliphatic, alicyclic and aromatic hydrocarbons (may be optionally chlorinated), for example, pentane, hexane, cyclohexane, petroleum ether, ligroine, benzene, toluene, xylene, dichloromethane, chlorobenzene, dichlorobenzene, etc.; ethers, for example, ethyl ether, methyl ethyl ether, isopropyl ether, butyl ether, dioxane, dimethoxyethane (DME), tetrahydrofuran (THF), diethylene glycol dimethyl ether (DGM), etc.; nitriles, for example, acetonitrile, propionitrile, acrylonitrile, etc.

The preparation process (f) can be conducted in a substantially wide range of temperature. There can be applied temperatures generally of about −40 to about 200° C., preferably about 0 to about 180° C. Although said reaction is conducted desirably under normal pressure, it can be conducted optionally under elevated pressure or under reduced pressure.

In conducting the preparation process (f), the aimed compound can be obtained, for example, by reacting 5 to 20 moles of sodium chlorodifluoroacetate to 1 mole of a compound of the formula (IAf) at about 180° C. in a diluent, for example, diglyme.

The reaction of the above-mentioned preparation process (g) can be conducted in an appropriate diluent. As examples of the diluent usable in that case there can be mentioned water; aromatic hydrocarbons, for example, benzene, toluene, xylene, etc.; alcohols, for example, methanol, ethanol, isopropanol, butanol, ethylene glycol, etc.; esters, for example, ethyl acetate, amyl acetate, etc.; acid amides, for example, dimethylformamide (DMF), dimethylacetamide (DMA), N-methylpyrrolidone, 1,3-dimethyl-2-imidazolidinone, hexamethylphosphoric triamide (HMPA), etc.; sulfones, sulfoxides, for example, dimethyl sulfoxide (DMSO), sulfolane, etc.; carboxylic acids, for example, acetic acid etc.

The preparation process (g) can be conducted in the presence of an appropriate catalyst and as said catalyst there can be mentioned, for example, palladium carbon etc.

The preparation process (g) can be conducted also by using an appropriate metal hydride and as said metal hydrides there can be mentioned, for example, sodium borohydride, lithium aluminium hydride, etc.

In conducting the preparation process (g), the aimed compound can be obtained, for example, by reacting a catalytic amount of palladium carbon to 1 mole of a compound of the formula (IAg) in a diluent, for example, ethanol, in hydrogen atmosphere.

The reaction of the first step and the second step of the above-mentioned preparation process (h) can be conducted continuously in one pot in an appropriate diluent. As examples of the diluent usable in that case there can be mentioned water; ketones, for example, acetone, methyl ethyl ketone (MEK), methyl isopropyl ketone, methyl isobutyl ketone (MIBK), etc.; nitriles, for example, acetonitrile, propionitrile, etc.; sulfones, sulfoxides, for example, dimethyl sulfoxide (DMSO), sulfolane, etc.; carboxylic acids, for example, acetic acid; mineral acids, for example, hydrochloric acid, sulfuric acid, etc.

The preparation process (h) can be conducted in the presence of an acid catalyst and as example of said acid catalyst there can be mentioned mineral acids, for example, nitric acid, hydrobromic acid, etc.

The preparation process (h) can be conducted in the presence of a catalyst and as example of such catalyst there can be mentioned copper halide compounds, for example, copper (I) chloride, copper (II) chloride, etc.

The reaction of the first step and the second step of the preparation process (h) can be conducted in a substantially wide range of temperature. There can be applied temperatures generally of about −40 to about 180° C., preferably about −20 to about 120° C. Although said reaction is conducted desirably under normal pressure, it can be conducted optionally under elevated pressure or under reduced pressure.

In conducting the preparation process (h), the aimed compound can be obtained, for example, by reacting 1.2 to 2.5 moles of tert-butyl nitrite to 1 mole of a compound of the formula (IAh) in a diluent, for example, acetonitrile, in the presence of copper (II) chloride.

The reaction of the first step of the above-mentioned preparation process (i) can be conducted in an appropriate diluent. As examples of the diluent usable in that case there can be mentioned aromatic hydrocarbons, for example, benzene, toluene, xylene, etc.; acid amides, for example, dimethylformamide (DMF), dimethylacetamide (DMA), N-methylpyrrolidone, 1,3-dimethyl-2-imidazolidinone, hexamethylphosphoric triamide (HMPA), etc

The first step of the preparation process (h) can be conducted in a substantially wide range of temperature. There can be applied temperatures generally of about −40 to about 180° C., preferably about 0 to about 140° C. Although said reaction is conducted desirably under normal pressure, it can be conducted optionally under elevated pressure or under reduced pressure.

In conducting the first step of the preparation process (i), the aimed compound of the formula (VI) can be obtained, for example, by reacting 1.1 to 2.0 moles of dimethylformamide dimethylacetal to 1 mole of a compound of the formula (IAh) in a diluent, for example, DMF.

The reaction of the second step of the above-mentioned preparation process (i) can also be conducted in an appropriate diluent. As examples of the diluent usable in that case there can be mentioned aliphatic, alicyclic and aromatic hydrocarbons (may be optionally chlorinated), for example, pentane, hexane, cyclohexane, petroleum ether, ligroine, benzene, toluene, xylene, dichloromethane, chloroform, carbon tetrachloride, 1,2-dichloroethane, chlorobenzene, dichlorobenzene, etc.; ethers, for example, ethyl ether, methyl ethyl ether, isopropyl ether, butyl ether, dioxane, dimethoxyethane (DME), tetrahydrofuran (THF), diethylene glycol dimethyl ether (DGM), etc.; alcohols, for example, methanol, ethanol, isopropanol, butanol, ethylene glycol, etc.

The second step of the preparation process (i) can be conducted in the presence of an acid binder, and as said acid binder there can be mentioned, for example, as inorganic bases, hydrides, hydroxides, carbonates and bicarbonates, etc. of alkali metals and alkaline earth metals, for example, sodium hydride, lithium hydride; sodium hydrogen carbonate, potassium hydrogen carbonate, sodium carbonate, potassium carbonate, lithium hydroxide, sodium hydroxide, potassium hydroxide, calcium hydroxide, etc.; as organic bases, alcoholates, tertiary amines, dialkylaminoanilines and pyridines, for example, triethylamine, 1,1,4,4-tetramethylethylenediamine (TMEDA), N,N-dimethylaniline, N,N-diethylaniline, pyridine, 4-dimethylaminopyridine (DMAP), 1,4-diazabicyclo[2,2,2]octane (DABCO) and 1,8-diazabicyclo[5,4,0]undec-7-ene (DBU), etc.

The second step of the preparation process (i) can also be conducted in the presence of an acid catalyst. As examples of said acid catalyst there can be mentioned organic acids, for example, formic acid, acetic acid, trifluoroacetic acid, propionic acid, methanesulfonic acid, benzenesulfonic acid, p-toluenesulfonic acid, etc.; organic amine hydrochlorides, for example, pyridine hydrochloride, triethylamine hydrochloride, etc.; amine sulfonates, for example, pyridine p-toluenesulfonate, triethylamine p-toluenesulfonate, etc

The second step of the preparation process (i) can be conducted in a substantially wide range of temperature. There can be applied temperatures generally of about −40 to about 180° C., preferably about 0 to about 140° C. Although said reaction is conducted desirably under normal pressure, it can be conducted optionally under elevated pressure or under reduced pressure.

In conducting the second step of the preparation process (i), the objective compound can be obtained, for example, by reacting 1.1 to 8.0 moles of the compound of the formula (VII) to 1 mole of a compound of the formula (VI) in a diluent, for example, toluene, in the presence of triethylamine

In conducting the second step of the preparation process (i), the compound of the formula (IA) can also be obtained by continuously conducting reactions starting from a compound of the formula (IAh) and without isolating and purifying the compound of the formula (VI) intermediately.

The reaction of the above-mentioned preparation process (j) can be conducted in an appropriate diluent. As examples of the diluent usable in that case there can be mentioned aliphatic, alicyclic and aromatic hydrocarbons (may be optionally chlorinated), for example, pentane, hexane, cyclohexane, petroleum ether, ligroine, benzene, toluene, xylene, dichloromethane, chloroform, carbon tetrachloride, 1,2-dichloroethane, chlorobenzene, dichlorobenzene, etc.; ethers, for example, ethyl ether, methyl ethyl ether, isopropyl ether, butyl ether, dioxane, dimethoxyethane (DME), tetrahydrofuran (THF), diethylene glycol dimethyl ether (DGM), etc.; bases, for example, pyridine etc.

The preparation process (j) can be conducted in the presence of an acid binder, and as said acid binder there can be mentioned, for example, as organic bases, alcoholates, tertiary amines, dialkylaminoanilines and pyridines, for example, triethylamine, 1,1,4,4-tetramethylethylenediamine (TMEDA), N,N-dimethylaniline, N,N-diethylaniline, pyridine, 4-dimethylaminopyridine (DMAP), 1,4-diazabicyclo[2,2,2]octane (DABCO) and 1,8-diazabicyclo[5,4,0]uradec-7-ene (DBU), etc.

The preparation process (j) can be conducted in a substantially wide range of temperature. There can be applied temperatures generally of about −78 to about 180° C., preferably about −20 to about 120° C. Although said reaction is conducted desirably under normal pressure, it can be conducted optionally under elevated pressure or under reduced pressure.

In conducting the preparation process (j), the aimed compound can be obtained, for example, by reacting 0.8 to 1.5 moles of a compound of the formula (VIII) to 1 mole of a compound of the formula (IAh) in a diluent, for example, pyridine.

The reaction of the above-mentioned preparation process (k) can be conducted in an appropriate diluent. As examples of the diluent usable in that case there can be mentioned ethers, for example, ethyl ether, methyl ethyl ether, isopropyl ether, butyl ether, dioxane, dimethoxyethane (DME), tetrahydrofuran (THF), diethylene glycol dimethyl ether (DGM), etc.

The preparation process (k) can be conducted in a substantially wide range of temperature. There can be applied temperatures generally of about −78 to about 180° C., preferably about −20 to about 120° C. Although said reaction is conducted desirably under normal pressure, it can be conducted optionally under elevated pressure or under reduced pressure.

In conducting the preparation process (k), the aimed compound can be obtained, for example, by reacting 1.1 to 3.3 moles of a compound of the formula (IX) to 1 mole of a compound of the formula (IAk) in a diluent, for example, ethyl ether.

The reaction of the above-mentioned preparation process (l) can be conducted in an appropriate diluent. As examples of the diluent usable in that case there can be mentioned water, aliphatic, alicyclic and aromatic hydrocarbons (may be optionally chlorinated), for example, pentane, hexane, cyclohexane, petroleum ether, ligroine, benzene, toluene, xylene, dichloromethane, chloroform, carbon tetrachloride, 1,2-dichloroethane, chlorobenzene, dichlorobenzene, etc.; ethers, for example, ethyl ether, methyl ethyl ether, isopropyl ether, butyl ether, dioxane, dimethoxyethane (DME), tetrahydrofuran (THF), diethylene glycol dimethyl ether (DGM), etc.; alcohols, for example, methanol, ethanol, isopropanol, butanol, ethylene glycol, etc

The preparation process (l) can be conducted in the presence of an acid binder, and as said acid binder there can be mentioned, for example, as inorganic bases, hydrides, hydroxides, carbonates and bicarbonates, etc. of alkali metals and alkaline earth metals, for example, sodium hydride, lithium hydride, sodium hydrogen carbonate, potassium hydrogen carbonate, sodium carbonate, potassium carbonate, lithium hydroxide, sodium hydroxide, potassium hydroxide, calcium hydroxide, etc.; as organic bases, alcoholates, tertiary amines, dialkylaminoanilines and pyridines, for example, triethylamine, 1,1,4,4-tetramethylethylenediamine (TMEDA), N,N-dimethylaniline, N,N-diethylaniline, pyridine, 4-dimethylaminopyridine (DMAP), 1,4-diazabicyclo[2,2,2]octane (DABCO) and 1,8-diazabicyclo[5,4,0]undec-7-ene (DBU), etc.

The preparation process (l) can also be conducted in the presence of an acid catalyst. As examples of said acid catalyst there can be mentioned p-toluenesulfonic acid, etc.; organic amine salts, for example, pyridine p-toluenesulfonate etc.

The preparation process (l) can be conducted in a substantially wide range of temperature. There can be applied temperatures generally of about −78 to about 180° C., preferably about −20 to about 120° C. Although said reaction is conducted desirably under normal pressure, it can be conducted optionally under elevated pressure or under reduced pressure.

In conducting the preparation process (l), the objective compound can be obtained, for example, by reacting 1.1 to 8.0 moles of a compound of the formula (X) to 1 mole of a compound of the formula (IAl) in a diluent, for example, ethanol, in the presence of sodium hydrogen carbonate.

The reaction of the above-mentioned preparation process (m) can be conducted in an appropriate diluent. As examples of the diluent usable in that case there can be mentioned water; aliphatic, alicyclic and aromatic hydrocarbons (may be optionally chlorinated), for example, pentane, hexane, cyclohexane, petroleum ether, ligroine, benzene, toluene, xylene, dichloromethane, chloroform, carbon tetrachloride, 1,2-dichloroethane, chlorobenzene, dichlorobenzene, etc.; ethers, for example, ethyl ether, methyl ethyl ether, isopropyl ether, butyl ether, dioxane, dimethoxyethane (DME), tetrahydrofuran (THF), diethylene glycol dimethyl ether (DGM), etc.; alcohols, for example, methanol, ethanol, isopropanol, butanol, ethylene glycol, etc.

The preparation process (m) can be conducted in the presence of an acid binder, and as said acid binder there can be mentioned, for example, as inorganic bases, hydrides, hydroxides, carbonates and bicarbonates, etc. of alkali metals and alkaline earth metals, for example, sodium hydride, lithium hydride, sodium hydrogen carbonate, potassium hydrogen carbonate, sodium carbonate, potassium carbonate, lithium hydroxide, sodium hydroxide, potassium hydroxide, calcium hydroxide, etc.; as organic bases, alcoholates, tertiary amines, dialkylaminoanilines and pyridines, for example, triethylamine,

1,1,4,4-tetramethylethylenediamine (TMEDA), N,N-dimethylaniline, N,N-diethylaniline, pyridine, 4-dimethylaminopyridine (DMAP), 1,4-diazabicyclo[2,2,2]octane (DABCO) and 1,8-diazabicyclo[5,4,0]undec-7-ene (DBU), etc.

The preparation process (m) can also be conducted in the presence of an acid catalyst. As examples of said acid catalyst there can be mentioned organic acids, for example, formic acid, acetic acid, trifluoroacetic acid, propionic acid, methanesulfonic acid, benzenesulfonic acid, p-toluenesulfonic acid, etc.; organic amine hydrochlorides, for example, pyridine hydrochloride, triethylamine hydrochloride, etc.; amine sulfonates, for example, pyridine p-toluenesulfonate, triethylamine p-toluenesulfonate, etc.

In conducting the preparation process (m), the aimed compound can be obtained, for example, by reacting 1.1 to 8.0 moles of a compound of the formula (XI) to 1 mole of a compound of the formula (IAk) in a diluent, for example, toluene in the presence of triethylamine.

The active component compounds of the formula (I) of the present invention show a strong fungicidal and bactericidal action and in fact, they can be used to control undesirable plant pathogens.

The active component compounds of the formula (I) of the present invention can be used generally as fungicidal and bacteriacidal agents against various plant diseases by Plasmodiophoromycetes, Oomycetes, Zygomycetes, Ascomycetes, Basidiomycetes and Deuteromycetes.

According to the present invention the active component compounds of the formula (II) show excellent controlling effect particularly against such plant pathogens as Sphaerotheca fuliginea, Gibberella fujikuroi, Alternaria mali, Pyricularia oryzae, Phytophthora infestans, Cochliobolus miyabeanus, Botrytis cinerea, etc.

The active component compounds of the formula (I) of the present invention show good compatibility to plants at the concentration of the active compound necessary to control plant pathogens and, in case of using, chemical treatment of aboveground parts of plant, chemical treatment of stocks and seeds, and soil treatment are possible.

The active component compounds of the formula (I) of the present invention can be used further, in the protection of various materials, to protect them from infection and destruction by undesirable microorganisms.

The materials in the present specification are understood to mean inanimate objects manufactured to be widely used.

As the materials to be able to be protected by the active compounds of the present invention from changes or destruction by attack of microorganisms they can be, for example, adhesives, sizes, paper and cardboard, textiles, leather, wood, (synthetic) paints, cooling lubricants, heat exchange liquid and other materials that can be infected and destructed by microorganisms, among which wood is particularly favorable. In the scope of materials to be protected there can be included a part of a manufacturing plant, for example, a cooling water circuit that can be damaged by proliferation of microorganisms.

As examples of the microorganisms that cause deterioration or changes of materials there can be mentioned bacteria, molds, yeasts, algae, slime organisms, etc. The active compounds of the formula (I) of the present invention show actions preferably against molds, molds that discolor wood and/or destruct wood (Basidiomycetes).

As controlling objects, microorganisms of the following genera can be mentioned as examples:

Alternaria, for example, Alternaria tenuis;

Aspergillus, for example, Aspergillus niger;

Chaetomium, for example, Chaetomium globosum;

Coniophora, for example, Coniophora puetana;

Lentinus, for example, Lentinus tigrinus;

Penicillium, for example, Penicillium glaucum;

Polyporus, for example, Polyporus versicolor;

Aureobasidium, for example, Aureobasidium pullulans;

Sclerophoma, for example, Sclerophoma pityophila;

Trichoderma, for example, Trichoderma viride.

Moreover, the active component compounds of the formula (I) of the present invention are low toxic against warm-blooded animals and can be used safely.

The active component compounds of the formula (I), according to the present invention, can be made into customary formulation forms, in case that they are used as agricultural chemicals. As formulation forms there can be mentioned, for example, solutions, wettable powders, emulsions, suspensions, powders, foaming agents, pastes, tablets, granules, aerosols, active compound-impregnated natural and synthetic substances, microcapsules, seed coating agents, ULV [cold mist, warm mist], etc.

These formulations can be prepared according to per se known methods, for example, by mixing the active compounds with extenders, namely liquid diluents, solid diluents or carriers, and optionally with surface-active agents, namely emulsifiers and/or dispersants and/or foam-forming agents

As liquid diluents or carriers there can be mentioned, for example, aromatic hydrocarbons (for example, xylene, toluene, alkylnaphthalene, etc.), chlorinated aromatic or chlorinated aliphatic hydrocarbons (for example, chlorobenzenes, ethylene chlorides, methylene chloride, etc.), aliphatic hydrocarbons [for example, cyclohexane etc. or paraffins (for example, mineral oil fractions etc.)], alcohols (for example, butanol, glycols etc.) and their ethers, esters, etc., ketones (for example, acetone, methyl ethyl ketone, methyl isobutyl ketone or cyclohexanone, etc.), strongly polar solvents (for example, dimethylformamide, dimethyl sulfoxide, etc.), water, etc. In case of using water as extender, for example, organic solvents can be used as auxiliary solvents.

As solid diluents there can be mentioned, for example, ground natural minerals (for example, kaolin, clay, talc, chalk, quartz, attapulgite, montmorillonite or diatomaceous earth, etc.), ground synthetic minerals (for example, highly dispersed silicic acid, alumina, silicates, etc.).

As solid carriers for granules there can be mentioned, for example, crushed and fractionated rocks (for example, calcite, marble, pumice, sepiolite, dolomite, etc.) synthetic granules of inorganic and organic meals, particles of organic materials (for example, saw dust, coconut shells, maize cobs, tobacco stalks, etc.), etc.

As emulsifiers and/or foam-forming agents there can be mentioned, for example, nonionic and anionic emulsifiers [for example, polyoxyethylene fatty acid esters, polyoxyethylene fatty acid alcohol ethers (for example, alkylaryl polyglycol ethers, alkylsulfonates, alkylsulfates, arylsulfonates, etc.)], albumin hydrolysis products, etc.

Dispersants include, for example, lignin sulfite waste liquor, methyl cellulose, etc.

Tackifiers can also be used in preparations (powders, granules, emulsifiable concentrates). As the tackifiers usable in that case there can be mentioned, for example, carboxymethyl cellulose, natural and synthetic polymers (for example, gum Arabic, polyvinyl alcohol, polyvinyl acetate, etc.).

Colorants can also be used. As said colorants there can be mentioned inorganic pigments (for example, iron oxide, titanium oxide, Prussian Blue, etc,), organic dyestuffs such as alizarin dyestuffs, azo dyestuffs or metal phthalocyanine dyestuffs, and further traces nutrients such as iron, manganese, boron, copper, cobalt, molybdenum, zinc and salts of such metals.

Said formulations can contain the active component compounds of the formula (I) of the present invention at the concentration in the range of generally 0.1 to 95% by weight, preferably 0.5 to 90% by weight.

The active component compounds of the formula (I), according to the present invention can exist, in the above-mentioned formulations or various application forms, together with other known active compounds, for example, germicides (fungicides, bactericides), insecticides, miticides, nematicides, herbicides, bird repellents, growth regulators, fertilizers and/or soil improvement agents.

The active component compounds of the formula (I), according to the present invention can be used directly as they are or used in such a form as ready-to use solutions, emulsifiable concentrates, suspensions, powders, tablets, pastes, microcapsules, granules, etc., or used in application forms prepared by further dilution, when they are practically used. And the active component compounds of the formula (I), according to the present invention can be applied in a usual way, for example, watering, soaking, spraying, atomizing, misting, drenching, suspension formation, painting, dusting, seed dressing, etc.

In case of treating each part of the plant, the concentration of the active component compounds in the actual application form can be varied in a substantial range and can be in the range of generally 0.0001 to 1% by weight, preferably 0.001 to 0.5% by weight.

In case of seed treatment, the active component compounds, according to the present invention can be used in the range of generally 0.001 to 50 g, preferably 0.01 to 10 g per 1 kg of seeds.

In case of soil treatment, the active component compounds, according to the present invention can be used in the range of concentration of generally 0.00001 to 0.1% by weight, particularly 0.0001 to 0.02% by weight at the application point.

Then the present invention is described more specifically by Examples. The present invention, however, should not be restricted to them in any way.

### SYNTHESIS EXAMPLE 1

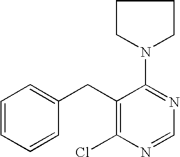

5-Benzyl-4,6-dichloropyrimidine (960 mg, 4.0 mmol) was dissolved in tetrahydrofuran (20 ml), to which then pyrrolidine (660 μl, 8.0 mmol) and triethylamine (1.2 ml, 8.6 mmol) were added and the mixture was refluxed for 3 hours. After finishing the reaction, the precipitation was removed and the filtrate was concentrated under reduced pressure. The residue was purified by flush column chromatography (eluent n-hexane:ethyl acetate 4:1) to obtain 5-benzyl-4-chloro-6-pyrrolidin-1-yl-pyrimidine (1.05 g).

¹H NMR (CDCl₃, 300 MHz) δ 1.80-1.85 (4H, m), 3.54-3.58 (4H, m), 4.27 (2H, s), 7.082H, d, J=6.9H, z), 7.21-7.31 (3H, m), 8.31 (1H, s).

### SYNTHESIS EXAMPLE 2

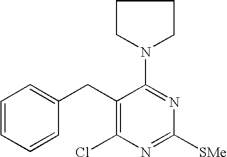

5-Benzyl-4,6-dichloro-2-methylthiopyrimidine (1.14 g, 4.0 mmol) was dissolved in tetrahydrofuran (20 ml), to which then pyrrolidine (660 μl, 8.0 mmol) and triethylamine (1.2 ml, 8.6 mmol) were added and the mixture was refluxed for 3 hours. After finishing the reaction, the precipitation was removed and the filtrate was concentrated under reduced pressure. The residue was purified by flush column chromatography (eluent n-hexane:ethyl acetate=4:1) to obtain 5-benzyl-4-chloro-2-methylthio-6-pyrrolidin-1-yl-pyrimidine (1.1 g).

mp 97-99° C.

### SYNTHESIS EXAMPLE 3

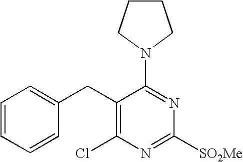

5-Benzyl-4-chloro-2-methylthio-6-pyrrolidin-yl-pyrimidine (1.9 g, 6 mmol) was dissolved in 30 ml of dichloromethane, to which m-chloroperbenzoic acid (3 g, 12 mmol) was added under ice cooling and the mixture was stirred at room temperature for 1 hour. After finishing the reaction, an aqueous solution of sodium thiosulfate was added thereto and the precipitation was filtered off. Then the reaction solution was washed with an aqueous solution of sodium hydrogen carbonate and a saturated aqueous solution of sodium chloride and the solvent was removed under reduced pressure. The residue was purified by flush column chromatography (eluent n-hexane:ethyl acetate=4:1) to obtain 2.0 g of 5-benzyl-4-chloro-2-methanesulfonyl-6-pyrrolidin-1-yl-pyrimidine.

mp 136-138° C.

### SYNTHESIS EXAMPLE 4

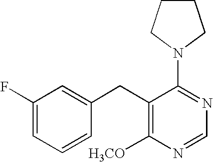

4-Chloro-5-(3-fluorobenzyl)-6-pyrrolidin-1-yl-pyrimidine (370 mg, 1.3 mmol) was dissolved in tetrahydrofuran (20 ml), to which 28% methanol solution of sodium methoxide (370 mg, 1.9 mmol) was added dropwise at room temperature and the mixture was stirred at room temperature for 2 hours. After finishing the reaction, the reaction solution was poured into ice water and extracted with ethyl acetate. The organic layer was dried with anhydrous magnesium sulfate, the solvent was distilled off under reduced pressure and the obtained crude product was purified by silica gel column chromatography (eluent n-hexane:ethyl acetate=5:1 (v/v)) to obtain 5-(3-fluorobenzyl)-4-methoxy-6-pyrrolidin-1-yl-pyrimidine (0.3 g).

mp 74-76° C.

### SYNTHESIS EXAMPLE 5

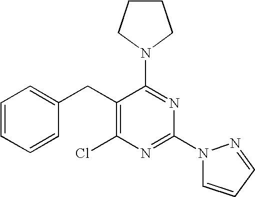

5-Benzyl-4-chloro-2-methanesulfonyl-6-pyrrolidin-1-yl-pyrimidine (500 mg, 1.42 mmol) was dissolved in N,N-dimethylformamide (50 ml), to which potassium carbonate (390 mg, 2.8 mmol) and pyrazole (145 mg, 2.1 mmol) were added and the mixture was stirred at 50° C. for 3 hours. After finishing the reaction, the reaction solution was poured into water and extracted with ethyl acetate. The solvent was distilled off under reduced pressure and the residue was purified by flush column chromatography (eluent n-hexane:ethyl acetate=4:1) to obtain 5-benzyl-4-chloro-2-pyrazol-1-yl-pyrimidine (400 mg).

mp 149-151° C.

### SYNTHESIS EXAMPLE 6

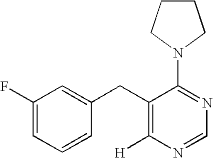

4-Chloro-5-(3-fluorobenzyl)-6-pyrrolidin-1-yl-pyrimidine (500 mg, 1.7 mmol) was dissolved in toluene (7 ml) and ethanol (5 ml), to which an aqueous solution prepared by dissolving sodium carbonate (0.1 g) in water (1 ml) was added. Further, 5% palladium carbon (0.15 g) was added thereto and the mixture was contacted with hydrogen gas at room temperature for 1 hour. After finishing the reaction, the catalyst was filtered off, and the filtrate was separated by adding chloroform and water. The organic layer was dried with anhydrous magnesium sulfate, the solvent was distilled off under reduced pressure and the obtained crude product was purified by silica gel column chromatography (eluent hexane:ethyl acetate=5:1 (v/v)) to obtain 5-(3-fluorobenzyl)-4-pyrrolidin-1-yl-pyrimidine (0.35 g).

mp 51-54° C.

Specific examples of the compounds obtained in the same manner to the above-mentioned Synthesis Examples 1-6 are shown, together with the compounds synthesized in Synthesis Examples 1-6, in the following Tables 1-3, and their physical and chemical properties are shown in Table 4.

In the compounds of the formula (IA) of the present invention, examples of the compounds in case that they represent the formula 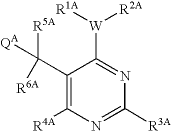
are shown in Table 1, examples of the of the compounds in case that they represent the formula 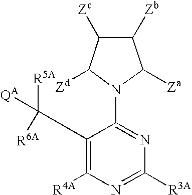
are shown in Table 1, examples of the compounds in case that they represent the formula 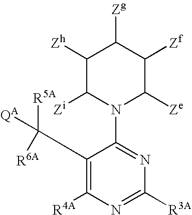
are shown in Table 3.

In Table 1, Table 2 and Table 3, Ph represents phenyl and Naph represents naphthyl.

TABLE 1

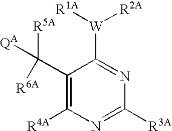

| | | | | | | | | |
|---|---|---|---|---|---|---|---|---|
| Comp. | | | | | | | | |
| No. | Q^{A} | W | R^{1A} | R^{2A} | R^{3A} | R^{4A} | R^{5A} | R^{6A} |

| | | | | | | | |
|---|---|---|---|---|---|---|---|
| 1-1 | Ph | N | —CH₂—CH(CH₃)— | H | Cl | H | H |
| 1-2 | 3,5-(F)₂—Ph | N | —CH₂—CH(CH₃)— | H | Cl | H | H |
| 1-3 | Ph | N | —CH₂—CH₂—CH₂— | H | Cl | H | H |
| 1-4 | 3,5-(F)₂—Ph | N | —CH₂—CH₂—CH₂— | H | Cl | H | H |
| 1-5 | Ph | N | —CH₂(CH₂)₂CH₂— | H | Cl | H | H |
| 1-6 | Ph | N | —CH₂(CH₂)₂CH₂— | H | Cl | CH₃ | H |
| 1-7 | Ph | N | —CH₂(CH₂)₂CH₂— | H | Cl | CH₂CH₃ | H |
| 1-8 | Ph | N | —CH₂(CH₂)₂CH₂— | H | Cl | CH(CH₃)CH₃ | H |
| 1-9 | Ph | N | —CH₂(CH₂)₂CH₂— | H | Cl | CF₃ | H |
| 1-10 | Ph | N | —CH₂(CH₂)₂CH₂— | H | Cl | Ph | H |
| 1-11 | Ph | N | —CH₂(CH₂)₂—CH(CH₃)— | H | Cl | H | H |
| 1-12 | Ph | N | —CH₂(CH₂)₂—CH(CH₃)— | H | Cl | CH₃ | H |
| 1-13 | Ph | N | —CH₂(CH₂)₂—CH(CH₃)— | C(CH₃)₃ | Cl | H | H |
| 1-14 | 3-F—Ph | N | —CH₂(CH₂)₂—CH(CH₃)— | H | Cl | H | H |
| 1-15 | 3-Cl—Ph | N | —CH₂(CH₂)₂—CH(CH₃)— | H | Cl | H | H |
| 1-16 | 3,4-(F)₂—Ph | N | —CH₂(CH₂)₂—CH(CH₃)— | H | Cl | H | H |
| 1-17 | 3,5-(F)₂—Ph | N | —CH₂(CH₂)₂—CH(CH₃)— | H | Cl | H | H |
| 1-18 | Ph | N | —CH₂(CH₂)₂—CH(CF₃)— | H | Cl | H | H |
| 1-19 | Ph | N | —CH₂(CH₂)₂—C(═O)— | H | Cl | H | H |
| 1-20 | Ph | N | —CH(CH₃)—(CH₂)₂—C(═O)— | H | Cl | H | H |
| 1-21 | Ph | N | H₂(CH₂)₂—CH(CH₂OH)— | H | Cl | H | H |
| | | | (S-configuration | | | | |
| 1-22 | Ph | N | H₂(CH₂)₂—CH(CH₂OH)— | H | Cl | H | H |
| | | | (R-configuration | | | | |
| 1-23 | Ph | N | —CH₂(CH₂)₂—CH(CH₂OCH₃)— | H | Cl | H | H |
| 1-24 | Ph | N | —CH₂(CH₂)₂—CH(COOH)— | H | Cl | H | H |
| 1-25 | Ph | N | —CH₂(CH₂)₂—CH(COOCH₃)— | H | Cl | H | H |
| 1-26 | Ph | N | —CH₂(CH₂)₂—CH(COOCH₂Ph)— | H | Cl | H | H |
| 1-27 | Ph | N | —CH₂(CH₂)₂—OH(CH₂NHPh)— | H | Cl | H | H |
| 1-28 | Ph | N | —CH(CH₃)—(CH₂)₂—OH(CH₃)— | H | Cl | H | H |
| 1-29 | Ph | N | —CH(CH₂OCH₃)—(CH₂)₂—CH(CH₂OCH₃)— | H | Cl | H | H |
| 1-30 | Ph | N | —(CH₂)₂—CH(OH)—CH₂— | H | Cl | H | H |
| 1-31 | Ph | N | —(CH₂)₂—OH(NHCOCH₃)—CH₂— | H | Cl | H | H |
| 1-32 | Ph | N | —(CH₂)₂—CH(NHOOCF₃)—CH₂— | H | Cl | H | H |
| 1-33 | Ph | N | —CH₂—CH═CH—CH₂— | H | Cl | H | H |
| 1-34 | Ph | N | —CH₂—CH═CH—C(COOH)— | H | Cl | H | H |
| 1-35 | Ph | N | —CH₂—CH═CH—C(COOCH₃)— | H | Cl | H | H |
| 1-36 | Ph | N | —CH(CH₃)—CHCH—CH(CH₃)— | H | Cl | H | H |
| 1-37 | Ph | N | —CH₂CH₂—S—CH₂— | H | Cl | H | H |
| 1-38 | Ph | N | —CH₂CH₂—S—CH₂— | H | Cl | CH₃ | H |
| 1-39 | Ph | N | —CH₂CH₂—S—CH₂— | C(CH₃)₃ | Cl | H | H |
| 1-40 | 3-F—Ph | N | —CH₂CH₂—S—CH₂— | H | Cl | H | H |
| 1-41 | 3-Cl—Ph | N | —CH₂CH₂—S—CH₂— | H | Cl | H | H |
| 1-42 | Ph | N | —CH₂CH₂—CH═N— | H | Cl | H | H |
| 1-43 | 1-Naph | N | —CH₂(CH₂)₂CH₂— | H | Cl | H | H |
| 1-44 | 2-Naph | N | —CH₂(CH₂)₂CH₂— | H | Cl | H | H |
| 1-45 | 2-F—Ph | N | —CH₂(CH₂)₂CH₂— | H | Cl | H | H |
| 1-46 | 2-Cl—Ph | N | —CH₂(CH₂)₂CH₂— | H | Cl | H | H |
| 1-47 | 2-Br—Ph | N | —CH₂(CH₂)₂CH₂— | H | Cl | H | H |
| 1-48 | 2-I—Ph | N | —CH₂(CH₂)₂CH₂— | H | Cl | H | H |
| 1-49 | 2-CH₃—Ph | N | —CH₂(CH₂)₂CH₂— | H | Cl | H | H |
| 1-50 | 3-CF₃—Ph | N | —CH₂(CH₂)₂CH₂— | H | Cl | H | H |
| 1-51 | 2-CN—Ph | N | —CH₂(CH₂)₂CH₂— | H | Cl | H | H |
| 1-52 | 2-OCH₃—Ph | N | —CH₂(CH₂)₂CH₂— | H | Cl | H | H |
| 1-53 | 2-NO₂—Ph | N | —CH₂(CH₂)₂CH₂— | H | Cl | H | H |
| 1-54 | 2-NH₂—Ph | N | —CH₂(CH₂)₂CH₂— | H | Cl | H | H |
| 1-55 | 2-Ph—Ph | N | —CH₂(CH₂)₂CH₂— | H | Cl | H | H |
| 1-56 | 3-F—Ph | N | —CH₂(CH₂)₂CH₂— | H | Cl | H | H |
| 1-57 | 3-Cl—Ph | N | —CH₂(CH₂)₂CH₂— | H | Cl | H | H |
| 1-58 | 3-Br—Ph | N | —CH₂(CH₂)₂CH₂— | H | Cl | H | H |
| 1-59 | 3-I—Ph | N | —CH₂(CH₂)₂CH₂— | H | Cl | H | H |
| 1-60 | 3-CH₃—Ph | N | —CH₂(CH₂)₂CH₂— | H | Cl | H | H |
| 1-61 | 3-CF₃—Ph | N | —CH₂(CH₂)₂CH₂— | H | Cl | H | H |
| 1-62 | 3-CN—Ph | N | —CH₂(CH₂)₂CH₂— | H | Cl | H | H |
| 1-63 | 3-OCH₃—Ph | N | —CH₂(CH₂)₂CH₂— | H | Cl | H | H |
| 1-64 | 3-OCF₃—Ph | N | —CH₂(CH₂)₂CH₂— | H | Cl | H | H |
| 1-65 | 3-NO₂—Ph | N | —CH₂(CH₂)₂CH₂— | H | Cl | H | H |
| 1-66 | 3-NH₂—Ph | N | —CH₂(CH₂)₂CH₂— | H | Cl | H | H |
| 1-67 | 3—Ph—Ph | N | —CH₂(CH₂)₂CH₂— | H | Cl | H | H |
| 1-68 | 4-F—Ph | N | —CH₂(CH₂)₂CH₂— | H | Cl | H | H |
| 1-69 | 4-Cl—Ph | N | —CH₂(CH₂)₂CH₂— | H | Cl | H | H |
| 1-70 | 4-Br—Ph | N | —CH₂(CH₂)₂CH₂— | H | Cl | H | H |
| 1-71 | 4-CH₃—Ph | N | —CH₂(CH₂)₂CH₂— | H | Cl | H | H |
| 1-72 | 4-C(CH₃)₃—Ph | N | —CH₂(CH₂)₂CH₂— | H | Cl | H | H |
| 1-73 | 4-CF₃—Ph | N | —CH₂(CH₂)₂CH₂— | H | Cl | H | H |
| 1-74 | 4-CN—Ph | N | —CH₂(CH₂)₂CH₂— | H | Cl | H | H |
| 1-75 | 4-OCH₃—Ph | N | —CH₂(CH₂)₂CH₂— | H | Cl | H | H |
| 1-76 | 4-OCF₃—Ph | N | —CH₂(CH₂)₂CH₂— | H | Cl | H | H |
| 1-77 | 4-NO₂—Ph | N | —CH₂(CH₂)₂CH₂— | H | Cl | H | H |
| 1-78 | 4-NH₂—Ph | N | —CH₂(CH₂)₂CH₂— | H | Cl | H | H |
| 1-79 | 4—Ph—Ph | N | —CH₂(CH₂)₂CH₂— | H | Cl | H | H |
| 1-80 | 2-Cl-4-F—Ph | N | —CH₂(CH₂)₂CH₂— | H | Cl | H | H |
| 1-81 | 2-Cl-6-F—Ph | N | —CH₂(CH₂)₂CH₂— | H | Cl | H | H |
| 1-82 | 2,3-(F)₂—Ph | N | —CH₂(CH₂)₂CH₂— | H | Cl | H | H |
| 1-83 | 2,4-(F)₂—Ph | N | —CH₂(CH₂)₂CH₂— | H | Cl | H | H |
| 1-84 | 2,5-(F)₂—Ph | N | —CH₂(CH₂)₂CH₂— | H | Cl | H | H |
| 1-85 | 2,6-(F)₂—Ph | N | —CH₂(CH₂)₂CH₂— | H | Cl | H | H |
| 1-86 | 3,4-(F)₂—Ph | N | —CH₂(CH₂)₂CH₂— | H | Cl | H | H |
| 1-87 | 3,5-(F)₂—Ph | N | —CH₂(CH₂)₂CH₂— | H | Cl | H | H |
| 1-88 | 2,3,4-(F)₃—Ph | N | —CH₂(CH₂)₂CH₂— | H | Cl | H | H |
| 1-89 | 2,3,6-(F)₃—Ph | N | —CH₂(CH₂)₂CH₂— | H | Cl | H | H |
| 1-90 | 2,3,6-(F)₃—Ph | N | —CH₂(CH₂)₂CH₂— | H | Cl | H | H |
| 1-91 | 2,3,4,5,6-(F)₅—Ph | N | —CH₂(CH₂)₂CH₂— | H | Cl | H | H |
| 1-92 | 2,3,4,5,6-(F)₅—Ph | N | —CH₂(CH₂)₂CH₂— | H | Cl | F | F |
| 1-93 | pyridin-2-yl | N | —CH₂(CH₂)₂CH₂— | H | Cl | H | H |
| 1-94 | 6-Cl-pyridin-2-yl | N | —CH₂(CH₂)₂CH₂— | H | Cl | H | H |
| 1-95 | 3-Cl-5-CF₃-pyridin-2-yl | N | —CH₂(CH₂)₂CH₂— | H | Cl | H | H |
| 1-96 | pyridin-3-yl | N | —CH₂(CH₂)₂CH₂— | H | Cl | H | H |
| 1-97 | 6-Cl-pyridin-3-yl | N | —CH₂(CH₂)₂CH₂— | H | Cl | H | H |
| 1-98 | pyridin-4-yl | N | —CH₂(CH₂)₂CH₂— | H | Cl | H | H |
| 1-99 | 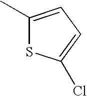 | N | —CH₂(CH₂)₂CH₂— | H | Cl | H | H |
| 1-100 | 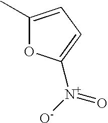 | N | —CH₂(CH₂)₂CH₂— | H | Cl | H | H |
| 1-101 | Ph | N | —CH₂(CH₂)₂CH₂— | H | H | H | H |
| 1-102 | 3-F—Ph | N | —CH₂(CH₂)₂CH₂— | H | H | H | H |
| 1-103 | Ph | N | —CH₂(CH₂)₂CH₂— | H | F | H | H |
| 1-104 | Ph | N | —CH₂(CH₂)₂CH₂— | H | CH₃ | H | H |
| 1-105 | Ph | N | —CH₂(CH₂)₂CH₂— | H | CH₂CH═CH₂ | H | H |
| 1-106 | Ph | N | —CH₂(CH₂)₂CH₂— | H | CF₃ | H | H |
| 1-107 | Ph | N | —CH₂(CH₂)₂CH₂— | H | OCH₃ | H | H |
| 1-108 | 3-F—Ph | N | —CH₂(CH₂)₂CH₂— | H | OCH₃ | H | H |
| 1-109 | Ph | N | —CH₂(CH₂)₂CH₂— | H | OCH₂CF₃ | H | H |
| 1-110 | Ph | N | —CH₂(CH₂)₂CH₂— | H | SCH₃ | H | H |
| 1-111 | Ph | N | —CH₂(CH₂)₂CH₂— | H | SCH₂CF₃ | H | H |
| 1-112 | Ph | N | —CH₂(CH₂)₂CH₂— | H | SOCH₃ | H | H |
| 1-113 | Ph | N | —CH₂(CH₂)₂CH₂— | H | SO₂CH₃ | H | H |
| 1-114 | Ph | N | —CH₂(CH₂)₂CH₂— | H | CN | H | H |
| 1-115 | Ph | N | —CH₂(CH₂)₂CH₂— | H | 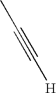 | H | H |
| 1-116 | Ph | N | —CH₂(CH₂)₂CH₂— | H | 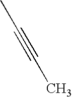 | H | H |
| 1-117 | Ph | N | —CH₂(CH₂)₂CH₂— | CH₃ | Cl | H | H |
| 1-118 | Ph | N | —CH₂(CH₂)₂CH₂— | CH₂CH₃ | Cl | H | H |
| 1-119 | Ph | N | —CH₂(CH₂)₂CH₂— | CH₂CH═CH₂ | Cl | H | H |
| 1-120 | Ph | N | —CH₂(CH₂)₂CH₂— | CF₃ | Cl | H | H |
| 1-121 | Ph | N | —CH₂(CH₂)₂CH₂— | C(CH₃)₃ | Cl | H | H |
| 1-122 | Ph | N | —CH₂(CH₂)₂CH₂— | CH(CH₃)₂ | Cl | H | H |
| 1-123 | Ph | N | —CH₂(CH₂)₂CH₂— | cyclopropyl | Cl | H | H |
| 1-124 | Ph | N | —CH₂(CH₂)₂CH₂— | Cl | Cl | H | H |
| 1-125 | Ph | N | —CH₂(CH₂)₂CH₂— | Ph | Cl | H | H |
| 1-126 | Ph | N | —CH₂(CH₂)₂CH₂— | 4-Cl—Ph | Cl | H | H |
| 1-127 | Ph | N | —CH₂(CH₂)₂CH₂— | 3,5-(Cl)₂—Ph | Cl | H | H |
| 1-128 | Ph | N | —CH₂(CH₂)₂CH₂— | CH₂—2,6-(Cl)₂—Ph | Cl | H | H |
| 1-129 | Ph | N | —CH₂(CH₂)₂CH₂— | CH₂O—4-Cl—Ph | Cl | H | H |
| 1-130 | Ph | N | —CH₂(CH₂)₂CH₂— | CH₂OCH₃ | Cl | H | H |
| 1-131 | Ph | N | —CH₂(CH₂)₂CH₂— | 4-CH₃—Ph | Cl | H | H |
| 1-132 | Ph | N | —CH₂(CH₂)₂CH₂— | 4-CF₃—Ph | Cl | H | H |
| 1-133 | Ph | N | —CH₂(CH₂)₂CH₂— | 3,5-(CF₃)₂—Ph | Cl | H | H |
| 1-134 | Ph | N | —CH₂(CH₂)₂CH₂— | 4-OCH₃—Ph | Cl | H | H |
| 1-135 | Ph | N | —CH₂(CH₂)₂CH₂— | OH | Cl | H | H |
| 1-136 | Ph | N | —CH₂(CH₂)₂CH₂— | OCH₃ | Cl | H | H |
| 1-137 | Ph | N | —CH₂(CH₂)₂CH₂— | OCH₂CH₃ | Cl | H | H |
| 1-138 | Ph | N | —CH₂(CH₂)₂CH₂— | OCH₂CF₃ | Cl | H | H |
| 1-139 | Ph | N | —CH₂(CH₂)₂CH₂— | OCH₂CH₂CH₃ | Cl | H | H |
| 1-140 | Ph | N | —CH₂(CH₂)₂CH₂— | O(CH₂)₃CH₃ | Cl | H | H |
| 1-141 | Ph | N | —CH₂(CH₂)₂CH₂— | OCH(CH₃)₂ | Cl | H | H |
| 1-142 | Ph | N | —CH₂(CH₂)₂CH₂— | OC(CH₃)₃ | Cl | H | H |
| 1-143 | Ph | N | —CH₂(CH₂)₂CH₂— | OCH(CH₃)CH₂CH₃ | Cl | H | H |
| 1-144 | Ph | N | —CH₂(CH₂)₂CH₂— | OCH₂CHCH₂ | Cl | H | H |
| 1-145 | Ph | N | —CH₂(CH₂)₂CH₂— | OCH₂CH═C(Cl)CF₃ | Cl | H | H |
| 1-146 | Ph | N | —CH₂(CH₂)₂CH₂— | OCH₂CH(CH₃)CH₂CHCH₂ | Cl | H | H |
| 1-147 | Ph | N | —CH₂(CH₂)₂CH₂— | OPh | Cl | H | H |
| 1-148 | Ph | N | —CH₂(CH₂)₂CH₂— | OCH₂Ph | Cl | H | H |
| 1-149 | Ph | N | —CH₂(CH₂)₂CH₂— | ON═CHCH₃ | Cl | H | H |
| 1-150 | Ph | N | —CH₂(CH₂)₂CH₂— | ON═C(CH₃)₂ | Cl | H | H |
| 1-151 | Ph | N | —CH₂(CH₂)₂CH₂— | ON═C(CN)CH₃ | Cl | H | H |
| 1-152 | Ph | N | —CH₂(CH₂)₂CH₂— | ON═C(CH₃)C(CH₃)₃ | Cl | H | H |
| 1-153 | Ph | N | —CH₂(CH₂)₂CH₂— | 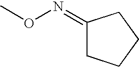 | Cl | H | H |
| 1-154 | Ph | N | —CH₂(CH₂)₂CH₂— | 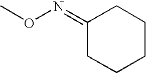 | Cl | H | H |
| 1-155 | Ph | N | —CH₂(CH₂)₂CH₂— | ON═(CH₃)Ph | Cl | H | H |
| 1-156 | Ph | N | —CH₂(CH₂)₂CH₂— | ON═CHPh | Cl | H | H |
| 1-157 | Ph | N | —CH₂(CH₂)₂CH₂— | ON═(CF₃)CH₃ | Cl | H | H |
| 1-158 | Ph | N | —CH₂(CH₂)₂CH₂— | ON═(CF₃)Ph | Cl | H | H |
| 1-159 | Ph | N | —CH₂(CH₂)₂CH₂— | ON═(CH₃)OCH₂CH₃ | Cl | H | H |
| 1-160 | Ph | N | —CH₂(CH₂)₂CH₂— | ON═(CH₃)CH₂CH(CH₃)₂ | Cl | H | H |
| 1-161 | Ph | N | —CH₂(CH₂)₂CH₂— | ON═(CH₃)CH₂C(CH₃)₃ | Cl | H | H |
| 1-162 | Ph | N | —CH₂(CH₂)₂CH₂— | NHOCH₃ | Cl | H | H |
| 1-163 | Ph | N | —CH₂(CH₂)₂CH₂— | NHOCH₂Ph | Cl | H | H |
| 1-164 | Ph | N | —CH₂(CH₂)₂CH₂— | NHOCH₂CH═CCl₂ | Cl | H | H |
| 1-165 | Ph | N | —CH₂(CH₂)₂CH₂— | SCH₃ | Cl | H | H |
| 1-166 | Ph | N | —CH₂(CH₂)₂CH₂— | SCH₂CH₃ | Cl | H | H |
| 1-167 | Ph | N | —CH₂(CH₂)₂CH₂— | SCH₂CH₂CH₃ | Cl | H | H |
| 1-168 | Ph | N | —CH₂(CH₂)₂CH₂— | S(CH₂)₃CH₃ | Cl | H | H |
| 1-169 | Ph | N | —CH₂(CH₂)₂CH₂— | SC(CH₃)₃ | Cl | H | H |
| 1-170 | Ph | N | —CH₂(CH₂)₂CH₂— | SCH(CH₃)₂ | Cl | H | H |
| 1-171 | Ph | N | —CH₂(CH₂)₂CH₂— | SCH(CH₃)CH₂CH₃ | Cl | H | H |
| 1-172 | Ph | N | —CH₂(CH₂)₂CH₂— | SCH₂CH═CH₂ | Cl | H | H |
| 1-173 | Ph | N | —CH₂(CH₂)₂CH₂— | SCH₂CH═C12 | Cl | H | H |
| 1-174 | Ph | N | —CH₂(CH₂)₂CH₂— | SOCH₃ | Cl | H | H |
| 1-175 | Ph | N | —CH₂(CH₂)₂CH₂— | SO₂CH₃ | Cl | H | H |
| 1-176 | Ph | N | —CH₂(CH₂)₂CH₂— | NHPh | Cl | H | H |
| 1-177 | Ph | N | —CH₂(CH₂)₂CH₂— | N3 | Cl | H | H |
| 1-178 | Ph | N | —CH₂(CH₂)₂CH₂— | NH₂ | Cl | H | H |
| 1-179 | Ph | N | —CH₂(CH₂)₂CH₂— | N(CH₃)₂ | Cl | H | H |
| 1-180 | Ph | N | —CH₂(CH₂)₂CH₂— | N(CH₂CH₃)₂ | Cl | H | H |
| 1-181 | Ph | N | —CH₂(CH₂)₂CH₂— | NHCH(CH₃)₂ | Cl | H | H |
| 1-182 | Ph | N | —CH₂(CH₂)₂CH₂— | NHCH₂CH₂OCH₃ | Cl | H | H |
| 1-183 | Ph | N | —CH₂(CH₂)₂CH₂— | NHCH₂CH₂N(CH₃)₂ | Cl | H | H |
| 1-184 | Ph | N | —CH₂(CH₂)₂CH₂— | NH₂CH₂Ph | Cl | H | H |
| 1-185 | Ph | N | —CH₂(CH₂)₂CH₂— | NHCN | Cl | H | H |
| 1-186 | Ph | N | —CH₂(CH₂)₂CH₂— | 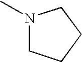 | Cl | H | H |
| 1-187 | Ph | N | —CH₂(CH₂)₂CH₂— | 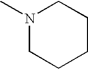 | Cl | H | H |
| 1-188 | Ph | N | —CH₂(CH₂)₂CH₂— | 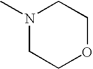 | Cl | H | H |
| 1-189 | Ph | N | —CH₂(CH₂)₂CH₂— | 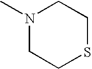 | Cl | H | H |
| 1-190 | | | | 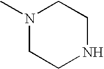 | | | |
| 1-191 | Ph | N | —CH₂(CH₂)₂CH₂— | NHC(═O)CH₃ | Cl | H | H |
| 1-192 | Ph | N | —CH₂(CH₂)₂CH₂— | NHC(═O)Ph | Cl | H | H |
| 1-193 | Ph | N | —CH₂(CH₂)₂CH₂— | NHC(═NOCH₃)H | Cl | H | H |
| 1-194 | Ph | N | —CH₂(CH₂)₂CH₂— | NHC(═NOCH₂Ph)H | Cl | H | H |
| 1-195 | Ph | N | —CH₂(CH₂)₂CH₂— | NHNH₂ | Cl | H | H |
| 1-196 | Ph | N | —CH₂(CH₂)₂CH₂— | N(CH₃)NH₂ | Cl | H | H |
| 1-197 | Ph | N | —CH₂(CH₂)₂CH₂— | NHNHCH₂CF₃ | Cl | H | H |
| 1-198 | Ph | N | —CH₂(CH₂)₂CH₂— | NHNHPh | Cl | H | H |
| 1-199 | Ph | N | —CH₂(CH₂)₂CH₂— | NHN═C(CH₃)₂ | Cl | H | H |
| 1-200 | Ph | N | —CH₂(CH₂)₂CH₂— | N(CH₃)N═C(CH₃)₂ | Cf | H | H |
| 1-201 | Ph | N | —CH₂(CH₂)₂CH₂— | N(CH₃)N═(Ph)CH₃ | Cl | H | H |
| 1-202 | Ph | N | —CH₂(CH₂)₂CH₂— | N(CH₃)N═CHPh | Cl | H | H |
| 1-203 | Ph | N | —CH₂(CH₂)₂CH₂— | N(CH₃)N═C(CH₃)CF₃ | Cl | H | H |
| 1-204 | Ph | N | —CH₂(CH₂)₂CH₂— | N(CH₃)N═C(Ph)CF₃ | Cl | H | H |
| 1-205 | Ph | N | —CH₂(CH₂)₂CH₂— | NHC(═NH)NHPh | Cl | H | H |
| 1-206 | Ph | N | —CH₂(CH₂)₂CH₂— | C(═NOH)NH₂ | Cl | H | H |
| 1-207 | Ph | N | —CH₂(CH₂)₂CH₂— | C(═NOCH₃)NH₂ | Cl | H | H |
| 1-208 | Ph | N | —CH₂(CH₂)₂CH₂— | CN | Cl | H | H |
| 1-209 | Ph | N | —CH₂(CH₂)₂CH₂— | 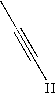 | Cl | H | H |
| 1-210 | Ph | N | —CH₂(CH₂)₂CH₂— | 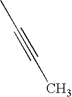 | Cl | H | H |
| 1-211 | Ph | N | —CH₂(CH₂)₂CH₂— | C(═O)CH₃ | Cl | H | H |
| 1-212 | Ph | N | —CH₂(CH₂)₂CH₂— | C(═O)CH₂CH₃ | Cl | H | H |
| 1-213 | Ph | N | —CH₂(CH₂)₂CH₂— | C(═O)CH(CH₃)₂ | Cl | H | H |
| 1-214 | Ph | N | —CH₂(CH₂)₂CH₂— | C(═O)CH₂CH₂CH₃ | Cl | H | H |
| 1-215 | Ph | N | —CH₂(CH₂)₂CH₂— | C(CH₃)═NOCH₃ | Cl | H | H |
| 1-216 | Ph | N | —CH₂(CH₂)₂CH₂— | C(CH₃)═NOCH₂CH₃ | Cl | H | H |
| 1-217 | Ph | N | —CH₂(CH₂)₂CH₂— | C(CH₃)═NOCH₂CH₂CH₃ | Cl | H | H |
| 1-218 | Ph | N | —CH₂(CH₂)₂CH₂— | C(CH₃)═NO(CH₂)₃CH₃ | Cl | H | H |
| 1-219 | Ph | N | —CH₂(CH₂)₂CH₂— | C(CH₂CH₃)NOCH₃ | Cl | H | H |
| 1-220 | Ph | N | —CH₂(CH₂)₂CH₂— | C(CH₃)═NOC(CH₃)₃ | Cl | H | H |
| 1-221 | Ph | N | —CH₂(CH₂)₂CH₂— | C(CH₃)═NOCH₂CHCH₂ | Cl | H | H |
| 1-222 | Ph | N | —CH₂(CH₂)₂CH₂— | C(CH₃)═NOCH₂CHCHCl | Cl | H | H |
| 1-223 | Ph | N | —CH₂(CH₂)₂CH₂— | C(CH₃)═NOCH₂CClCH₂ | Cl | H | H |
| 1-224 | Ph | N | —CH₂(CH₂)₂CH₂— | C(CH₃)═NOCH₂CHCl₂ | Cl | H | H |
| 1-225 | Ph | N | —CH₂(CH₂)₂CH₂— | C(CH₃)═NOCH₂CH₂OCH₃ | Cl | H | H |
| 1-226 | Ph | N | —CH₂(CH₂)₂CH₂— | C(CH₃)═NOCH₂CH(CH₃)OPh | Cl | H | H |
| 1-227 | Ph | N | —CH₂(CH₂)₂CH₂— | C(CH₃)═NOCH₂C(═O)OC(CH₃)₃ | Cl | H | H |
| 1-228 | Ph | N | —CH₂(CH₂)₂CH₂— | C(CH₃)═NNHCH(CH₃)₂ | Cl | H | H |
| 1-229 | Ph | N | —CH₂(CH₂)₂CH₂— | C(CH₃)═NNHPh | Cl | H | H |
| 1-230 | Ph | N | —CH₂(CH₂)₂CH₂— | C(CH₃)═NN(CH₃)Ph | Cl | H | H |
| 1-231 | Ph | N | —CH₂(CH₂)₂CH₂— | 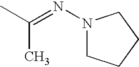 | Cl | H | H |
| 1-232 | Ph | N | —CH₂(CH₂)₂CH₂— | 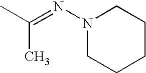 | Cl | H | H |
| 1-233 | Ph | N | —CH₂(CH₂)₂CH₂— | 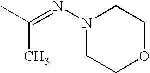 | Cl | H | H |
| 1-234 | Ph | N | —CH₂(CH₂)₂CH₂— | 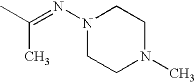 | Cl | H | H |
| 1-235 | Ph | N | —CH₂(CH₂)₂CH₂— | CH₃ | OCH₃ | H | H |
| 1-236 | Ph | N | —CH₂(CH₂)₂CH₂— | 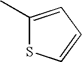 | Cl | H | H |
| 1-237 | Ph | N | —CH₂(CH₂)₂CH₂— | 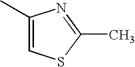 | Cl | H | H |
| 1-238 | Ph | N | —CH₂(CH₂)₂CH₂— | pyridin-2-yl | Cl | H | H |
| 1-239 | 1-NaPh | N | —CH₂(CH₂)₂CH₂— | pyridin-2-yl | Cl | H | H |
| 1-240 | 2-NaPh | N | —CH₂(CH₂)₂CH₂— | pyridin-2-yl | Cl | H | H |
| 1-241 | 2-F—Ph | N | —CH₂(CH₂)₂CH₂— | pyridin-2-yl | Cl | H | H |
| 1-242 | 2-Cl—Ph | N | —CH₂(CH₂)₂CH₂— | pyridin-2-yl | Cl | H | H |
| 1-243 | 2-Br—Ph | N | —CH₂(CH₂)₂CH₂ | pyridin-2-yl | Cl | H | H |
| 1-244 | 2-I—Ph | N | —CH₂(CH₂)₂CH₂ | pyridin-2-yl | Cl | H | H |
| 1-245 | 2-CH₃—Ph | N | —CH₂(CH₂)₂CH₂ | pyridin-2-yl | Cl | H | H |
| 1-246 | 2-CF₃—Ph | N | —CH₂(CH₂)₂CH₂— | pyridin-2-yl | Cl | H | H |
| 1-247 | 2-CN—Ph | N | —CH₂(CH₂)₂CH₂— | pyridin-2-yl | Cl | H | H |
| 1-248 | 2-OCH₃—Ph | N | —CH₂(CH₂)₂CH₂— | pyridin-2-yl | Cl | H | H |
| 1-249 | 2-NO₂—Ph | N | —CH₂(CH₂)₂CH₂ | pyridin-2-yl | Cl | H | H |
| 1-250 | 2-NH₂Ph | N | —CH₂(CH₂)₂CH₂ | pyridin-2-yl | Cl | H | H |
| 1-251 | 2-Ph—Ph | N | —CH₂(CH₂)₂CH₂ | pyridin-2-yl | Cl | H | H |
| 1-252 | 3-F—Ph | N | —CH₂(CH₂)₂CH₂— | pyridin-2-yl | Cl | H | H |
| 1-253 | 3-Cl—Ph | N | —CH₂(CH₂)₂CH₂— | pyridin-2-yl | Cl | H | H |
| 1-254 | 3-Br—Ph | N | —CH₂(CH₂)₂CH₂— | pyridin-2-yl | Cl | H | H |
| 1-255 | 3-I—Ph | N | —CH₂(CH₂)₂CH₂ | pyridin-2-yl | Cl | H | H |
| 1-256 | 3-CH3Ph | N | —CH₂(CH₂)₂CH₂ | pyridin-2-yl | Cl | H | H |
| 1-257 | 3-CF₃—Ph | N | —CH₂(CH₂)₂CH₂ | pyridin-2-yl | Cl | H | H |
| 1-258 | 3-CN—Ph | N | —CH₂(CH₂)₂CH₂— | pyridin-2-yl | Cl | H | H |
| 1-259 | 3-OCH₃—Ph | N | —CH₂(CH₂)₂CH₂— | pyridin-2-yl | Cl | H | H |
| 1-260 | 3-OCF₃—Ph | N | —CH₂(CH₂)₂CH₂— | pyridin-2-yl | Cl | H | H |
| 1-261 | 3-NO₂—Ph | N | —CH₂(CH₂)₂CH₂— | pyridin-2-yl | Cl | H | H |
| 1-262 | 3-NH₂—Ph | N | —CH₂(CH₂)₂CH₂— | pyridin-2-yl | Cl | H | H |
| 1-263 | 3-Ph—Ph | N | —CH₂(CH₂)₂CH₂— | pyridin-2-yl | Cl | H | H |
| 1-264 | 4-F—Ph | N | —CH₂(CH₂)₂CH₂— | pyridin-2-yl | Cl | H | H |
| 1-265 | 4-Cl—Ph | N | —CH₂(CH₂)₂CH₂— | pyridin-2-yl | Cl | H | H |
| 1-266 | 4-Br—Ph | N | —CH₂(CH₂)₂CH₂— | pyridin-2-yl | Cl | H | H |
| 1-267 | 4-CH₃—Ph | N | —CH₂(CH₂)₂CH₂— | pyridin-2-yl | Cl | H | H |
| 1-268 | 4-C(CH₃)₃—Ph | N | —CH₂(CH₂)₂CH₂— | pyridin-2-yl | Cl | H | H |
| 1-269 | 4-CF₃—Ph | N | —CH₂(CH₂)₂CH₂— | pyridin-2-yl | Cl | H | H |
| 1-270 | 4-CN—Ph | N | —CH₂(CH₂)₂CH₂— | pyridin-2-yl | Cl | H | H |
| 1-271 | 4-OCH₃—Ph | N | —CH₂(CH₂)₂CH₂— | pyridin-2-yl | Cl | H | H |
| 1-272 | 4-OCF₃—Ph | N | —CH₂(CH₂)₂CH₂— | pyridin-2-yl | Cl | H | H |
| 1-273 | 4-NO₂—Ph | N | —CH₂(CH₂)₂CH₂— | pyridin-2-yl | Cl | H | H |
| 1-274 | 4-NH₂—Ph | N | —CH₂(CH₂)₂CH₂— | pyridin-2-yl | Cl | H | H |
| 1-275 | 4-Ph—Ph | N | —CH₂(CH₂)₂CH₂— | pyridin-2-yl | Cl | H | H |
| 1-276 | 2-Cl-4-F—Ph | N | —CH₂(CH₂)₂CH₂— | pyridin-2-yl | Cl | H | H |
| 1-277 | 2-Cl-6-F—Ph | N | —CH₂(CH₂)₂CH₂— | pyridin-2-yl | Cl | H | H |
| 1-278 | 2,3-(F)₂—Ph | N | —CH₂(CH₂)₂CH₂— | pyriClin-2-yl | Cl | H | H |
| 1-279 | 2,4-(F)₂—Ph | N | —CH₂(CH₂)₂CH₂— | pyridin-2-yl | Cl | H | H |
| 1-280 | 2,5-(F)₂—Ph | N | —CH₂(CH₂)₂CH₂— | pyridin-2-yl | Cl | H | H |
| 1-281 | 2,6-(F)₂—Ph | N | —CH₂(CH₂)₂CH₂— | pyridin-2-yl | Cl | H | H |
| 1-282 | 3,4-(F)₂—Ph | N | —CH₂(CH₂)₂CH₂— | pyridin-2-yl | Cl | H | H |
| 1-283 | 3,5-(F)₂—Ph | N | —CH₂(CH₂)₂CH₂— | pyridin-2-yl | Cl | H | H |
| 1-284 | 2,3,4-(F)₃—Ph | N | —CH₂(CH₂)₂CH₂— | pyridin-2-yl | Cl | H | H |
| 1-285 | 2,3,6-(F)₃—Ph | N | —CH₂(CH₂)₂CH₂— | pyridin-2-yl | Cl | H | H |
| 1-286 | 2,3,6-(F)₃—Ph | N | —CH₂(CH₂)₂CH₂— | pyridin-2-yl | Cl | H | H |
| 1-287 | 2,3,4,5,6-(F)₅—Ph | N | —CH₂(CH₂)₂CH₂— | pyridin-2-yl | Cl | H | H |
| 1-288 | 2,3,4,5,6-(F)₅—Ph | N | —CH₂(CH₂)₂CH₂— | pyridin-2-yl | Cl | F | F |
| 1-289 | pyridin-2-yl | N | —CH₂(CH₂)₂CH₂— | pyridin-2-yl | Cl | H | H |
| 1-290 | 6-Cl-pyridin-2-yl | N | —CH₂(CH₂)₂CH₂— | pyridin-2-yl | Cl | H | H |
| 1-291 | 3-Cl-5-CF₃-pyridin-2-yl | N | —CH₂(CH₂)₂CH₂— | pyridin-2-yl | Cl | H | H |
| 1-292 | pyridin-3-yl | N | —CH₂(CH₂)₂CH₂— | pyridin-2-yl | Cl | H | H |
| 1-293 | 6-Cl-pyridin-3-yl | N | —CH₂(CH₂)₂CH₂— | pyridin-2-yl | Cl | H | H |
| 1-294 | pyridin-4-yl | N | —CH₂(CH₂)₂CH₂— | pyridin-2-yl | Cl | H | H |
| 1-295 | 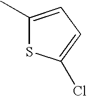 | N | —CH₂(CH₂)₂CH₂— | pyridin-2-yl | Cl | H | H |
| 1-296 | 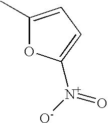 | N | —CH₂(CH₂)₂CH₂— | pyridin-2-yl | Cl | H | H |
| 1-297 | Ph | N | —CH₂(CH₂)₂CH₂— | pyridin-2-yl | H | H | H |
| 1-298 | Ph | N | —CH₂(CH₂)₂CH(CF₃)— | pyridin-2-yl | Cl | H | H |
| 1-299 | Ph | N | —CH₂(CH₂)₂CH₂— | 3-CH₃-pyridin-2-yl | Cl | H | H |
| 1-300 | Ph | N | —CH₂(CH₂)₂CH₂— | 4-CH₃-pyridin-2-yl | Cl | H | H |
| 1-301 | Ph | N | —CH₂(CH₂)₂CH₂— | 5-CF₃-pyridin-2-yl | Cl | H | H |
| 1-302 | Ph | N | —CH₂(CH₂)₂CH₂— | 6-CH₃-pyridin-2-yl | Cl | H | H |
| 1-303 | Ph | N | —CH₂(CH₂)₂CH₂— | pyridin-3-yl | Cl | H | H |
| 1-304 | Ph | N | —CH₂(CH₂)₂CH₂— | pyridin-4-yl | Cl | H | H |
| 1-305 | Ph | N | —CH₂(CH₂)₂CH₂— | 2,6-(Cl)₂-pyridin-4-yl | Cl | H | H |
| 1-306 | Ph | N | —CH₂(CH₂)₂CH₂— | 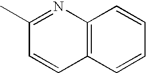 | Cl | H | H |
| 1-307 | Ph | N | —CH₂(CH₂)₂CH₂— | 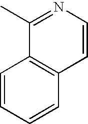 | Cl | H | H |
| 1-308 | Ph | N | —CH₂(CH₂)₂CH₂— | 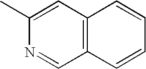 | Cl | H | H |
| 1-309 | Ph | N | —CH₂(CH₂)₂CH₂— | pyrimidin-2-yl | Cl | H | H |
| 1-310 | Ph | N | —CH₂(CH₂)₂CH₂— | pyridazin-3-yl | Cl | H | H |
| 1-311 | Ph | N | —CH₂(CH₂)₂CH₂— | pyrazin-2-yl | Cl | H | H |
| 1-312 | 1-NaPh | N | —CH₂(CH₂)₂CH₂— | pyrazin-2-yl | Cl | H | H |
| 1-313 | 2-NaPh | N | —CH₂(CH₂)₂CH₂— | pyrazin-2-yl | Cl | H | H |
| 1-314 | 2-F—Ph | N | —CH₂(CH₂)₂CH₂— | pyrazin-2-yl | Cl | H | H |
| 1-315 | 2-Cl—Ph | N | —CH₂(CH₂)₂CH₂— | pyrazin-2-yl | Cl | H | H |
| 1-316 | 2-Br—Ph | N | —CH₂(CH₂)₂CH₂— | pyrazin-2-yl | Cl | H | H |
| 1-317 | 2-I—Ph | N | —CH₂(CH₂)₂CH₂— | pyrazin-2-yl | Cl | H | H |
| 1-318 | 2-CH₃—Ph | N | —CH₂(CH₂)₂CH₂— | pyrazin-2-yl | Cl | H | H |
| 1-319 | 2-CF₃—Ph | N | —CH₂(CH₂)₂CH₂— | pyrazin-2-yl | Cl | H | H |
| 1-320 | 2-CN—Ph | N | —CH₂(CH₂)₂CH₂— | pyrazin-2-yl | Cl | H | H |
| 1-321 | 2-OCH₃—Ph | N | —CH₂(CH₂)₂CH₂— | pyrazin-2-yl | Cl | H | H |
| 1-322 | 2-NO₂—Ph | N | —CH₂(CH₂)₂CH₂— | pyrazin-2-yl | Cl | H | H |
| 1-323 | 2-NH₂—Ph | N | —CH₂(CH₂)₂CH₂— | pyrazin-2-yl | Cl | H | H |
| 1-324 | 2-Ph—Ph | N | —CH₂(CH₂)₂CHr | pyrazin-2-yl | Cl | H | H |
| 1-325 | 3-F—Ph | N | —CH₂(CH₂)₂CH₂— | pyrazin-2-yl | Cl | H | H |
| 1-326 | 3-Cl—Ph | N | —CH₂(CH₂)₂CH₂— | pyrazin-2-yl | Cl | H | H |
| 1-327 | 3-Br—Ph | N | —CH₂(CH₂)₂CH₂— | pyrazin-2-yl | Cl | H | H |
| 1-328 | 3-I—Ph | N | —CH₂(CH₂)₂CH₂— | pyrazin-2-yl | Cl | H | H |
| 1-329 | 3-CH₃—Ph | N | —CH₂(CH₂)₂CH₂— | pyrazin-2-yl | Cl | H | H |
| 1-330 | 3-CF₃—Ph | N | —CH₂(CH₂)₂CH₂— | pyrazin-2-yl | Cl | H | H |
| 1-331 | 3-CN—Ph | N | —CH₂(CH₂)₂CH₂— | pyrazin-2-yl | Cl | H | H |
| 1-332 | 3-OCH₃—Ph | N | —CH₂(CH₂)₂CH₂— | pyrazin-2-yl | Cl | H | H |
| 1-333 | 3-OCF₃—Ph | N | —CH₂(CH₂)₂CH₂— | pyrazin-2-yl | Cl | H | H |
| 1-334 | 3-NO₂Ph | N | —CH₂(CH₂)₂CH₂— | pyrazin-2-yl | Cl | H | H |
| 1-335 | 3-NH₂—Ph | N | —CH₂(CH₂)₂CH₂— | pyrazin-2-yl | Cl | H | H |
| 1-336 | 3-Ph—Ph | N | —CH₂(CH₂)₂CH₂— | pyrazin-2-yl | Cl | H | H |
| 1-337 | 4-F—Ph | N | —CH₂(CH₂)₂CH₂— | pyrazin-2-yl | Cl | H | H |
| 1-338 | 4-Cl—Ph | N | —CH₂(CH₂)₂CHz | pyrazin-2-yl | Cl | H | H |
| 1-339 | 4-Br—Ph | N | —CH₂(CH₂)₂CH₂— | pyrazin-2-yl | Cl | H | H |
| 1-340 | 4-CH3Ph | N | —CH₂(CH₂)₂CH₂— | pyrazin-2-yl | Cl | H | H |
| 1-341 | 4-C(CH₃)₃Ph | N | —CH₂(CH₂)₂CH₂— | pyrazin-2-yl | Cl | H | H |
| 1-342 | 4-CF3Ph | N | —CH₂(CH₂)₂CH₂— | pyrazin-2-yl | Cl | H | H |
| 1-343 | 4-CN—Ph | N | —CH₂(CH₂)₂CH₂— | pyrazin-2-yl | Cl | H | H |
| 1-344 | 4-OCH3Ph | N | —CH₂(CH₂)₂CH₂— | pyrazin-2-yl | Cl | H | H |
| 1-345 | 4-OCF₃—Ph | N | —CH₂(CH₂)₂CH₂— | pyrazin-2-yl | Cl | H | H |
| 1-346 | 4-NO₂—Ph | N | —CH₂(CH₂)₂CH₂— | pyrazin-2-yl | Cl | H | H |
| 1-347 | 4-NH₂Ph | N | —CH₂(CH₂)₂CH₂— | pyrazin-2-yl | Cl | H | H |
| 1-348 | 4-Ph—Ph | N | —CH₂(CH₂)₂CH₂— | pyrazin-2-yl | Cl | H | H |
| 1-349 | 2-Cl-4-F—Ph | N | —CH₂(CH₂)₂CH₂— | pyrazin-2-yl | Cl | H | H |
| 1-350 | 2-Cl-6-F—Ph | N | —CH₂(CH₂)₂CH₂— | pyrazin-2-yl | Cl | H | H |
| 1-351 | 2,3-(F)₂—Ph | N | —CH₂(CH₂)₂CH₂— | pyrazin-2-yl | Cl | H | H |
| 1-352 | 2,4-(F)₂—Ph | N | —CH₂(CH₂)₂CH₂— | pyrazin-2-yl | Cl | H | H |
| 1-353 | 2,5-(F)₂—Ph | N | —CH₂(CH₂)₂CH₂— | pyrazin-2-yl | Cl | H | H |
| 1-354 | 2,6-(F)₂—Ph | N | —CH₂(CH₂)₂CH₂— | pyrazin-2-yl | Cl | H | H |
| 1-355 | 3,4-(F)₂—Ph | N | —CH₂(CH₂)₂CH₂— | pyrazin-2-yl | Cl | H | H |
| 1-356 | 3,5-(F)₂—Ph | N | —CH₂(CH₂)₂CH₂— | pyrazin-2-yl | Cl | H | H |
| 1-357 | 2,3,4-(F)₃Ph | N | —CH₂(CH₂)₂CH₂— | pyrazin-2-yl | Cl | H | H |
| 1-358 | 2,3,6-(F)₃Ph | N | —CH₂(CH₂)₂CH₂— | pyrazin-2-yl | Cl | H | H |
| 1-359 | 2,3,6-(F)₃—Ph | N | —CH₂(CH₂)₂CH₂— | pyrazin-2-yl | Cl | H | H |
| 1-360 | 2,3,4,5,6-(F)₅—Ph | N | —CH₂(CH₂)₂CH₂— | pyrazin-2-yl | Cl | H | H |
| 1-361 | 2,3,4,5,6(F)₅Ph | N | —CH₂(CH₂)₂CH₂— | pyrazin-2-yl | Cl | F | F |
| 1-362 | pyridin-2-yl | N | —CH₂(CH₂)₂CH₂— | pyrazin-2-yl | Cl | H | H |
| 1-363 | 6-Cl-pyridin-2-yl | N | —CH₂(CH₂)₂CH₂— | pyrazin-2-yl | Cl | H | H |
| 1-364 | 3-Cl-5-CF₃-pyridin-2-yl | N | —CH₂(CH₂)₂CH₂— | pyrazin-2-yl | Cl | H | H |
| 1-365 | pyridin-3-yl | N | —CH₂(CH₂)₂CH₂— | pyrazin-2-yl | Cl | H | H |
| 1-366 | 6-Cl-pyridin-3-yl | N | —CH₂(CH₂)₂CH₂— | pyrazin-2-yl | Cl | H | H |
| 1-367 | pyridin-4-yl | N | —CH₂(CH₂)₂CH₂— | pyrazin-2-yl | Cl | H | H |
| 1-368 | 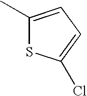 | N | —CH₂(CH₂)₂CH₂— | pyrazin-2-yl | Cl | H | H |
| 1-369 | 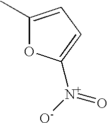 | N | —CH₂(CH₂)₂CH₂— | pyrazin-2-yl | Cl | H | H |
| 1-370 | 1-NaPh | N | —CH₂(CH₂)₂CH₂— | pyrazol-1-yl | Cl | H | H |
| 1-371 | 2-NaPh | N | —CH₂(CH₂)₂CH₂— | pyrazol-1-yl | Cl | H | H |
| 1-372 | 2-F—Ph | N | —CH₂(CH₂)₂CH₂— | pyrazol-1-yl | Cl | H | H |
| 1-373 | 2-Cl—Ph | N | —CH₂(CH₂)₂CH₂— | pyrazol-1-yl | Cl | H | H |
| 1-374 | 2-Br—Ph | N | —CH₂(CH₂)₂CH₂— | pyrazol-1-yl | Cl | H | H |
| 1-375 | 2-I—Ph | N | —CH₂(CH₂)₂CH₂— | pyrazol-1-yl | Cl | H | H |
| 1-376 | 2-CH3Ph | N | —CH₂(CH₂)₂CH₂— | pyrazol-1-yl | Cl | H | H |
| 1-377 | 2-CF₃—Ph | N | —CH₂(CH₂)₂CH₂— | pyrazol-1-yl | Cl | H | H |
| 1-378 | 2-CN—Ph | N | —CH₂(CH₂)₂CH₂— | pyrazol-1-yl | Cl | H | H |
| 1-379 | 2-OCH₃—Ph | N | —CH₂(CH₂)₂CH₂— | pyrazol-1-yl | Cl | H | H |
| 1-380 | 2-NO₂—Ph | N | —CH₂(CH₂)₂CH₂— | pyrazol-1-yl | Cl | H | H |
| 1-381 | 2-NH₂—Ph | N | —CH₂(CH₂)₂CH₂— | pyrazol-1-yl | Cl | H | H |
| 1-382 | 2-Ph—Ph | N | —CH₂(CH₂)₂CH₂— | pyrazol-1-yl | Cl | H | H |
| 1-383 | 3-F—Ph | N | —CH₂(CH₂)₂CH₂— | pyrazol-1-yl | Cl | H | H |
| 1-384 | 3-Cl—Ph | N | —CH₂(CH₂hCH₂— | pyrazol-1-yl | Cl | H | H |
| 1-385 | 3-Br—Ph | N | —CH₂(CH₂)₂CH₂— | pyrazol-1-yl | Cl | H | H |
| 1-386 | 3-I—Ph | N | —CH₂(CH₂)₂CH₂— | pyrazol-1-yl | Cl | H | H |
| 1-387 | 3-CH₃—Ph | N | —CH₂(CH₂)₂CH₂— | pyrazol-1-yl | Cl | H | H |
| 1-388 | 3-CF₃—Ph | N | —CH₂(CH₂)₂CH₂— | pyrazol-1-yl | Cl | H | H |
| 1-389 | 3-CN—Ph | N | —CH₂(CH₂)₂CH₂— | pyrazol-1-yl | Cl | H | H |
| 1-390 | 3-OCH₃—Ph | N | —CH₂(CH₂)₂CH₂— | pyrazol-1-yl | Cl | H | H |
| 1-391 | 3-OCF₃—Ph | N | —CH₂(CH₂)₂CH₂— | pyrazol-1-yl | Cl | H | H |
| 1-392 | 3-NO₂—Ph | N | —CH₂(CH₂)₂CH₂— | pyrazol-1-yl | Cl | H | H |
| 1-393 | 3-NH₂—Ph | N | —CH₂(CH₂)₂CH₂— | pyrazol-l-yl | Cl | H | H |
| 1-394 | 3-Ph—Ph | N | —CH₂(CH₂)₂CH₂— | pyrazol-1-yl | Cl | H | H |
| 1-395 | 4-F—Ph | N | —CH₂(CH₂)₂CH₂— | pyrazol-1-yl | Cl | H | H |
| 1-396 | 4-Cl—Ph | N | —CH₂(CH₂)₂CH₂— | pyrazol-1-yl | Cl | H | H |
| 1-397 | 4-Br—Ph | N | —CH₂(CH₂)₂CH₂— | pyrazol-1-yl | Cl | H | H |
| 1-398 | 4-CH₃—Ph | N | —CH₂(CH₂)₂CH₂— | pyrazol-1-yl | Cl | H | H |
| 1-399 | 4-C(CH₃)₃—Ph | N | —CH₂(CH₂)₂CH₂— | pyrazol-1-yl | Cl | H | H |
| 1-400 | 4-CF₃—Ph | N | —CH₂(CH₂)₂CH₂— | pyrazol-1-yl | Cl | H | H |
| 1-401 | 4-CN—Ph | N | —CH₂(CH₂)₂CH₂— | pyrazol-1-yl | Cl | H | H |
| 1-402 | 4-OCH₃—Ph | N | —CH₂(CH₂)₂CH₂— | pyrazol-1-yl | Cl | H | H |
| 1-403 | 4-OCF₃—Ph | N | —CH₂(CH₂)₂CH₂— | pyrazol-1-yl | Cl | H | H |
| 1-404 | 4-NO₂—Ph | N | —CH₂(CH₂)₂CH₂— | pyrazol-1-yl | Cl | H | H |
| 1-405 | 4-NH₂—Ph | N | —CH₂(CH₂)₂CH₂— | pyrazol-1-yl | Cl | H | H |
| 1-406 | 4-Ph—Ph | N | —CH₂(CH₂)₂CH₂— | pyrazol-l-yl | Cl | H | H |
| 1-407 | 2-Cl-4-F—Ph | N | —CH₂(CH₂)₂CH₂— | pyrazol-1-yl | Cl | H | H |
| 1-408 | 2-Cl-6-F—Ph | N | —CH₂(CH₂)₂CH₂— | pyrazol-1-yl | Cl | H | H |
| 1-409 | 2,3-(F)₂—Ph | N | —CH₂(CH₂)₂CH₂— | pyrazol-1-yl | Cl | H | H |
| 1-410 | 2,4(F)₂—Ph | N | —CH₂(CH₂)₂CH₂— | pyrazol-1-yl | Cl | H | H |
| 1-411 | 2,5(F)₂—Ph | N | —CH₂(CH₂)₂CH₂— | pyrazol-1-yl | Cl | H | H |
| 1-412 | 2,6-(F)₂—Ph | N | —CH₂(CH₂)₂CH₂— | pyrazol-1-yl | Cl | H | H |
| 1-413 | 3,4-(F)₂—Ph | N | —CH₂(CH₂)₂CH₂— | pyrazol-1-yl | Cl | H | H |
| 1-414 | 3,5-(F)₂—Ph | N | —CH₂(CH₂)₂CH₂— | pyrazol-1-yl | Cl | H | H |
| 1-415 | 2,3,4-(F)₃—Ph | N | —CH₂(CH₂)₂CH₂— | pyrazol-1-yl | Cl | H | H |
| 1-416 | 2,3,6-(F)₃—Ph | N | —CH₂(CH₂)₂CH₂— | pyrazol-1-yl | Cl | H | H |
| 1-417 | 2,3,6-(F)₃—Ph | N | —CH₂(CH₂)₂CH₂— | pyrazol-1-yl | Cl | H | H |
| 1-418 | 2,3,4,5,6-(F)₅—Ph | N | —CH₂(CH₂)₂CH₂— | pyrazol-1-yl | Cl | H | H |
| 1-419 | 2,3,4,5,6-(F)₅—Ph | N | —CH₂(CH₂)₂CH₂— | pyrazol-1-yl | Cl | F | F |
| 1-420 | pyridin-2-yl | N | —CH₂(CH₂)₂CH₂— | pyrazol-1-yl | Cl | H | H |
| 1-421 | 6-Cl-pyridin-2-yl | N | —CH₂(CH₂)₂CH₂— | pyrazol-1-yl | Cl | H | H |
| 1-422 | 3-Cl-5-CF₃-pyridin-2-yl | N | —CH₂(CH₂)₂CH₂— | pyrazol-1-yl | Cl | H | H |
| 1-423 | pyridin-3-yl | N | —CH₂(CH₂)₂CH₂— | pyrazol-1-yl | Cl | H | H |
| 1-424 | 6-Cl-pyridin-3-yl | N | —CH₂(CH₂)₂CH₂— | pyrazol-1-yl | Cl | H | H |
| 1-425 | pyridin-4-yl | N | —CH₂(CH₂)₂CH₂— | pyrazol-1-yl | Cl | H | H |
| 1-426 | 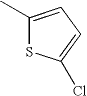 | N | —CH₂(CH₂)₂CH₂— | pyrazol-1-yl | Cl | H | H |
| 1-427 | 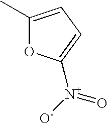 | N | —CH₂(CH₂)₂CH₂— | pyrazol-1-yl | Cl | H | H |
| 1-428 | Ph | N | —CH₂(CH₂)₂CH₂— | 3,5-(CH₃)₂-pyrazol-1-yl | Cl | H | H |
| 1-429 | Ph | N | —CH₂(CH₂)₂CH₂— | 3-CF₃-pyrazol-1-yl | Cl | H | H |
| 1-430 | Ph | N | —CH₂(CH₂)₂CH₂— | 3,5-(CF₃)₂-pyrazol-1-yl | Cl | H | H |
| 1-431 | Ph | N | —CH₂(CH₂)₂CH₂— | 4-Br-pyrazol-1-yl | Cl | H | H |
| 1-432 | Ph | N | —CH₂(CH₂)₂CH₂— | 4-CH₃-pyrazol-1-yl | Cl | H | H |
| 1-433 | Ph | N | —CH₂(CH₂)₂CH₂— | imidazol-1-yl | Cl | H | H |
| 1-434 | Ph | N | —CH₂(CH₂)₂CH₂— | 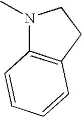 | Cl | H | H |
| 1-435 | Ph | N | —CH₂(CH₂)₂CH₂— | 1,2,4-triazol-1-yl | Cl | H | H |
| 1-436 | 1-NaPh | N | —CH₂(CH₂)₂CH₂— | 1,2,4-triazol-1-yl | Cl | H | H |
| 1-437 | 2-NaPh | N | —CH₂(CH₂)₂CH₂— | 1,2,4-triazol-1-yl | Cl | H | H |
| 1-438 | 2-F—Ph | N | —CH₂(CH₂)₂CH₂— | 1,2,4-triazol-1-yl | Cl | H | H |
| 1-439 | 2-Cl—Ph | N | —CH₂(CH₂)₂CH₂— | 1,2,4-triazol-1-yl | Cl | H | H |
| 1-440 | 2-Br—Ph | N | —CH₂(CH₂)₂CH₂— | 1,2,4-triazol-1-yl | Cl | H | H |
| 1-441 | 2-1—Ph | N | —CH₂(CH₂)₂CH₂— | 1,2,4-triazol-1-yl | Cl | H | H |
| 1-442 | 2-CH₃—Ph | N | —CH₂(CH₂)₂CH₂— | 1,2,4-triazol-1-yl | Cl | H | H |
| 1-443 | 2-CF₃—Ph | N | —CH₂(CH₂)₂CH₂— | 1,2,4-triazol-1-yl | Cl | H | H |
| 1-444 | 2-CN—Ph | N | —CH₂(CH₂)₂CH₂— | 1,2,4-triazol-1-yl | Cl | H | H |
| 1-445 | 2-OCH₃—Ph | N | —CH₂(CH₂)₂CH₂— | 1,2,4-triazol-1-yl | Cl | H | H |
| 1-446 | 2-NO₂—Ph | N | —CH₂(CH₂)₂CH₂— | 1,2,4-triazol-1-yl | Cl | H | H |
| 1-447 | 2-NH₂—Ph | N | —CH₂(CH₂)₂CH₂— | 1,2,4-triazol-1-yl | C | | |
| | H | H | | | | | |
| 1-448 | 2-Ph—Ph | N | —CH₂(CH₂)₂CH₂— | 1,2,4-triazol-1-yl | Cl | H | H |
| 1-449 | 3-F—Ph | N | —CH₂(CH₂)₂CH₂— | 1,2,4-triazol-1-yl | Cl | H | H |
| 1-450 | 3-Cl—Ph | N | —CH₂(CH₂)₂CH₂— | 1,2,4-triazol-1-yl | Cl | H | H |
| 1-451 | 3-Br—Ph | N | —CH₂(CH₂)₂CH₂— | 1,2,4-triazol-1-yl | Cl | H | H |
| 1-452 | 3-I—Ph | N | —CH₂(CH₂)₂CH₂— | 1,2,4-triazol-1-yl | Cl | H | H |
| 1-453 | 3-CH₃—Ph | N | —CH₂(CH₂)₂CH₂— | 1,2,4-triazol-1-yl | Cl | H | H |
| 1-454 | 3-CF₃—Ph | N | —CH₂(CH₂)₂CH₂— | 1,2,4-triazol-1-yl | Cl | H | H |
| 1-455 | 3-CN—Ph | N | —CH₂(CH₂)₂CH₂— | 1,2,4-triazol-1-yl | Cl | H | H |
| 1-456 | 3-OCH₃—Ph | N | —CH₂(CH₂)₂CH₂— | 1,2,4-triazol-1-yl | Cl | H | H |
| 1-457 | 3-OCF₃—Ph | N | —CH₂(CH₂)₂CH₂— | 1,2,4-triazol-1-yl | Cl | H | H |
| 1-458 | 3-NO₂—Ph | N | —CH₂(CH₂)₂CH₂— | 1,2,4-triazol-1-yl | Cl | H | H |
| 1-459 | 3-NH₂—Ph | N | —CH₂(CH₂)₂CH₂— | 1,2,4-triazol-1-yl | Cl | H | H |
| 1-460 | 3-Ph—Ph | N | —CH₂(CH₂)₂CH₂— | 1,2,4-triazol-1-yl | Cl | H | H |
| 1-461 | 4-F—Ph | N | —CH₂(CH₂)₂CH₂— | 1,2,4-triazol-1-yl | Cl | H | H |
| 1-462 | 4-Cl—Ph | N | —CH₂(CH₂)₂CH₂— | 1,2,4-triazol-1-yl | Cl | H | H |
| 1-463 | 4-Br—Ph | N | —CH₂(CH₂)₂CH₂— | 1,2,4-triazol-1-yl | Cl | H | H |
| 1-464 | 4-CH₃—Ph | N | —CH₂(CH₂)₂CH₂— | 1,2,4-triazol-1-yl | Cl | H | H |
| 1-465 | 4-C(CH₃)₃—Ph | N | —CH₂(CH₂)₂CH₂— | 1,2,4-triazol-1-yl | Cl | H | H |
| 1-466 | 4-CF3Ph | N | —CH₂(CH₂)₂CH₂— | 1,2,4-triazol-1-yl | Cl | H | H |
| 1-467 | 4-CN—Ph | N | —CH₂(CH₂)₂CH₂— | 1,2,4-triazol-1-yl | Cl | H | H |
| 1-468 | 4-OCH₃—Ph | N | —CH₂(CH₂)₂CH₂— | 1,2,4-triazol-1-yl | Cl | H | H |
| 1-469 | 4-OCF₃—Ph | N | —CH₂(CH₂)₂CH₂— | 1,2,4-triazol-1-yl | Cl | H | H |
| 1-470 | 4-NO₂—Ph | N | —CH₂(CH₂)₂CH₂— | 1,2,4-triazol-1-yl | Cl | H | H |
| 1-471 | 4-NH₂—Ph | N | —CH₂(CH₂)₂CH₂— | 1,2,4-triazol-1-yl | Cl | H | H |
| 1-472 | 4-Ph—Ph | N | —CH₂(CH₂)₂CH₂— | 1,2,4-triazol-1-yl | Cl | H | H |
| 1-473 | 2-Cl-4-F—Ph | N | —CH₂(CH₂)₂CH₂— | 1,2,4-triazol-1-yl | Cl | H | H |
| 1-474 | 2-Cl-6-F—Ph | N | —CH₂(CH₂)₂CH₂— | 1,2,4-triazol-1-yl | Cl | H | H |
| 1-475 | 2,3-(F)₂—Ph | N | —CH₂(CH₂)₂CH₂— | 1,2,4-triazol-1-yl | Cl | H | H |
| 1-476 | 2,4-(F)₂—Ph | N | —CH₂(CH₂)₂CH₂— | 1,2,4-triazol-1-yl | Cl | H | H |
| 1-477 | 2,5-(F)₂—Ph | N | —CH₂(CH₂)₂CH₂— | 1,2,4-triazol-1-yl | Cl | H | H |
| 1-478 | 2~6-(F)₂—Ph | N | —CH₂(CH₂)₂CH₂— | 1,2,4-triazol-1-yl | Cl | H | H |
| 1-479 | 3,4-(F)₂—Ph | N | —CH₂(CH₂)₂CH₂— | 1,2,4-triazol-1-yl | Cl | H | H |
| 1-480 | 3,5-(F)₂—Ph | N | —CH₂(CH₂)₂CH₂— | 1,2,4-triazol-1-yl | Cl | H | H |
| 1-481 | 2,3,4-(F)₃—Ph | N | —CH₂(CH₂)₂CH₂— | 1,2,4-triazol-1-yl | Cl | H | H |
| 1-482 | 2,3,6-(F)₃—Ph | N | —CH₂(CH₂)₂CH₂— | 1,2,4-triazol-1-yl | Cl | H | H |
| 1-483 | 2,3,6-(F)₃—Ph | N | —CH₂(CH₂)₂CH₂— | 1,2,4-triazol-1-yl | Cl | H | H |
| 1-484 | 2,3,4,5,6-(F)₅—Ph | N | —CH₂(CH₂)₂CH₂— | 1,2,4-triazol-1-yl | Cl | H | H |
| 1-485 | 2,3,4,5,6-(F)₅—Ph | N | —CH₂(CH₂)₂CH₂— | 1,2,4-triazol-1-yl | Cl | F | F |
| 1-486 | pyridin-2-yl | N | —CH₂(CH₂)₂CH₂— | 1,2,4-triazol-1-yl | Cl | H | H |
| 1-487 | 6-Cl-pylidin-2-yl | N | —CH₂(CH₂)₂CH₂— | 1,2,4-triazol-1-yl | Cl | H | H |
| 1-488 | 3-Cl-5-CF₃-pyridin-2-yl | N | —CH₂(CH₂)₂CH₂— | 1,2,4-triazol-1-yl | Cl | H | H |
| 1-489 | pyridin-3-yl | N | —CH₂(CH₂)₂CH₂— | 1,2,4-triazol-1-yl | Cl | H | H |
| 1-490 | 6-Cl-pyridin-3-yl | N | —CH₂(CH₂)₂CH₂— | 1,2,4-triazol-1-yl | Cl | H | H |
| 1-491 | pyridin-4-yl | N | —CH₂(CH₂)₂CH₂— | 1,2,4-triazol-1-yl | Cl | H | H |
| 1-492 | 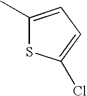 | N | —CH₂(CH₂)₂CH₂— | 1,2,4-triazol-1-yl | Cl | H | H |
| 1-493 | 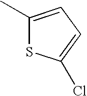 | N | —CH₂(CH₂)₂CH₂— | 1,2,4-triazol-1-yl | Cl | H | H |
| 0 | | | | | | | |
| 1-494 | Ph | N | —CH₂(CH₂)₂CH₂— | 1,2,4-triazol-1-yl | H | H | H |
| 1-495 | Ph | N | —CH₂(CH₂)₂CH₂— | 1,2,3-triazol-1-yl | Cl | H | H |
| 1-496 | Ph | N | —CH₂(CH₂)₂CH₂— | 1,2,5-triazol-1-yl | Cl | H | H |
| 1-497 | Ph | N | —CH₂(CH₂)₂CH₂— | tetrazol-1-yl | Cl | H | H |
| 1-498 | Ph | N | —CH₂(CH₂)₂CH₂— | tetrazol-2-yl | Cl | H | H |

| | | | | | | |
|---|---|---|---|---|---|---|
| 1-499 | Ph | pyrrol-1-yl | H | Cl | H | H |
| 1-500 | Ph | 2-C(═O)CF₃-pyrrol-1-yl | H | Cl | H | H |
| 1-501 | Ph | 2-C(═O)Cl3-pyrrol-1-yl | H | Cl | H | H |
| 1-502 | Ph | 2-CN-pyrrol-1-yl | H | Cl | H | H |
| 1-503 | Ph | 3-CH₃-pyrrol-1-yl | H | Cl | H | H |
| 1-504 | Ph | 2,4—(CH₃)₂-pyrrol-1-yl | H | Cl | H | H |
| 1-505 | Ph | pyrazol-1-yl | H | Cl | H | H |
| 1-506 | 3-F—Ph | pyrazol-1-yl | H | Cl | H | H |
| 1-507 | 3-F—Ph | pyrazol-1-yl | pyrazol-1-yl | Cl | H | H |
| 1-508 | Ph | pyrazol-1-yl | pyridin-2-yl | Cl | H | H |
| 1-509 | Ph | pyrazol-1-yl | pyrazin-2-yl | Cl | H | H |
| 1-510 | Ph | pyrazol-1-yl | pyrazol-1-yl | Cl | H | H |
| 1-511 | Ph | pyrazol-1-yl | 1,2,4-triazol-1-yl | Cl | H | H |
| 1-512 | Ph | imidazol-1-yl | H | Cl | H | H |
| 1-513 | Ph | imidazol-1-yl | pyridin-2-yl | Cl | H | H |
| 1-514 | Ph | imidazol-1-yl | pyrazin-2-yl | Cl | H | H |
| 1-515 | Ph | imidazol-1-yl | pyrazol-1-yl | Cl | H | H |
| 1-516 | Ph | imidazol-1-yl | 1,2,4-triazol-1-yl | Cl | H | H |
| 1-517 | Ph | 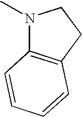 | H | Cl | H | H |
| 1-518 | Ph | 3-CF₃-pyrazol-1-yl | H | Cl | H | H |
| 1-519 | 3-CF₃—Ph | 3-CF₃-pyrazol-1-yl | H | Cl | H | H |
| 1-520 | 3-CF₃—Ph | 3-CF₃-pyrazol-1-yl | H | 3-CF₃-pyrazol-1-yl | H | H |
| 1-521 | Ph | 3,5-(CH₃)₂-pyrazol-1-yl | H | Cl | H | H |
| 1-522 | 3-F—Ph | 3,5-(CH₃)₂-pyrazol-1-yl | H | Cl | H | H |
| 1-523 | Ph | 3~5-(CH₃)₂-pyrazol-1-yl | CH₃ | Cl | H | H |
| 1-524 | Ph | 35-(CH₃)₂-pyrazol-1-yl | CH₃ | 3,5-(CH₃)₂-pyrazoyl-1 | H | H |
| 1-525 | Ph | 3-C 5.GF~pyr~oI-1-yl | H | Cl | H | H |
| 1-526 | Ph | 3,5-(CF₃)₂-pyrazol-1-yl | H | Cl | N | N |
| 1-527 | Ph | 3-CF3-5-OCH₃-pyrazol-1-yl | H | Cl | H | H |
| 1-528 | Ph | 4-Br-pyrazol-1-yl | H | Cl | H | H |
| 1-529 | Ph | 4-CH₃-pyrazol-1-yl | H | Cl | H | H |
| 1-530 | Ph | 1,2,3-triazol-1-yl | H | Cl | H | H |
| 1-531 | Ph | 1,2,4-triazol-1-yl | H | Cl | H | H |
| 1-532 | 3-F—Ph | 1,2,4-triazol-1-yl | H | Cl | H | H |
| 1-533 | Ph | 1,2,4-triazol-1-yl | pyridyl-2 | Cl | H | H |
| 1-534 | Ph | 1,2,4-triazol-1-yl | pyrazin-2-yl | Cl | H | H |
| 1-535 | Ph | 1,2,4-triazol-l-yl | pyrazol-1-yl | Cl | H | H |
| 1-536 | Ph | 1,2,4-triazol-1-yl | 1,2,4-triazol-1-yl | Cl | H | H |
| 1-537 | Ph | 3-SCH3-1,2,4-triazol-1-yl | H | Cl | H | H |
| 1-538 | Ph | 3-SCH₂Ph-1,2,4-triazol-1-yl | H | Cl | H | H |
| 1-539 | Ph | 1,2,5-triazol-1-yl | H | Cl | H | H |
| 1-540 | Ph | 1,3,4-triazol-1-yl | H | Cl | H | H |
| 1-541 | Ph | tetrazol-1-yl | H | Cl | H | H |
| 1-542 | Ph | tetrazol-2-yl | H | Cl | H | H |

| | | | | | | | | |
|---|---|---|---|---|---|---|---|---|
| 1-543 | Ph | N | —CH₂(CH₂)₃CH₂— | H | Cl | H | H | |
| 1-544 | Ph | N | —CH₂(CH₂)4CH₂— | H | Cl | H | H | |
| 1-545 | 3,5-(F)₂—Ph | N | —CH₂(CH₂)4CH₂— | H | Cl | H | H | |
| 1-546 | Ph | N | —(CH₂)₅-NH- | H | Cl | H | H | |
| 1-547 | Ph | N | —(CH₂)₂—O—(CH₂)2-NH- | H | Cl | H | H | |
| 1-548 | Ph | N | —CH₂(CH₂)₅CH₂— | H | Cl | H | H | |
| 1-549 | 3,5-(F)₂—Ph | N | —CH₂(CH₂)₅CH₂— | H | Cl | H | H | |
| 1-550 | Ph | N | —CH₂(CH₂)₃CH₂— | H | Cl | CH₃ | H | |
| 1-551 | Ph | N | —CH₂(CH₂)₃CH₂— | H | Cl | CH₂CH₃ | H | |
| 1-552 | Ph | N | —CH₂(CH₂)₃CH₂— | H | Cl | CH(CH₃)CH₃ | H | |
| 1-553 | Ph | N | —CH₂(CH₂)₃CH₂— | H | Cl | Ph | H | |
| 1-554 | Ph | N | —(CH₂)₂—CH═CH—CH₂— | H | Cl | H | H | |
| 1-555 | 3,5-(F)₂—Ph | N | —(CH₂)₂—CH═CH—CH₂— | H | Cl | H | H | |
| 1-556 | Ph | N | —(CH₂)₂—C(Ph)═CH—CH₂— | H | Cl | H | H | |
| 1-557 | Ph | N | —(CH₂)₂—C(4-Cl—Ph)CH—CH₂— | H | Cl | H | H | |
| 1-558 | Ph | N | —(CH₂)₂—C(COOH)═CH—CH₂— | H | Cl | H | H | |
| 1-559 | Ph | N | —(CH₂)₂—CHC(COOH)—CH₂— | H | Cl | H | H | |
| 1-560 | Ph | N | —(CH₂)₃-CH═N- | H | Cl | H | H | |
| 1-561 | 1-Naph | N | —CH₂(CH₂)₃CH₂— | H | Cl | H | H | |
| 1-562 | 2-NaPh | N | —CH₂(CH₂)₃CH₂— | H | Cl | N | H | |
| 1-563 | 2-F—Ph | N | —CH₂(CH₂)₃CH₂— | H | Cl | H | H | |
| 1-564 | 2-Cl—Ph | N | —CH₂(CH₂)₃CH₂— | H | Cl | H | H | |
| 1-565 | 2-Br—Ph | N | —CH₂(CH₂)₃CH₂— | H | Cl | H | H | |
| 1-566 | 2-I—Ph | N | —CH₂(CH₂)₃CH₂— | H | Cl | H | H | |
| 1-567 | 2-CH₃—Ph | N | —CH₂(CH₂)₃CH₂— | H | Cl | H | H | |
| 1-568 | 2-CF₃—Ph | N | —CH₂(CH₂)₃CH₂— | H | Cl | H | H | |
| 1-569 | 2-CN—Ph | N | —CH₂(CH₂)₃CH₂— | H | Cl | H | H | |
| 1-570 | 2-OCH₃—Ph | N | —CH₂(CH₂)₃CH₂— | H | Cl | H | H | |
| 1-571 | 2-NO₂—Ph | N | —CH₂(CH₂)₃CH₂— | H | Cl | H | H | |
| 1-572 | 2-NH₂—Ph | N | —CH₂(CH₂)₃CH₂— | H | Cl | H | H | |
| 1-573 | 2-Ph—Ph | N | —CH₂(CH₂)₃CH₂— | H | Cl | H | H | |
| 1-574 | 3-F—Ph | N | —CH₂(CH₂)₃CH₂— | H | Cl | H | H | |
| 1-575 | 3-Cl—Ph | N | —CH₂(CH₂)₃CH₂— | H | Cl | H | H | |
| 1-576 | 3-Br—Ph | N | —CH₂(CH₂)₃CH₂— | H | Cl | H | H | |
| 1-577 | 3-I—Ph | N | —CH₂(CH₂)₃CH₂— | H | Cl | H | H | |
| 1-578 | 3-CH₃—Ph | N | —CH₂(CH₂)₃CH₂— | H | Cl | H | H | |
| 1-579 | 3-CF₃—Ph | N | —CH₂(CH₂)₃CH₂— | H | Cl | H | H | |
| 1-580 | 3-CN—Ph | N | —CH₂(CH₂)₃CH₂— | H | Cl | H | H | |
| 1-581 | 3-OCH₃—Ph | N | —CH₂(CH₂)₃CH₂— | H | Cl | H | H | |
| 1-582 | 3-OCF₃—Ph | N | —CH₂(CH₂)₃CH₂— | H | Cl | H | H | |
| 1-583 | 3-NO₂—Ph | N | —CH₂(CH₂)₃CH₂— | H | Cl | H | H | |
| 1-584 | 3-NH₂—Ph | N | —CH₂(CH₂)₃CH₂— | H | Cl | H | H | |
| 1-585 | 3-Ph—Ph | N | —CH₂(CH₂)₃CH₂— | H | Cl | H | H | |
| 1-586 | 4-F—Ph | N | —CH₂(CH₂)₃CH₂— | H | Cl | H | H | |
| 1-587 | 4-Cl—Ph | N | —CH₂(CH₂)₃CH₂— | H | Cl | H | H | |
| 1-588 | 4-Br—Ph | N | —CH₂(CH₂)₃CH₂— | H | Cl | H | H | |
| 1-589 | 4-CH₃—Ph | N | —CH₂(CH₂)₃CH₂— | H | Cl | H | H | |
| 1-590 | 4-C(CH₃)₃—Ph | N | —CH₂(CH₂)₃CH₂— | H | Cl | H | H | |
| 1-591 | 4-CF₃—Ph | N | —CH₂(CH₂)₃CH₂— | H | Cl | H | H | |
| 1-592 | 4-CN—Ph | N | —CH₂(CH₂)₃CH₂— | H | Cl | H | H | |
| 1-593 | 4-OCH₃—Ph | N | —CH₂(CH₂)₃CH₂— | H | Cl | H | H | |
| 1-594 | 4-OCF₃—Ph | N | —CH₂(CH₂)₃CH₂— | H | Cl | H | H | |
| 1-595 | 4-NO₂—Ph | N | —CH₂(CH₂)₃CH₂— | H | Cl | H | H | |
| 1-596 | 4-NH₂—Ph | N | —CH₂(CH₂)₃CH₂— | H | Cl | H | H | |
| 1-597 | 4-Ph—Ph | N | —CH₂(CH₂)₃CH₂— | H | Cl | H | H | |
| 1-598 | 2-Cl-4-F—Ph | N | —CH₂(CH₂)₃CH₂— | H | Cl | H | H | |
| 1-599 | 2-Cl-6-F—Ph | N | —CH₂(CH₂)₃CH₂— | H | Cl | H | H | |
| 1-600 | 2,3-(F)₂—Ph | N | —CH₂(CH₂)₃CH₂— | H | Cl | H | H | |
| 1-601 | 2,4-(F)₂—Ph | N | —CH₂(CH₂)₃CH₂— | H | Cl | H | H | |
| 1-602 | 2,5-(F)₂—Ph | N | —CH₂(CH₂)₃CH₂— | H | Cl | H | H | |
| 1-603 | 2,6-(F)₂—Ph | N | —CH₂(CH₂)₃CH₂— | H | Cl | H | H | |
| 1-604 | 3,4-(F)₂—Ph | N | —CH₂(CH₂)₃CH₂— | H | Cl | H | H | |
| 1-605 | 3,5-(F)₂—Ph | N | —CH₂(CH₂)₃CH₂— | H | Cl | H | H | |
| 1-606 | 2,3,4-(F)₃—Ph | N | —CH₂(CH₂)₃CH₂— | H | Cl | H | H | |
| 1-607 | 2,3,6-(F)₃—Ph | N | —CH₂(CH₂)₃CH₂— | H | Cl | H | H | |
| 1-608 | 2,4,6-(F)₃—Ph | N | —CH₂(CH₂)₃CH₂— | H | Cl | H | H | |
| 1-609 | 2,3,4,5,6-(F)6—Ph | N | —CH₂(CH₂)₃CH₂— | H | Cl | H | H | |
| 1-610 | 2,3,4,5,6-(F)₅—Ph | N | —CH₂(CH₂)₃CH₂— | H | Cl | F | F | |
| 1-611 | pyridin-2-yl | N | —CH₂(CH₂)₃CH₂— | H | Cl | H | H | |
| 1-612 | 6-Cl-pyridin-2-yl | N | —CH₂(CH₂)₃CH₂— | H | Cl | H | H | |
| 1-613 | 3-Cl-5-CF₃-pyridin-2-yl | N | —CH₂(CH₂)₃CH₂— | H | Cl | H | H | |
| 1-614 | pyridin-3-yl | N | —CH₂(CH₂)₃CH₂— | H | Cl | H | H | |
| 1-615 | 6-Cl-pyridin-3-yl | N | —CH₂(CH₂)₃CH₂— | H | Cl | H | H | |
| 1-616 | pyridin-4-yl | N | —CH₂(CH₂)₃CH₂— | H | Cl | H | H | |
| 1-617 | 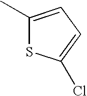 | N | —CH₂(CH₂)₃CH₂— | H | Cl | H | H | |
| 1-618 | 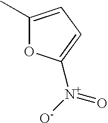 | N | —CH₂(CH₂)₃CH₂— | H | Cl | H | H | |
| 1-619 | Ph | N | —CH₂(CH₂)₃CH₂— | H | H | H | H | |
| 1-620 | Ph | N | —CH₂(CH₂)₃CH₂— | H | F | H | H | |
| 1-621 | Ph | N | —CH₂(CH₂)₃CH₂— | H | CH₃ | H | H | |
| 1-622 | Ph | N | —CH₂(CH₂)₃CH₂— | H | CF₃ | H | H | |
| 1-623 | Ph | N | —CH₂(CH₂)₃CH₂— | H | OCH₃ | H | H | |
| 1-624 | Ph | N | —CH₂(CH₂)₃CH₂— | H | SCH₃ | H | H | |
| 1-625 | Ph | N | —CH₂(CH₂)₃CH₂— | H | SO₂CH₃ | H | H | |
| 1-626 | Ph | N | —CH₂(CH₂)₃CH₂— | H | CN | H | H | |
| 1-627 | Ph | N | —CH₂(CH₂)₃CH₂— | H | 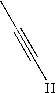 | H | H | |
| 1-628 | Ph | N | —CH₂(CH₂)₃CH₂— | H | 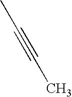 | H | H | |
| 1-629 | Ph | N | —CH₂(CH₂)₃CH₂— | CH₃ | Cl | H | H | |
| 1-630 | Ph | N | —CH₂(CH₂)₃CH₂— | CF₃ | Cl | H | H | |
| 1-631 | Ph | N | —CH₂(CH₂)₃CH₂— | C(CH₃)₃ | Cl | H | H | |
| 1-632 | Ph | N | —CH₂(CH₂)₃CH₂— | cyclopropyl | Cl | H | H | |
| 1-633 | Ph | N | —CH₂(CH₂)₃CH₂— | OCH₃ | Cl | H | H | |
| 1-634 | Ph | N | —CH₂(CH₂)₃CH₂— | O(CH₂)₃CH₃ | Cl | H | H | |
| 1-635 | Ph | N | —CH₂(CH₂)₃CH₂— | ON═O(CH₃)₂ | Cl | H | H | |
| 1-636 | Ph | N | —CH₂(CH₂)₃CH₂— | SCH₃ | Cl | H | H | |
| 1-637 | Ph | N | —CH₂(CH₂)₃CH₂— | SO₂CH₃ | Cl | H | H | |
| 1-638 | Ph | N | —CH₂(CH₂)₃CH₂— | NHPh | Cl | H | H | |
| 1-639 | Ph | N | —CH₂(CH₂)₃CH₂— | NH(CH₃)NH₂ | Cl | H | H | |
| 1-640 | Ph | N | —CH₂(CH₂)₃CH₂— | N(CH₃)N═C(CH₃)₂ | Cl | H | H | |
| 1-641 | Ph | N | —CH₂(CH₂)₃CH₂— | N(CH₃)N═(Ph)CH₃ | Cl | H | H | |
| 1-642 | Ph | N | —CH₂(CH₂)₃CH₂— | CN | Cl | H | H | |
| 1-643 | Ph | N | —CH₂(CH₂)₃CH₂— | C(═NOH)NH₂ | Cl | H | H | |
| 1-644 | Ph | N | —CH₂(CH₂)₃CH₂— | C(═NOCH₃)NH₂ | Cl | H | H | |
| 1-645 | Ph | N | —CH₂(CH₂)₃CH₂— | C(CH₃)═NOCH₂CH₃ | Cl | H | H | |
| 1-646 | Ph | N | —CH₂(CH₂)₃CH₂— | C(CH₃)═NOCH₂OH═CHCl | Cl | H | H | |
| 1-647 | Ph | N | —CH₂(CH₂)₃CH₂— | C(CH₃)═NOCH₂CH₂OCH₃ | Cl | H | H | |
| 1-648 | Ph | N | —CH₂(CH₂)₃CH₂— | CH₃ | OCH₃ | H | H | |
| 1-649 | Ph | N | —CH₂(CH₂)₃CH₂— | 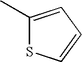 | Cl | H | H | |
| 1-650 | Ph | N | —CH₂(CH₂)₃CH₂— | 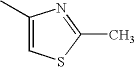 | Cl | H | H | |
| 1-651 | Ph | N | —CH₂(CH₂)₃CH₂— | pyridin-2-yl | Cl | H | H | |
| 1-652 | 1-NaPh | N | —CH₂(CH₂)₃CH₂— | pyridin-2-yl | Cl | H | H | |
| 1-653 | 2-NaPh | N | —CH₂(CH₂)₃CH₂— | pyridin-2-yl | Cl | H | H | |
| 1-654 | 2-F—Ph | N | —CH₂(CH₂)₃CH₂— | pyridin-2-yl | Cl | H | H | |
| 1-655 | 2-Cl—Ph | N | —CH₂(CH₂)₃CH₂— | pyridin-2-yl | Cl | H | H | |
| 1-656 | 2-Br—Ph | N | —CH₂(CH₂)₃CH₂— | pyridin-2-yl | Cl | H | H | |
| 1-657 | 2-I—Ph | N | —CH₂(CH₂)₃CH₂— | pyridin-2-yl | Cl | H | H | |
| 1-658 | 2-CH₃—Ph | N | —CH₂(CH₂)₃CH₂— | pyridin-2-yl | Cl | H | H | |
| 1-659 | 2-CF₃—Ph | N | —CH₂(CH₂)₃CH₂— | pyridin-2-yl | Cl | H | H | |
| 1-660 | 2-CN—Ph | N | —CH₂(CH₂)₃CH₂— | pyridin-2-yl | Cl | H | H | |
| 1-661 | 2-OCH₃—Ph | N | —CH₂(CH₂)₃CH₂— | pyridin-2-yl | Cl | H | H | |
| 1-662 | 2-NO₂—Ph | N | —CH₂(CH₂)₃CH₂— | pyridin-2-yl | Cl | H | H | |
| 1-663 | 2-NH₂—Ph | N | —CH₂(CH₂)₃CH₂— | pyridin-2-yl | Cl | H | H | |
| 1-664 | 2-Ph—Ph | N | —CH₂(CH₂)₃CH₂— | pyridin-2-yl | Cl | H | H | |
| 1-665 | 3-F—Ph | N | —CH₂(CH₂)₃CH₂— | pyridin-2-yl | Cl | H | H | |
| 1-666 | 3-Cl—Ph | N | —CH₂(CH₂)₃CH₂— | pyridin-2-yl | Cl | H | H | |
| 1-667 | 3-Br—Ph | N | —CH₂(CH₂)₃CH₂— | pyridin-2-yl | Cl | H | H | |
| 1-668 | 3-I—Ph | N | —CH₂(CH₂)₃CH₂— | pyridin-2-yl | Cl | H | H | |
| 1-669 | 3-CH₃—Ph | N | —CH₂(CH₂)₃CH₂— | pyridin-2-yl | Cl | H | H | |
| 1-670 | 3-CF₃—Ph | N | —CH₂(CH₂)₃CH₂— | pyridin-2-yl | Cl | H | H | |
| 1-671 | 3-CN—Ph | N | —CH₂(CH₂)₃CH₂— | pyridin-2-yl | Cl | H | H | |
| 1-672 | 3-OCH₃—Ph | N | —CH₂(CH₂)₃CH₂— | pyridin-2-yl | Cl | H | H | |
| 1-673 | 3-OCF₃—Ph | N | —CH₂(CH₂)₃CH₂— | pyridin-2-yl | Cl | H | H | |
| 1-674 | 3-NO₂—Ph | N | —CH₂(CH₂)₃CH₂— | pyridin-2-yl | Cl | H | H | |
| 1-675 | 3-NH₂—Ph | N | —CH₂(CH₂)₃CH₂— | pyridin-2-yl | Cl | H | H | |
| 1-676 | 3-Ph—Ph | N | —CH₂(CH₂)₃CH₂— | pyridin-2-yl | Cl | H | H | |
| 1-677 | 4-F—Ph | N | —CH₂(CH₂)₃CH₂— | pyridin-2-yl | Cl | H | H | |
| 1-678 | 4-Cl—Ph | N | —CH₂(CH₂)₃CH₂— | pyridin-2-yl | Cl | H | H | |
| 1-679 | 4-Br—Ph | N | —CH₂(CH₂)₃CH₂— | pyridin-2-yl | Cl | H | H | |
| 1-680 | 4-CH₃—Ph | N | —CH₂(CH₂)₃CH₂— | pyridin-2-yl | Cl | H | H | |
| 1-681 | 4-C(CH₃)₃—Ph | N | —CH₂(CH₂)₃CH₂— | pyridin-2-yl | Cl | H | H | |
| 1-682 | 4-CF₃—Ph | N | —CH₂(CH₂)₃CH₂— | pyridin-2-yl | Cl | H | H | |
| 1-683 | 4-CN—Ph | N | —CH₂(CH₂)₃CH₂— | pyridin-2-yl | Cl | H | H | |
| 1-684 | 4-OCH₃—Ph | N | —CH₂(CH₂)₃CH₂— | pyridin-2-yl | Cl | H | H | |
| 1-685 | 4-OCF₃—Ph | N | —CH₂(CH₂)₃CH₂— | pyridin-2-yl | Cl | H | H | |
| 1-686 | 4-NO₂—Ph | N | —CH₂(CH₂)₃CH₂— | pyridin-2-yl | Cl | H | H | |
| 1-687 | 4-NH₂—Ph | N | —CH₂(CH₂)₃CH₂— | pyridin-2-yl | Cl | H | H | |
| 1-688 | 4-Ph—Ph | N | —CH₂(CH₂)₃CH₂— | pyridin-2-yl | Cl | H | H | |
| 1-689 | 2-Cl-4-F—Ph | N | —CH₂(CH₂)₃CH₂— | pyridin-2-yl | Cl | H | H | |
| 1-690 | 2-Cl-6-F—Ph | N | —CH₂(CH₂)₃CH₂— | pyridin-2-yl | Cl | H | H | |
| 1-691 | 2,3-(F)₂—Ph | N | —CH₂(CH₂)₃CH₂— | pyridin-2-yl | Cl | H | H | |
| 1-692 | 2,4(F)2Ph | N | —CH₂(CH₂)₃CH₂— | pyridin-2-yl | Cl | H | H | |
| 1-693 | 2,5-(F)₂—Ph | N | —CH₂(CH₂)₃CH₂— | pyridin-2-yl | Cl | H | H | |
| 1-694 | 2,6(F)2Ph | N | —CH₂(CH₂)₃CH₂— | pyridin-2-yl | Cl | H | H | |
| 1-695 | 3,4-(F)₂—Ph | N | —CH₂(CH₂)₃CH₂— | pyridin-2-yl | Cl | H | H | |
| 1-696 | 3,5-(F)₂—Ph | N | —CH₂(CH₂)₃CH₂— | pyridin-2-yl | Cl | H | H | |
| 1-697 | 2,3,4-(F)₃—Ph | N | —CH₂(CH₂)₃CH₂— | pyridin-2-yl | Cl | H | H | |
| 1-698 | 2,3,6-(F)₃—Ph | N | —CH₂(CH₂)₃CH₂— | pyridin-2-yl | Cl | H | H | |
| 1-699 | 2,4,6-(F)₃—Ph | N | —CH₂(CH₂)₃CH₂— | pyridin-2-yl | Cl | H | H | |
| 1-700 | 2,3,4,5,6-(F)₅—Ph | N | —CH₂(CH₂)₃CH₂— | pyridin-2-yl | Cl | H | H | |
| 1-701 | 2,3,4,5,6-(F)₅—Ph | N | —CH₂(CH₂)₃CH₂— | pyridin-2-yl | Cl | F | F | |
| 1-702 | pyridin-2-yl | N | —CH₂(CH₂)₃CH₂— | pyridin-2-yl | Cl | H | H | |
| 1-703 | 6-Cl-pyridin-2-yl | N | —CH₂(CH₂)₃CH₂— | pyridin-2-yl | Cl | H | H | |
| 1-704 | 3-Cl-5-CF₃-pyridin-2-yl | N | —CH₂(CH₂)₃CH₂— | pyridin-2-yl | Cl | H | H | |
| 1-705 | pyridin-3-yl | N | —CH₂(CH₂)₃CH₂— | pyridin-2-yl | Cl | H | H | |
| 1-706 | 6-Cl-pyridin-3-yl | N | —CH₂(CH₂)₃CH₂— | pyridin-2-yl | Cl | H | H | |
| 1-707 | pyridin-4-yl | N | —CH₂(CH₂)₃CH₂— | pyridin-2-yl | Cl | H | H | |
| 1-708 | 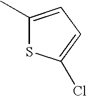 | N | —CH₂(CH₂)₃CH₂— | pyridin-2-yl | Cl | H | H | |
| 1-709 | 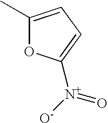 | N | —CH₂(CH₂)₃CH₂— | pyridin-2-yl | Cl | H | H | |
| 1-710 | Ph | N | —CH₂(CH₂)₃CH₂— | pyridin-2-yl | H | H | H | |
| 1-711 | Ph | N | —CH₂(CH₂)₃CH₂— | 3-CH₃-pyridin-2-yl | Cl | H | H | |
| 1-712 | Ph | N | —CH₂(CH₂)₃CH₂— | 4-CH₃-pyridin-2-yl | Cl | H | H | |
| 1-713 | Ph | N | —CH₂(CH₂)₃CH₂— | 6-CH₃-pyridin-2-yl | Cl | H | H | |
| 1-714 | Ph | N | —CH₂(CH₂)₃CH₂— | pyridin-3-yl | Cl | H | H | |
| 1-715 | Ph | N | —CH₂(CH₂)₃CH₂— | pyridin-4-yl | Cl | H | H | |
| 1-716 | Ph | N | —CH₂(CH₂)₃CH₂— | 2,6-(Cl)₂-pyridin-4-yl | Cl | H | H | |
| 1-717 | Ph | N | —CH₂(CH₂)₃CH₂— | 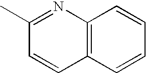 | Cl | H | H | |
| 1-718 | Ph | N | —CH₂(CH₂)₃CH₂— | 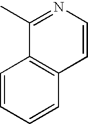 | Cl | H | H | |
| 1-719 | Ph | N | —CH₂(CH₂)₃CH₂— | 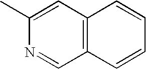 | Cl | H | H | |
| 1-720 | Ph | N | —CH₂(CH₂)₃CH₂— | pyrimidin-2-yl | Cl | H | H | |
| 1-721 | Ph | N | —CH₂(CH₂)₃CH₂— | pyridazin-3-yl | Cl | H | H | |
| 1-722 | Ph | N | —CH₂(CH₂)₃CH₂— | pyrazin-2-yl | Cl | H | H | |
| 1-723 | 1-NaPh | N | —CH₂(CH₂)₃CH₂— | pyrazin-2-yl | Cl | H | H | |
| 1-724 | 2-NaPh | N | —CH₂(CH₂)₃CH₂— | pyrazin-2-yl | Cl | H | H | |
| 1-725 | 2-F—Ph | N | —CH₂(CH₂)₃CH₂— | pyrazin-2-yl | Cl | H | H | |
| 1-726 | 2-Cl—Ph | N | —CH₂(CH₂)₃CH₂— | pyrazin-2-yl | Cl | H | H | |
| 1-727 | 2-Br—Ph | N | —CH₂(CH₂)₃CH₂— | pyrazin-2-yl | Cl | H | H | |
| 1-728 | 2-I—Ph | N | —CH₂(CH₂)₃CH₂— | pyrazin-2-yl | Cl | H | H | |
| 1-729 | 2-CH₃—Ph | N | —CH₂(CH₂)₃CH₂— | pyrazin-2-yl | Cl | H | H | |
| 1-730 | 2-CF₃—Ph | N | —CH₂(CH₂)₃CH₂— | pyrazin-2-yl | Cl | H | H | |
| 1-731 | 2-CN—Ph | N | —CH₂(CH₂)₃CH₂— | pyrazin-2-yl | Cl | H | H | |
| 1-732 | 2-OCH₃—Ph | N | —CH₂(CH₂)₃CH₂— | pyrazin-2-yl | Cl | H | H | |
| 1-733 | 2-NO₂—Ph | N | —CH₂(CH₂)₃CH₂— | pyrazin-2-yl | Cl | H | H | |
| 1-734 | 2-NH₂—Ph | N | —CH₂(CH₂)₃CH₂— | pyrazin-2-yl | Cl | H | H | |
| 1-735 | 2-Ph—Ph | N | —CH₂(CH₂)₃CH₂— | pyrazin-2-yl | Cl | H | H | |
| 1-736 | 3-F—Ph | N | —CH₂(CH₂)₃CH₂— | pyrazin-2-yl | Cl | H | H | |
| 1-737 | 3-Cl—Ph | N | —CH₂(CH₂)₃CH₂— | pyrazin-2-yl | Cl | H | H | |
| 1-738 | 3-Br—Ph | N | —CH₂(CH₂)₃CH₂— | pyrazin-2-yl | Cl | H | H | |
| 1-739 | 3-I—Ph | N | —CH₂(CH₂)₃CH₂— | pyrazin-2-yl | Cl | H | H | |
| 1-740 | 3-CH₃—Ph | N | —CH₂(CH₂)₃CH₂— | pyrazin-2-yl | Cl | H | H | |
| 1-741 | 3-CF₃—Ph | N | —CH₂(CH₂)₃CH₂— | pyrazin-2-yl | Cl | H | H | |
| 1-742 | 3-CN—Ph | N | —CH₂(CH₂)₃CH₂— | pyrazin-2-yl | Cl | H | H | |
| 1-743 | 3-OCH₃—Ph | N | —CH₂(CH₂)₃CH₂— | pyrazin-2-yl | Cl | H | H | |
| 1-744 | 3-OCF₃—Ph | N | —CH₂(CH₂)₃CH₂— | pyrazln-2-yl | Cl | H | H | |
| 1-745 | 3-NO₂—Ph | N | —CH₂(CH₂)₃CH₂— | pyrazin-2-yl | Cl | H | H | |
| 1-746 | 3-NH₂—Ph | N | —CH₂(CH₂)₃CHz | pyrazin-2-yl | Cl | H | H | |
| 1-747 | 3-Ph—Ph | N | —CH₂(CH₂)₃CH₂— | pyrazin-2-yl | Cl | H | H | |
| 1-748 | 4-F—Ph | N | —CH₂(CH₂)₃CH₂— | pyrazin-2-yl | Cl | H | H | |
| 1-749 | 4-Cl—Ph | N | —CH₂(CH₂)₃CH₂— | pyrazin-2-yl | Cl | H | H | |
| 1-750 | 4-Br—Ph | N | —CH₂(CH₂)₃CH₂— | pyrazin-2-yl | Cl | H | H | |
| 1-751 | 4-CH₃—Ph | N | —CH₂(CH₂)₃CH₂— | pyrazin-2-yl | Cl | H | H | |
| 1-752 | 4-C(CH₃)₃—Ph | N | —CH₂(CH₂)₃CH₂— | pyrazin-2-yl | Cl | H | H | |
| 1-753 | 4-CF₃—Ph | N | —CH₂(CH₂)₃CH₂— | pyrazin-2-yl | Cl | H | H | |
| 1-754 | 4-CN—Ph | N | —CH₂(CH₂)₃CH₂— | pyrazin-2-yl | Cl | H | H | |
| 1-755 | 4-OCH₃—Ph | N | —CH₂(CH₂)₃CH₂— | pyrazin-2-yl | Cl | H | H | |
| 1-756 | 4-OCF₃—Ph | N | —CH₂(CH₂)₃CH₂— | pyrazin-2-yl | Cl | H | H | |
| 1-757 | 4-NO₂—Ph | N | —CH₂(CH₂)₃CH₂— | pyrazin-2-yl | Cl | H | H | |
| 1-758 | 4-NH₂—Ph | N | —CH₂(CH₂)₃CH₂— | pyrazin-2-yl | Cl | H | H | |
| 1-759 | 4-Ph—Ph | N | —CH₂(CH₂)₃CH₂— | pyrazin-2-yl | Cl | H | H | |
| 1-760 | 2-Cl-4-F—Ph | N | —CH₂(CH₂)₃CH₂— | pyrazin-2-yl | Cl | H | H | |
| 1-761 | 2-Cl-6-F—Ph | N | —CH₂(CH₂)₃CH₂— | pyrazin-2-yl | Cl | H | H | |
| 1-762 | 2,3-(F)₂—Ph | N | —CH₂(CH₂)₃CH₂— | pyrazin-2-yl | Cl | H | H | |
| 1-763 | 2,4-(F)₂—Ph | N | —CH₂(CH₂)₃CH₂— | pyrazin-2-yl | Cl | H | H | |
| 1-764 | 2,5-(F)₂—Ph | N | —CH₂(CH₂)₃CH₂— | pyrazin-2-yl | Cl | H | H | |
| 1-765 | 2,6-(F)₂—Ph | N | —CH₂(CH₂)₃CH₂— | pyrazin-2-yl | Cl | H | H | |
| 1-766 | 3,4-(F)₂—Ph | N | —CH₂(CH₂)₃CH₂— | pyrazin-2-yl | Cl | H | H | |
| 1-767 | 3,5-(F)₂—Ph | N | —CH₂(CH₂)₃CH₂— | pyrazin-2-yl | Cl | H | H | |
| 1-768 | 2,3,4-(F)₃—Ph | N | —CH₂(CH₂)₃CH₂— | pyrazin-2-yl | Cl | H | H | |
| 1-769 | 2,3,6(F)₃—Ph | N | —CH₂(CH₂)₃CH₂— | pyrazin-2-yl | Cl | H | H | |
| 1-770 | 2,4,6-(F)₃—Ph | N | —CH₂(CH₂)₃CH₂— | pyrazin-2-yl | Cl | H | H | |
| 1-771 | 2,3,4,5,6-(F)₅—Ph | N | —CH₂(CH₂)₃CH₂— | pyrazin-2-yl | Cl | H | H | |
| 1-772 | 2,3,4,5,6-(F)₅—Ph | N | —CH₂(CH₂)₃CH₂— | pyrazin-2-yl | Cl | F | F | |
| 1-773 | pyridin-2-yl | N | —CH₂(CH₂)₃CH₂— | pyrazin-2-yl | Cl | H | H | |
| 1-774 | 6-Cl-pyridin-2-yl | N | —CH₂(CH₂)₃CH₂— | pyrazin-2-yl | Cl | H | H | |
| 1-775 | 3-Cl-5-CF₃-pyridin-2-yl | N | —CH₂(CH₂)₃CH₂— | pyrazin-2-yl | Cl | H | H | |
| 1-776 | pyridin-3-yl | N | —CH₂(CH₂)₃CH₂— | pyrazin-2-yl | Cl | H | H | |
| 1-777 | 6-Cl-pyridin-3-yl | N | —CH₂(CH₂)₃CH₂— | pyrazin-2-yl | Cl | H | H | |
| 1-778 | pyridin-4-yl | N | —CH₂(CH₂)₃CH₂— | pyrazin-2-yl | Cl | H | H | |
| 1-779 | 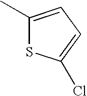 | N | —CH₂(CH₂)₃CH₂— | pyrazin-2-yl | Cl | H | H | |
| 1-780 | 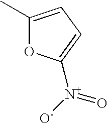 | N | —CH₂(CH₂)₃CH₂— | pyrazin-2-yl | Cl | H | H | |
| 1-781 | Ph | N | —CH₂(CH₂)₃CH₂— | pyrazin-2-yl | H | H | H | |
| 1-782 | Ph | N | —CH₂(CH₂)₃CH₂— | pyrazol-1-yl | Cl | H | H | |
| 1-783 | 1-NaPh | N | —CH₂(CH₂)₃CH₂— | pyrazol-1-yl | Cl | H | H | |
| 1-784 | 2-NaPh | N | —CH₂(CH₂)₃CH₂— | pyrazol-1-yl | Cl | H | H | |
| 1-785 | 2-F—Ph | N | —CH₂(CH₂)₃CH₂— | pyrazol-1-yl | Cl | H | H | |
| 1-786 | 2-Cl—Ph | N | —CH₂(CH₂)₃CH₂— | pyrazol-1-yl | Cl | H | H | |
| 1-787 | 2-Br—Ph | N | —CH₂(CH₂)₃CH₂— | pyrazol-1-yl | Cl | H | H | |
| 1-788 | 2-I—Ph | N | —CH₂(CH₂)₃CH₂— | pyrazol-1-yl | Cl | H | H | |
| 1-789 | 2-CH₃—Ph | N | —CH₂(CH₂)₃CH₂— | pyrazol-1-yl | Cl | H | H | |
| 1-790 | 2-CF₃—Ph | N | —CH₂(CH₂)₃CH₂— | pyrazol-1-yl | Cl | H | H | |
| 1-791 | 2-CN—Ph | N | —CH₂(CH₂)₃CH₂— | pyrazol-1-yl | Cl | H | H | |
| 1-792 | 2-OCH₃—Ph | N | —CH₂(CH₂)₃CH₂— | pyrazol-1-yl | Cl | H | H | |
| 1-793 | 2-NO₂—Ph | N | —CH₂(CH₂)₃CH₂— | pyrazol-1-yl | Cl | H | H | |
| 1-794 | 2-NH₂—Ph | N | —CH₂(CH₂)₃CH₂— | pyrazol-1-yl | Cl | H | H | |
| 1-795 | 2-Ph—Ph | N | —CH₂(CH₂)₃CH₂— | pyrazol-1-yl | Cl | H | H | |
| 1-796 | 3-F—Ph | N | —CH₂(CH₂)₃CH₂— | pyrazol-1-yl | Cl | H | H | |
| 1-797 | 3-Cl—Ph | N | —CH₂(CH₂)₃CH₂— | pyrazol-1-yl | Cl | H | H | |
| 1-798 | 3-Br—Ph | N | —CH₂(CH₂)₃CH₂— | pyrazol-1-yl | Cl | H | H | |
| 1-799 | 3-I—Ph | N | —CH₂(CH₂)₃CH₂— | pyrazol-1-yl | Cl | H | H | |
| 1-800 | 3-CH₃—Ph | N | —CH₂(CH₂)₃CH₂— | pyrazol-1-yl | Cl | H | H | |
| 1-801 | 3-CF₃—Ph | N | —CH₂(CH₂)₃CH₂— | pyrazol-1-yl | Cl | H | H | |
| 1-802 | 3-CN—Ph | N | —CH₂(CH₂)₃CH₂— | pyrazol-1-yl | Cl | H | H | |
| 1-803 | 3-OCH₃—Ph | N | —CH₂(CH₂)₃CH₂— | pyrazol-1-yl | Cl | H | H | |
| 1-804 | 3-OCF₃—Ph | N | —CH₂(CH₂)₃CH₂— | pyrazol-1-yl | Cl | H | H | |
| 1-805 | 3-NO₂—Ph | N | —CH₂(CH₂)₃CH₂— | pyrazol-1-yl | Cl | H | H | |
| 1-806 | 3-NH₂—Ph | N | —CH₂(CH₂)₃CH₂— | pyrazol-1-yl | Cl | H | H | |
| 1-807 | 3-Ph—Ph | N | —CH₂(CH₂)₃CH₂— | pyrazol-1-yl | Cl | H | H | |
| 1-808 | 4-F—Ph | N | —CH₂(CH₂)₃CH₂— | pyrazol-1-yl | Cl | H | H | |
| 1-809 | 4-Cl—Ph | N | —CH₂(CH₂)₃CH₂— | pyrazol-1-yl | Cl | H | H | |
| 1-810 | 4-Br—Ph | N | —CH₂(CH₂)₃CH₂— | pyrazol-1-yl | Cl | H | H | |
| 1-811 | 4-CH₃—Ph | N | —CH₂(CH₂)₃CH₂— | pyrazol-1-yl | Cl | H | H | |
| 1-812 | 4-C(CH₃)₃—Ph | N | —CH₂(CH₂)₃CH₂— | pyrazol-1-yl | Cl | H | H | |
| 1-813 | 4-CF₃—Ph | N | —CH₂(CH₂)₃CH₂— | pyrazol-1-yl | Cl | H | H | |
| 1-814 | 4-CN—Ph | N | —CH₂(CH₂)₃CH₂— | pyrazol-1-yl | Cl | H | H | |
| 1-815 | 4-OCH₃—Ph | N | —CH₂(CH₂)₃CH₂— | pyrazol-1-yl | Cl | H | H | |
| 1-816 | 4-OCF₃—Ph | N | —CH₂(CH₂)₃CH₂— | pyrazol-1-yl | Cl | H | H | |
| 1-817 | 4-NO₂—Ph | N | —CH₂(CH₂)₃CH₂— | pyrazol-1-yl | Cl | H | H | |
| 1-818 | 4-NH₂—Ph | N | —CH₂(CH₂)₃CH₂— | pyrazol-1-yl | Cl | H | H | |
| 1-819 | 4-Ph—Ph | N | —CH₂(CH₂)₃CH₂— | pyrazol-1-yl | Cl | H | H | |
| 1-820 | 2-Cl-4-F—Ph | N | —CH₂(CH₂)₃CH₂— | pyrazol-1-yl | Cl | H | H | |
| 1-821 | 2-Cl-6-F—Ph | N | —CH₂(CH₂)₃CH₂— | pyrazol-1-yl | Cl | H | H | |
| 1-822 | 2,3-(F)₂—Ph | N | —CH₂(CH₂)₃CH₂— | pyrazol-1-yl | Cl | H | H | |
| 1-823 | 2,4-(F)₂—Ph | N | —CH₂(CH₂)₃CH₂— | pyrazol-1-yl | Cl | H | H | |
| 1-824 | 2,5-(F)₂—Ph | N | —CH₂(CH₂)₃CH₂— | pyrazol-1-yl | Cl | H | H | |
| 1-825 | 2,6-(F)₂—Ph | N | —CH₂(CH₂)₃CH₂— | pyrazol-1-yl | Cl | H | H | |
| 1-826 | 3,4-(F)₂—Ph | N | —CH₂(CH₂)₃CH₂— | pyrazol-1-yl | Cl | H | H | |
| 1-827 | 3,5-(F)₂—Ph | N | —CH₂(CH₂,3CH₂— | pyrazol-1-yl | Cl | H | H | |
| 1-828 | 2,3,4-(F)₃—Ph | N | —CH₂(CH₂)₃CH₂— | pyrazol-1-yl | Cl | H | H | |
| 1-829 | 2,3,6-(F)₃—Ph | N | —CH₂(CH₂)₃CH₂— | pyrazol-1-yl | Cl | H | H | |
| 1-830 | 2,4,6-(F)₃—Ph | N | —CH₂(CH₂)₃CH₂— | pyrazol-1-yl | Cl | H | H | |
| 1-831 | 2,3,4,5,6-(F)₅—Ph | N | —CH₂(CH₂)₃CH₂— | pyrazol-1-yl | Cl | H | H | |
| 1-832 | 2,3,4,5,6-(F)₅—Ph | N | —CH₂(CH₂)₃CH₂— | pyrazol-1-yl | Cl | F | F | |
| 1-833 | pyridin-2-yl | N | —CH₂(CH₂)₃CH₂— | pyrazol-1-yl | Cl | H | H | |
| 1-834 | 6-Cl-pyridin-2-yl | N | —CH₂(CH₂)₃CH₂— | pyrazol-1-yl | Cl | H | H | |
| 1-835 | 3-Cl-5-CF₃-pyridin-2-yl | N | —CH₂(CH₂)₃CH₂— | pyrazol-1-yl | Cl | H | H | |
| 1-836 | pyridin-3-yl | N | —CH₂(CH₂)₃CH₂— | pyrazol-1-yl | Cl | H | H | |
| 1-837 | 6-Cl-pyridin-3-yl | N | —CH₂(CH₂)₃CH₂— | pyrazol-1-yl | Cl | H | H | |
| 1-838 | pyridin-4-yl | N | —CH₂(CH₂)₃CH₂— | pyrazol-1-yl | Cl | H | H | |
| 1-839 | 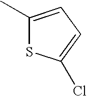 | N | —CH₂(CH₂)₃CH₂— | pyrazol-1-yl | Cl | H | H | |
| 1-840 | 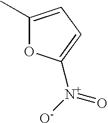 | N | —CH₂(CH₂)₃CH₂— | pyrazol-1-yl | Cl | H | H | |
| 1-841 | Ph | N | —CH₂(CH₂)₃CH₂— | pyrazol-1-yl | H | H | H | |
| 1-842 | Ph | N | —CH₂(CH₂)₃CH₂— | 3-CH₃-pyrazol-1-yl | Cl | H | H | |
| 1-843 | Ph | N | —CH₂(CH₂)₃CH₂— | 3,5-(CH₃)₂-pyrazol-1-yl | Cl | H | H | |
| 1-844 | Ph | N | —CH₂(CH₂)₃CH₂— | 3-CF₃-pyrazol-1-yl | Cl | H | H | |
| 1-845 | Ph | N | —CH₂(CH₂)₃CH₂— | 3,5-(CF₃)₂-pyrazol-1-yl | Cl | H | H | |
| 1-846 | Ph | N | —CH₂(CH₂)₃CH₂— | 4-Br-pyrazol-1-yl | Cl | H | H | |
| 1-847 | Ph | N | —CH₂(CH₂)₃CH₂— | 4-CH₃-pyrazol-1-yl | Cl | H | H | |
| 1-848 | Ph | N | —CH₂(CH₂)₃CH₂— | imidazol-1-yl | Cl | H | H | |
| 1-849 | Ph | N | —CH₂(CH₂)₃CH₂— | 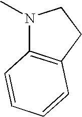 | Cl | H | H | |
| 1-850 | Ph | N | —CH₂(CH₂)₃CH₂— | 1,2,4-triazol-1-yl | Cl | H | H | |
| 1-851 | 1-NaPh | N | —CH₂(CH₂)₃CH₂— | 1,2,4-triazol-1-yl | Cl | H | H | |
| 1-852 | 2-NaPh | N | —CH₂(CH₂)₃CH₂— | 1,2,4-triazol-1-yl | Cl | H | H | |
| 1-853 | 2-F—Ph | N | —CH₂(CH₂)₃CH₂— | 1,2,4-triazol-1-yl | Cl | H | H | |
| 1-854 | 2-Cl—Ph | N | —CH₂(CH₂)₃CH₂— | 1,2,4-triazol-1-yl | Cl | H | H | |
| 1-855 | 2-Br—Ph | N | —CH₂(CH₂)₃CH₂— | 1,2,4-triazol-1-yl | Cl | H | H | |
| 1-856 | 2-I—Ph | N | —CH₂(CH₂)₃CH₂— | 1,2,4-triazol-1-yl | Cl | H | H | |
| 1-857 | 2-CH₃—Ph | N | —CH₂(CH₂)₃CH₂— | 1,2,4-triazol-1-yl | Cl | H | H | |
| 1-858 | 2-CF₃—Ph | N | —CH₂(CH₂)₃CH₂— | 1,2,4-triazol-1-yl | Cl | H | H | |
| 1-859 | 2-CN—Ph | N | —CH₂(CH₂)₃CH₂— | 1,2,4-triazol-1-yl | Cl | H | H | |
| 1-860 | 2-OCH₃—Ph | N | —CH₂(CH₂)₃CH₂- 1,2,4-triazol-1-yl | Cl | H | H | | |
| 1-861 | 2-NO₂—Ph | N | —CH₂(CH₂)₃CH₂— | 1,2,4-triazol-1-yl | Cl | H | H | |
| 1-862 | 2-NH₂—Ph | N | —CH₂(CH₂)₃CH₂— | 1,2,4-triazol-1-yl | Cl | H | H | |
| 1-863 | 2-Ph—Ph | N | —CH₂(CH₂)₃CH₂— | 1,2,4-triazol-1-yl | Cl | H | H | |
| 1-864 | 3-F—Ph | N | —CH₂(CH₂)₃CH₂— | 1,2,4-triazol-1-yl | Cl | H | H | |
| 1-865 | 3-Cl—Ph | N | —CH₂(CH₂)₃CH₂— | 1,2,4-triazol-1-yl | Cl | H | H | |
| 1-866 | 3-Br—Ph | N | —CH₂(CH₂)₃CH₂— | I 2,4-triazol-1-yl | Cl | H | H | |
| 1-867 | 3-I—Ph | N | —CH₂(CH₂)₃CH₂— | 1,2,4-triazol-1-yl | Cl | H | H | |
| 1-868 | 3-CH₃—Ph | N | —CH₂(CH₂)₃CH₂— | 1,2,4-triazol-1-yl | Cl | H | H | |
| 1-869 | 3-CF₃—Ph | N | —CH₂(CH₂)₃CH₂— | 1,2,4-triazol-1-yl | Cl | H | H | |
| 1-870 | 3-CN—Ph | N | —CH₂(CH₂)₃CH₂— | 1,2,4-triazol-1-yl | Cl | H | H | |
| 1-871 | 3-OCH₃—Ph | N | —CH₂(CH₂)₃CH₂— | 1,2,4-triazol-1-yl | Cl | H | H | |
| 1-872 | 3-OCF₃—Ph | N | —CH₂(CH₂)₃CH₂— | 1,2,4-triazol-1-yl | Cl | H | H | |
| 1-873 | 3-NO₂—Ph | N | —CH₂(CH₂)₃CH₂— | 1,2,4-triazol-1-yl | Cl | H | H | |
| 1-874 | 3-NH₂—Ph | N | —CH₂(CH₂)₃CH₂— | 1,2,4-triazol-1-yl | Cl | H | H | |
| 1-875 | 3-Ph—Ph | N | —CH₂(CH₂)₃CH₂— | 1,2,4-triazol-1-yl | Cl | H | H | |
| 1-876 | 4-F—Ph | N | —CH₂(CH₂)₃CH₂— | 1,2,4-triazol-1-yl | Cl | H | H | |
| 1-877 | 4-Cl—Ph | N | —CH₂(CH₂)₃CH₂- 1,2,4-triazol-1-yl | Cl | H | H | | |
| 1-878 | 4-Br—Ph | N | —CH₂(CH₂)₃CH₂— | 1,2,4-triazol-1-yl | Cl | H | H | |
| 1-879 | 4-CH₃—Ph | N | —CH₂(CH₂)₃CH₂— | 12,4-triazol-1-yl | Cl | H | H | |
| 1-880 | 4-C(CH₃)₃—Ph | N | —CH₂(CH₂)₃CH₂— | 1,2,4-triazol-1-yl | Cl | H | H | |
| 1-881 | 4-CF₃—Ph | N | —CH₂(CH₂)₃CH₂— | 1,2,4-triazol-1-yl | Cl | H | H | |
| 1-882 | 4-CN—Ph | N | —CH₂(CH₂)₃CH₂— | 1,2,4-triazol-1-yl | Cl | H | H | |
| 1-883 | 4-OCH₃—Ph | N | —CH₂(CH₂)₃CH₂— | 1,2,4-triazol-1-yl | Cl | H | H | |
| 1-884 | 4-OCF₃—Ph | N | —CH₂(CH₂)₃CH₂— | 1,2,4-triazol-1-yl | Cl | H | H | |
| 1-885 | 4-NO₂—Ph | N | —CH₂(CH₂)₃CH₂— | 1,2,4-triazol-1-yl | Cl | H | H | |
| 1-886 | 4-NH₂—Ph | N | —CH₂(CH₂)₃CH₂— | 1,2,4-triazol-1-yl | Cl | H | H | |
| 1-887 | 4-Ph—Ph | N | —CH₂(CH₂)₃CH₂— | 1,2,4-triazol-1-yl | Cl | H | H | |
| 1-888 | 2-Cl-4-F—Ph | N | —CH₂(CH₂)₃CH₂— | 1,2,4-triazol-1-yl | Cl | H | H | |
| 1-889 | 2-Cl-6-F—Ph | N | —CH₂(CH₂)₃CH₂— | 1,2,4-triazol-1-yl | Cl | H | H | |
| 1-890 | 2,3-(F)₂—Ph | N | —CH(CH₂)₃CH₂— | 1,2,4-triazol-1-yl | Cl | H | H | |
| 1-891 | 2,4-(F)₂—Ph | N | —CH₂(CH₂)₃CH₂— | 1,2,4-triazol-1-yl | Cl | H | H | |
| 1-892 | 2,5-(F)₂—Ph | N | —CH₂(CH₂)₃CH₂— | 1,2,4-triazol-1-yl | Cl | H | H | |
| 1-893 | 2,6-(F)₂—Ph | N | —CH₂(CH₂)₃CH₂— | 1,2,4-triazol-1-yl | Cl | H | H | |
| 1-894 | 3,4-(F)₂—Ph | N | —CH₂(CH₂)₃CH₂— | 1,2,4-triazol-1-yl | Cl | H | H | |
| 1-895 | 3,5-(F)₂—Ph | N | —CH₂(CH₂)₃CH₂— | 1,2,4-triazol-1-yl | Cl | H | H | |
| 1-896 | 2,3,4-(F)₃—Ph | N | —CH₂(CH₂)₃CH₂— | 1,2,4-triazol-1-yl | Cl | H | H | |
| 1-897 | 2,3,6-(F)₃—Ph | N | —CH₂(CH₂)₃CH₂— | 1,2,4-triazol-1-yl | Cl | H | H | |
| 1-898 | 2,4,6-(F)₃—Ph | N | —CH₂(CH₂)₃CH₂— | 1,2,4-triazol-1-yl | Cl | H | H | |
| 1-899 | 2,3,4,5,8-(F)6—Ph | N | —CH₂(CH₂)₃CH₂— | 1,2,4-triazol-1-yl | Cl | H | H | |
| 1-900 | 2,3,4,5,6-(F)₅—Ph | N | —CH₂(CH₂)₃CH₂— | 1,2,4-triazol-1-yl | Cl | F | F | |
| 1-901 | pyridin-2-yl | N | —CH₂(CH₂)₃CH₂— | 1,2,4-triazol-1-yl | Cl | H | H | |
| 1-902 | 6-Cl-pyridin-2-yl | N | —CH₂(CH₂)₃CH₂— | 1,2,4-triazol-1-yl | Cl | H | H | |
| 1-903 | 3-Cl-5-CF₃-pyridin-2-yl | N | —CH₂(CH₂)₃CH₂— | 1,2,4-triazol-1-yl | Cl | H | H | |
| 1-904 | pyridin-3-yl | N | —CH₂(CH₂)₃CH₂— | 1,2,4-triazol-1-yl | Cl | H | H | |
| 1-905 | 6-Cl-pyridin-3-yl | N | —CH₂(CH₂)₃CH₂— | 1,2,4-triazol-1-yl | Cl | H | H | |
| 1-906 | pyridin-4-yl | N | —CH₂(CH₂)₃CH₂— | 1,2,4-triazol-1-yl | Cl | H | H | |
| 1-907 | 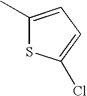 | N | —CH₂(CH₂)₃CH₂— | 1,2,4-triazol-1-yl | Cl | H | H | |
| 1-908 | 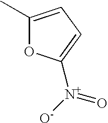 | N | —CH₂(CH₂)₃CH₂— | 1,2,4-triazol-1-yl | Cl | H | H | |
| 1-909 | Ph | N | —CH₂(CH₂)₃CH₂— | 1,2,4-triazol-1-yl | H | H | H | |
| 1-910 | Ph | N | —CH₂(CH₂)₃CH₂— | 1,2,3-triazol-1-yl | Cl | H | H | |
| 1-911 | Ph | N | —CH₂(CH₂)₃CH₂— | 1,2,5-triazol-1-yl | Cl | H | H | |
| 1-912 | Ph | N | —CH₂(CH₂)₃CH₂— | tetrazol-1-yl | Cl | H | H | |
| 1-913 | Ph | N | —CH₂(CH₂)₃CH₂— | tetrazol-2-yl | Cl | H | H | |
| 1-914 | Ph | N | —(CH₂)4-CH(CH₃)— | H | Cl | H | H | |
| 1-915 | Ph | N | —(CH₂)4-CH(CF₃)— | H | Cl | H | H | |
| 1-916 | Ph | N | —(CH₂)4-CH(CH₂CH₃)— | H | Cl | H | H | |
| 1-917 | Ph | N | —(CH₂)4-CH(CH₂CH₂CH₃)— | H | Cl | H | H | |
| 1-918 | Ph | N | —(CH₂)4-CH(CH₂OH)— | H | Cl | H | H | |
| 1-919 | Ph | N | —(CH₂)4-CH(CH₂CH₂OH)— | H | Cl | H | H | |
| 1-920 | Ph | N | —(CH₂)4-CH(COOH)— | H | Cl | H | H | |
| 1-921 | Ph | N | —(CH₂)4-CH(COOCH₃)— | H | Cl | H | H | |
| 1-922 | Ph | N | —(CH₂)4-CH(COOCH₂CH₃)— | H | Cl | H | H | |
| 1-923 | Ph | N | —(CH₂)4-CH(═O)— | H | Cl | H | H | |
| 1-924 | Ph | N | —(CH₂)₃-CH(CH₃)—CH₂— | H | Cl | H | H | |
| 1-925 | Ph | N | —(CH₂)₃-C(CH₃)CH3-CH₂— | H | Cl | H | H | |
| 1-926 | Ph | N | —(CH₂)₃-CH(CH₂OH)—CH₂— | H | Cl | H | H | |
| 1-927 | Ph | N | —(CH₂)₃-CH(COOH)—CH₂— | H | Cl | H | H | |
| 1-928 | Ph | N | —(CH₂)₃-CH(COOCH₂CH₃)—CH₂— | H | Cl | H | H | |
| 1-929 | Ph | N | —(CH₂)₃-CH(F)—CH₂— | H | Cl | H | H | |
| 1-930 | Ph | N | —(CH₂)₃-CH(OH)—CH₂— | H | Cl | H | H | |
| 1-931 | Ph | N | —(CH₂)₂—CH(CH₃)—(CH₂)2- | H | Cl | H | H | |
| 1-932 | Ph | N | —(CH₂)₂—CH(CH₃)—(CH₂)2- | H | Cl | CH₃ | H | |
| 1-933 | Ph | N | —(CH₂)₂—CH(CH₃)—(CH₂)2- | H | Cl | CH₂CH₃ | H | |
| 1-934 | Ph | N | —(CH₂)₂—CH(CH₃)—(CH₂)2- | H | Cl | CH(CH₃)CH₃ | H | |
| 1-935 | Ph | N | —(CH₂)₂—CH(CH₃)—(CH₂)2- | H | Cl | Ph | H | |
| 1-936 | Ph | N | —(CH₂)₂—CH(CF₃)—(CH₂)2- | H | Cl | H | H | |
| 1-937 | Ph | N | —(CH₂)₂—CH(CF₃)—C(CCNH₂)CH- | H | Cl | H | H | |
| 1-938 | Ph | N | —(CH₂)₂—CH(CFH₂)—(CH₂)2- | H | Cl | H | H | |
| 1-939 | Ph | N | —(CH₂)₂—CH(CH₂CH₃)—(CH₂)2- | H | Cl | H | H | |
| 1-940 | Ph | N | —(CH₂)₂—CH(CH₂OH)—(CH₂)2- | H | Cl | H | H | |
| 1-941 | Ph | N | —(CH₂)₂—CH(CH₂CH₂OH)—(CH₂)2- | H | Cl | H | H | |
| 1-942 | Ph | N | —(CH₂)₂—CH(COOH)—(CH₂)2- | H | Cl | H | H | |
| 1-943 | Ph | N | —(CH₂)₂—CH(COOCH₂CH₃)—(CH₂)2- | H | Cl | H | H | |
| 1-944 | Ph | N | —(CH₂)₂—CH(Ph)—(CH₂)2- | H | Cl | H | H | |
| 1-945 | Ph | N | —(CH₂)₂—CH(CH₂Ph)—(CH₂)2- | H | Cl | H | H | |
| 1-946 | Ph | N | —(CH₂)₂—CH(F)—(CH₂)2- | H | Cl | H | H | |
| 1-947 | Ph | N | —(CH₂)₂—CF2—(CH₂)2- | H | Cl | H | H | |
| 1-948 | Ph | N | —(CH₂)₂—CH(Br)—(CH₂)2- | H | Cl | H | H | |
| 1-949 | Ph | N | —(CH₂)₂—CH&O)—(CH₂)2- | H | Cl | H | H | |
| 1-950 | Ph | N | —(CH₂)₂—CH(OH)—(CH₂)2- | H | Cl | H | H | |
| 1-951 | Ph | N | —CH(CH₃)—(CH₂)₃-CH(CH₃)— | H | Cl | H | H | |
| 1-952 | 3,5-(F)₂—Ph | N | —CH(CH₃)—(CH₂)₃-CH(CH₃)— | H | Cl | H | H | |
| 1-953 | Ph | N | —CH₂—CH(CH₃)—CH₂—CH(CH₃)—CH₂— | H | Cl | H | H | |
| 1-954 | Ph | N | —CH(CH₃)—CH₂—CH(CH₃)—CH₂—CH(CH₃) | H | Cl | H | H | |
| 1-955 | 1-NaPh | N | —(CH₂)₂—CH(CH₃)—(CH₂)₂ | H | Cl | H | H | |
| 1-956 | 2-NaPh | N | —(CH₂)₂—CH(CH₃)—(CH₂)2- | H | Cl | H | H | |
| 1-957 | 2-F—Ph | N | —(CH₂)₂—CH(CH₃)—(CH₂)2- | H | Cl | H | H | |
| 1-958 | 2-Cl—Ph | N | —(CH₂)₂—CH(CH₃)—(CH₂)2- | H | Cl | H | H | |
| 1-959 | 2-Br—Ph | N | —(CH₂)₂—CH(CH₃)—(CH₂)2- | H | Cl | H | H | |
| 1-960 | 2-I—Ph | N | —(CH₂)₂—CH(CH₃)—(CH₂)2- | H | Cl | H | H | |
| 1-961 | 2-CH₃—Ph | N | —(CH₂)₂—CH(CH₃)—(CH₂)2- | H | Cl | H | H | |
| 1-962 | 2-CF₃—Ph | N | —(CH₂)₂—CH(CH₃)—(CH₂)2- | H | Cl | H | H | |
| 1-963 | 2-CN—Ph | N | —(CH₂)₂—CH(CH₃)—(CH₂)2- | H | Cl | H | H | |
| 1-964 | 2-OCH₃—Ph | N | —(CH₂)₂—CH(CH₃)—(CH₂)2- | H | Cl | H | H | |
| 1-965 | 2-NO₂—Ph | N | —(CH₂)₂—CH(CH₃)—(CH₂)2- | H | Cl | H | H | |
| 1-966 | 2-NH₂—Ph | N | —(CH₂)₂—CH(CH₃)—(CH₂)2- | H | Cl | H | H | |
| 1-967 | 2-Ph—Ph | N | —(CH₂)₂—CH(CH₃)—(CH₂)2- | H | Cl | H | H | |
| 1-968 | 3-F—Ph | N | —(CH₂)₂—CH(CH₃)—(CH₂)2- | H | Cl | H | H | |
| 1-969 | 3-Cl—Ph | N | —(CH₂)₂—CH(CH₃)—(CH₂)2- | H | Cl | H | H | |
| 1-970 | 3-Br—Ph | N | —(CH₂)₂—CH(CH₃)—(CH₂)2- | H | Cl | H | H | |
| 1-971 | 3-I—Ph | N | —(CH₂)₂—CH(CH₃)—(CH₂)2- | H | Cl | H | H | |
| 1-972 | 3-CH₃—Ph | N | —(CH₂)₂—CH(CH₃)—(CH₂)2- | H | Cl | H | H | |
| 1-973 | 3-CF₃—Ph | N | —(CH₂)₂—CH(CH₃)—(CH₂)2- | H | Cl | H | H | |
| 1-974 | 3-CN—Ph | N | —(CH₂)₂—CH(CH₃)—(CH₂)2- | H | Cl | H | H | |
| 1-975 | 3-OCH₃—Ph | N | —(CH₂)₂—CH(CH₃)—(CH₂)2- | H | Cl | H | H | |
| 1-976 | 3-OCF₃—Ph | N | —(CH₂)₂—CH(CH₃)—(CH₂)2- | H | Cl | H | H | |
| 1-977 | 3-NO₂—Ph | N | —(CH₂)₂—CH(CH₃)—(CH₂)2- | H | Cl | H | H | |
| 1-978 | 3-NH₂—Ph | N | —(CH₂)₂—CH(CH₃)—(CH₂)2- | H | Cl | H | H | |
| 1-979 | 3-Ph—Ph | N | —(CH₂)₂—CH(CH₃)—(CH₂)2- | H | Cl | H | H | |
| 1-980 | 4-F—Ph | N | —(CH₂)₂—CH(CH₃)—(CH₂)2- | H | Cl | H | H | |
| 1-981 | 4-Cl—Ph | N | —(CH₂)₂—CH(CH₃)—(CH₂)2- | H | Cl | H | H | |
| 1-982 | 4-Br—Ph | N | —(CH₂)₂—CH(CH₃)—(CH₂)2- | H | Cl | H | H | |
| 1-983 | 4-CH₃—Ph | N | —(CH₂)₂—CH(CH₃)—(CH₂)2- | H | Cl | H | H | |
| 1-984 | 4-C(CH₃)₃—Ph | N | —(CH₂)₂—CH(CH₃)—(CH₂)2- | H | Cl | H | H | |
| 1-985 | 4-CF₃—Ph | N | —(CH₂)₂—CH(CH₃)—(CH₂)2- | H | Cl | H | H | |
| 1-986 | 4-CN—Ph | N | —(CH₂)₂—CH(CH₃)—(CH₂)2- | H | Cl | H | H | |
| 1-987 | 4-OCH₃—Ph | N | —(CH₂)₂—CH(CH₃)—(CH₂)2- | H | Cl | H | H | |
| 1-988 | 4-OCF₃—Ph | N | —(CH₂)₂—CH(CH₃)—(CH₂)2- | H | Cl | H | H | |
| 1-989 | 4-NO₂—Ph | N | —(CH₂)₂—CH(CH₃)—(CH₂)2- | H | Cl | H | H | |
| 1-990 | 4-NH₂—Ph | N | —(CH₂)₂—CH(CH₃)—(CH₂)2- | H | Cl | H | H | |
| 1-991 | 4-Ph—Ph | N | —(CH₂)₂—CH(CH₃)—(CHj2- | H | Cl | H | H | |
| 1-992 | 2-Cl-4-F—Ph | N | —(CH₂)₂—CH(CH₃)—(CH₂)2- | H | Cl | H | H | |
| 1-993 | 2-Cl-6-F—Ph | N | —(CH₂)₂—CH(CH₃)—(CH₂)2- | H | Cl | H | H | |
| 1-994 | 2,3-(F)₂—Ph | N | —(CH₂)₂—CH(CH₃)—(CH₂)2- | H | Cl | H | H | |
| 1-995 | 2,4-(F)₂—Ph | N | —(CH₂)₂—CH(CH₃)—(CH₂)2- | H | Cl | H | H | |
| 1-996 | 2,5-(F)₂—Ph | N | —(CH₂)₂—CH(CH₃)—(CH₂)2- | H | Cl | H | H | |
| 1-997 | 2,6-(F)₂—Ph | N | —(CH₂)₂—CH(CH₃)—(CH₂)2- | H | Cl | H | H | |
| 1-998 | 3,4-(F)₂—Ph | N | —(CH₂)₂—CH(CH₃)—(CH₂)2- | H | Cl | H | H | |
| 1-999 | 3,5-(F)₂—Ph | N | —(CH₂)₂—CH(CH₃)—(CH₂)2- | H | Cl | H | H | |
| 1-1000 | 2,3,4-(F)₃—Ph | N | —(CH₂)₂—CH(CH₃)—(CH₂)2- | H | Cl | H | H | |
| 1-1001 | 2,3,6-(F)₃—Ph | N | —(CH₂)₂—OH(CH₃)—(CH₂)2- | H | Cl | H | H | |
| 1-1002 | 2,4,6-(F)₃—Ph | N | —(CH₂)₂—OH(CH₃)—(CH₂)2- | H | Cl | H | H | |
| 1-1003 | 2,3,4,5,6-(F)₅—Ph | N | —(CH₂)₂—CH(CH₃)—(CH₂)2- | H | Cl | H | H | |
| 1-1004 | 2,3,4,5,6-(F)₅—Ph | N | —(CH₂)₂—CH(CH₃)—(CH₂)2- | H | Cl | F | F | |
| 1-1005 | pyridin-2-yl | N | —(CH₂)₂—CH(CH₃)—(CH₂)2- | H | Cl | H | H | |
| 1-1006 | 6-Cl-pyridin-2-yl | N | —(CH₂)₂—CH(CH₃)—(CH₂)2- | H | Cl | H | H | |
| 1-1007 | 3-Cl-5-CF₃-pyridin-2-yl | N | —(CH₂)₂—CH(CH₃)—(CH₂)2- | H | Cl | H | H | |
| 1-1008 | pyridin-3-yl | N | —(CH₂)₂—CH(CH₃)—(CH₂)2- | H | Cl | H | H | |
| 1-1009 | 6-Cl-pyridin-3-yl | N | —(CH₂)₂—CH(CH₃)—(CH₂)2- | H | Cl | H | H | |
| 1-1010 | pyridin-4-yl | N | —(CH₂)₂—CH(CH₃)—(CH₂)2- | H | Cl | H | H | |
| 1-1011 | 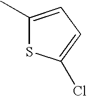 | N | —(CH₂)₂—CH(CH₃)—(CH₂)2- | H | Cl | H | H | |
| 1-1012 | 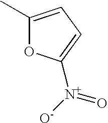 | N | —(CH₂)₂—CH(CH₃)—(CH₂)2- | H | Cl | H | H | |
| 1-1013 | Ph | N | —(CH₂)₂—CH(CH₃)—(CH₂)2- | H | H | H | H | |
| 1-1014 | Ph | N | —(CH₂)₂—CH(CH₃)—(CH₂)2- | H | F | H | H | |
| 1-1015 | Ph | N | —(CH₂)₂—CH(CH₃)—(CH₂)2- | H | CH₃ | H | H | |
| 1-1016 | Ph | N | —(CH₂)₂—CH(CH₃)—(CH₂)2- | H | OF₃ | H | H | |
| 1-1017 | Ph | N | —(CH₂)₂—CH(CH₃)—(CH₂)2- | H | OCH₃ | H | H | |
| 1-1018 | Ph | N | —(CH₂)₂—CH(CH₃)—(CH₂)2- | H | SCH₃ | H | H | |
| 1-1019 | Ph | N | —(CH₂)₂—CH(CH₃)—(CH₂)2- | H | SO₂CH₃ | H | H | |
| 1-1020 | Ph | N | —(CH₂)₂—CH(CH₃)—(CH₂)2- | H | CN | H | H | |
| 1-1021 | Ph | N | —(CH₂)₂—OH(CH₃)—(CH₂)2- | H | 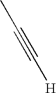 | H | H | |
| 1-1022 | Ph | N | —(CH₂)₂—CH(CH₃)—(CH₂)2- | H | 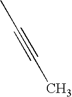 | H | H | |
| 1-1023 | Ph | N | —(CH₂)₂—CH(CH₃)—(CH₂)2- | CH₃ | Cl | H | H | |
| 1-1024 | Ph | N | —(CH₂)₂—CH(CH₃)—(CH₂)2- | CF₃ | Cl | H | H | |
| 1-1025 | Ph | N | —(CH₂)₂—CH(CH₃)—(CH₂)2- | C(CH₃)₃ | Cl | H | H | |
| 1-1026 | Ph | N | —(CH₂)₂—CH(CH₃)—(CH₂)2- | cyclopropyl | Cl | H | H | |
| 1-1027 | Ph | N | —(CH₂)₂—CH(CH₃)—(CH₂)2- | OCH₃ | Cl | H | H | |
| 1-1028 | Ph | N | —(CH₂)₂—CH(CH₃)—(CH₂)2- | O(CH₂)₃CH₃ | Cl | H | H | |
| 1-1029 | Ph | N | —(CH₂)₂—CH(CH₃)—(CH₂)2- | ON═C(CH₃)₂ | Cl | H | H | |
| 1-1030 | Ph | N | —(CH₂)₂—CH(CH₃)—(CH₂)2- | SCH₃ | Cl | H | H | |
| 1-1031 | Ph | N | —(CH₂)₂—CH(CH₃)—(CH₂)2- | SO₂CH₃ | Cl | H | H | |
| 1-1032 | Ph | N | —(CH₂)₂—CH(CH₃)—(CH₂)2- | NHPh | Cl | H | H | |
| 1-1033 | Ph | N | —(CH₂)₂—CH(CH₃)—(CH₂)2- | NH(CH₃)NH₂ | Cl | H | H | |
| 1-1034 | Ph | N | —(CH₂)₂—CH(CH₃)—(CH₂)2- | N(CH₃)NC(CH₃)₂ | Cl | H | H | |
| 1-1035 | Ph | N | —(CH₂)₂—CH(CH₃)—(CH₂)2- | N(CH₃)N═(Ph)CH₃ | Cl | H | H | |
| 1-1036 | Ph | N | —(CH₂)₂—CH(CH₃)—(CH₂)2- | CN | Cl | H | H | |
| 1-1037 | Ph | N | —(CH₂)₂—CH(CH₃)—(CH₂)2- | C(═NOH)NH₂ | Cl | H | H | |
| 1-1038 | Ph | N | —(CH₂)₂—CH(CH₃)—(CH₂)2- | C(═NOCH₃)NH₂ | Cl | H | H | |
| 1-1039 | Ph | N | —(CH₂)₂—CH(CH₃)—(CH₂)2- | C(CH₃)═NOCH₂CH₃ | Cl | H | H | |
| 1-1040 | Ph | N | —(CH₂)₂—CH(CH₃)—(CH₂)2- | C(CH₃)═NOCH₂CHCHCl | Cl | H | H | |
| 1-1041 | Ph | N | —(CH₂)₂—CH(CH₃)—(CH₂)2- | C(CH₃)═NOCH₂CH₂OCH₃ | Cl | H | H | |
| 1-1042 | Ph | N | —(CH₂)₂—CH(CH₃)—(CH₂)2- | CH₃ | OCH₃ | H | H | |
| 1-1043 | Ph | N | —(CH₂)₂—CH(CH₃)—(CH₂)2- | 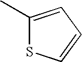 | Cl | H | H | |
| 1-1044 | Ph | N | —(CH₂)₂—CH(CH₃)—(CH₂)2- | 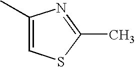 | Cl | H | H | |
| 1-1045 | Ph | N | —(CH₂)₂—CH(CH₃)—(CH₂)2- | pyridin-2-yl | Cl | H | H | |
| 1-1046 | 1-NaPh | N | —(CH₂)₂—CH(CH₃)—(CH₂)2- | pyrldin-2-yl | Cl | H | H | |
| 1-1047 | 2-NaPh | N | —(CH₂)₂—CH(CH₃)—(CH₂)2- | pyridin-2-yl | Cl | H | H | |
| 1-1048 | 2-F—Ph | N | —(CH₂)₂—CH(CH₃)—(CH₂)2- | pyridin-2-yl | Cl | H | H | |
| 1-1049 | 2-Cl—Ph | N | —(CH₂)₂—CH(CH₃)—(CH₂)2- | pyridin-2-yl | Cl | H | H | |
| 1-1050 | 2-Br—Ph | N | —(CH₂)₂—CH(CH₃)—(CH₂)2- | pyridin-2-yl | Cl | H | H | |
| 1-1051 | 2-I—Ph | N | —(CH₂)₂—CH(CH₃)—(CH₂)2- | pyridin-2-yl | Cl | H | H | |
| 1-1052 | 2-CH₃—Ph | N | —(CH₂)₂—CH(CH₃)—(CH₂)2- | pyridin-2-yl | Cl | H | H | |
| 1-1053 | 2-CF₃—Ph | N | —(CH₂)₂—CH(CH₃)—(CH₂)2- | pyridin-2-yl | Cl | H | H | |
| 1-1054 | 2-CN | —Ph | N | —(CH₂)₂—CH(CH₃)—(CH₂)2- | pyridin-2-yl | Cl | H | H |
| 1-1055 | 2OCH~rPh | N | —(CH₂)₂—CH(CH₃)—(CH₂)2- | pyridin-2-yl | Cl | H | H | |
| 1-1056 | 2-NO₂—Ph | N | —(CH₂)₂—CH(CH₃)—(CH₂)2- | pyridin-2-yl | Cl | H | H | |
| 1-1057 | 2-NH₂—Ph | N | —(CH₂)₂—CH(CH₃)—(CH₂)2- | pyridin-2-yl | Cl | H | H | |
| 1-1058 | 2-Ph—Ph | N | —(CH₂)₂—CH(CH₃)—(CH₂)2- | pyridin-2-yl | Cl | H | H | |
| 1-1059 | 3-F—Ph | N | —(CH₂)₂—CH(CH₃)—(CH₂)2- | pyridin-2-yl | Cl | H | H | |
| 1-1060 | 3-Cl—Ph | N | —(CH₂)₂—CH(CH₃)—(CH₂)2- | pyridin-2-yl | Cl | H | H | |
| 1-1061 | 3-Br—Ph | N | —(CH₂)₂—CH(CH₃)—(CH₂)2- | pyridin-2-yl | Cl | H | H | |
| 1-1062 | 3-I—Ph | N | —(CH₂)₂—CH(CH₃)—(CH₂)2- | pyridin-2-yl | Cl | H | H | |
| 1-1063 | 3-CH₃—Ph | N | —(CH₂)₂—CH(CH₃)—(CH₂)2- | pyridin-2-yl | Cl | H | H | |
| 1-1064 | 3-CF₃—Ph | N | —(CH₂)₂—CH(CH₃)—(CH₂)2- | pyridin-2-yl | Cl | H | H | |
| 1-1065 | 3-CN—Ph | N | —(CH₂)₂—CH(CH₃)—(CH₂)2- | pyridin-2-yl | Cl | H | H | |
| 1-1066 | 3-OCH3Ph | N | —(CH₂)₂—CH(CH₃)—(CH₂)2- | pyridin-2-yl | Cl | H | H | |
| 1-1067 | 3-OCF3Ph | N | —(CH₂)₂—CH(CH₃)—(CH₂)2- | pyridin-2-yl | Cl | H | H | |
| 1-1068 | 3-NO₂—Ph | N | —(CH₂)₂—CH(CH₃)—(CH₂)2- | pyridin-2-yl | Cl | H | H | |
| 1-1069 | 3-NH₂—Ph | N | —(CH₂)₂—CH(CH₃)—(CH₂)2- | pyridin-2-yl | Cl | H | H | |
| 1-1070 | 3-Ph—Ph | N | —(CH₂)₂—CH(CH₃)—(CH₂)2- | pyridin-2-yl | Cl | H | H | |
| 1-1071 | 4-F—Ph | N | —(CH₂)₂—CH(CH₃)—(CH₂)2- | pyridin-2-yl | Cl | H | H | |
| 1-1072 | 4-Cl—Ph | N | —(CH₂)₂—CH(CH₃)—(CH₂)2- | pyridin-2-yl | Cl | H | H | |
| 1-1073 | 4-Br—Ph | N | —(CH₂)₂—CH(CH₃)—(CH₂)2- | pyridin-2-yl | Cl | H | H | |
| 1-1074 | 4-CH₃—Ph | N | —(CH₂)₂—CH(CH₃)—(CH₂)2- | pyridin-2-yl | Cl | H | H | |
| 1-1075 | 4-C(CH₃)₃Ph | N | —(CH₂)₂—CH(CH₃)—(CH₂)2- | pyridin-2-yl | Cl | H | H | |
| 1-1076 | 4-CF3Ph | N | —(CH₂)₂—CH(CH₃)—(CH₂)2- | pyridin-2-yl | Cl | H | H | |
| 1-1077 | 4-CN—Ph | N | —(CH₂)₂—CH(CH₃)—(CH₂)2- | pyridin-2-yl | Cl | H | H | |
| 1-1078 | 4-OCH₃—Ph | N | —(CH₂)₂—CH(CH₃)—(CH₂)2- | pyridin-2-yl | Cl | H | H | |
| 1-1079 | 4-OCF₃—Ph | N | —(CH₂)₂—CH(CH₃)—(CH₂)2- | pyridin-2-yl | Cl | H | H | |
| 1-1080 | 4-NO₂—Ph | N | —(CH₂)₂CH(CH₃)—(CH₂)2- | pyridin-2-yl | Cl | H | H | |
| 1-1081 | 4-NH₂—Ph | N | —(CH₂)₂CH(CH₃)—(CH₂)2- | pyridin-2-yl | Cl | H | H | |
| 1-1082 | 4-Ph—Ph | N | —(CH₂)₂—CH(CH₃)—(CH₂)2- | pyridin-2-yl | Cl | H | H | |
| 1-1083 | 2-Cl-4-F—Ph | N | —(CH₂)₂—CH(CH₃)—(CH₂)2- | pyridin-2-yl | Cl | H | H | |
| 1-1084 | 2-Cl-6-F—Ph | N | —(CH₂)₂—CH(CH₃)—(CH₂)2- | pyridin-2-yl | Cl | H | H | |
| 1-1085 | 2,3-(F)₂—Ph | N | —(CH₂)₂—CH(CH₃)—(CH₂)2- | pyridin-2-yl | Cl | H | H | |
| 1-1086 | 2,4-(F)₂—Ph | N | —(CH₂)₂—CH(CH₃)—(CH₂)2- | pyridin-2-yl | Cl | H | H | |
| 1-1087 | 2,5-(F)₂—Ph | N | —(CH₂)₂—CH(CH₃)—(CH₂)2- | pyridin-2-yl | Cl | H | H | |
| 1-1088 | 2,6-(F)₂—Ph | N | —(CH₂)₂—CH(CH₃)—(CH₂)2- | pyridin-2-yl | Cl | H | H | |
| 1-1089 | 3,4-(F)₂—Ph | N | —(CH₂)₂—CH(CH₃)—(CH₂)2- | pyridin-2-yl | Cl | H | H | |
| 1-1090 | 3,5-(F)₂—Ph | N | —(CH₂)₂—CH(CH₃)—(CH₂)2- | pyridin-2-yl | Cl | H | H | |
| 1-1091 | 2,3,4-(F)₃—Ph | N | —(CH₂)₂—CH(CH₃)—(CH₂)2- | pyridin-2-yl | Cl | H | H | |
| 1-1092 | 2,3,6-(F)₃—Ph | N | —(CH₂)₂—CH(CH₃)—(CH₂)2- | pyridin-2-yl | Cl | H | H | |
| 1-1093 | 2,4,6-(F)₃—Ph | N | —(CH₂)₂—CH(CH₃)—(CH₂)2- | pyridin-2-yl | Cl | H | H | |
| 1-1094 | 2,3,4,5,6-(F)₅—Ph | N | —(CH₂)₂—CH(CH₃)—(CH₂)2- | pyridin-2-yl | Cl | H | H | |
| 1-1095 | 2,3,4,5,6-(F)₅—Ph | N | —(CH₂)₂—CH(CH₃)—(CH₂)2- | pyridin-2-yl | Cl | F | F | |
| 1-1096 | pyridin-2-yl | N | —(CH₂)₂—CH(CH₃)—(CH₂)2- | pyridin-2-yl | Cl | H | H | |
| 1-1097 | 6-Cl-pyridin-2-yl | N | —(CH₂)₂—CH(CH₃)—(CH₂)2- | pyridin-2-yl | Cl | H | H | |
| 1-1098 | 3-Cl-5-CF₃-pyridin-2-yl | N | —(CH₂)₂—CH(CH₃)—(CH₂)2- | pyridin-2-yl | Cl | H | H | |
| 1-1099 | pyridin-3-yl | N | —(CH₂)₂—CH(CH₃)—(CH₂)2- | pyridin-2-yl | Cl | H | H | |
| 1-1100 | 6-Cl-pyridin-3-yl | N | —(CH₂)₂—CH(CH₃)—(CH₂)2- | pyridin-2-yl | Cl | H | H | |
| 1-1101 | pyridin-4-yl | N | —(CH₂)₂—CH(CH₃)—(CH₂)2- | pyridin-2-yl | Cl | H | H | |
| 1-1102 | 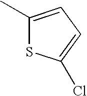 | N | —(CH₂)₂—CH(CH₃)—(CH₂)2- | pyridin-2-yl | Cl | H | H | |
| 1-1103 | 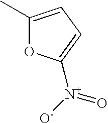 | N | —(CH₂)₂—CH(CH₃)—(CH₂)2- | pyridin-2-yl | Cl | H | H | |
| 1-1104 | Ph | N | —(CH₂)₂—CH(CH₃)—(CH₂)2- | pyridin-2-yl | H | H | H | |
| 1-1105 | Ph | N | —(CH₂)4-CH(CF₃)— | pyridin-2-yl | Cl | H | H | |
| 1-1106 | Ph | N | —(CH₂)₂—CH(CH₃)—(CH₂)2- | 3-CH₃-pyridin-2-yl | Cl | H | H | |
| 1-1107 | Ph | N | —(CH₂)₂—CH(CH₃)—(CH₂)2- | 4-CH₃-pyridin-2-yl | Cl | H | H | |
| 1-1108 | Ph | N | —(CH₂)₂—CH(CH₃)—(CH₂)2- | 6-CH₃-pyridin-2-yl | Cl | H | H | |
| 1-1109 | Ph | N | —(CH₂)₂—CH(CH₃)—(CH₂)2- | pyridin-3-yl | Cl | H | H | |
| 1-1110 | Ph | N | —(CH₂)₂—CH(CH₃)—(CH₂)2- | pyridin-4-yl | Cl | H | H | |
| 1-1111 | Ph | N | —(CH₂)₂—CH(CH₃)—(CH₂)2- | 2,6-(Cl)₂-pyridin-4-yl | Cl | H | H | |
| 1-1112 | Ph | N | —(CH₂)₂—CH(CH₃)—(CH₂)2- | 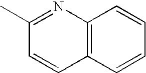 | Cl | H | H | |
| 1-1113 | Ph | N | —(CH₂)₂—CH(CH₃)—(CH₂)2- | 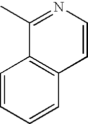 | Cl | H | H | |
| 1-1114 | Ph | N | —(CH₂)₂—CH(CH₃)—(CH₂)2- | 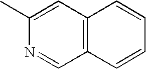 | Cl | H | H | |
| 1-1115 | Ph | N | —(CH₂)₂—CH(CH₃)—(CH₂)2- | pyrimidin-2-yl | Cl | H | H | |
| 1-1116 | Ph | N | —(CH₂)₂—CH(CH₃)—(CH₂)2- | pyridazin-3-yl | Cl | H | H | |
| 1-1117 | Ph | N | —(CH₂)₂—CH(CH₃)—(CH₂)2- | pyrazin-2-yl | Cl | H | H | |
| 1-1118 | 1-NaPh | N | —(CH₂)₂—CH(CH₃)—(CH₂)2- | pyrazin-2-yl | Cl | H | H | |
| 1-1119 | 2-NaPh | N | —(CH₂)₂—CH(CH₃)—(CH₂)2- | pyrazin-2-yl | Cl | H | H | |
| 1-1120 | 2-F—Ph | N | —(CH₂)₂—CH(CH₃)—(CH₂)2- | pyrazin-2-yl | Cl | H | H | |
| 1-1121 | 2-Cl—Ph | N | —(CH₂)₂—CH(CH₃)—(CH₂)2- | pyrazin-2-yl | Cl | H | H | |
| 1-1122 | 2-Br—Ph | N | —(CH₂)₂—CH(CH₃)—(CH₂)2- | pyrazin-2-yl | Cl | H | H | |
| 1-1123 | 2-I—Ph | N | —(CH₂)₂—CH(CH₃)—(CH₂)2- | pyrazin-2-yl | Cl | H | H | |
| 1-1124 | 2-CH₃—Ph | N | —(CH₂)₂—CH(CH₃)—(CH₂)2- | pyrazin-2-yl | Cl | H | H | |
| 1-1125 | 2-CF₃—Ph | N | —(CH₂)₂—CH(CH₃)—(CH₂)2- | pyrazin-2-yl | Cl | H | H | |
| 1-1126 | 2-CN—Ph | N | —(CH₂)₂—CH(CH₃)—(CH₂)2- | pyrazin-2-yl | Cl | H | H | |
| 1-1127 | 2-OCH₃—Ph | N | —(CH₂)₂—CH(CH₃)—(CH₂)2- | pyrazin-2-yl | Cl | H | H | |
| 1-1128 | 2-NO₂—Ph | N | —(CH₂)₂—CH(CH₃)—(CH₂)2- | pyrazin-2-yl | Cl | H | H | |
| 1-1129 | 2-NH₂—Ph | N | —(CH₂)₂—CH(CH₃)—(CH₂)2- | pyrazin-2-yl | Cl | H | H | |
| 1-1130 | 2-Ph—Ph | N | —(CH₂)₂—CH(CH₃)—(CH₂)2- | pyrazin-2-yl | Cl | H | H | |
| 1-1131 | 3-F—Ph | N | —(CH₂)₂—CH(CH₃)—(CH₂)2- | pyrazin-2-yl | Cl | H | H | |
| 1-1132 | 3-Cl—Ph | N | —(CH₂)₂—CH(CH₃)—(CH₂)2- | pyrazin-2-yl | Cl | H | H | |
| 1-1133 | 3-Br—Ph | N | —(CH₂)₂CH(CH₃)—(CH₂)2- | pyrazin-2-yl | Cl | H | H | |
| 1-1134 | 3-I—Ph | N | —(CH₂)₂—CH(CH₃)—(CH₂)2- | pyrazin-2-yl | Cl | H | H | |
| 1-1135 | 3-CH₃—Ph | N | —(CH₂)₂—CH(CH₃)—(CH₂)2- | pyrazin-2-yl | Cl | H | H | |
| 1-1136 | 3-CF₃—Ph | N | —(CH₂)₂CH(CH₃)—(CH₂)2- | pyrazin-2-yl | Cl | H | H | |
| 1-1137 | 3-CN—Ph | N | —(CH₂)₂—CH(CH₃)—(CH₂)2- | pyrazin-2-yl | Cl | H | H | |
| 1-1138 | 3-OCH₂—Ph | N | —(CH₂)₂CH(CH₃)—(CH₂)2- | pyrazin-2-yl | Cl | H | H | |
| 1-1139 | 3-OCF₃—Ph | N | —(CH₂)₂CH(CH₃)—(CH₂)2- | pyrazin-2-yl | Cl | H | H | |
| 1-1140 | 3-NO₂—Ph | N | —(CH₂)₂CH(CH₃)—(CH₂)2- | pyrazin-2-yl | Cl | H | H | |
| 1-1141 | 3-NH₂—Ph | N | —(CH₂)₂CH(CH₃)—(CH₂)2- | pyrazin-2-yl | Cl | H | H | |
| 1-1142 | 3-Ph—Ph | N | —(CH₂)₂—CH(CH₃)—(CH₂)2- | pyrazin-2-yl | Cl | H | H | |
| 1-1143 | 4-F—Ph | N | —(CH₂)₂—CH(CH₃)—(CH₂)2- | pyrazin-2-yl | Cl | H | H | |
| 1-1144 | 4-Cl—Ph | N | —(CH₂)₂—CH(CH₃)—(CH₂)2- | pyrazin-2-yl | Cl | H | H | |
| 1-1145 | 4-Br—Ph | N | —(CH₂)₂—CH(CH₃)—(CH₂)2- | pyrazin-2-yl | Cl | H | H | |
| 1-1146 | 4-CH₃—Ph | N | —(CH₂)₂—CH(CH₃)—(CH₂)2- | pyrazin-2-yl | Cl | H | H | |
| 1-1147 | 4-C(CH₃)₃—Ph | N | —(CH₂)₂—CH(CH₃)—(CH₂)2- | pyrazin-2-yl | Cl | H | H | |
| 1-1148 | 4-CF₃—Ph | N | —(CH₂)₂—CH(CH₃)—(CH₂)2- | pyrazin-2-yl | Cl | H | H | |
| 1-1149 | 4-CN—Ph | N | —(CH₂)₂—CH(CH₃)—(CH₂)2- | pyrazin-2-yl | Cl | H | H | |
| 1-1150 | 4-OCH₃—Ph | N | —(CH₂)₂—CH(CH₃)—(CH₂)2- | pyrazin-2-yl | Cl | H | H | |
| 1-1151 | 4-OCF₃—Ph | N | —(CH₂)₂—CH(CH₃)—(CH₂)2- | pyrazin-2-yl | Cl | H | H | |
| 1-1152 | 4-NO₂—Ph | N | —(CH₂)₂—CH(CH₃)—(CH₂)2- | pyrazin-2-yl | Cl | H | H | |
| 1-1153 | 4-NH₂—Ph | N | —(CH₂)₂—CH(CH₃)—(CH₂)2- | pyrazin-2-yl | Cl | H | H | |
| 1-1154 | 4-Ph—Ph | N | —(CH₂)₂—CH(CH₃)—(CH₂)2- | pyrazin-2-yl | Cl | H | H | |
| 1-1155 | 2-Cl-4-F—Ph | N | —(CH₂)₂—CH(CH₃)—(CH₂)2- | pyrazin-2-yl | Cl | H | H | |
| 1-1156 | 2-Cl-6-F—Ph | N | —(CH₂)₂—CH(CH₃)—(CH₂)2- | pyrazin-2-yl | Cl | H | H | |
| 1-1157 | 2,3(F)₂—Ph | N | —(CH₂)₂—CH(CH₃)—(CH₂)2- | pyrazin-2-yl | Cl | H | H | |
| 1-1158 | 2,4(F)₂—Ph | N | —(CH₂)₂—CH(CH₃)—(CH₂)2- | pyrazin-2-yl | Cl | H | H | |
| 1-1159 | 2,5-(F)₂—Ph | N | —(CH₂)₂—CH(CH₃)—(CH₂)2- | pyrazin-2-yl | Cl | H | H | |
| 1-1160 | 2,6-(F)₂—Ph | N | —(CH₂)₂—CH(CH₃)—(CH₂)2- | pyrazin-2-yl | Cl | H | H | |
| 1-1161 | 3,4-(F)₂—Ph | N | —(CH₂)₂—CH(CH₃)—(CH₂)2- | pyrazin-2-yl | Cl | H | H | |
| 1-1162 | 3,5-(F)₂—Ph | N | —(CH₂)₂—CH(CH₃)—(CH₂)2- | pyrazin-2-yl | Cl | H | H | |
| 1-1163 | 2,3,4-(F)₃—Ph | N | —(CH₂)₂—CH(CH₃)—(CH₂)2- | pyrazin-2-yl | Cl | H | H | |
| 1-1164 | 2,3,6-(F)₃—Ph | N | —(CH₂)₂—CH(CH₃)—(CH₂)2- | pyrazin-2-yl | Cl | H | H | |
| 1-1165 | 2,4,6-(F)₃—Ph | N | —(CH₂)₂—CH(CH₃)—(CH₂)2- | pyrazin-2-yl | Cl | H | H | |
| 1-1166 | 2,3,4,5,6-(F)₅—Ph | N | —(CH₂)₂—CH(CH₃)—(CH₂)2- | pyrazin-2-yl | Cl | H | H | |
| 1-1167 | 2,3,4,5,6-(F)₅—Ph | N | —(CH₂)₂—CH(CH₃)—(CH₂)2- | pyrazin-2-yl | Cl | F | F | |
| 1-1168 | pyridin-2-yl | N | —(CH₂)₂—CH(CH₃)—(CH₂)2- | pyrazin-2-yl | Cl | H | H | |
| 1-1169 | 6-Cl-pyridin-2-yl | N | —(CH₂)₂—CH(CH₃)—(CH₂)2- | pyrazin-2-yl | Cl | H | H | |
| 1-1170 | 3-Cl-5-CF₃-pyridin-2-yl | N | —(CH₂)₂—CH(CH₃)—(CH₂)2- | pyrazin-2-yl | Cl | H | H | |
| 1-1171 | pyridin-3-yl | N | —(CH₂)₂—CH(CH₃)—(CH₂)2- | pyrazin-2-yl | Cl | H | H | |
| 1-1172 | 6-Cl-pyridin-3-yl | N | —(CH₂)₂—CH(CH₃)—(CH₂)2- | pyrazin-2-yl | Cl | H | H | |
| 1-1173 | pyridin-4-yl | N | —(CH₂)₂—CH(CH₃)—(CH₂)2- | pyrazin-2-yl | Cl | H | H | |
| 1-1174 | 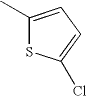 | N | —(CH₂)₂—CH(CH₃)—(CH₂)2- | pyrazin-2-yl | Cl | H | H | |
| 1-1175 | 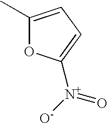 | N | —(CH₂)₂—CH(CH₃)—(CH₂)2- | pyrazin-2-yl | Cl | H | H | |
| 1-1176 | Ph | N | —(CH₂)₂—CH(CH₃)—(CH₂)2- | pyrazin-2-yl | H | H | H | |
| 1-1177 | Ph | N | —(CH₂)₂—CH(CH₃)—(CH₂)2- | pyrazol-1-yl | Cl | H | H | |
| 1-1178 | 1-NaPh | N | —(CH₂)₂—CH(CH₃)—(CH₂)2- | pyrazol-1-yl | Cl | H | H | |
| 1-1179 | 2-NaPh | N | —(CH₂)₂—CH(CH₃)—(CH₂)2- | pyrazol-1-yl | Cl | H | H | |
| 1-1180 | 2-F—Ph | N | —(CH₂)₂—CH(CH₃)—(CH₂)2- | pyrazol-1-yl | Cl | H | H | |
| 1-1181 | 2-Cl—Ph | N | —(CH₂)₂—CH(CH₃)—(CH₂)2- | pyrazol-1-yl | Cl | H | H | |
| 1-1182 | 2-Br—Ph | N | —(CH₂)₂—CH(CH₃)—(CH₂)2- | pyrazol-1-yl | Cl | H | H | |
| 1-1183 | 2-I—Ph | N | —(CH₂)₂—CH(CH₃)—(CH₂)2- | pyrazol-1-yl | Cl | H | H | |
| 1-1184 | 2-CH₃—Ph | N | —(CH₂)₂—CH(CH₃)—(CH₂)2- | pyrazol-1-yl | Cl | H | H | |
| 1-1185 | 2-CF₃—Ph | N | —(CH₂)₂—CH(CH₃)—(CH₂)2- | pyrazol-1-yl | Cl | H | H | |
| 1-1186 | 2-CN—Ph | N | —(CH₂)₂—CH(CH₃)—(CH₂)2- | pyrazol-1-yl | Cl | H | H | |
| 1-1187 | 2-OCH₃—Ph | N | —(CH₂)₂—CH(CH₃)—(CH₂)2- | pyrazol-1-yl | Cl | H | H | |
| 1-1188 | 2-NO₂—Ph | N | —(CH₂)₂—CH(CH₃)—(CH₂)2- | pyrazol-1-yl | Cl | H | H | |
| 1-1189 | 2-NH₂—Ph | N | —(CH₂)₂—CH(CH₃)—(CH₂)2- | pyrazol-1-yl | Cl | H | H | |
| 1-1190 | 2-Ph—Ph | N | —(CH₂)₂—CH(CH₃)—(CH₂)2- | pyrazol-1-yl | Cl | H | H | |
| 1-1191 | 3-F—Ph | N | —(CH₂)₂—CH(CH₃)—(CH₂)2- | pyrazol-1-yl | Cl | H | H | |
| 1-1192 | 3-Cl—Ph | N | —(CH₂)₂—CH(CH₃)—(CH₂)2- | pyrazol-1-yl | Cl | H | H | |
| 1-1193 | 3-Br—Ph | N | —(CH₂)₂—CH(CH₃)—(CH₂)2- | pyrazol-1-yl | Cl | H | H | |
| 1-1194 | 3-I—Ph | N | —(CH₂)₂—CH(CH₃)—(CH₂)2- | pyrazol-1-yl | Cl | H | H | |
| 1-1195 | 3-CH₃—Ph | N | —(CH₂)₂—CH(CH₃)—(CH₂)2- | pyrazol-1-yl | Cl | H | H | |
| 1-1196 | 3-CF₃—Ph | N | —(CH₂)₂—CH(CH₃)—(CH₂)2- | pyrazol-1-yl | Cl | H | H | |
| 1-1197 | 3-CN—Ph | N | —(CH₂)₂—CH(CH₃)—(CH₂)2- | pyrazol-1-yl | Cl | H | H | |
| 1-1198 | 3-OCH₃—Ph | N | —(CH₂)₂—CH(CH₃)—(CH₂)2- | pyrazol-1-yl | Cl | H | H | |
| 1-1199 | 3-OCF₃—Ph | N | —(CH₂)₂—CH(CH₃)—(CH₂)2- | pyrazol-1-yl | Cl | H | H | |
| 1-1200 | 3-NO₂—Ph | N | —(CH₂)₂—CH(CH₃)—(CH₂)2- | pyrazol-1-yl | Cl | H | H | |
| 1-1201 | 3-NH₂—Ph | N | —(CH₂)₂—CH(CH₃)—(CH₂)2- | pyrazol-1-yl | Cl | H | H | |
| 1-1202 | 3-Ph—Ph | N | —(CH₂)₂—CH(CH₃)—(CH₂)2- | pyrazol-1-yl | Cl | H | H | |
| 1-1203 | 4-F—Ph | N | —(CH₂)₂—CH(CH₃)—(CH₂)2- | pyrazol-1-yl | Cl | H | H | |
| 1-1204 | 4-Cl—Ph | N | —(CH₂)₂—CH(CH₃)—(CH₂)2- | pyrazol-1-yl | Cl | H | H | |
| 1-1205 | 4-Br—Ph | N | —(CH₂)₂—CH(CH₃)—(CH₂)2- | pyrazol-1-yl | Cl | H | H | |
| 1-1206 | 4-CH₃—Ph | N | —(CH₂)₂—CH(CH₃)—(CH₂)2- | pyrazol-1-yl | Cl | H | H | |
| 1-1207 | 4-C(CH₃)₃—Ph | N | —(CH₂)₂—CH(CH₃)—(CH₂)2- | pyrazol-1-yl | Cl | H | H | |
| 1-1208 | 4-CF₃—Ph | N | —(CH₂)₂—CH(CH₃)—(CH₂)2- | pyrazol-1-yl | Cl | H | H | |
| 1-1209 | 4-CN—Ph | N | —(CH₂)₂—CH(CH₃)—(CH₂)2- | pyrazol-1-yl | Cl | H | H | |
| 1-1210 | 4-OCH₃—Ph | N | —(CH₂)₂—CH(CH₃)—(CH₂)2- | pyrazol-1-yl | Cl | H | H | |
| 1-1211 | 4-OCF₃—Ph | N | —(CH₂)₂—CH(CH₃)—(CH₂)2- | pyrazol-1-yl | Cl | H | H | |
| 1-1212 | 4-NO₂—Ph | N | —(CH₂)₂—CH(CH₃)—(CH₂)2- | pyrazol-1-yl | Cl | H | H | |
| 1-1213 | 4-NH₂—Ph | N | —(CH₂)₂—CH(CH₃)—(CH₂)2- | pyrazol-1-yl | Cl | H | H | |
| 1-1214 | 4-Ph—Ph | N | —(CH₂)₂—CH(CH₃)—(CH₂)2- | pyrazol-1-yl | Cl | H | H | |
| 1-1215 | 2-Cl-4-F—Ph | N | —(CH₂)₂—OH(CH₃)—(CH₂)2- | pyrazol-1-yl | Cl | H | H | |
| 1-1216 | 2-Cl-6-F—Ph | N | —(CH₂)₂—CH(CH₃)—(CH₂)2- | pyrazol-1-yl | Cl | H | H | |
| 1-1217 | 2,3-(F)₂—Ph | N | —(CH₂)₂—CH(CH₃)—(CH₂)2- | pyrazol-1-yl | Cl | H | H | |
| 1-1218 | 2,4-(F)₂—Ph | N | —(CH₂)₂—CH(CH₃)—(CH₂)2- | pyrazol-1-yl | Cl | H | H | |
| 1-1219 | 2,5-(F)₂—Ph | N | —(CH₂)₂—CH(CH₃)—(CH₂)2- | pyrazol-1-yl | Cl | H | H | |
| 1-1220 | 2,6-(F)₂—Ph | N | —(CH₂)₂—CH(CH₃)—(CH₂)2- | pyrazol-1-yl | Cl | H | H | |
| 1-1221 | 3,4-(F)₂—Ph | N | —(CH₂)₂—CH(CH₃)—(CH₂)2- | pyrazol-1-yl | Cl | H | H | |
| 1-1222 | 3,5-(F)₂—Ph | N | —(CH₂)₂—CH(CH₃)—(CH₂)2- | pyrazol-1-yl | Cl | H | H | |
| 1-1223 | 2,3,4-(F)₃—Ph | N | —(CH₂)₂—CH(CH₃)—(CH₂)2- | pyrazol-1-yl | Cl | H | H | |
| 1-1224 | 2,3,6-(F)₃—Ph | N | —(CH₂)₂—CH(CH₃)—(CH₂)2- | pyrazol-1-yl | Cl | H | H | |
| 1-1225 | 2,4,6-(F)₃—Ph | N | —(CH₂)₂—CH(CH₃)—(CH₂)2- | pyrazol-1-yl | Cl | H | H | |
| 1-1226 | 2,3,4,5,6-(F)₅—Ph | N | —(CH₂)₂—CH(CH₃)—(CH₂)2- | pyrazol-1-yl | Cl | H | H | |
| 1-1227 | 2,3,4,5,6-(F)₅—Ph | N | —(CH₂)₂—CH(CH₃)—(CH₂)2- | pyrazol-1-yl | Cl | F | F | |
| 1-1228 | pyridin-2-yl | N | —(CH₂)₂—CH(CH₃)—(CH₂)2- | pyrazol-1-yl | Cl | H | H | |
| 1-1229 | 6-Cl-pyridin-2-yl | N | —(CH₂)₂—CH(CH₃)—(CH₂)2- | pyrazol-1-yl | Cl | H | H | |
| 1-1230 | 3-Cl-5-CF₃-pyridin-2-yl | N | —(CH₂)₂—CH(CH₃)—(CH₂)2- | pyrazol-1-yl | Cl | H | H | |
| 1-1231 | pyridin-3-yl | N | —(CH₂)₂—CH(CH₃)—(CH₂)2- | pyrazol-1-yl | Cl | H | H | |
| 1-1232 | 6-Cl-pyridin-3-yl | N | —(CH₂)₂—CH(CH₃)—(CH₂)2- | pyrazol-1-yl | Cl | H | H | |
| 1-1233 | pyridin-4-yl | N | —(CH₂)₂—CH(CH₃)—(CH₂)2- | pyrazol-1-yl | Cl | H | H | |
| 1-1234 | 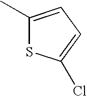 | N | —(CH₂)₂—CH(CH₃)—(CH₂)2- | pyrazol-1-yl | Cl | H | H | |
| 1-1235 | 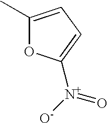 | N | —(CH₂)₂—CH(CH₃)—(CH₂)2- | pyrazol-1-yl | Cl | H | H | |
| 1-1236 | Ph | N | —(CH₂)₂—CH(CH₃)—(CH₂)2- | pyrazol-1-yl | H | H | H | |
| 1-1237 | Ph | N | —(CH₂)₂—CH(CH₃)—(CH₂)2- | 3-CH₃-pyrazol-1-yl | Cl | H | H | |
| 1-1238 | Ph | N | —(CH₂)₂—CH(CH₃)—(CH₂)2- | 3,5-(CH₃)₂-pyrazol-1-yl | Cl | H | H | |
| 1-1239 | Ph | N | —(CH₂)₂—CH(CH₃)—(CH₂)2- | 3-CF₃-pyrazol-1-yl | Cl | H | H | |
| 1-1240 | Ph | N | —(CH₂)₂—CH(CH₃)—(CH₂)2- | 3,5-(CF₃)₂-pyrazol-1-yl | Cl | H | H | |
| 1-1241 | Ph | N | —(CH₂)₂—CH(CH₃)—(CH₂)2- | 4-Br-pyrazol-1-yl | Cl | H | H | |
| 1-1242 | Ph | N | —(CH₂)₂—CH(CH₃)—(CH₂)2- | 4-CH₃-pyrazol-1-yl | Cl | H | H | |
| 1-1243 | Ph | N | —(CH₂)₂—CH(CH₃)—(CH₂)2- | imidazol-1-yl | Cl | H | H | |
| 1-1244 | Ph | N | —(CH₂)₂—CH(CH₃)—(CH₂)2- | 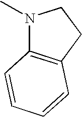 | Cl | H | H | |
| 1-1245 | Ph | N | —(CH₂)₂—CH(CH₃)—(CH₂)2- | 1,2,4-triazol-1-yl | Cl | H | H | |
| 1-1246 | 1-NaPh | N | —(CH₂)₂—CH(CH₃)—(CH₂)2- | 1,2,4-triazol-1-yl | Cl | H | H | |
| 1-1247 | 2-NaPh | N | —(CH₂)₂—CH(CH₃)—(CH₂)2- | 1,2,4-triazol-1-yl | Cl | H | H | |
| 1-1248 | 2-F—Ph | N | —(CH₂)₂—CH(CH₃)—(CH₂)2- | 1,2,4-triazol-1-yl | Cl | H | H | |
| 1-1249 | 2-Cl—Ph | N | —(CH₂)₂—CH(CH₃)—(CH₂)2- | 1,2,4-triazol-1-yl | Cl | H | H | |
| 1-1250 | 2-Br—Ph | N | —(CH₂)₂—CH(CH₃)—(CH₂)2- | 1,2,4-triazol-1-yl | Cl | H | H | |
| 1-1251 | 2-I—Ph | N | —(CH₂)₂—CH(CH₃)—(CH₂)2- | 1,2,4-triazol-1-yl | Cl | H | H | |
| 1-1252 | 2-CH₃—Ph | N | —(CH₂)₂—CH(CH₃)—(CH₂)2- | 1,2,4-triazol-1-yl | Cl | H | H | |
| 1-1253 | 2-CF₃—Ph | N | —(CH₂)₂—CH(CH₃)—(CH₂)2- | 1,2,4-triazol-1-yl | Cl | H | H | |
| 1-1254 | 2-CN—Ph | N | —(CH₂)₂—CH(CH₃)—(CH₂)2- | 1,2,4-triazol-1-yl | Cl | H | H | |
| 1-1255 | 2-OCH₃—Ph | N | —(CH₂)₂—CH(CH₃)—(CH₂)2- | 1,2,4-triazol-1-yl | Cl | H | H | |
| 1-1256 | 2-NO₂—Ph | N | —(CH₂)₂—CH(CH₃)—(CH₂)2- | 1,2,4-triazol-1-yl | Cl | H | H | |
| 1-1257 | 2-NH₂—Ph | N | —(CH₂)₂—CH(CH₃)—(CH₂)2- | 1,2,4-triazol-1-yl | Cl | H | H | |
| 1-1258 | 2-Ph—Ph | N | —(CH₂)₂—CH(CH₃)—(CH₂)2- | 1,2,4-triazol-1-yl | Cl | H | H | |
| 1-1259 | 3-F—Ph | N | —(CH₂)₂—CH(CH₃)—(CH₂)2- | 1,2,4-triazol-1-yl | Cl | H | H | |
| 1-1260 | 3-Cl—Ph | N | —(CH₂)₂—CH(CH₃)—(CH₂)2- | 1,2,4-triazol-1-yl | Cl | H | H | |
| 1-1261 | 3-Br—Ph | N | —(CH₂)₂—CH(CH₃)—(CH₂)2- | 1,2,4-triazol-1-yl | Cl | H | H | |
| 1-1262 | 3-I—Ph | N | —(CH₂)₂—CH(CH₃)—(CH₂)2- | 1,2,4-triazol-1-yl | Cl | H | H | |
| 1-1263 | 3-CH₃—Ph | N | —(CH₂)₂—CH(CH₃)—(CH₂)2- | 1,2,4-triazol-1-yl | Cl | H | H | |
| 1-1264 | 3-CF₃—Ph | N | —(CH₂)₂—CH(CH₃)—(CH₂)2- | 1,2,4-triazol-1-yl | Cl | H | H | |
| 1-1265 | 3-CN—Ph | N | —(CH₂)₂—CH(CH₃)—(CH₂)2- | 1,2,4-triazol-1-yl | Cl | H | H | |
| 1-1266 | 3-OCH₃—Ph | N | —(CH₂)₂—CH(CH₃)—(CH₂)2- | 1,2,4-triazol-1-yl | Cl | H | H | |
| 1-1267 | 3-OCF₃—Ph | N | —(CH₂)₂—CH(CH₃)—(CH₂)2- | 1,2,4-triazol-1-yl | Cl | H | H | |
| 1-1268 | 3-NO₂—Ph | N | —(CH₂)₂—CH(CH₃)—(CH₂)2- | 1,2,4-triazol-1-yl | Cl | H | H | |
| 1-1269 | 3-NH₂—Ph | N | —(CH₂)₂—CH(CH₃)—(CH₂)2- | 1,2,4-triazol-1-yl | Cl | H | H | |
| 1-1270 | 3-Ph—Ph | N | —(CH₂)₂—CH(CH₃)—(CH₂)2- | 1,2,4-triazol-1-yl | Cl | H | H | |
| 1-1271 | 4-F—Ph | N | —(CH₂)₂—CH(CH₃)—(CH₂)2- | 1,2,4-triazol-1-yl | Cl | H | H | |
| 1-1272 | 4-Cl—Ph | N | —(CH₂)₂—CH(CH₃)—(CH₂)2- | 1,2,4-triazol-1-yl | Cl | H | H | |
| 1-1273 | 4-Br—Ph | N | —(CH₂)₂—CH(CH₃)—(CH₂)2- | 1,2,4-triazol-1-yl | Cl | H | H | |
| 1-1274 | 4-CH₃—Ph | N | —(CH₂)₂—CH(CH₃)—(CH₂)2- | 1,2,4-triazol-1-yl | Cl | H | H | |
| 1-1275 | 4-C(CH₃)₃—Ph | N | —(CH₂)₂—CH(CH₃)—(CH₂)2- | 1,2,4-triazol-1-yl | Cl | H | H | |
| 1-1276 | 4-CF₃—Ph | N | —(CH₂)₂—CH(CH₃)—(CH₂)2- | 1,2,4-triazol-1-yl | Cl | H | H | |
| 1-1277 | 4-CN—Ph | N | —(CH₂)₂—CH(CH₃)—(CH₂)2- | 1,2,4-triazol-1-yl | Cl | H | H | |
| 1-1278 | 4-OCH₃—Ph | N | —(CH₂)₂—CH(CH₃)—(CH₂)2- | 1,2,4-triazol-1-yl | Cl | H | H | |
| 1-1279 | 4-OCF₃—Ph | N | —(CH₂)₂—CH(CH₃)—(CH₂)2- | 1,2,4-triazol-1-yl | Cl | H | H | |
| 1-1280 | 4-NO₂—Ph | N | —(CH₂)₂—CH(CH₃)—(CH₂)2- | 1,2,4-triazol-1-yl | Cl | H | H | |
| 1-1281 | 4-NH₂—Ph | N | —(CH₂)₂—CH(CH₃)—(CH₂)2- | 1,2,4-triazol-1-yl | Cl | H | H | |
| 1-1282 | 4-Ph—Ph | N | —(CH₂)₂—CH(CH₃)—(CH₂)2- | 1,2,4-triazol-1-yl | Cl | H | H | |
| 1-1283 | 2-Cl4FPh | N | —(CH₂)₂—CH(CH₃)—(CH₂)2- | 1,2,4-triazol-1-yl | Cl | H | H | |
| 1-1284 | 2-Cl-6-F—Ph | N | —(CH₂)₂—CH(CH₃)—(CH₂)2- | 1,2,4-triazol-1-yl | Cl | H | H | |
| 1-1285 | 2,3-(F)₂—Ph | N | —(CH₂)₂—CH(CH₃)—(CH₂)2- | 1,2,4-triazol-1-yl | Cl | H | H | |
| 1-1286 | 2,4-(F)₂—Ph | N | —(CH₂)₂—CH(CH₃)—(CH₂)2- | 1,2,4-triazol-1-yl | Cl | H | H | |
| 1-1287 | 2,5-(F)₂—Ph | N | —(CH₂)₂—CH(CH₃)—(CH₂)2- | 1,2,4-triazol-1-yl | Cl | H | H | |
| 1-1288 | 2,6-(F)₂—Ph | N | —(CH₂)₂—CH(CH₃)—(CH₂)2- | 1,2,4-triazol-1-yl | Cl | H | H | |
| 1-1289 | 3,4-(F)₂—Ph | N | —(CH₂)₂—CH(CH₃)—(CH₂)2- | 1,2,4-triazol-1-yl | Cl | H | H | |
| 1-1290 | 3,5-(F)₂—Ph | N | —(CH₂)₂—CH(CH₃)—(CH₂)2- | 1,2,4-triazol-1-yl | Cl | H | H | |
| 1-1291 | 2,3,4-(F)₃—Ph | N | —(CH₂)₂—CH(CH₃)—(CH₂)2- | 1,2,4-triazol-1-yl | Cl | H | H | |
| 1-1292 | 2,3,6-(F)₃—Ph | N | —(CH₂)₂—CH(CH₃)—(CH₂)2- | 1,2,4-triazol-1-yl | Cl | H | H | |
| 1-1293 | 2,4,6-(F)₃—Ph | N | —(CH₂)₂—CH(CH₃)—(CH₂)2- | 1,2,4-triazol-1-yl | Cl | H | H | |
| 1-1294 | 2,3,4,5,6-(F)₅—Ph | N | —(CH₂)₂—CH(CH₃)—(CH₂)2- | 1,2,4-triazol-1-yl | Cl | H | H | |
| 1-1295 | 2,3,4,5,6-(F)₅—Ph | N | —(CH₂)₂—CH(CH₃)—(CH₂)2- | 1,2,4-triazol-1-yl | Cl | F | F | |
| 1-1296 | pyridin-2-yl | N | —(CH₂)₂—CH(CH₃)—(CH₂)2- | 1,2,4-triazol-1-yl | Cl | H | H | |
| 1-1297 | 6-Cl-pyridin-2-yl | N | —(CH₂)₂—CH(CH₃)—(CH₂)2- | 1,2,4-triazol-1-yl | Cl | H | H | |
| 1-1298 | 3-Cl-5-CF₃-pyridin-2-yl | N | —(CH₂)₂—CH(CH₃)—(CH₂)2- | 1,2,4-triazol-1-yl | Cl | H | H | |
| 1-1299 | pyridin-3-yl | N | —(CH₂)₂—CH(CH₃)—(CH₂)2- | 1,2,4-triazol-1-yl | Cl | H | H | |
| 1-1300 | 6-Cl-pyridin-3-yl | N | —(CH₂)₂—CH(CH₃)—(CH₂)2- | 1,2,4-triazol-1-yl | Cl | H | H | |
| 1-1301 | pyridin-4-yl | N | —(CH₂)₂—CH(CH₃)—(CH₂)2- | 1,2,4-triazol-1-yl | Cl | H | H | |
| 1-1302 | 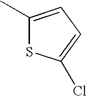 | N | —(CH₂)₂—CH(CH₃)—(CH₂)2- | 1,2,4-triazol-1-yl | Cl | H | H | |
| 1-1303 | 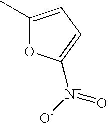 | N | —(CH₂)₂—CH(CH₃)—(CH₂)2- | 1,2,4-triazol-1-yl | Cl | H | H | |
| 1-1304 | Ph | N | —(CH₂)₂—CH(CH₃)—(CH₂)2- | 1,2,4-triazol-1-yl | H | H | H | |
| 1-1305 | Ph | N | —(CH₂)₂—CH(CH₃)—(CH₂)2- | 1,2,3-triazol-1-yl | Cl | H | H | |
| 1-1306 | Ph | N | —(CH₂)₂—CH(CH₃)—(CH₂)2- | 1,2,5-triazol-1-yl | Cl | H | H | |
| 1-1307 | Ph | N | —(CH₂)₂—CH(CH₃)—(CH₂)2- | tetrazol-1-yl | Cl | H | H | |
| 1-1308 | Ph | N | —(CH₂)₂—CH(CH₃)—(CH₂)2- | tetrazol-2-yl | Cl | H | H | |
| 1-1309 | Ph | N | —CH₂CH₂—O—CH₂CH₂— | H | Cl | H | H | |
| 1-1310 | Ph | N | —CH₂CH₂—O—CH₂CH₂— | H | Cl | CH₃ | H | |
| 1-1311 | Ph | N | —CH₂CH₂—O—CH₂CH₂— | H | Cl | CH₂CH₃ | H | |
| 1-1312 | Ph | N | —CH₂CH₂—O—CH₂CH₂— | H | Cl | CH(CH₃)CH₃ | H | |
| 1-1313 | Ph | N | —CH₂CH₂—O—CH₂CH₂— | H | Cl | Ph | H | |
| 1-1314 | Ph | N | —CH₂—CH(CH₃)-O—CH(CH₃)—CH₂— | H | Cl | H | H | |
| 1-1315 | 3,5-(F)₂—Ph | N | —CH₂—CH(CH₃)-O—CH(CH₃)—CH₂— | H | Cl | H | H | |
| 1-1316 | Ph | N | —CH₂CH₂—S—CH₂CH₂— | H | Cl | H | H | |
| 1-1317 | 3,5-(F)₂—Ph | N | —CH₂CH₂—S—CH₂CH₂— | H | Cl | H | H | |
| 1-1318 | Ph | N | —CH₂CH₂-SO₂-CH₂CH₂— | H | Cl | H | H | |
| 1-1319 | Ph | N | —CH₂CH₂-NH—CH₂CH₂— | H | Cl | H | H | |
| 1-1320 | 3,5-(F)₂—Ph | N | —CH₂CH₂-NH—CH₂CH₂— | H | Cl | H | H | |
| 1-1321 | Ph | N | —CH₂CH₂-NH—CH₂—CH(CH₃)— | H | Cl | H | H | |
| 1-1322 | Ph | N | —CH(CH₃)—CH₂-NH—CH₂—CH(CH₃)— | H | Cl | H | H | |
| 1-1323 | Ph | N | —CH₂—CH(CH₃)-NHCH(CH₃)CH₂— | H | Cl | H | H | |
| 1-1324 | Ph | N | —CH₂—CH(CH₃)-NHCH₂—CH(CH₃)— | H | Cl | H | H | |
| 1-1325 | Ph | N | —CH₂CH₂-N(CH₃)—CH₂CH₂— | H | Cl | H | H | |
| 1-1326 | Ph | N | —CH₂CH₂-N(CH₂—CH₃)—CH₂CH₂— | H | Cl | H | H | |
| 1-1327 | Ph | N | —CH₂CH₂-N(CH₂CF₃)—CH₂CH₂— | H | Cl | H | H | |
| 1-1328 | Ph | N | —CH₂CH₂-N(CH₂CH₂OH)—CH₂CH₂— | H | Cl | H | H | |
| 1-1329 | Ph | N | —CH₂CH₂-N(COOCH₂—CH₃)—CH₂CH₂— | H | Cl | H | H | |
| 1-1330 | Ph | N | —CH₂CH₂-N(CH₂COOCH₂—CH₃)—CH₂CH₂— | H | Cl | H | H | |
| 1-1331 | Ph | N | —CH₂CH₂-N(Ph)—CH₂CH₂— | H | Cl | H | H | |
| 1-1332 | Ph | N | —CH₂CH₂-N(CH₂—Ph)—CH₂CH₂— | H | Cl | H | H | |
| 1-1333 | Ph | N | —CH₂CH₂-N(pyridyl-2)—CH₂CH₂— | H | Cl | H | H | |
| 1-1334 | Ph | N | —CH₂CH₂-N(COCH₃)—CH₂CH₂— | H | Cl | H | H | |
| 1-1335 | Ph | N | —CH₂CH₂-N(COCF₃)—CH₂CH₂— | H | Cl | H | H | |
| 1-1336 | Ph | N | —CH₂CH₂-N(COCF₂CF2H)—CH₂CH₂— | H | Cl | H | H | |
| 1-1337 | Ph | N | —CH₂CH₂-N(COCF₂CF₃)—CH₂CH₂— | H | Cl | H | H | |
| 1-1338 | Ph | N | —CH₂CH₂-N(COCF₂CF₂CF₂CF₂CF₂CF₂CF₃)—CH₂CH₂— | H | Cl | H | H | |
| 1-1339 | 1-NaPh | N | —CH₂CH₂—O—CH₂CH₂— | H | Cl | H | H | |
| 1-1340 | 2-NaPh | N | —CH₂CH₂—O—CH₂CH₂— | H | Cl | H | H | |
| 1-1341 | 2-F—Ph | N | —CH₂CH₂—O—CH₂CH₂— | H | Cl | H | H | |
| 1-1342 | 2-Cl—Ph | N | —CH₂CH₂—O—CH₂CH₂— | H | Cl | H | H | |
| 1-1343 | 2-Br—Ph | N | —CH₂CH₂—O—CH₂CH₂— | H | Cl | H | H | |
| 1-1344 | 2-I—Ph | N | —CH₂CH₂—O—CH₂CH₂— | H | Cl | H | H | |
| 1-1345 | 2-CH₃—Ph | N | —CH₂CH₂—O—CH₂CH₂— | H | Cl | H | H | |
| 1-1346 | 2-CF₃—Ph | N | —CH₂CH₂—O—CH₂CH₂— | H | Cl | H | H | |
| 1-1347 | 2-CN—Ph | N | —CH₂CH₂—O—CH₂CH₂— | H | Cl | H | H | |
| 1-1348 | 2-OCH₃—Ph | N | —CH₂CH₂—O—CH₂CH₂— | H | Cl | H | H | |
| 1-1349 | 2-NO₂—Ph | N | —CH₂CH₂—O—CH₂CH₂— | H | Cl | H | H | |
| 1-1350 | 2-NH₂—Ph | N | —CH₂CH₂—O—CH₂CH₂— | H | Cl | H | H | |
| 1-1351 | 2-Ph—Ph | N | —CH₂CH₂—O—CH₂CH₂— | H | Cl | H | H | |
| 1-1352 | 3-F—Ph | N | —CH₂CH₂—O—CH₂CH₂— | H | Cl | H | H | |
| 1-1353 | 3-Cl—Ph | N | —CH₂CH₂—O—CH₂CH₂— | H | Cl | H | H | |
| 1-1354 | 3-Br—Ph | N | —CH₂CH₂—O—CH₂CH₂— | H | Cl | H | H | |
| 1-1355 | 3-I—Ph | N | —CH₂CH₂—O—CH₂CH₂— | H | Cl | H | H | |
| 1-1356 | 3-CH₃—Ph | N | —CH₂CH₂—O—CH₂CH₂— | H | Cl | H | H | |
| 1-1357 | 3-CF₃—Ph | N | —CH₂CH₂—O—CH₂CH₂— | H | Cl | H | H | |
| 1-1358 | 3-CN—Ph | N | —CH₂CH₂—O—CH₂CH₂— | H | Cl | H | H | |
| 1-1359 | 3-OCH₃—Ph | N | —CH₂CH₂—O—CH₂CH₂— | H | Cl | H | H | |
| 1-1360 | 3-OCF₃—Ph | N | —CH₂CH₂—O—CH₂CH₂— | H | Cl | H | H | |
| 1-1361 | 3-NO₂—Ph | N | —CH₂CH₂—O—CH₂CH₂— | H | Cl | H | H | |
| 1-1362 | 3-NH₂—Ph | N | —CH₂CH₂—O—CH₂CH₂— | H | Cl | H | H | |
| 1-1363 | 3-Ph—Ph | N | —CH₂CH₂—O—CH₂CH₂— | H | Cl | H | H | |
| 1-1354 | 4-F—Ph | N | —CH₂CH₂—O—CH₂CH₂— | H | Cl | H | H | |
| 1-1365 | 4-Cl—Ph | N | —CH₂CH₂—O—CH₂CH₂— | H | Cl | H | H | |
| 1-1366 | 4-Br—Ph | N | —CH₂CH₂—O—CH₂CH₂— | H | Cl | H | H | |
| 1-1367 | 4-CH₃—Ph | N | —CH₂CH₂—O—CH₂CH₂— | H | Cl | H | H | |
| 1-1368 | 4-C(CH₃)₃—Ph | N | —CH₂CH₂—O—CH₂CH₂— | H | Cl | H | H | |
| 1-1369 | 4-CF₃—Ph | N | —CH₂CH₂—O—CH₂CH₂— | H | Cl | H | H | |
| 1-1370 | 4-CN—Ph | N | —CH₂CH₂—O—CH₂CH₂— | H | Cl | H | H | |
| 1-1371 | 4-OCH₃—Ph | N | —CH₂CH₂—O—CH₂CH₂— | H | Cl | H | H | |
| 1-1372 | 4-OCF₃—Ph | N | —CH₂CH₂—O—CH₂CH₂— | H | Cl | H | H | |
| 1-1373 | 4-NO₂—Ph | N | —CH₂CH₂—O—CH₂CH₂— | H | Cl | H | H | |
| 1-1374 | 4-NH₂—Ph | N | —CH₂CH₂—O—CH₂CH₂— | H | Cl | H | H | |
| 1-1375 | 4-Ph—Ph | N | —CH₂CH₂—O—CH₂CH₂— | H | Cl | H | H | |
| 1-1376 | 2-Cl-4-F—Ph | N | —CH₂CH₂—O—CH₂CH₂— | H | Cl | H | H | |
| 1-1377 | 2-Cl-6-F—Ph | N | —CH₂CH₂—O—CH₂CH₂— | H | Cl | H | H | |
| 1-1378 | 2,3-(F)₂—Ph | N | —CH₂CH₂—O—CH₂CH₂— | H | Cl | H | H | |
| 1-1379 | 2,4-(F)₂—Ph | N | —CH₂CH₂—O—CH₂CH₂— | H | Cl | H | H | |
| 1-1380 | 2,5-(F)₂—Ph | N | —CH₂CH₂—O—CH₂CH₂— | Cl | H | H | | |
| 1-1381 | 2,6-(F)₂—Ph | N | —CH₂CH₂—O—CH₂CH₂— | H | Cl | H | H | |
| 1-1382 | 3,4-(F)₂—Ph | N | —CH₂CH₂—O—CH₂CH₂— | H | Cl | H | H | |
| 1-1383 | 3,5-(F)₂—Ph | N | —CH₂CH₂—O—CH₂CH₂— | H | Cl | H | H | |
| 1-1384 | 2,3,4-(F)₃—Ph | N | —CH₂CH₂—O—CH₂CH₂— | H | Cl | H | H | |
| 1-1385 | 2,3,6-(F)₃—Ph | N | —CH₂CH₂—O—CH₂CH₂— | H | Cl | H | H | |
| 1-1386 | 2,4,6-(F)₃—Ph | N | —CH₂CH₂—O—CH₂CH₂— | H | Cl | H | H | |
| 1-1387 | 2,3,4,5,6-(F)₅—Ph | N | —CH₂CH₂—O—CH₂CH₂— | H | Cl | H | H | |
| 1-1388 | 2,3,4,5,6-(F)₅—Ph | N | —CH₂CH₂—O—CH₂CH₂— | H | Cl | F | F | |
| 1-1389 | pyridin-2-yl | N | —CH₂CH₂—O—CH₂CH₂— | H | Cl | H | H | |
| 1-1390 | 6-Cl-pyridin-2-yl | N | —CH₂CH₂—O—CH₂CH₂— | H | Cl | H | H | |
| 1-1391 | 3-Cl-5-CF₃-pyridn-2-yl | N | —CH₂CH₂—O—CH₂CH₂— | H | Cl | H | N | |
| 1-1392 | pyridin-3-yl | N | —CH₂CH₂—O—CH₂CH₂— | H | Cl | H | H | |
| 1-1393 | 6-Cl-pyridin-3-yl | N | —CH₂CH₂—O—CH₂CH₂-H Cl | H | H | | | |
| 1-1394 | pyridin-4-yl | N | —CH₂CH₂—O—CH₂CH₂— | H | Cl | H | H | |
| 1-1395 | 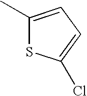 | N | —CH₂CH₂—O—CH₂CH₂— | H | Cl | H | H | |
| 1-1396 | 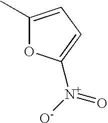 | N | —CH₂CH₂—O—CH₂CH₂— | H | Cl | H | H | |
| 1-1397 | Ph | N | —CH₂CH₂—O—CH₂CH₂— | H | H | H | H | |
| 1-1398 | Ph | N | —CH₂CH₂—O—CH₂CH₂— | H | F | H | H | |
| 1-1399 | Ph | N | —CH₂CH₂—O—CH₂CH₂— | H | CH₃ | H | H | |
| 1-1400 | Ph | N | —CH₂CH₂—O—CH₂CH₂— | H | CF₃ | H | H | |
| 1-1401 | Ph | N | —CH₂CH₂—O—CH₂CH₂— | H | OCH₃ | H | H | |
| 1-1402 | Ph | N | —CH₂CH₂—O—CH₂CH₂— | H | SCH₃ | H | H | |
| 1-1403 | Ph | N | —CH₂CH₂—O—CH₂CH₂— | H | SO₂-CH₃ | H | H | |
| 1-1404 | Ph | N | —CH₂CH₂—O—CH₂CH₂— | H | CN | H | H | |
| 1-1405 | Ph | N | —CH₂CH₂—O—CH₂CH₂— | H | 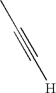 | H | H | |
| 1-1406 | Ph | N | —CH₂CH₂—O—CH₂CH₂— | H | 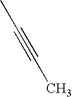 | H | H | |
| 1-1407 | Ph | N | —CH₂CH₂—O—CH₂CH₂— | CH₃ | Cl | H | H | |
| 1-1408 | Ph | N | —CH₂CH₂—O—CH₂CH₂— | CF₃ | Cl | H | H | |
| 1-1409 | Ph | N | —CH₂CH₂—O—CH₂CH₂— | C(CH₃)₃ | Cl | H | H | |
| 1-1410 | Ph | N | —CH₂CH₂—O—CH₂CH₂— | cyclopropyl | Cl | H | H | |
| 1-1411 | Ph | N | —CH₂CH₂—O—CH₂CH₂— | OCH₃ | Cl | H | H | |
| 1-1412 | Ph | N | —CH₂CH₂—O—CH₂CH₂— | O(CH₂)₃CH₃ | Cl | H | H | |
| 1-1413 | Ph | N | —CH₂CH₂—O—CH₂CH₂— | ON═C(CH₃)₂ | Cl | H | H | |
| 1-1414 | Ph | N | —CH₂CH₂—O—CH₂CH₂— | SCH₃ | Cl | H | H | |
| 1-1415 | Ph | N | —CH₂CH₂—O—CH₂CH₂— | SO₂CH₃ | Cl | H | H | |
| 1-1416 | Ph | N | —CH₂CH₂—O—CH₂CH₂— | NHPh | Cl | H | H | |
| 1-1417 | Ph | N | —CH₂CH₂—O—CH₂CH₂— | NH(CH₃)NH₂ | Cl | H | H | |
| 1-1418 | Ph | N | —CH₂CH₂—O—CH₂CH₂— | N(CH₃)N═C(CH₃)₂ | Cl | H | H | |
| 1-1419 | Ph | N | —CH₂CH₂—O—CH₂CH₂— | N(CH₃)N═(Ph)CH₃ | Cl | H | H | |
| 1-1420 | Ph | N | —CH₂CH₂—O—CH₂CH₂— | CN | Cl | H | H | |
| 1-1421 | Ph | N | —CH₂CH₂—O—CH₂CH₂— | C(═NOH)NH₂ | Cl | H | H | |
| 1-1422 | Ph | N | —CH₂CH₂—O—CH₂CH₂— | C(═NOCH₃)NH₂ | Cl | H | H | |
| 1-1423 | Ph | N | —CH₂CH₂—O—CH₂CH₂— | C(CH₃)═NOCH₂CH₃ | Cl | H | H | |
| 1-1424 | Ph | N | —CH₂CH₂—O—CH₂CH₂— | C(CH₃)═NOCH₂CH═CHCl | Cl | H | H | |
| 1-1425 | Ph | N | —CH₂CH₂—O—CH₂CH₂— | C(CH₃)═NOCH₂CH₂OCH₃ | Cl | H | H | |
| 1-1426 | Ph | N | —CH₂CH₂—O—CH₂CH₂— | CH₃ | OCH₃ | H | H | |
| 1-1427 | Ph | N | —CH₂CH₂—O—CH₂CH₂— | 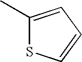 | Cl | H | H | |
| 1-1428 | Ph | N | —CH₂CH₂—O—CH₂CH₂— | 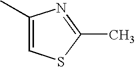 | Cl | H | H | |
| 1-1429 | Ph | N | —CH₂CH₂—O—CH₂CH₂— | pyridin-2-yl | Cl | H | H | |
| 1-1430 | 1-NaPh | N | —CH₂CH₂—O—CH₂CH₂— | pyridi~-2-yl | Cl | H | H | |
| 1-1431 | 2-NaPh | N | —CH₂CH₂—O—CH₂CH₂— | pyridih-2-yl | Cl | H | H | |
| 1-1432 | 2-F—Ph | N | —CH₂CH₂—O—CH₂CH₂— | pyridin-2-yl | Cl | H | H | |
| 1-1433 | 2-Cl—Ph | N | —CH₂CH₂—O—CH₂CH₂— | pyridin-2-yl | Cl | H | H | |
| 1-1434 | 2-5r-F N | —CH₂CH₂—O—CH₂CH₂— | pyridin-2-yl | Cl | H | H | | |
| 1-1435 | 2-I—Ph | N | —CH₂CH₂—O—CH₂CH₂— | pyridin-2-yl | Cl | H | H | |
| 1-1436 | 2-CH₃—Ph | N | —CH₂CH₂—O—CH₂CH₂— | pyridin-2-yl | Cl | H | H | |
| 1-1437 | 2-CF₃—Ph | N | —CH₂CH₂—O—CH₂CH₂— | pyridin-2-yl | Cl | H | H | |
| 1-1438 | 2-CN—Ph | N | —CH₂CH₂—O—CH₂CH₂— | pyridin-2-yl | Cl | H | H | |
| 1-1439 | 2-OCH₃—Ph | N | —CH₂CH₂—O—CH₂CH₂— | pyridin-2-yl | Cl | H | H | |
| 1-1440 | 2-NO₂—Ph | N | —CH₂CH₂—O—CH₂CH₂— | pyrldin-2-yl | Cl | H | H | |
| 1-1441 | 2-NH₂—Ph | N | —CH₂CH₂—O—CH₂CH₂— | pyridin-2-yl | Cl | H | H | |
| 1-1442 | 2-Ph—Ph | N | —CH₂CH₂—O—CH₂CH₂— | pyridin-2-yl | Cl | H | H | |
| 1-1443 | 3-F—Ph | N | —CH₂CH₂—O—CH₂CH₂— | pyridin-2-yl | Cl | H | H | |
| 1-1444 | 3-Cl—Ph | N | —CH₂CH₂—O—CH₂CH₂— | pyridin-2-yl | Cl | H | H | |
| 1-1445 | 3-Br—Ph | N | —CH₂CH₂—O—CH₂CH₂— | pyridin-2-yl | Cl | H | H | |
| 1-1446 | 3-I—Ph | N | —CH₂CH₂—O—CH₂CH₂— | pyridin-2-yl | Cl | H | H | |
| 1-1447 | 3-CH₃—Ph | N | —CH₂CH₂—O—CH₂CH₂— | pyridin-2-yl | Cl | H | H | |
| 1-1448 | 3-CF₃—Ph | N | —CH₂CH₂—O—CH₂CH₂— | pyridin-2-yl | Cl | H | H | |
| 1-1449 | 3-CN—Ph | N | —CH₂CH₂—O—CH₂CH₂— | pyridin-2-yl | Cl | H | H | |
| 1-1450 | 3-OCH₃—Ph | N | —CH₂CH₂—O—CH₂CH₂— | pyridin-2-yl | Cl | H | H | |
| 1-1451 | 3-OCF₃—Ph | N | —CH₂CH₂—O—CH₂CH₂— | pyridin-2-yl | Cl | H | H | |
| 1-1452 | 3-NO₂—Ph | N | —CH₂CH₂—O—CH₂CH₂— | pyridin-2-yl | Cl | H | H | |
| 1-1453 | 3-NH₂—Ph | N | —CH₂CH₂—O—CH₂CH₂— | pyridin-2-yl | Cl | H | H | |
| 1-1454 | 3-Ph—Ph | N | —CH₂CH₂—O—CH₂CH₂— | pyridin-2-yl | Cl | H | H | |
| 1-1455 | 4-F—Ph | N | —CH₂CH₂—O—CH₂CH₂— | pyridin-2-yl | Cl | H | H | |
| 1-1456 | 4-Cl—Ph | N | —CH₂CH₂—O—CH₂CH₂— | pyridin-2-yl | Cl | H | | |
| 1-1457 | 4-Br—Ph | N | —CH₂CH₂—O—CH₂CH₂— | pyridin-2-yl | Cl | H | H | |
| 1-1458 | 4-CH₃—Ph | N | —CH₂CH₂—O—CH₂CH₂— | pyridin-2-yl | Cl | H | H | |
| 1-1459 | 4-C(CH₃)₃—Ph | —CH₂CH₂—O—CH₂CH₂— | pyridin-2-yl | Cl | H | H | | |
| 1-1460 | 4-CF₃—Ph | N | —CH₂CH₂—O—CH₂CH₂— | pyridin-2-yl | Cl | H | H | |
| 1-1461 | 4-CN—Ph | N | —CH₂CH₂—O—CH₂CH₂— | pyridin-2-yl | Cl | H | H | |
| 1-1462 | 4-OCH₃—Ph | N | —CH₂CH₂—O—CH₂CH₂— | pyridin-2-yl | Cl | H | H | |
| 1-1463 | 4-OCF₃—Ph | N | —CH₂CH₂—O—CH₂CH₂— | pyridin-2-yl | Cl | H | H | |
| 1-1464 | 4-NO₂—Ph | N | —CH₂CH₂—O—CH₂CH₂— | pyridin-2-yl | Cl | H | H | |
| 1-1465 | 4-NH₂—Ph | N | —CH₂CH₂—O—CH₂CH₂— | pyridin-2-yl | Cl | H | H | |
| 1-1466 | 4-Ph—Ph | N | —CH₂CH₂—O—CH₂CH₂— | pyridin-2-yl | Cl | H | H | |
| 1-1467 | 2-Cl-4-F—Ph | N | —CH₂CH₂—O—CH₂CH₂— | pyridin-2-yl | Cl | H | H | |
| 1-1468 | 2-Cl-6-F—Ph | N | —CH₂CH₂—O—CH₂CH₂— | pyridin-2-yl | Cl | H | H | |
| 1-1469 | 2,3-(F)₂—Ph | N | —CH₂CH₂—O—CH₂CH₂— | pyridin-2-yl | Cl | H | H | |
| 1-1470 | 2,4-(F)₂—Ph | N | —CH₂CH₂—O—CH₂CH₂— | pyridin-2-yl | Cl | H | H | |
| 1-1471 | 2,5-(F)₂—Ph | N | —CH₂CH₂—O—CH₂CH₂— | pyridin-2-yl | Cl | H | H | |
| 1-1472 | 2,6-(F)₂—Ph | N | —CH₂CH₂—O—CH₂CH₂— | pyridin-2-yl | Cl | H | H | |
| 1-1473 | 3,4-(F)₂—Ph | N | —CH₂CH₂—O—CH₂CH₂— | pyridin-2-yl | Cl | H | H | |
| 1-1474 | 3,5-(F)₂—Ph | N | —CH₂CH₂—O—CH₂CH₂— | pyridin-2-yl | Cl | H | H | |
| 1-1475 | 2,34-(F)₃—Ph | N | —CH₂CH₂—O—CH₂CH₂— | pyridin-2-yl | Cl | H | H | |
| 1-1476 | 2,3,6-(F)aPh | N | —CH₂CH₂—O—CH₂CH₂— | pyridin-2-yl | Cl | H | H | |
| 1-1477 | 2,4,6-(F)₃—Ph | N | —CH₂CH₂—O—CH₂CH₂— | pyridin-2-yl | Cl | H | H | |
| 1-1478 | 2,3,4,56-(FTh—Ph | N | —CH₂CH₂—O—CH₂CH₂— | pyridin-2-yl | Cl | H | H | |
| 1-1479 | 2,34,5,6-(F)₅—Ph | N | —CH₂CH₂—O—CH₂CH₂— | pyridin-2-yl | Cl | F | F | |
| 1-1480 | pyridin-2-yl | N | —CH₂CH₂—O—CH₂CH₂— | pyridin-2-yl | Cl | H | H | |
| 1-1481 | 6-Cl-pyridin-2-yl | N | —CH₂CH₂—O—CH₂CH₂— | pyridin-2-yl | Cl | H | H | |
| 1-1482 | 3-Cl-5-CF₃-pyridn-2-yl | N | —CH₂CH₂—O—CH₂CH₂— | pyridin-2-yl | Cl | H | H | |
| 1-1483 | pyridin-3-yl | N | —CH₂CH₂—O—CH₂CH₂— | pyridin-2-yl | Cl | H | H | |
| 1-1484 | 6-Cl-pyridin-3-yl | N | —CH₂CH₂—O—CH₂CH₂— | pyridin-2-yl | Cl | H | H | |
| 1-1485 | pyridin-4-yl | N | —CH₂CH₂—O—CH₂CH₂— | pyridin-2-yl | Cl | H | H | |
| 1-1486 | 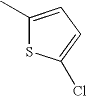 | N | —CH₂CH₂—O—CH₂CH₂— | pyridin-2-yl | Cl | H | H | |
| 1-1487 | 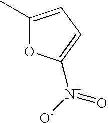 | N | —CH₂CH₂—O—CH₂CH₂— | pyridin-2-yl | Cl | H | H | |
| 1-1488 | Ph | N | —CH₂CH₂—O—CH₂CH₂— | pyridin-2-yl | H | H | H | |
| 1-1489 | Ph | N | —CH₂CH₂—O—CH₂CH₂— | 3-CH₃-pyridin-2-yl | Cl | H | H | |
| 1-1490 | Ph | N | —CH₂CH₂—O—CH₂CH₂— | 4-CH₃-pyridin-2-yl | Cl | H | H | |
| 1-1491 | Ph | N | —CH₂CH₂—O—CH₂CH₂— | 6-CH₃-pyridin-2-yl | Cl | H | H | |
| 1-1492 | Ph | N | —CH₂CH₂—O—CH₂CH₂— | pyridin-3-yl | Cl | H | H | |
| 1-1493 | Ph | N | —CH₂CH₂—O—CH₂CH₂— | pyridin-4-yl | Cl | H | H | |
| 1-1494 | Ph | N | —CH₂CH₂—O—CH₂CH₂— | 2,6-(Cl)2- | pyridin-4-yl | Cl | H | H |
| 1-1495 | Ph | N | —CH₂CH₂—O—CH₂CH₂— | 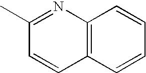 | Cl | H | H | |
| 1-1496 | Ph | N | —CH₂CH₂—O—CH₂CH₂— | 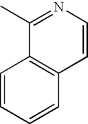 | Cl | H | H | |
| 1-1497 | Ph | N | —CH₂CH₂—O—CH₂CH₂— | 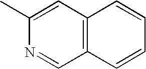 | Cl | H | H | |
| 1-1498 | Ph | N | —CH₂CH₂—O—CH₂CH₂— | pyrimidin-2-yl | Cl | H | H | |
| 1-1499 | Ph | N | —CH₂CH₂—O—CH₂CH₂— | pyridazin-3-yl | Cl | H | H | |
| 1-1500 | Ph | N | —CH₂CH₂—O—CH₂CH₂— | pyrazin-2-yl | Cl | H | H | |
| 1-1501 | 1-NaPh | N | —CH₂CH₂—O—CH₂CH₂— | pyrazin-2-yl | Cl | H | H | |
| 1-1502 | 2-NaPh | N | —CH₂CH₂—O—CH₂CH₂— | pyrazin-2-yl | Cl | H | H | |
| 1-1503 | 2-F—Ph | N | —CH₂CH₂—O—CH₂CH₂— | pyrazin-2-yl | Cl | H | H | |
| 1-1504 | 2-Cl—Ph | N | —CH₂CH₂—O—CH₂CH₂— | pyrazin-2-yl | Cl | H | H | |
| 1-1505 | 2-Br—Ph | N | —CH₂CH₂—O—CH₂CH₂— | pyrazin-2-yl | Cl | H | H | |
| 1-1506 | 2-I—Ph | N | —CH₂CH₂—O—CH₂CH₂— | pyrazin-2-yl | Cl | H | H | |
| 1-1507 | 2-CH₃—Ph | N | —CH₂CH₂—O—CH₂CH₂— | pyrazin-2-yl | Cl | H | H | |
| 1-1508 | 2-CF₃—Ph | N | —CH₂CH₂—O—CH₂CH₂— | pyrazin-2-yl | Cl | H | H | |
| 1-1509 | 2-CN—Ph | N | —CH₂CH₂—O—CH₂CH₂— | pyrazin-2-yl | Cl | H | H | |
| 1-1510 | 2-OCH₃—Ph | N | —CH₂CH₂—O—CH₂CH₂— | pyrazin-2-yl | Cl | H | H | |
| 1-1511 | 2-NO₂—Ph | N | —CH₂CH₂—O—CH₂CH₂— | pyrazin-2-yl | Cl | H | H | |
| 1-1512 | 2-NH₂—Ph | N | —CH₂CH₂—O—CH₂CH₂— | pyrazin-2-yl | Cl | H | H | |
| 1-1513 | 2-Ph—Ph | N | —CH₂CH₂—O—CH₂CH₂— | pyrazin-2-yl | Cl | H | H | |
| 1-1514 | 3-F—Ph | N | —CH₂CH₂—O—CH₂CH₂— | pyrazin-2-yl | Cl | H | H | |
| 1-1515 | 3-Cl—Ph | N | —CH₂CH₂—O—CH₂CH₂— | pyrazin-2-yl | Cl | H | H | |
| 1-1516 | 3-Br—Ph | N | —CH₂CH₂—O—CH₂CH₂— | pyrazin-2-yl | Cl | H | H | |
| 1-1517 | 3-I—Ph | N | —CH₂CH₂—O—CH₂CH₂— | pyrazin-2-yl | Cl | H | H | |
| 1-1518 | 3CH₃—Ph | N | —CH₂CH₂—O—CH₂CH₂— | pyrazin-2-yl | Cl | H | H | |
| 1-1519 | 3-CF₃—Ph | N | —CH₂CH₂—O—CH₂CH₂— | pyrazin-2-yl | Cl | H | H | |
| 1-1520 | 3-CN—Ph | N | —CH₂CH₂—O—CH₂CH₂— | pyrazin-2-yl | Cl | H | H | |
| 1-1521 | 3-OCH₃—Ph | N | —CH₂CH₂—O—CH₂CH₂— | pyrazin-2-yl | Cl | H | H | |
| 1-1522 | 3-OCF₃—Ph | N | —CH₂CH₂—O—CH₂CH₂— | pyrazin-2-yl | Cl | H | H | |
| 1-1523 | 3-NO₂—Ph | N | —CH₂CH₂—O—CH₂CH₂— | pyrazin-2-yl | Cl | H | H | |
| 1-1524 | 3-NH₂—Ph | N | —CH₂CH₂—O—CH₂CH₂— | pyrazin-2-yl | Cl | H | H | |
| 1-1525 | 3-Ph—Ph | N | —CH₂CH₂—O—CH₂CH₂— | pyrazin-2-yl | Cl | H | H | |
| 1-1526 | 4-F—Ph | N | —CH₂CH₂—O—CH₂CH₂— | pyrazin-2-yl | Cl | H | H | |
| 1-1527 | 4-Cl—Ph | N | —CH₂CH₂—O—CH₂CH₂— | pyrazin-2-yl | Cl | H | H | |
| 1-1528 | 4-Br—Ph | N | —CH₂CH₂—O—CH₂CH₂— | pyrazin-2-yl | Cl | H | H | |
| 1-1529 | 4-CH₃—Ph | N | —CH₂CH₂—O—CH₂CH₂— | pyrazin-2-yl | Cl | H | H | |
| 1-1530 | 4-C(CH₃)₃—Ph | N | —CH₂CH₂—O—CH₂CH₂— | pyrazin-2-yl | Cl | H | H | |
| 1-1531 | 4-CF₃—Ph | N | —CH₂CH₂—O—CH₂CH₂— | pyrazin-2-yl | Cl | H | H | |
| 1-1532 | 4-CN—Ph | N | —CH₂CH₂—O—CH₂CH₂— | pyrazin-2-yl | Cl | H | H | |
| 1-1533 | 4-OCH₃—Ph | N | —CH₂CH₂—O—CH₂CH₂— | pyrazin-2-yl | Cl | H | H | |
| 1-1534 | 4-OCF₃—Ph | N | —CH₂CH₂—O—CH₂CH₂— | pyrazin-2-yl | Cl | H | H | |
| 1-1535 | 4-NO₂—Ph | N | —CH₂CH₂—O—CH₂CH₂— | pyrazin-2-yl | Cl | H | H | |
| 1-1536 | 4-NH₂—Ph | N | —CH₂CH₂—O—CH₂CH₂— | pyrazin-2-yl | Cl | H | H | |
| 1-1537 | 4-Ph—Ph | N | —CH3CH₂—O—CH₂CH₂— | pyrazin-2-yl | Cl | H | H | |
| 1-1538 | 2-Cl-4-F—Ph | N | —CH₂CH₂—O—CH₂CH₂— | pyrazin-2-yl | Cl | H | H | |
| 1-1539 | 2-Cl-6-F—Ph | N | —CH₂CH₂—O—CH₂CH₂— | pyrazin-2-yl | Cl | H | H | |
| 1-1540 | 2,3-(F)₂—Ph | N | —CH₂CH₂—O—CH₂CH₂— | pyrazin-2-yl | Cl | H | H | |
| 1-1541 | 2,4-(F)₂—Ph | N | —CH₂CH₂—O—CH₂CH₂— | pyrazin-2-yl | Cl | H | H | |
| 1-1542 | 2,5-(F)₂—Ph | N | —CH₂CH₂—O—CH₂CH₂— | pyrazin-2-yl | Cl | H | H | |
| 1-1543 | 2,6-(F)₂—Ph | N | —CH₂CH₂—O—CH₂CH₂— | pyrazln-2-yl | Cl | H | H | |
| 1-1544 | 3,4-(F)₂—Ph | N | —CH₂CH₂—O—CH₂CH₂— | pyrazin-2-yl | Cl | H | H | |
| 1-1545 | 3,5-(F)₂—Ph | N | —CH₂CH₂—O—CH₂CH₂— | pyrazin-2-yl | Cl | H | H | |
| 1-1546 | 2,3,4-(F)₃—Ph | N | —CH₂CH₂—O—CH₂CH₂— | pyrazin-2-yl | Cl | H | H | |
| 1-1547 | 2,3,6-(F)₃—Ph | N | —CH₂CH₂—O—CH₂CH₂— | pyrazin-2-yl | Cl | H | H | |
| 1-1548 | 2,4,6-(F)₃—Ph | N | —CH₂CH₂—O—CH₂CH₂— | pyrazin-2-yl | Cl | H | H | |
| 1-1549 | 2,3,4,5,6-(F)₅—Ph | N | —CH₂CH₂—O—CH₂CH₂— | pyrazin-2-yl | Cl | H | H | |
| 1-1550 | 2,3,4,5,6-(F)₅—Ph | N | —CH₂CH₂—O—CH₂CH₂— | pyrazin-2-yl | Cl | F | F | |
| 1-1551 | pyridin-2-yl | N | —CH₂CH₂—O—CH₂CH₂— | pyrazin-2-yl | Cl | H | H | |
| 1-1552 | 6-Cl-pyridin-2-yl | N | —CH₂CH₂—O—CH₂CH₂— | pyrazin-2-yl | Cl | H | H | |
| 1-1553 | 3-Cl-5-CF₃-pyridn-2-yl | N | —CH₂CH₂—O—CH₂CH₂— | pyrazin-2-yl | Cl | H | H | |
| 1-1554 | pyridin-3-yl | N | —CH₂CH₂—O—CH₂CH₂— | pyrazin-2-yl | Cl | H | H | |
| 1-1555 | 6-Cl-pyridin-3-yl | N | —CH₂CH₂—O—CH₂CH₂— | pyrazin-2-yl | Cl | H | H | |
| 1-1556 | pyridin-4-yl | N | —CH₂CH₂—O—CH₂CH₂— | pyrazin-2-yl | Cl | H | H | |
| 1-1557 | 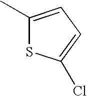 | N | —CH₂CH₂—O—CH₂CH₂— | pyrazin-2-yl | Cl | H | H | |
| 1-1558 | 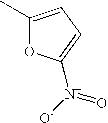 | N | —CH₂CH₂—O—CH₂CH₂— | pyrazin-2-yl | Cl | H | H | |
| 1-1559 | Ph | N | —CH₂CH₂—O—CH₂CH₂— | pyrazin-2-yl | H | H | H | |
| 1-1560 | Ph | N | —CH₂CH₂—O—CH₂CH₂— | pyrazol-1-yl | Cl | H | H | |
| 1-1561 | 1-NaPh | N | —CH₂CH₂—O—CH₂CH₂— | pyrazol-1-yl | Cl | H | H | |
| 1-1562 | 2-NaPh | N | —CH₂CH₂—O—CH₂CH₂— | pyrazol-1-yl | Cl | H | H | |
| 1-1563 | 2-F—Ph | N | —CH₂CH₂—O—CH₂CH₂— | pyrazol-1-yl | Cl | H | H | |
| 1-1564 | 2-Cl—Ph | N | —CH₂CH₂—O—CH₂CH₂— | pyrazol-1-yl | Cl | H | H | |
| 1-1565 | 2-Br—Ph | N | —CH₂CH₂—O—CH₂CH₂— | pyrazol-1-yl | Cl | H | H | |
| 1-1566 | 2-I—Ph | N | —CH₂CH₂—O—CH₂CH₂— | pyrazol-1-yl | Cl | H | H | |
| 1-1567 | 2-CH₃—Ph | N | —CH₂CH₂—O—CH₂CH₂— | pyrazol-1-yl | Cl | H | H | |
| 1-1568 | 2-CF₃—Ph | N | —CH₂CH₂—O—CH₂CH₂— | pyrazol-1-yl | Cl | H | H | |
| 1-1569 | 2-CN—Ph | N | —CH₂CH₂—O—CH₂CH₂— | pyrazol-1-yl | Cl | H | H | |
| 1-1570 | 2-OCH₃—Ph | N | —CH₂CH₂—O—CH₂CH₂— | pyrazol-1-yl | Cl | H | H | |
| 1-1571 | 2-NO₂—Ph | N | —CH₂CH₂—O—CH₂CH₂— | pyrazol-1-yl | Cl | H | H | |
| 1-1572 | 2-NH₂—Ph | N | —CH₂CH₂—O—CH₂CH₂— | pyrazol-1-yl | Cl | H | H | |
| 1-1573 | 2-Ph—Ph | N | —CH₂CH₂—O—CH₂CH₂— | pyrazol-1-yl | Cl | H | H | |
| 1-1574 | 3-F—Ph | N | —CH₂CH₂—O—CH₂CH₂— | pyrazol-1-yl | Cl | H | H | |
| 1-1575 | 3-Cl—Ph | N | —CH₂CH₂—O—CH₂CH₂— | pyrazol-1-yl | Cl | H | H | |
| 1-1576 | 3-Br—Ph | N | —CH₂CH₂—O—CH₂CH₂— | pyrazol-1-yl | Cl | H | H | |
| 1-1577 | 3-I—Ph | N | —CH₂CH₂—O—CH₂CH₂— | pyrazol-1-yl | Cl | H | H | |
| 1-1578 | 3-CH₃—Ph | N | —CH₂CH₂—O—CH₂CH₂— | pyrazol-1-yl | Cl | H | H | |
| 1-1579 | 3-CF₃—Ph | N | —CH₂CH₂—O—CH₂CH₂— | pyrazol-1-yl | Cl | H | H | |
| 1-1580 | 3-CN—Ph | N | CH₂CH₂—O—CH₂CH₂— | pyrazol-1-y Cl | H | H | | |
| 1-1581 | 3-OCH₃—Ph | N | —CH₂CH₂—O—CH₂CH₂— | pyrazol-1-yl | Cl | H | H | |
| 1-1582 | 3-OCF₃—Ph | N | —CH₂CH₂—O—CH₂CH₂— | pyrazol-1-yl | Cl | H | H | |
| 1-1583 | 3-NO₂—Ph | N | —CH₂CH₂—O—CH₂CH₂— | pyrazol-1-y | Cl | H | H | |
| 1-1584 | 3-NH₂—Ph | N | —CH₂CH₂—O—CH₂CH₂— | pyrazol-1-yl | Cl | H | H | |
| 1-1585 | 3-Ph—Ph | N | —CH₂CH₂—O—CH₂CH₂— | pyrazol-1-yl | Cl | H | H | |
| 1-1586 | 4-F—Ph | N | —CH₂CH₂—O—CH₂CH₂— | pyrazol-1-yl | Cl | H | H | |
| 1-1587 | 4-Cl—Ph | N | —CH₂CH₂—O—CH₂CH₂— | pyrazol-1-yl | Cl | H | H | |
| 1-1588 | 4-Br—Ph | N | —CH₂CH₂—O—CH₂CH₂— | pyrazol-1-yl | Cl | H | H | |
| 1-1589 | 4-CH₃—Ph | N | —CH₂CH₂—O—CH₂CH₂— | pyrazol-1-yl | Cl | H | H | |
| 1-1590 | 4-C(CH₃)₃—Ph | N | —CH₂CH₂—O—CH₂CH₂— | pyrazol-1-yl | Cl | H | H | |
| 1-1591 | 4-CF₃—Ph | N | —CH₂CH₂—O—CH₂CH₂— | pyrazol-1-yl | Cl | H | H | |
| 1-1592 | 4-CN—Ph | N | —CH₂CH₂—O—CH₂CH₂— | pyrazol-1-yl | Cl | H | H | |
| 1-1593 | 4-OCH₃—Ph | N | —CH₂CH₂—O—CH₂CH₂— | pyrazol-1-yl | Cl | H | H | |
| 1-1594 | 4-OCF₃—Ph | N | —CH₂CH₂—O—CH₂CH₂— | pyrazol-1-yl | Cl | H | H | |
| 1-1595 | 4-NO₂—Ph | N | —CH₂CH₂—O—CH₂CH₂— | pyrazol-1-yl | Cl | H | H | |
| 1-1596 | 4-NH₂—Ph | N | —CH₂CH₂—O—CH₂CH₂— | pyrazol-1-yl | Cl | H | H | |
| 1-1597 | 4-Ph—Ph | N | —CH₂CH₂—O—CH₂CH₂— | pyrazol-1-yl | Cl | H | H | |
| 1-1598 | 2-Cl-4-F—Ph | N | —CH₂CH₂—O—CH₂CH₂— | pyrazol-1-yl | Cl | H | H | |
| 1-1599 | 2-Cl-6-F—Ph | N | —CH₂CH₂—O—CH₂CH₂— | pyrazol-1-yl | Cl | H | H | |
| 1-1600 | 2,3-(F)₂—Ph | N | —CH₂CH₂—O—CH₂CH₂— | pyrazol-1-yl | Cl | H | H | |
| 1-1601 | 2,4-(F)₂—Ph | N | —CH₂CH₂—O—CH₂CH₂— | pyrazol-1-yl | Cl | H | H | |
| 1-1602 | 2,5-(F)₂—Ph | N | —CH₂CH₂—O—CH₂CH₂— | pyrazol-1-yl | Cl | H | H | |
| 1-1603 | 2,6-(F)₂—Ph | N | —CH₂CH₂—O—CH₂CH₂— | pyrazol-1-yl | Cl | H | H | |
| 1-1604 | 3,4-(F)₂—Ph | N | —CH₂CH₂—O—CH₂CH₂— | pyrazol-1-yl | Cl | H | H | |
| 1-1605 | 3,5-(F)₂—Ph | N | —CH₂CH₂—O—CH₂CH₂— | pyrazol-1-yl | Cl | H | H | |
| 1-1606 | 2,3,4-(F)₃—Ph | N | —CH₂CH₂—O—CH₂CH₂— | pyrazol-1-yl | Cl | H | H | |
| 1-1607 | 2,3,6-(F)₃—Ph | N | —CH₂CH₂—O—CH₂CH₂— | pyrazol-1-yl | Cl | H | H | |
| 1-1608 | 2,4,6-(F)₃—Ph | N | —CH₂CH₂—O—CH₂CH₂— | pyrazol-1-yl | Cl | H | H | |
| 1-1609 | 2,3,4,5,6-(F)₅—Ph | N | —CH₂CH₂—O—CH₂CH₂— | pyrazol-1-yl | Cl | H | H | |
| 1-1610 | 2,3,4,5,6-(F)₅—Ph | N | —CH₂CH₂—O—CH₂CH₂— | pyrazol-1-yl | Cl | F | F | |
| 1-1611 | pyridin-2-yl | N | —CH₂CH₂—O—CH₂CH₂— | pyrazol-1-yl | Cl | H | H | |
| 1-1612 | 6-Cl-pyridin-2-yl | N | —CH₂CH₂—O—CH₂CH₂— | pyrazol-1-yl | Cl | H | H | |
| 1-1613 | 3-Cl-5-CF₃-pyridn-2-yl | N | —CH₂CH₂—O—CH₂CH₂— | pyrazol-1-yl | Cl | H | H | |
| 1-1614 | pyridin-3-yl | N | —CH₂CH₂—O—CH₂CH₂— | pyrazol-1-yl | Cl | H | H | |
| 1-1615 | 6-Cl-pyridin-3-yl | N | —CH₂CH₂—O—CH₂CH₂— | pyrazol-1-yl | Cl | H | H | |
| 1-1616 | pyridin-4-yl | N | —CH₂CH₂—O—CH₂CH₂— | pyrazol-1-yl | Cl | H | H | |
| 1-1617 | 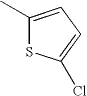 | N | —CH₂CH₂—O—CH₂CH₂— | pyrazol-1-yl | Cl | H | H | |
| 1-1618 | 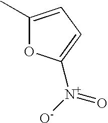 | N | —CH₂CH₂—O—CH₂CH₂— | pyrazol-1-yl | Cl | H | H | |
| 1-1619 | Ph | N | —CH₂CH₂—O—CH₂—CH~- | pyrazol-1-yl | H | H | H | |
| 1-1620 | Ph | N | —CH₂CH₂—O—CH₂CH₂— | 3-CH₃-pyrazol-1-yl | Cl | H | H | |
| 1-1621 | Ph | N | —CH₂CH₂—O—CH₂CH₂— | 3,5-(CH₃)₂-pyrazol-1-yl | Cl | H | H | |
| 1-1622 | Ph | N | —CH₂CH₂—O—CH₂CH₂— | 3-CF₃-pyrazol-1-yl | Cl | H | H | |
| 1-1623 | Ph | N | —CH₂CH₂—O—CH₂CH₂— | 3,5-(CF₃)₂-pyrazol-1-yl | Cl | H | H | |
| 1-1624 | Ph | N | —CH₂CH₂—O—CH₂CH₂— | 4-Br-pyrazol-1-yl | Cl | H | H | |
| 1-1625 | Ph | N | —CH₂CH₂—O—CH₂CH₂— | 4-CH₃-pyrazol-1-yl | Cl | H | H | |
| 1-1626 | Ph | N | —CH₂CH₂—O—CH₂CH₂— | imidazol-1-yl | Cl | H | H | |
| 1-1627 | Ph | N | —CH₂CH₂—O—CH₂CH₂— | 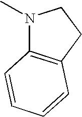 | Cl | H | H | |
| 1-1628 | Ph | N | —CH₂CH₂—O—CH₂CH₂— | 1,2,4-triazol-1-yl | Cl | H | H | |
| 1-1629 | 1-NaPh | N | —CH₂CH₂—O—CH₂CH₂— | 1,2,4-triazol-1-yl | Cl | H | H | |
| 1-1630 | 2-NaPh | N | —CH₂CH₂—O—CH₂CH₂— | 1,2,4-triazol-1-yl | Cl | H | H | |
| 1-1631 | 2-F—Ph | N | —CH₂CH₂—O—CH₂CH₂— | 1,2,4-triazol-1-yl | Cl | H | H | |
| 1-1632 | 2-Cl—Ph | N | —CH₂CH₂—O—CH₂CH₂— | 1,2,4-triazol-1-yl | Cl | H | H | |
| 1-1633 | 2-Br—Ph | N | —CH₂CH₂—O—CH₂CH₂— | 1,2,4-triazol-1-yl | Cl | H | H | |
| 1-1634 | 2-I—Ph | N | —CH₂CH₂—O—CH₂CH₂— | 1,2,4-triazol-1-yl | Cl | H | H | |
| 1-1635 | 2-CH₃—Ph | N | —CH₂CH₂—O—CH₂CH₂— | 1,2,4-triazol-1-yl | Cl | H | H | |
| 1-1636 | 2-CF₃—Ph | N | —CH₂CH₂—O—CH₂CH₂— | 1,2,4-triazol-1-yl | Cl | H | H | |
| 1-1637 | 2-CN—Ph | N | —CH₂CH₂—O—CH₂CH₂— | 1,2,4-triazol-1-yl | Cl | H | H | |
| 1-1638 | 2-OCH₃—Ph | N | —CH₂CH₂—O—CH₂CH₂— | 1,2,4-triazol-1-yl | Cl | H | H | |
| 1-1639 | 2-NO₂—Ph | N | —CH₂CH₂—O—CH₂CH₂— | 1,2,4-triazol-1-yl | Cl | H | H | |
| 1-1640 | 2-NH₂—Ph | N | —CH₂CH₂—O—CH₂CH₂— | 1,2,4-triazol-1-yl | Cl | H | H | |
| 1-1641 | 2-Ph—Ph | N | —CH₂CH₂—O—CH₂CH₂— | 1,2,4-triazol-1-yl | Cl | H | H | |
| 1-1642 | 3-F—Ph | N | —CH₂CH₂—O—CH₂CH₂— | 1,2,4-triazol-1-yl | Cl | H | H | |
| 1-1643 | 3-Cl—Ph | N | —CH₂CH₂—O—CH₂CH₂— | 1,2,4-triazol-1-yl | Cl | H | H | |
| 1-1644 | 3-Br—Ph | N | —CH₂CH₂—O—CH₂CH₂— | 1,2,4-triazol-1-yl | Cl | H | H | |
| 1-1645 | 3-I—Ph | N | —CH₂CH₂—O—CH₂CH₂— | 1,2,4-triazol-1-yl | Cl | H | H | |
| 1-1646 | 3-CH₃—Ph | N | —CH₂CH₂—O—CH₂CH₂— | 1,2,4-triazol-1-yl | Cl | H | H | |
| 1-1647 | 3-CF₃—Ph | N | —CH₂CH₂—O—CH₂CH₂— | 1,2,4-triazol-1-yl | Cl | H | H | |
| 1-1648 | 3-CN—Ph | N | —CH₂CH₂—O—CH₂CH₂— | 1,2,4-triazol-1-yl | Cl | H | H | |
| 1-1649 | 3-OCH₃—Ph | N | —CH₂CH₂—O—CH₂CH₂— | 1,2,4-triazol-1-yl | Cl | H | H | |
| 1-1650 | 3-OCF₃—Ph | N | —CH₂CH₂—O—CH₂CH₂— | 1,2,4-triazol-1-yl | Cl | H | H | |
| 1-1651 | 3-NO₂—Ph | N | —CH₂CH₂—O—CH₂CH₂— | 1,2,4-triazol-1-yl | Cl | H | H | |
| 1-1652 | 3-NH₂—Ph | N | —CH₂CH₂—O—CH₂CH₂— | 1,2,4-triazol-1-yl | Cl | H | H | |
| 1-1653 | 3-Ph—Ph | N | —CH₂CH₂—O—CH₂CH₂— | 1,2,4-triazol-1-yl | Cl | H | H | |
| 1-1654 | 4-F—Ph | N | —CH₂CH₂—O—CH₂CH₂— | 1,2,4-triazol-1-yl | Cl | H | H | |
| 1-1655 | 4-Cl—Ph | N | —CH₂CH₂—O—CH₂CH₂— | 1,2,4-triazol-1-yl | Cl | H | H | |
| 1-1656 | 4-Br—Ph | N | —CH₂CH₂—O—CH₂CH₂— | 1,2,4-triazol-1-yl | Cl | H | H | |
| 1-1657 | 4-CH₃—Ph | N | —CH₂CH₂—O—CH₂CH₂— | 1,2,4-triazol-1-yl | Cl | H | H | |
| 1-1658 | 4-C(CH₃)₃—Ph | N | —CH₂CH₂OCH₂CH₂— | 1,2,4-triazol-1-yl | Cl | H | H | |
| 1-1659 | 4-CF₃—Ph | N | —CH₂CH₂—O—CH₂CH₂— | 1,2,4-triazol-1-yl | Cl | H | H | |
| 1-1660 | 4-CN—Ph | N | —CH₂CH₂—O—CH₂CH₂— | 1,2,4-triazol-1-yl | Cl | H | H | |
| 1-1661 | 4-OCH₃—Ph | N | —CH₂CH₂—O—CH₂CH₂— | 1,2,4-triazol-1-yl | Cl | H | H | |
| 1-1662 | 4-OCF₃—Ph | N | —CH₂CH₂—O—CH₂CH₂— | 1,2,4-triazol-1-yl | Cl | H | H | |
| 1-1663 | 4-NO₂—Ph | N | —CH₂CH₂—O—CH₂CH₂— | 1,2,4-triazol-1-yl | Cl | H | H | |
| 1-1664 | 4-NH₂—Ph | N | —CH₂CH₂—O—CH₂CH₂— | 1,2,4-triazol-1-yl | Cl | H | H | |
| 1-1665 | 4-Ph—Ph | N | —CH₂CH₂—O—CH₂CH₂— | 1,2,4-triazol-1-yl | Cl | H | H | |
| 1-1666 | 2-Cl-4-F—Ph | N | —CH₂CH₂—O—CH₂CH₂— | 1,2,4-triazol-1-yl | Cl | H | H | |
| 1-1667 | 2-Cl-6-F—Ph | N | —CH₂CH₂—O—CH₂CHa | 1,2,4-triazol-1-yl | Cl | H | H | |
| 1-1668 | 2,3-(F)₂—Ph | N | —CH₂CH₂—O—CH₂CH₂— | 1,2,4-triazol-1-yl | Cl | H | H | |
| 1-1669 | 2,4-(F)₂—Ph | N | —CH₂CH₂—O—CH₂CH₂— | 1,2,4-triazol-1-yl | Cl | H | H | |
| 1-1670 | 2,5-(F)₂—Ph | N | —CH₂CH₂—O—CH₂CH₂— | 1,2,4-triazol-1-yl | Cl | H | H | |
| 1-1671 | 2,6-(F)₂—Ph | N | —CH₂CH₂—O—CH₂CH₂— | 1,2,4-triazol-1-yl | Cl | H | H | |
| 1-1672 | 3,4-(F)₂—Ph | N | —CH₂CH₂—O—CH₂CH₂— | 1,2,4-triazol-1-yl | Cl | H | H | |
| 1-1673 | 3,5-(F)₂—Ph | N | —CH₂CH₂—O—CH₂CH₂— | 1,2,4-triazol-1-yl | Cl | H | H | |
| 1-1674 | 2,34-(F)₃—Ph | N | —CH₂CH₂—O—CH₂CH₂— | 1,2,4-triazol-1-yl | Cl | H | H | |
| 1-1675 | 2,3,6-(F)₃—Ph | N | —CH₂CH₂—O—CH₂CH₂— | 1,2,4-triazol-1-yl | Cl | H | H | |
| 1-1676 | 2,4,6-(F)₃—Ph | N | —CH₂CH₂—O—CH₂CH₂— | 1,2,4-triazol-1-yl | Cl | H | H | |
| 1-1677 | 2,3,4,5,6-(F)₅—Ph | N | —CH₂CH₂—O—CH₂CH₂— | 1,2,4-triazol-1-yl | Cl | H | H | |
| 1-1678 | 23,4,5,6-(F)₅—Ph | N | —CH₂CH₂—O—CH₂CH₂— | 1,2,4-triazol-1-yl | Cl | F | F | |
| 1-1679 | pyridin-2-yl | N | —CH₂CH₂—O—CH₂CH₂— | 1,2,4-triazol-1-yl | Cl | H | H | |
| 1-1680 | 6-Cl-pyridin-2-yl | N | —CH₂CH₂—O—CH₂CH₂— | 1,2,4-triazol-1-yl | Cl | H | H | |
| 1-1681 | 3-Cl-5-CF₃-pyridin-2-yl | N | —CH₂CH₂—O—CH₂CH₂— | 1,2,4-triazol-1-yl | Cl | H | H | |
| 1-1682 | pyridin-3-yl | N | —CH₂CH₂—O—CH₂CH₂— | 1,2,4-triazol-1-yl | Cl | H | H | |
| 1-1683 | 6-Cl-pyridin-3-yl | N | —CH₂CH₂—O—CH₂CH₂— | 1,2,4-triazol-1-yl | Cl | H | H | |
| 1-1684 | pyridin-4-yl | N | —CH₂CH₂—O—CH₂CH₂— | 1,2,4-triazol-1-yl | Cl | H | H | |
| 1-1685 | 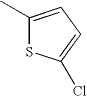 | N | —CH₂CH₂—O—CH₂CH₂— | 1,2,4-triazol-1-yl | Cl | H | H | |
| 1-1686 | 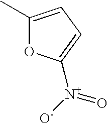 | N | —CH₂CH₂—O—CH₂CH₂— | 1,2,4-triazol-1-yl | Cl | H | H | |
| 1-1687 | Ph | N | —CH₂CH₂—O—CH₂CH₂— | 1,2,4-triazol-1-yl | H | H | H | |
| 1-1688 | Ph | N | —CH₂CH₂—O—CH₂CH₂— | 1,2,3-triazol-1-yl | Cl | H | H | |
| 1-1689 | Ph | N | —CH₂CH₂—O—CH₂CH₂— | 1,25-triazol-1-yl | Cl | H | H | |
| 1-1690 | Ph | N | —CH₂CH₂—O—CH₂CH₂— | tetrazol-1-yl | Cl | H | H | |
| 1-1691 | Ph | N | —CH₂CH₂—O—CH₂CH₂— | tetrazol-2-yl | Cl | H | H | |
| 1-1692 | Ph | N | —CH₂(CH₂)₂CH₂— | pyrazol-2-yl | H | H | H | |
| 1-1693 | Ph | N | —CH₂(CH₂)₂CH₂— | pyrazol-1-yl | Cl | H | H | |

TABLE 2

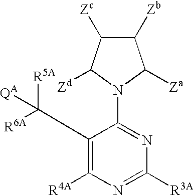

| | | | | | | | | | |
|---|---|---|---|---|---|---|---|---|---|
| Comp.No. | Q^{A} | Z^{a} | Z^{b} | Z^{c} | Z^{d} | R^{3A} | R^{4A} | R^{5A} | R^{6A} |

| | | | | | | | | |
|---|---|---|---|---|---|---|---|---|
| 2-1 | Ph | H | —CF₂— | H | H | Cl | H | H |
| 2-2 | Ph | H | —CF₂— | H | H | Cl | CH₃ | H |
| 2-3 | 1-Naph | H | —CF₂— | H | H | Cl | H | H |
| 2-4 | 2-Naph | H | —CF₂— | H | H | Cl | H | H |
| 2-5 | 2-F—Ph | H | —CF₂— | H | H | Cl | H | H |
| 2-6 | 2-Cl—Ph | H | —CF₂— | H | H | Cl | H | H |
| 2-7 | 2-Br—Ph | H | —CF₂— | H | H | Cl | H | H |
| 2-8 | 2-I—Ph | H | —CF₂— | H | H | Cl | H | H |
| 2-9 | 2-CH₃—Ph | H | —CF₂— | H | H | Cl | H | H |
| 2-10 | 2-CF₃—Ph | H | —CF₂— | H | H | Cl | H | H |
| 2-11 | 2-CN—Ph | H | —CF₂— | H | H | Cl | H | H |
| 2-12 | 2-OCH₃—Ph | H | —CF₂— | H | H | Cl | H | H |
| 2-13 | 2-NO₂—Ph | H | —CF₂— | H | H | Cl | H | H |
| 2-14 | 2-NH₂—Ph | H | —CF₂— | H | H | Cl | H | H |
| 2-15 | 2-Ph—Ph | H | —CF₂— | H | H | Cl | H | H |
| 2-16 | 3-F—Ph | H | —CF₂— | H | H | Cl | H | H |
| 2-17 | 3-Cl—Ph | H | —CF₂— | H | H | Cl | H | H |
| 2-18 | 3-Br—Ph | H | —CF₂— | H | H | Cl | H | H |
| 2-19 | 3-I—Ph | H | —CF₂— | H | H | Cl | H | H |
| 2-20 | 3-CH₃—Ph | H | —CF₂— | H | H | Cl | H | H |
| 2-21 | 3-CF₃—Ph | H | —CF₂— | H | H | Cl | H | H |
| 2-22 | 3-CN—Ph | H | —CF₂— | H | H | Cl | H | H |
| 2-23 | 3-OCH₃—Ph | H | —CF₂— | H | H | Cl | H | H |
| 2-24 | 3-OCF₃—Ph | H | —CF₂— | H | H | Cl | H | H |
| 2-25 | 3-NO₂—Ph | H | —CF₂— | H | H | Cl | H | H |
| 2-26 | 3-NH₂—Ph | H | —CF₂— | H | H | Cl | H | H |
| 2-27 | 3-Ph—Ph | H | —CF₂— | H | H | Cl | H | H |
| 2-28 | 4-F—Ph | H | —CF₂— | H | H | Cl | H | H |
| 2-29 | 4-Cl—Ph | H | —CF₂— | H | H | Cl | H | H |
| 2-30 | 4-Br—Ph | H | —CF₂— | H | H | Cl | H | H |
| 2-31 | 4-CH₃—Ph | H | —CF₂— | H | H | Cl | H | H |
| 2-32 | 4-C(CH₃)₃—Ph | H | —CF₂— | H | H | Cl | H | H |
| 2-33 | 4-CF₃—Ph | H | —CF₂— | H | H | Cl | H | H |
| 2-34 | 4-CN—Ph | H | —CF₂— | H | H | Cl | H | H |
| 2-35 | 4-OCH₃—Ph | H | —CF₂— | H | H | Cl | H | H |
| 2-36 | 4-OCF₃—Ph | H | —CF₂— | H | H | Cl | H | H |
| 2-37 | 4-NO₂—Ph | H | —CF₂— | H | H | Cl | H | H |
| 2-38 | 4-NH₂—Ph | H | —CF₂— | H | H | Cl | H | H |
| 2-39 | 4—Ph—Ph | H | —CF₂— | H | H | Cl | H | H |
| 2-40 | 2-Cl-4-F—Ph | H | —CF₂— | H | H | Cl | H | H |
| 2-41 | 2-Cl-6-F—Ph | H | —CF₂— | H | H | Cl | H | H |
| 2-42 | 2,3-(F)₂—Ph | H | —CF₂— | H | H | Cl | H | H |
| 2-43 | 2,4-(F)₂—Ph | H | —CF₂— | H | H | Cl | H | H |
| 2-44 | 2,5-(F)₂—Ph | H | —CF₂— | H | H | Cl | H | H |
| 2-45 | 2,6-(F)₂—Ph | H | —CF₂— | H | H | Cl | H | H |
| 2-46 | 3,4-(F)₂—Ph | H | —CF₂— | H | H | Cl | H | H |
| 2-47 | 3,5-(F)₂—Ph | H | —CF₂— | H | H | Cl | H | H |
| 2-48 | 2,3,4-(F)₃—Ph | H | —CF₂— | H | H | Cl | H | H |
| 2-49 | 2,3,6-(F)₃—Ph | H | —CF₂— | H | H | Cl | H | H |
| 2-50 | 2,4,6-(F)₃—Ph | H | —CF₂— | H | H | Cl | H | H |
| 2-51 | 2,3,4,5,6-(F)₅—Ph | H | —CF₂— | H | H | Cl | H | H |
| 2-52 | 2,3,4,5,6-(F)₅—Ph | H | —CF₂— | H | H | Cl | F | F |
| 2-53 | pyridyl-2-yl | H | —CF₂— | H | H | Cl | H | H |
| 2-54 | 6-Cl-pyridyl-2-yl | H | —CF₂— | H | H | Cl | H | H |
| 2-55 | 3-Cl-5-CF₃-pyridyl-2-yl | H | —CF₂— | H | H | Cl | H | H |
| 2-56 | pyridyl-3-yl | H | —CF₂— | H | H | Cl | H | H |
| 2-57 | 6-Cl-pyridyl-3-yl | H | —CF₂— | H | H | Cl | H | H |
| 2-58 | pyridyl-4-yl | H | —CF₂— | H | H | Cl | H | H |
| 2-59 | 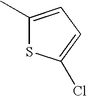 | H | —CF₂— | H | H | Cl | H | H |
| 2-60 | 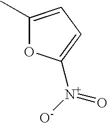 | H | —CF₂— | H | H | Cl | H | H |
| 2-61 | Ph | H | —CF₂— | H | OCH₃ | Cl | H | H |
| 2-62 | Ph | H | —CF₂— | H | O(CH₂)₃CH₃ | Cl | H | H |
| 2-63 | Ph | H | —CF₂— | H | CN | Cl | H | H |
| 2-64 | Ph | H | —CF₂— | H | ON═c(CH₃)₂ | Cl | H | H |
| 2-65 | Ph | H | —CF₂— | H | NH(CH₃)NH₂ | Cl | H | H |
| 2-66 | Ph | H | —CF₂— | H | N(CH₃)N═C(CH₃)₂ | Cl | H | H |
| 2-67 | Ph | H | —CF₂— | H | N(CH₃)N═(Ph)CH₃ | Cl | H | H |
| 2-68 | Ph | H | —CF₂— | H | C(═NOH)NH₂ | Cl | H | H |
| 2-69 | Ph | H | —CF₂— | H | C(═NOCH₃)NH₂ | Cl | H | H |
| 2-70 | Ph | H | —CF₂— | H | C(CH₃)═NOCH₂CH₃ | Cl | H | H |
| 2-71 | Ph | H | —CF₂— | H | C(CH₃)═NOCH₂CH═CHCl | Cl | H | H |
| 2-72 | Ph | H | —CF₂— | H | C(CH₃)═NOCH₂CH₂OCH₃ | Cl | H | H |
| 2-73 | Ph | H | —CF₂— | H | pyridyl-2-yl | Cl | H | H |
| 2-74 | Ph | H | —CF₂— | H | pyrazin-2-yl | Cl | H | H |
| 2-75 | Ph | H | —CF₂— | H | pyrazol-1-yl | Cl | H | H |
| 2-76 | Ph | H | —CF₂— | H | 1,2,4-triazol-1-yl | Cl | H | H |
| 2-77 | Ph | H | —CF₂— | H | H | H | H | H |
| 2-78 | Ph | CH₃ | —CF₂— | H | H | Cl | H | H |
| 2-79 | Ph | CH₃ | —CF₂— | CH₃ | H | Cl | H | H |
| 2-80 | Ph | H | —CCl₂— | H | H | Cl | H | H |
| 2-81 | Ph | H | —CCl₂— | H | H | Cl | CH₃ | H |
| 2-82 | 1-Naph | H | —CCl₂— | H | H | Cl | H | H |
| 2-83 | 2-Naph | H | —CCl₂— | H | H | Cl | H | H |
| 2-84 | 2-F—Ph | H | —CCl₂— | H | H | Cl | H | H |
| 2-85 | 2-Cl—Ph | H | —CCl₂— | H | H | Cl | H | H |
| 2-86 | 2-Br—Ph | H | —CCl₂— | H | H | Cl | H | H |
| 2-87 | 2-I—Ph | H | —CCl₂— | H | H | Cl | H | H |
| 2-88 | 2-CH₃—Ph | H | —CCl₂— | H | H | Cl | H | H |
| 2-89 | 2-CF₃—Ph | H | —CCl₂— | H | H | Cl | H | H |
| 2-90 | 2-CN—Ph | H | —CCl₂— | H | H | Cl | H | H |
| 2-91 | 2-OCH₃—Ph | H | —CCl₂— | H | H | Cl | H | H |
| 2-92 | 2-NO₂—Ph | H | —CCl₂— | H | H | Cl | H | H |
| 2-93 | 2-NH₂—Ph | H | —CCl₂— | H | H | Cl | H | H |
| 2-94 | 2-Ph—Ph | H | —CCl₂— | H | H | Cl | H | H |
| 2-95 | 3-F—Ph | H | —CCl₂— | H | H | Cl | H | H |
| 2-96 | 3-Cl—Ph | H | —CCl₂— | H | H | Cl | H | H |
| 2-97 | 3-Br—Ph | H | —CCl₂— | H | H | Cl | H | H |
| 2-98 | 3-I—Ph | H | —CCl₂— | H | H | Cl | H | H |
| 2-99 | 3-CH₃—Ph | H | —CCl₂— | H | H | Cl | H | H |
| 2-100 | 3-CF₃—Ph | H | —CCl₂— | H | H | Cl | H | H |
| 2-101 | 3-CN—Ph | H | —CCl₂— | H | H | Cl | H | H |
| 2-102 | 3-OCH₃—Ph | H | —CCl₂— | H | H | Cl | H | H |
| 2-103 | 3-OCF₃—Ph | H | —CCl₂— | H | H | Cl | H | H |
| 2-104 | 3-NO₂—Ph | H | —CCl₂— | H | H | Cl | H | H |
| 2-105 | 3-NH₂—Ph | H | —CCl₂— | H | H | Cl | H | H |
| 2-106 | 3-Ph—Ph | H | —CCl₂— | H | H | Cl | H | H |
| 2-107 | 4-F—Ph | H | —CCl₂— | H | H | Cl | H | H |
| 2-108 | 4-Cl—Ph | H | —CCl₂— | H | H | Cl | H | H |
| 2-109 | 4-Br—Ph | H | —CCl₂— | H | H | Cl | H | H |
| 2-110 | 4-CH₃—Ph | H | —CCl₂— | H | H | Cl | H | H |
| 2-111 | 4-C(CH₃)₃—Ph | H | —CCl₂— | H | H | Cl | H | H |
| 2-112 | 4-CF₃—Ph | H | —CCl₂— | H | H | Cl | H | H |
| 2-113 | 4-CN—Ph | H | —CCl₂— | H | H | Cl | H | H |
| 2-114 | 4-OCH₃—Ph | H | —CCl₂— | H | H | Cl | H | H |
| 2-115 | 4-OCF₃—Ph | H | —CCl₂— | H | H | Cl | H | H |
| 2-116 | 4-NO₂—Ph | H | —CCl₂— | H | H | Cl | H | H |
| 2-117 | 4-NH₂—Ph | H | —CCl₂— | H | H | Cl | H | H |
| 2-118 | 4-Ph—Ph | H | —CCl₂— | H | H | Cl | H | H |
| 2-119 | 2-Cl-4-F—Ph | H | —CCl₂— | H | H | Cl | H | H |
| 2-120 | 2-Cl-6-F—Ph | H | —CCl₂— | H | H | Cl | H | H |
| 2-121 | 2,3-(F)₂—Ph | H | —CCl₂— | H | H | Cl | H | H |
| 2-122 | 2,4-(F)₂—Ph | H | —CCl₂— | H | H | Cl | H | H |
| 2-123 | 2,5-(F)₂—Ph | H | —CCl₂— | H | H | Cl | H | H |
| 2-124 | 2,6-(F)₂—Ph | H | —CCl₂— | H | H | Cl | H | H |
| 2-125 | 3,4-(F)₂—Ph | H | —CCl₂— | H | H | Cl | H | H |
| 2-126 | 3,5-(F)₂—Ph | H | —CCl₂— | H | H | Cl | H | H |
| 2-127 | 2,3,4-(F)₃—Ph | H | —CCl₂— | H | H | Cl | H | H |
| 2-128 | 2,3,6-(F)₃—Ph | H | —CCl₂— | H | H | Cl | H | H |
| 2-129 | 2,4,6-(F)₃—Ph | H | —CCl₂— | H | H | Cl | H | H |
| 2-130 | 2,3,4,5,6-(F)₅—Ph | H | —CCl₂— | H | H | Cl | H | H |
| 2-131 | 2,3,4,5,6-(F)₅—Ph | H | —CCl₂— | H | H | Cl | F | F |
| 2-132 | pyridin-2-yl | H | —CCl₂— | H | H | Cl | H | H |
| 2-133 | 6-Cl-pyridin-2-yl | H | —CCl₂— | H | H | Cl | H | H |
| 2-134 | 3-Cl-5-CF₃-pyridin-2-yl | H | —CCl₂— | H | H | Cl | H | H |
| 2-135 | pyridin-3-yl | H | —CCl₂— | H | H | Cl | H | H |
| 2-136 | 6-Cl-pyridin-3-yl | H | —CCl₂— | H | H | Cl | H | H |
| 2-137 | pyridin-4-yl | H | —CCl₂— | H | H | Cl | H | H |
| 2-138 | 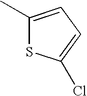 | H | —CCl₂— | H | H | Cl | H | H |
| 2-139 | 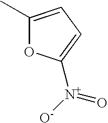 | H | —CCl₂— | H | H | Cl | H | H |
| 2-140 | Ph | H | —CCl₂— | H | OCH₃ | Cl | H | H |
| 2-141 | Ph | H | —CCl₂— | H | O(CH₂)₃CH₃ | Cl | H | H |
| 2-142 | Ph | H | —CCl₂— | H | CN | Cl | H | H |
| 2-143 | Ph | H | —CCl₂— | H | ON═(CH₃)₂ | Cl | H | H |
| 2-144 | Ph | H | —CCl₂— | H | NH(CH₃)NH₂ | Cl | H | H |
| 2-145 | Ph | H | —CCl₂— | H | N(CH₃)N═C(CH₃)₂ | Cl | H | H |
| 2-146 | Ph | H | —CCl₂— | H | N(CH₃)N═(Ph)CH₃ | Cl | H | H |
| 2-147 | Ph | H | —CCl₂— | H | C(═NOH)NH₂ | Cl | H | H |
| 2-148 | Ph | H | —CCl₂— | H | C(═NOCH₃)NH₂ | Cl | H | H |
| 2-149 | Ph | H | —CCl₂— | H | C(CH₃)═NOCH₂CH₃ | Cl | H | H |
| 2-150 | Ph | H | —CCl₂— | H | C(CH₃)═NOCH₂CH═CHCl | Cl | H | H |
| 2-151 | Ph | H | —CCl₂— | H | C(CH₃)═NOCH₂CH₂OCH₃ | Cl | H | H |
| 2-152 | Ph | H | —CCl₂— | H | pyridyl-2-yl | Cl | H | H |
| 2-153 | Ph | H | —CCl₂— | H | pyrazin-2-yl | Cl | H | H |
| 2-154 | Ph | H | —CCl₂— | H | pyrazol-1-yl | Cl | H | H |
| 2-155 | Ph | H | —CCl₂— | H | 1,2,4-triazol-1-yl | Cl | H | H |
| 2-156 | Ph | H | —CCl₂— | H | H | H | H | H |
| 2-157 | Ph | CH₃ | —CCl₂— | H | H | Cl | H | H |
| 2-158 | Ph | CH₃ | —CCl₂— | CH₃ | H | Cl | H | H |
| 2-159 | Ph | ═O | —CH₂—CH═CH—CH₂— | ═O | H | H | H | H |

| | | | | | | | | |
|---|---|---|---|---|---|---|---|---|
| 2-160 | Ph | —CH₂(CH₂)₂CH₂— | H | H | H | Cl | H | H |

TABLE 3

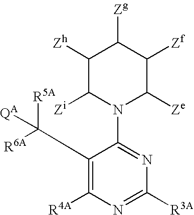

| | | | | | | | | | | |
|---|---|---|---|---|---|---|---|---|---|---|
| Comp.No. | Q^{A} | Z^{e} | Z^{f} | Z^{g} | Z^{h} | Zⁱ | R^{3A} | R^{4A} | R^{5A} | R^{6A} |

| | | | | | | | | | |
|---|---|---|---|---|---|---|---|---|---|
| 3-1 | Ph | H | —CF₂— | H | H | H | Cl | H | H |
| 3-2 | Ph | H | —CF₂— | H | H | H | Cl | CH₃ | H |
| 3-3 | 1-Naph | H | —CF₂— | H | H | H | Cl | H | H |
| 3-4 | 2-Naph | H | —CF₂— | H | H | H | Cl | H | H |
| 3-5 | 2-F—Ph | H | —CF₂— | H | H | H | Cl | H | H |
| 3-6 | 2-Cl—Ph | H | —CF₂— | H | H | H | Cl | H | H |
| 3-7 | 2-Br—Ph | H | —CF₂— | H | H | H | Cl | H | H |
| 3-8 | 2-I—Ph | H | —CF₂— | H | H | H | Cl | H | H |
| 3-9 | 2-CH₃—Ph | H | —CF₂— | H | H | H | Cl | H | H |
| 3-10 | 2-CF₃—Ph | H | —CF₂— | H | H | H | Cl | H | H |
| 3-11 | 2-CN—Ph | H | —CF₂— | H | H | H | Cl | H | H |
| 3-12 | 2-OCH₃—Ph | H | —CF₂— | H | H | H | Cl | H | H |
| 3-13 | 2-NO₂—Ph | H | —CF₂— | H | H | H | Cl | H | H |
| 3-14 | 2-NH₂—Ph | H | —CF₂— | H | H | H | Cl | H | H |
| 3-15 | 2-Ph—Ph | H | —CF₂— | H | H | H | Cl | H | H |
| 3-16 | 3-F—Ph | H | —CF₂— | H | H | H | Cl | H | H |
| 3-17 | 3-Cl—Ph | H | —CF₂— | H | H | H | Cl | H | H |
| 3-18 | 3-Br—Ph | H | —CF₂— | H | H | H | Cl | H | H |
| 3-19 | 3-I—Ph | H | —CF₂— | H | H | H | Cl | H | H |
| 3-20 | 3-CH₃—Ph | H | —CF₂— | H | H | H | Cl | H | H |
| 3-21 | 3-CF₃—Ph | H | —CF₂— | H | H | H | Cl | H | H |
| 3-22 | 3-CN—Ph | H | —CF₂— | H | H | H | Cl | H | H |
| 3-23 | 3-OCH₃—Ph | H | —CF₂— | H | H | H | Cl | H | H |
| 3-24 | 3-OCF₃—Ph | H | —CF₂— | H | H | H | Cl | H | H |
| 3-25 | 3-NO₂—Ph | H | —CF₂— | H | H | H | Cl | H | H |
| 3-26 | 3-NH₂—Ph | H | —CF₂— | H | H | H | Cl | H | H |
| 3-27 | 3-Ph—Ph | H | —CF₂— | H | H | H | Cl | H | H |
| 3-28 | 4-F—Ph | H | —CF₂— | H | H | H | Cl | H | H |
| 3-29 | 4-Cl—Ph | H | —CF₂— | H | H | H | Cl | H | H |
| 3-30 | 4-Br—Ph | H | —CF₂— | H | H | H | Cl | H | H |
| 3-31 | 4-CH₃—Ph | H | —CF₂— | H | H | H | Cl | H | H |
| 3-32 | 4-C(CH₃)₃—Ph | H | —CF₂— | H | H | H | Cl | H | H |
| 3-33 | 4-CF₃—Ph | H | —CF₂— | H | H | H | Cl | H | H |
| 3-34 | 4-CN—Ph | H | —CF₂— | H | H | H | Cl | H | H |
| 3-35 | 4-OCH₃—Ph | H | —CF₂— | H | H | H | Cl | H | H |
| 3-36 | 4-OCF₃—Ph | H | —CF₂— | H | H | H | Cl | H | H |
| 3-37 | 4-NO₂—Ph | H | —CF₂— | H | H | H | Cl | H | H |
| 3-38 | 4-NH₂—Ph | H | —CF₂— | H | H | H | Cl | H | H |
| 3-39 | 4-Ph—Ph | H | —CF₂— | H | H | H | Cl | H | H |
| 3-40 | 2-Cl-4-F—Ph | H | —CF₂— | H | H | H | Cl | H | H |
| 3-41 | 2-Cl-6-F—Ph | H | —CF₂— | H | H | H | Cl | H | H |
| 3-42 | 2,3-(F)₂—Ph | H | —CF₂— | H | H | H | Cl | H | H |
| 3-43 | 2,4-(F)₂—Ph | H | —CF₂— | H | H | H | Cl | H | H |
| 3-44 | 2,5-(F)₂—Ph | H | —CF₂— | H | H | H | Cl | H | H |
| 3-45 | 2,6-(F)₂—Ph | H | —CF₂— | H | H | H | Cl | H | H |
| 3-46 | 3,4-(F)₂—Ph | H | —CF₂— | H | H | H | Cl | H | H |
| 3-47 | 3,5-(F)₂—Ph | H | —CF₂— | H | H | H | Cl | H | H |
| 3-48 | 2,3,4-(F)₃—Ph | H | —CF₂— | H | H | H | Cl | H | H |
| 3-49 | 2,3,6-(F)₃—Ph | H | —CF₂— | H | H | H | Cl | H | H |
| 3-50 | 2,4,6-(F)₃—Ph | H | —CF₂— | H | H | H | Cl | H | H |
| 3-51 | 2,3,4,5,6-(F)₅—Ph | H | —CF₂— | H | H | H | Cl | H | H |
| 3-52 | 2,3,4,5,6-(F)₅—Ph | H | —CF₂— | H | H | H | Cl | F | F |
| 3-53 | pyridin-2-yl | H | —CF₂— | H | H | H | Cl | H | H |
| 3-54 | 6-Cl-pyridin-2-yl | H | —CF₂— | H | H | H | Cl | H | H |
| 3-55 | 3-Cl-5-CF₃-pyridin-2-yl | H | —CF₂— | H | H | H | Cl | H | H |
| 3-56 | pyridin-3-yl | H | —CF₂— | H | H | H | Cl | H | H |
| 3-57 | 6-Cl-pyridin-3-yl | H | —CF₂— | H | H | H | Cl | H | H |
| 3-58 | pyridin-4-yl | H | —CF₂— | H | H | H | Cl | H | H |
| 3-59 | 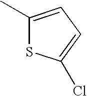 | H | —CF₂— | H | H | H | Cl | H | H |
| 3-60 | 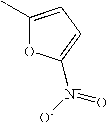 | H | —CF₂— | H | H | H | Cl | H | H |
| 3-61 | Ph | H | —CF₂— | H | H | OCH₃ | Cl | H | H |
| 3-62 | Ph | H | —CF₂— | H | H | O(CH₂)₃CH₃ | Cl | H | H |
| 3-63 | Ph | H | —CF₂— | H | H | CN | Cl | H | H |
| 3-64 | Ph | H | —CF₂— | H | H | CN═(CH₃)₂ | Cl | H | H |
| 3-65 | Ph | H | —CF₂— | H | H | NH(CH₃)NH₂ | Cl | H | H |
| 3-66 | Ph | H | —CF₂— | H | H | N(CH₃)N═C(CH₃)₂ | Cl | H | H |
| 3-67 | Ph | H | —CF₂— | H | H | N(CH₃)N═(Ph)CH₃ | Cl | H | H |
| 3-68 | Ph | H | —CF₂— | H | H | C(═NOH)NH₂ | Cl | H | H |
| 3-69 | Ph | H | —CF₂— | H | H | C(═NOCH₃)NH₂ | Cl | H | H |
| 3-70 | Ph | H | —CF₂— | H | H | C(CH₃)═NOCH₂CH₃ | Cl | H | H |
| 3-71 | Ph | H | —CF₂— | H | H | C(CH₃)═NOCH₂CH═CHCl | Cl | H | H |
| 3-72 | Ph | H | —CF₂— | H | H | C(CH₃)═NOCH₂CH₂OCH₃ | Cl | H | H |
| 3-73 | Ph | H | —CF₂— | H | H | pyridin-2-yl | Cl | H | H |
| 3-74 | Ph | H | —CF₂— | H | H | pyrazin-2-yl | Cl | H | H |
| 3-75 | Ph | H | —CF₂— | H | H | pyrazol-1-yl | Cl | H | H |
| 3-76 | Ph | H | —CF₂— | H | H | 1,2,4-triazol-1-yl | Cl | H | H |
| 3-77 | Ph | H | —CF₂— | H | H | H | H | H | H |
| 3-78 | Ph | H | —CCl₂— | H | H | H | Cl | H | H |
| 3-79 | Ph | H | —CCl₂— | H | H | H | Cl | CH₃ | H |
| 3-80 | 1-Naph | H | —CCl₂— | H | H | H | Cl | H | H |
| 3-81 | 2-Naph | H | —CCl₂— | H | H | H | Cl | H | H |
| 3-82 | 2-F—Ph | H | —CCl₂— | H | H | H | Cl | H | H |
| 3-83 | 2-Cl—Ph | H | —CCl₂— | H | H | H | Cl | H | H |
| 3-84 | 2-Br—Ph | H | —CCl₂— | H | H | H | Cl | H | H |
| 3-85 | 2-I—Ph | H | —CCl₂— | H | H | H | Cl | H | H |
| 3-86 | 2-CH₃—Ph | H | —CCl₂— | H | H | H | Cl | H | H |
| 3-87 | 2-CF₃—Ph | H | —CCl₂— | H | H | H | Cl | H | H |
| 3-88 | 2-CN—Ph | H | —CCl₂— | H | H | H | Cl | H | H |
| 3-89 | 2-OCH₃—Ph | H | —CCl₂— | H | H | H | Cl | H | H |
| 3-90 | 2-NO₂—Ph | H | —CCl₂— | H | H | H | Cl | H | H |
| 3-91 | 2-NH₂—Ph | H | —CCl₂— | H | H | H | Cl | H | H |
| 3-92 | 2-Ph—Ph | H | —CCl₂— | H | H | H | Cl | H | H |
| 3-93 | 3-F—Ph | H | —CCl₂— | H | H | H | Cl | H | H |
| 3-94 | 3-Cl—Ph | H | —CCl₂— | H | H | H | Cl | H | H |
| 3-95 | 3-Br—Ph | H | —CCl₂— | H | H | H | Cl | H | H |
| 3-96 | 3-I—Ph | H | —CCl₂— | H | H | H | Cl | H | H |
| 3-97 | 3-CH₃—Ph | H | —CCl₂— | H | H | H | Cl | H | H |
| 3-98 | 3-CF₃—Ph | H | —CCl₂— | H | H | H | Cl | H | H |
| 3-99 | 3-CN—Ph | H | —CCl₂— | H | H | H | Cl | H | H |
| 3-100 | 3-OCH₃—Ph | H | —CCl₂— | H | H | H | Cl | H | H |
| 3-101 | 3-OCF₃—Ph | H | —CCl₂— | H | H | H | Cl | H | H |
| 3-102 | 3-NO₂—Ph | H | —CCl₂— | H | H | H | Cl | H | H |
| 3-103 | 3-NH₂—Ph | H | —CCl₂— | H | H | H | Cl | H | H |
| 3-104 | 3—Ph—Ph | H | —CCl₂— | H | H | H | Cl | H | H |
| 3-105 | 4-F—Ph | H | —CCl₂— | H | H | H | Cl | H | H |
| 3-106 | 4-Cl—Ph | H | —CCl₂— | H | H | H | Cl | H | H |
| 3-107 | 4-Br—Ph | H | —CCl₂— | H | H | H | Cl | H | H |
| 3-108 | 4-CH₃—Ph | H | —CCl₂— | H | H | H | Cl | H | H |
| 3-109 | 4-C(CH₃)₃—Ph | H | —CCl₂— | H | H | H | Cl | H | H |
| 3-110 | 4-CF₃—Ph | H | —CCl₂— | H | H | H | Cl | H | H |
| 3-111 | 4-CN—Ph | H | —CCl₂— | H | H | H | Cl | H | H |
| 3-112 | 4-OCH₃—Ph | H | —CCl₂— | H | H | H | Cl | H | H |
| 3-113 | 4-OCF₃—Ph | H | —CCl₂— | H | H | H | Cl | H | H |
| 3-114 | 4-NO₂—Ph | H | —CCl₂— | H | H | H | Cl | H | H |
| 3-115 | 4-NH₂—Ph | H | —CCl₂— | H | H | H | Cl | H | H |
| 3-116 | 4—Ph—Ph | H | —CCl₂— | H | H | H | Cl | H | H |
| 3-117 | 2-Cl-4-F—Ph | H | —CCl₂— | H | H | H | Cl | H | H |
| 3-118 | 2-Cl-6-F—Ph | H | —CCl₂— | H | H | H | Cl | H | H |
| 3-119 | 2,3-(F)₂—Ph | H | —CCl₂— | H | H | H | Cl | H | H |
| 3-120 | 2,4-(F)₂—Ph | H | —CCl₂— | H | H | H | Cl | H | H |
| 3-121 | 2,5-(F)₂—Ph | H | —CCl₂— | H | H | H | Cl | H | H |
| 3-122 | 2,6-(F)₂—Ph | H | —CCl₂— | H | H | H | Cl | H | H |
| 3-123 | 3,4-(F)₂—Ph | H | —CCl₂— | H | H | H | Cl | H | H |
| 3-124 | 3,5-(F)₂—Ph | H | —CCl₂— | H | H | H | Cl | H | H |
| 3-125 | 2,3,4-(F)₃—Ph | H | —CCl₂— | H | H | H | Cl | H | H |
| 3-126 | 2,3,6-(F)₃—Ph | H | —CCl₂— | H | H | H | Cl | H | H |
| 3-127 | 2,4,6-(F)₃—Ph | H | —CCl₂— | H | H | H | Cl | H | H |
| 3-128 | 2,3,4,5,6-(F)₅—Ph | H | —CCl₂— | H | H | H | Cl | H | H |
| 3-129 | 2,3,4,5,6-(F)₅—Ph | H | —CCl₂— | H | H | H | F | H | H |
| 3-130 | pyridin-2-yl | H | —CCl₂— | H | H | H | Cl | H | H |
| 3-131 | 6-Cl-pyridin-2-yl | H | —CCl₂— | H | H | H | Cl | H | H |
| 3-132 | 3-Cl-5-CF₃—pyridin-2-yl | H | —CCl₂— | H | H | H | Cl | H | H |
| 3-133 | pyridin-3-yl | H | —CCl₂— | H | H | H | Cl | H | H |
| 3-134 | 6-Cl-pyridin-3-yl | H | —CCl₂— | H | H | H | Cl | H | H |
| 3-135 | pyridin-4-yl | H | —CCl₂— | H | H | H | Cl | H | H |
| 3-136 | 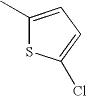 | H | —CCl₂— | H | H | H | Cl | H | H |
| 3-137 | 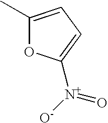 | H | —CCl₂— | H | H | H | Cl | H | H |
| 3-138 | Ph | H | —CCl₂— | H | H | OCH₃ | Cl | H | H |
| 3-139 | Ph | H | —CCl₂— | H | H | O(CH₂)₃CH₃ | Cl | H | H |
| 3-140 | Ph | H | —CCl₂— | H | H | CN | Cl | H | H |
| 3-141 | Ph | H | —CCl₂— | H | H | ON═C(CH₃)₂ | Cl | H | H |
| 3-142 | Ph | H | —CCl₂— | H | H | NH(CH₃)NH₂ | Cl | H | H |
| 3-143 | Ph | H | —CCl₂— | H | H | N(CH₃)N═C(CH₃)₂ | Cl | H | H |
| 3-144 | Ph | H | —CCl₂— | H | H | N(CH₃)N═(Ph)CH₃ | Cl | H | H |
| 3-145 | Ph | H | —CCl₂— | H | H | C(═NOH)NH₂ | Cl | H | H |
| 3-146 | Ph | H | —CCl₂— | H | H | C(═NOCH₃)NH₂ | Cl | H | H |
| 3-147 | Ph | H | —CCl₂— | H | H | C(CH₃)═NOCH₂CH₃ | Cl | H | H |
| 3-148 | Ph | H | —CCl₂— | H | H | C(CH₃)═NOCH₂CH═CHCl | Cl | H | H |
| 3-149 | Ph | H | —CCl₂— | H | H | C(CH₃)═NOCH₂CH₂OCH₃ | Cl | H | H |
| 3-150 | Ph | H | —CCl₂— | H | H | pyridin-2-yl | Cl | H | H |
| 3-151 | Ph | H | —CCl₂— | H | H | pyrazin-2-yl | Cl | H | H |
| 3-152 | Ph | H | —CCl₂— | H | H | pyrazol-1-yl | Cl | H | H |
| 3-153 | Ph | H | —CCl₂— | H | H | 1,2,4-triazol-1-yl | Cl | H | H |
| 3-154 | Ph | H | —CCl₂— | H | H | H | H | H | H |

| | | | | | | | | | |
|---|---|---|---|---|---|---|---|---|---|
| 3-155 | Ph | —CH₂(CH₂)₂CH₂— | H | H | H | H | Cl | H | H |

| | | | | | | | | | |
|---|---|---|---|---|---|---|---|---|---|
| 3-156 | Ph | H | —CH₂(CH₂)₂CH₂— | H | H | H | Cl | H | H |

TABLE 4

| Comp. | m. p. (° C.) or | |
|---|---|---|
| No. | nD20 | |
| 1-5 | * 1 | |
| 1-6 | 1,6119 | |
| 1-11 | 1,6050 | |
| 1-12 | 95˜98° | C. |
| 1-13 | 1,5639 | |
| 1-14 | 1,5914 | |
| 1-15 | 1,6112 | |
| 1-21 | 1,6140 | |
| 1-22 | 1,6150 | |
| 1-25 | 1,5950 | |
| 1-30 | 144˜147° | C. |
| 1-31 | 165° | C. |
| 1-32 | 166˜167° | C. |
| 1-33 | * 2 | |
| 1-36 | 87˜90° | C. |
| 1-37 | 88˜89° | C. |
| 1-38 | 1,6388 | |
| 1-39 | 1,5907 | |
| 1-40 | 90˜94° | C. |
| 1-41 | 83˜85° | C. |
| 1-42 | 112˜115° | C. |
| 1-45 | 106˜107° | C. |
| 1-46 | 123˜124° | C. |
| 1-56 | * 3 | |
| 1-57 | * 4 | |
| 1-61 | 97˜98° | C. |
| 1-62 | 137˜139° | C. |
| 1-68 | 108˜109° | C. |
| 1-69 | 97˜98° | C. |
| 1-73 | 119˜120° | C. |
| 1-85 | 86˜89° | C. |
| 1-87 | 99˜100° | C. |
| 1-93 | 51˜52° | C. |
| 1-102 | 51˜54° | C. |
| 1-104 | 1,5935 | |
| 1-108 | 74˜76° | C. |
| 1-117 | 1,6027 | |
| 1-121 | 111˜112° | C. |
| 1-165 | 97˜99° | C. |
| 1-175 | 136˜138° | C. |
| 1-235 | 98˜99° | C. |
| 1-238 | 128˜130° | C. |
| 1-303 | 169˜170° | C. |
| 1-304 | 206˜207° | C. |
| 1-311 | 183˜184° | C. |
| 1-435 | 158˜160° | C. |
| 1-506 | 1,5915 | |
| 1-507 | 73˜74° | C. |
| 1-522 | 1,5765 | |
| 1-523 | 1,5825 | |
| 1-524 | 1,5850 | |
| 1-532 | 82˜83° | C. |
| 1-543 | 66˜68° | C. |
| 1-550 | 1,5962 | |
| 1-563 | 97˜98° | C. |
| 1-564 | 82˜85° | C. |
| 1-574 | 52˜54° | C. |
| 1-575 | 68˜71° | C. |
| 1-579 | * 5 | |
| 1-580 | 1,6088 | |
| 1-586 | 1,5830 | |
| 1-587 | 103˜105° | C. |
| 1-591 | 82˜83° | C. |
| 1-629 | 1,5923 | |
| 1-631 | 1,5682 | |
| 1-651 | 129˜130° | C. |
| 1-714 | 156° | C. |
| 1-715 | 134˜135° | C. |
| 1-722 | 132˜134° | C. |
| 1-1045 | 118˜119° | C. |
| 1-1309 | 92˜93° | C. |
| 1-1429 | 153˜155° | C. |
| 1-1693 | 149˜151° | C. |

* 1: 1H NMR(CDCl3, 300MHz)δ 1.80-1.85(4H, m), 3.54-3.58(4H, m), 4.278(2H, s), 7.082H, d, J=6.9Hz), 7.21-7.31(3H, m), 8.31, 8.31(1H, s).
* 2: 1H NMR(CDCl3, 300MHz)δ 4.34(2H, S), 4.43(4H, S), 5.76(2H, S), 7.07(2H, d), 7.21-7.31(3H, m), 8.34(1H, S).
* 3: 1H NMR(CDCl3, 300MHz)δ 1.77-1.85(4H, m), 3.51-3.56(4H, m), 4.24(2H, s), 6.76-6.93(3H, m), 7.22-7.29(1H, m), 8.30(1H, s).
* 4: 1H NMR(CDCl3, 300MHz)δ 1.83-1.87(4H, m), 3.52-3.57(4H, m), 4.24(2H, s), 6.94-7.25(4H, m), 8.32(1H, s).
* 5: 1H NMR(CDCl3, 300MHz)δ 1.59-1.65(6H, m), 3.29-3.31(4H, m), 4.12(2H, s), 7.29-7.48(4H, m), 8.46(1H, s).

### SYNTHESIS EXAMPLE 7 (SYNTHESIS OF AN INTERMEDIATE

First Step

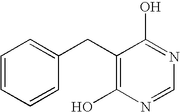

To a suspension of formamidine acetate (46 g, 0.44 mol) and ethanol (300 ml), 28% methanol solution of sodium methoxide (250 g, 1.3 mol) was added under ice cooling and, after stirring the mixture for 1 hour under continuous ice cooling diethyl benzylmalonate (10 g, 0.4 mol) was added thereto. After stirring the mixture for 2 hours under ice cooling and for 19 hours at room temperature and then refluxed for 4 hours. After finishing the reaction, the precipitation, formed by adding concentrated hydrochloric acid (130 g) under ice cooling, was filtered, washed with ethanol and then with diethyl ether, and dried in a desiccator to obtain 5-benzyl-1H-purimidine-4,6-dione (145 g) which was used in the next reaction without purification.
Second Step

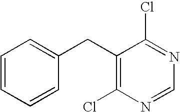

To 5-benzyl-1H-pyrimidine-4,6-dione (145 g), phosphorus oxychloride (300 ml) and dichloroethane (200 ml) were added and the mixture was refluxed for 3 hours. After finishing the reaction, the solvent and an excess of phosphorus oxychloride were removed under reduced pressure. After adding ice water and dichloromethane to the reaction mixture, the precipitation was removed and the filtrate was extracted with dichloromethane. The dichloromethane layer was dried with anhydrous magnesium sulfate, and filtered with a glass filter, filled with silica gel, by using ethyl acetate. The filtrate was concentrated under reduced pressure and the obtained crude product was dissolved in ethanol, to which ice water was added, and the formed precipitation was filtered, washed with water and then with diethyl ether, and dried in a desiccator to obtain 5-benzyl-4,6-dichloropyrimidine (51.8 g)

mp 91-92° C.

### SYNTHESIS EXAMPLE 8 (SYNTHESIS OF AN INTERMEDIATE

First Step

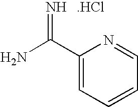

Ethyl 2-pyridylimidate (45 g, 0.3 mol) and ammonium chloride (19.3 g, 0.36 mol) were suspended in ethanol (150 ml) and the mixture was refluxed for 4 hours. After finishing the reaction, the reaction solution was concentrated to about ⅓ of the volume under reduced pressure. The precipitation, formed by adding diethyl ether (100 ml) thereto, was filtered, washed with diethyl ether and then with acetone, and dried in a desiccator to obtain 2-amidinopyridine hydrochloride (42.15 g).
Second Step

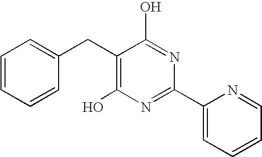

To a suspension of 2-amidinopyridine hydrochloride (25 g, 0.1 mol) and ethanol, 28% methanol solution of sodium methoxide (60 g, 0.31 mol) was added under ice cooling and, after stirring the mixture for 15 minutes under continuous ice cooling, diethyl benzylmalonate (100 g, 0.4 mol) was added thereto. After stirring the mixture for 1.5 hours under ice cooling and for 1 hour at room temperature, it was refluxed for 4 hours. After finishing the reaction, the precipitation, formed by adding concentrated hydrochloric acid (32 g) under ice cooling, was filtered, washed with ethanol and then with diethyl ether, and dried in a desiccator to obtain 5-benzyl-2-pyridin-2-yl-1H-pyrimidine-4,6-dione hydrochloride (38.7 g) which was used in the next reaction without purification.
Third Step

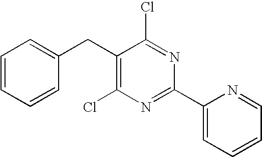

To 5-benzyl-2-pyridin-2-yl-1H-pyrimidine-4,6-dione hydrochloride (39.7 g), phosphorus oxychloride (200 ml) was added and the mixture was refluxed for 3 hours. After finishing the reaction, an excess of phosphorus oxychloride was removed under reduced pressure. After adding ice water and dichloromethane to the reaction mixture, the precipitation was removed and the filtrate was extracted with dichloromethane. The dichloromethane layer was dried with anhydrous magnesium sulfate, and filtered with a glass filter, filled with silica gel, by using ethyl acetate. The filtrate was concentrated under reduced pressure and the obtained product was dried in a desiccator to obtain 5-benzyl-4,6-dichloro-2-pyridin-2-yl-pyrimidine (15.8 g) which was used in the next reaction without purification.

mp 96-97° C.

### SYNTHESIS EXAMPLE 9 (SYNTHESIS OF AN INTERMEDIATE)

First Step

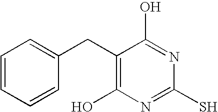

To a suspension of thiourea (25 g, 0.1 mol) and ethanol (300 ml), 28% methanol solution of sodium methoxide (58 g, 0.3 mol) and diethyl benzylmalonate (25 g, 0.1 mol) were added under ice cooling and, after stirring for 1 hour at room temperature, the mixture was refluxed for 4 hours. After finishing the reaction, the precipitation, formed by acidifying the mixture through addition of concentrated hydrochloric acid under ice cooling, was filtered, washed with ethanol and then with diethyl ether, and dried in a desiccator to obtain 5-benzyl-2-mercaptopyrimidine-4,6-dione (23 g) which was used in the next reaction without purification.
Second Step

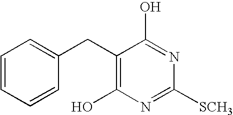

To a solution of 5-benzyl-2-mercaptopyrimidine-4,6-dione (23 g, 0.1 mol) in methanol (300 ml), 28% methanol solution of sodium methoxide (29 g, 0.15 mol) was added dropwise under ice cooling. Then methyl iodide (7.5 ml, 0.12 mol) was added to the mixture, which was stirred at room temperature for 1 hour. After finishing the reaction, the reaction solution was poured into ice water, acidified with hydrochloric acid, and the formed crystals were filtered and dried in a desiccator to obtain 5-benzyl-2-methylthiopyrimidine-4,6-dion (24.8 g).
Third Step

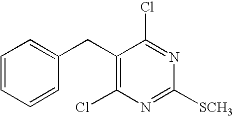

To 5-benzyl-2-methylthiopyrimidine-4,6-dione (24.8 g), phosphorusoxychloride (200 ml) was added and the mixture was refluxed for 3 hours. After finishing the reaction, an excess of phosphorus oxychloride was removed under reduced pressure. After adding ice water and dichloromethane to the reaction mixture, the precipitation was removed and the filtrate was extracted with dichloromethane. The dichloromethane layer was dried with anhydrous magnesium sulfate, and filtered with a glass filter, filled with silica gel, by using ethyl acetate. The filtrate was concentrated under reduced pressure and the obtained product was dried in a desiccator to obtain 5-benzyl-4,6-dichloro-2-methylthiopyrimidine (20.2 g) which was used in further reaction without purification.

### SYNTHESIS EXAMPLE 10 (SYNTHESIS OF AN INTERMEDIATE)

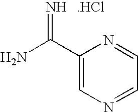

To pyrazinecarbonitrile (11.7 g, 0.11 mol), 28% methanol solution of sodium methoxide (2.0 g, 10 mmol) was added and the mixture was refluxed for 4 hours and, after adding ammonium chloride (6.4 g, 0.12 mol), for further 6 hours. After finishing the reaction, the precipitation, formed by adding diethyl ether (50 ml) to the mixture, was filtered, washed with diethyl ether and then with acetone, and dried in a desiccator to obtain amidinopyrazine hydrochloride (17.2 g), which was used in further reaction without purification.

### SYNTHESIS EXAMPLE 11 (SYNTHESIS OF AN INTERMEDIATE)

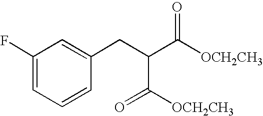

3-Fluorobenzyl bromide (18.9, 0.1 mol), diethyl malonate (120 ml, 0.8 mol) and potassium carbonate (30 g, 0.22 mol) were suspended in acetone (60 μl) and stirred at room temperature for 10 hours. After finishing the reaction, the precipitation was faltered and washed with acetone. The solvent and an excess of diethyl malonate were removed under reduced pressure and the residue was purified by flush column chromatography (eluent n-hexane:ethyl acetate=4:1) to obtain diethyl 3-fluorobenzylmalonate (23.6 g), which was used in further reaction without purification.

### TEST EXAMPLE 1

### Test for Effect of Foliage Application Against Pyricularia oryzae

Preparation of Testing Compound

Active compound: 5 parts by weight

Organic solvent: Acetone 142.5 parts by weight

Emulsifier: Polyoxyethylene alkyl phenyl ether 7.5 parts by weight.

The above-mentioned active compound, acetone and emulsifier were mixed, diluted to a prescribed concentration with water and used for test.

Test Method

Paddy rice (variety: KOSHIHIKARI) was cultivated in a plastic pot of 4 cm diameter. At its 1.5-2 leaf stage a previously prepared diluted solution of an active compound of the prescribed concentration was sprayed in an amount of 6 ml per 3 pots. One day after spraying, a suspension of spores of artificially cultured Pyricularia oryzae was inoculated by spraying (once) and infected in keeping at 25° C. and 100% relative humidity, Seven days after the inoculation, the contraction rate per pot was classified and evaluated to obtain the controlling value (%). Phytotoxicity was also studied at the same time. This test is an average of the results of 1 section 3 pots.

Evaluation of contraction rate and calculation method of controlling value are as follows

| Contraction rate | Lesion area ratio (%) |
|---|---|
| 0 | 0 |
| 0.5 | less than 2 |
| 1 | 2-less than 5 |
| 2 | 5-less than 10 |
| 3 | 10-less than 20 |
| 4 | 20-less than 40 |
| 5 | more than 40 |

Test Results

Compounds of the compound numbers 1-5, 1-11, 1-14, 1-15, 1-16, 1-17, 1-22, 1-33, 1-36, 1-37, 1-45, 1-56, 1-57, 1-68, 1-86, 1-87, 1-102 and 1-238 showed controlling values of more than 80% at the chemical concentration (500 ppm). No phytotoxicity was observed.

### TEST EXAMPLE 2

### Test for Effect of Foliage Application Against Sphaerotheca fuliginea

Test Method

Cucumber (variety: SAGAMI HANPAKU) was cultivated in a plastic pot of 4 cm diameter. A diluted solution of an active compound of the prescribed concentration, prepared in a similar manner as in the above-mentioned Test Example 1, was sprayed to seedlings reached to cotyledon in an amount of 6 ml per 3 pots. One day after the spraying, a suspension of spores, prepared by washing spores of Sphaerotheca fuliginea taken from previously infected cucumber into distilled water, was inoculated to the plant to be treated by spraying (once) and infected in a green house. Seven days after the inoculation, the contraction rate per pot was classified and evaluated to obtain the controlling value (%). Phytotoxicity was also studied at the same time. This test is an average of the results of 1 section 3 pots.

Evaluation of contraction rate and calculation method of controlling value are as follows

| Contraction rate | Lesion area ratio (%) |
|---|---|
| 0 | 0 |
| 0.5 | less than 2 |
| 1 | 2-less than 5 |
| 2 | 5-less than 10 |
| 3 | 10-less than 20 |
| 4 | 20-less than 40 |
| 5 | more than 40 |

Test Results

Compounds of the compound numbers 1-5, 1-6, 1-11, 1-14, 1-15, 1-16, 1-17, 1-46, 1-56, 1-57, 1-68, 1-86 and 1-87 showed controlling values of more than 80% at the chemical concentration (500 ppm). No phytotoxicity was observed.

### TEST EXAMPLE 3

### Test for Effect of Foliage Application Against Phytophthora infestams

Test Method

Tomato (variety: REGINA) was cultivated in a plastic pot of 4 cm diameter. A diluted solution of an active compound of the prescribed concentration, prepared in a similar manner as in the above-mentioned Test Example 1, was sprayed to seedlings reached to 2-3 leaf stage in an amount of 6 ml per 3 pots. One day after the spraying, a suspension of zoosporangia, prepared by washing zoosporangia of Phytophthora infestans formed on the lesion of the previously infected tomato into distilled water by using a brush, was inoculated to the plant to be treated by spraying (once) and infected in keeping at 20° C. and 100% relative humidity. Four days after the inoculation, the contraction rate per pot was classified and evaluated to obtain the controlling value (%). Phytotoxicity was studied at the same time. This test is an average of the results of 1 section 3 pots.

Evaluation of contraction rate and calculation method of controlling value are as follows

| Contraction rate | Lesion area ratio (%) |
|---|---|
| 0 | 0 |
| 0.5 | less than 2 |
| 1 | 2-less than 5 |
| 2 | 5-less than 10 |
| 3 | 10-less than 20 |
| 4 | 20-less than 40 |
| 5 | more than 40 |

Test Results

Compounds of the compound numbers 1-5, 1-165 and 1-238 showed controlling values of more than 80% at the chemical concentration (500 ppm). No phytotoxicity was observed.

### TEST EXAMPLE 4

### Test for Effect of Foliage Application Against Alternaria mali

Test Method

A nursery stock (variety: OREGON SUPER DELICIOUS) was cultivated in a plastic pot of 30 cm diameter and its leaves, which had reached at perfect extension stage, were detached from the petiole, were cultivated under hydroponic condition by using a water-holding carrier. After that, a diluted solution of an active compound of the prescribed concentration, prepared in a similar manner as in the above-mentioned Test Example 1, was sprayed to the leaves in an amount of 6 ml per 3 leaves. One day after the spraying, a suspension of spores of artificially cultured Alternaria mali was inoculated to the leaves by spraying (once) and infected by transferring them into a moisturizing box and keeping at 20° C. Four days after the inoculation, the contraction rate per pot was classified and evaluated according to the following standard and the controlling value (%) was obtained. Phytotoxicity was also studied at the same time. This test is an average of the results of 1 section 3 leaves.

Evaluation of contraction rate and calculation method of controlling value are as follows

| Contraction rate | Lesion area ratio (%) |
|---|---|
| 0 | 0 |
| 0.5 | less than 2 |
| 1 | 2-less than 5 |
| 2 | 5-less than 10 |
| 3 | 10-less than 20 |
| 4 | 20-less than 40 |
| 5 | more than 40 |

Test Results

Compounds of the compound numbers 1-5, 1-14, 1-33, 1-36, 1-41, 1-42, 1-46, 1-56, 1-102, 1-121, 1-304, 1-311, 1-435, 1-520 and 1-523 showed controlling values of more than 80% at the chemical concentration (500 ppm). No phytotoxicity was observed.

### FORMULATION EXAMPLE 1 (GRANULE)

To a mixture of the compound of the present invention No. 1-5 (10 parts), bentonite (montmorillonite) (30 parts), talc (58 parts) and ligninsulfonate salt (2 parts), water (25 parts) is added, well kneaded, made into granules of 10-40 mesh by an extrusion granulator and dried at 40-50° C. to obtain granules.

### FORMULATION EXAMPLE 2 (GRANULES)

Clay mineral particles having particle size distribution in the range of 0.2-2 mm

(95 parts) are put in a rotary mixer. While rotating it, the compound of the present invention No. 1-56 (5 parts) are sprayed together with a liquid diluent, wetted uniformly and dried at 40-50° C. to obtain granules.

### FORMULATION EXAMPLE 3 (EMULSIFIABLE CONCENTRATE)

The compound of the present invention No. 1-57 (30 parts), xylene (55 parts), polyoxyethylene alkyl phenyl ether (8 parts) and calcium alkylbenzenesulfonate (7 parts) are mixed and stirred to obtain an emulsifiable concentrate.

### FORMULATION EXAMPLE 4 (WETTABLE POWDER)

The compound of the present invention No. 1-238 (15 parts), a mixture of white carbon (hydrous amorphous silicon oxide fine powder) and powder clay (1:5) (80 parts), sodium alkylbenzenesulfonate (2 parts) and sodium alkylnaphthalenesulfonate-formalin-condensate (3 parts) are crushed and mixed to make a wettable powder.

### FORMULATION EXAMPLE 5 (WATER DISPERSIBLE GRANULE)

The compound of the present invention No. 1-14 (20 parts), sodium ligninsulfonate (30 parts), bentonite (15 parts) and calcined diatomaceous earth powder (35 parts) are well mixed, added with water, extruded with 0.3 mm screen and dried to obtain water dispersible granules.

What is claimed is:

1-8. (canceled)

9. A method of protecting plants or materials from undesired microorganisms in agriculture and horticulture comprising applying to the microorganisms and/or their habitat a benzylpyrimidine derivative represented by formula (I) 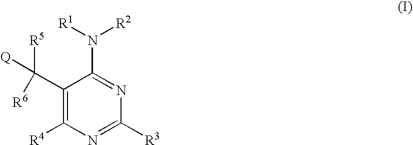
wherein
R¹ and R², together with the nitrogen atom to which they are bonded, form a 3- to 10-membered heterocyclic group that is optionally substituted and optionally contains one to three further hetero atoms selected from the group consisting of nitrogen atom, oxygen atom, and S(O)ₙ in addition to the nitrogen atom to which R¹ and R² are bonded,
n represents 0, 1, or 2,
R³ represents hydrogen, halogen, cyano, hydroxy, amino, azido, alkyl, haloalkyl, alkoxyalkyl, cycloalkyl, alkenyl, alkynyl, alkoxy, haloalkoxy, alkenyloxy, haloalkenyloxy, alkylthio, alkenylthio, haloalkenylthio, alkylsulfinyl, alkylsulfonyl, phenoxy that is optionally substituted, benzyloxy that is optionally substituted, phenyl that is optionally substituted, phenylalkyl that is optionally substituted, phenoxyalkyl that is optionally substituted, or 5- to 10-membered heterocyclic group that contains one to four hetero atoms selected from the group consisting of nitrogen atom, oxygen atom, and sulfur atom and is optionally substituted with a group selected from the group consisting of halogen, alkyl, and haloalkyl; or represents a moiety selected from the group consisting of formulas A to H and J to M 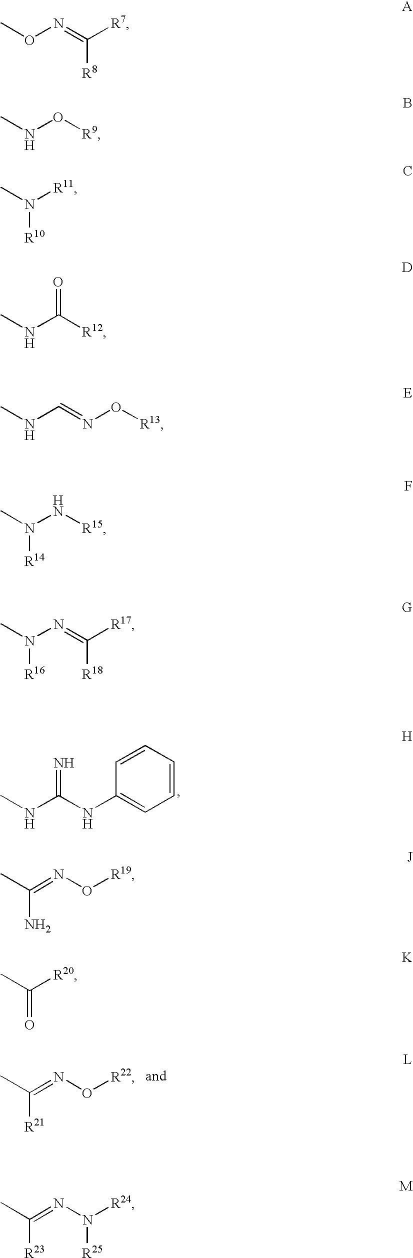
in which
R⁷ represents hydrogen atom, alkyl, or haloalkyl, and
R⁸ represents alkyl, phenyl, alkoxy or cyano, or
R⁷ and R⁸, together with the carbon atom to which they are bonded, form a cycloalkylidene,
R⁹ represents alkyl, haloalkenyl, or benzyl,
R¹⁰ represents hydrogen atom or alkyl,
R¹¹ represents alkyl, alkoxyalkyl, dialkylaminoalkyl, phenyl, benzyl, or cyano,
R¹² represents alkyl or phenyl,
R¹³ represents alkyl or benzyl,
R¹⁴ represents hydrogen atom or alkyl,
R¹⁵ represents hydrogen atom, haloalkyl, or phenyl,
R¹⁶ represents hydrogen atom or alkyl,
R¹⁷ represents hydrogen atom, alkyl, or haloalkyl,
R¹⁸ represents alkyl or phenyl,
R¹⁹ represents hydrogen atom or alkyl,
R²⁰ represents alkyl,
R²¹ represents alkyl,
R²² represents alkyl, alkenyl, haloalkenyl, alkoxyalkyl, phenoxyalkyl, or alkoxycarbonylalkyl,
R²³ represents alkyl, and
R²⁴ represents hydrogen atom or alkyl, and
R²⁵ represents alkyl or phenyl, or
R²⁴ and R²⁵, together with the nitrogen atom to which they are bonded, form a 5- to 8-membered saturated monoheterocyclic group that is optionally substituted and optionally contains one or two further hetero atoms selected from the group consisting of nitrogen atom, oxygen atom, and S(O)ₙ in addition to the nitrogen atom to which R²⁴ and R²⁵ are bonded,
R⁴ represents hydrogen atom, halogen, cyano, alkyl, haloalkyl, alkenyl, alkynyl, alkoxy, haloalkoxy, alkylthio, haloalkylthio, alkylsulfinyl, alkylsulfonyl, or a group 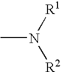
where R¹ and R² are as defined above,
R⁵ and R⁶ each independently represents hydrogen atom, halogen, alkyl, haloalkyl, or phenyl that is optionally substituted, and
Q represents aryl that is optionally substituted or a 5- or 6-membered heterocyclic group that contains one hetero atom selected from the group consisting of nitrogen atom, oxygen atom, and sulfur atom and is optionally substituted.

10. A benzylpyrimidine derivative represented by formula (IA) 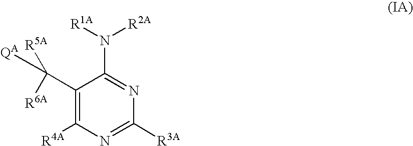
wherein
R^{1A} and R^{2A}, together with the nitrogen atom to which they are bonded, form a 3- to 10-membered heterocyclic group that is optionally substituted and optionally contains one to three further hetero atoms selected from the group consisting of nitrogen atom, oxygen atom, and S(O)ₘ in addition to the nitrogen atom to which R^{1A} and R^{2A} are bonded,
m represents 0, 1, or 2,
R^{3A} represents hydrogen, halogen, cyano, hydroxy, amino, azido, alkyl, haloalkyl, alkoxyalkyl, cycloalkyl, alkenyl, alkynyl, alkoxy, haloalkoxy, alkenyloxy, haloalkenyloxy, alkylthio, alkenylthio, haloalkenylthio, alkylsulfinyl, alkylsulfonyl, phenoxy that is optionally substituted, benzyloxy that is optionally substituted, phenyl that is optionally substituted, phenylalkyl that is optionally substituted, phenoxyalkyl that is optionally substituted, or 5- to 10-membered heterocyclic group that contains one to four hetero atoms selected from the group consisting of nitrogen atom, oxygen atom, and sulfur atom and is optionally substituted with a group selected from the group consisting of halogen, alkyl, and haloalkyl; or represents a moiety selected from the group consisting of A to H and J to M 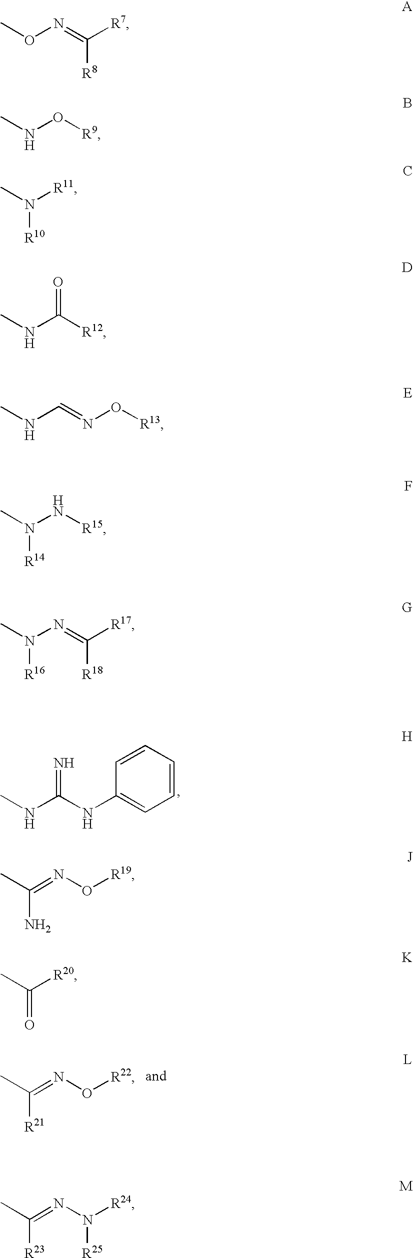
in which
R^{7A} represents hydrogen atom, alkyl, or haloalkyl, and
R^{8A} represents alkyl, phenyl, alkoxy, or cyano, or
R^{7A} and R^{8A}, together with the carbon atom to which they are bonded, form a cycloalkylidene,
R^{9A} represents alkyl, haloalkenyl, or benzyl,
R^{10A} represents hydrogen atom or alkyl,
R^{11A} represents alkyl, alkoxyalkyl, dialkylaminoalkyl, phenyl, benzyl, or cyano,
R^{12A} represents alkyl or phenyl,
R^{13A} represents alkyl or benzyl,
R^{14A} represents hydrogen atom or alkyl,
R^{15A} represents hydrogen atom, haloalkyl, or phenyl,
R^{16A} represents hydrogen atom or alkyl,
R^{17A} represents hydrogen atom, alkyl, or haloalkyl,
R^{18A} represents alkyl or phenyl,
R^{19A} represents hydrogen atom or alkyl,
R^{20A} represents alkyl,
R^{21A} represents alkyl,
R^{22A} represents alkyl, alkenyl, haloalkenyl, alkoxyalkyl, phenoxyalkyl, or alkoxycarbonylalkyl,
R^{23A} represents alkyl, and
R^{24A} represents hydrogen atom or alkyl, and
R^{25A} represents alkyl or phenyl, or
R^{24A} and R^{25A}, together with the nitrogen atom to which they are bonded, form a 5- to 8-membered saturated monoheterocyclic group that is optionally substituted and optionally contains one or two further hetero atoms selected from the group consisting of nitrogen atom, oxygen atom, and S(O)ₙ in addition to the nitrogen atom to which R^{24A} and R^{25A} are bonded,
R^{4A} represents hydrogen atom, halogen, cyano, alkyl, haloalkyl, alkenyl, alkynyl, alkoxy, haloalkoxy, alkylthio, haloalkylthio, alkylsulfinyl, alkylsulfonyl, or a group 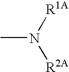
where R^{1A} and R^{2A} are as defined above,
R^{5A} and R^{6A} each independently represents hydrogen atom, halogen, alkyl, haloalkyl, or phenyl that is optionally substituted, and
Q^{A} represents aryl that is optionally substituted, a 5 or 6-membered heterocyclic group that contains one hetero atom selected from the group consisting of nitrogen atom, oxygen atom, and sulfur atom and is optionally substituted,
with the proviso that the following cases (T-1) to (T-6) are excluded:
(T-1) benzylpyrimidine derivatives in which
the group 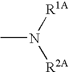
represents 1-pyrrolyl, 1-imidazolyl, 3-oxopiperidino, or 4-oxopiperidino,
R^{3A} represents hydrogen atom,
R^{4A} represents hydrogen atom, and
Q^{A} represents 1-naphthyl or phenyl group that is optionally substituted by one or two groups selected from the group consisting of chloro, methyl, ethyl, and trifluoromethyl,
(T-2) benzylpyrimidine derivatives in which
the group 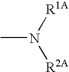
represents 3-oxopiperidino, 4-oxopiperidino, 4-hydroxypiperidino, 4-carbamoylpiperidino, 4-methylpiperazino, 4-ethylpiperazino, 4-(2-hydroxyethyl)piperazino, or morpholino,
R^{3A} represents amino,
R^{4A} represents hydrogen atom, and
Q^{A} represents 3-pyridyl or phenyl group that is optionally substituted by one to three groups selected from the group consisting of fluoro, chloro, bromo, methyl, ethyl, isopropyl, trifluoromethyl, hydroxy, methoxy, and 4-chlorobenzyloxy,
(T-3) benzylpyrimidine derivatives in which
the group 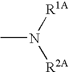
represents piperidino, 4-hydroxypiperidino, 4-methylpiperazino, morpholino, 6,7-dimethoxy-1,2,3,4-tetrahydroisoquinolin-2-yl or 6,7-dimethoxy-1-(3,4-dimethoxybenzyl)-1,2,3,4-tetrahydroisoquinolin-2-yl,
R^{3A} represents chloro, dimethylamino, anilino, 2-(2-hydroxyethoxy)ethylamino, piperidino, 4-hydroxypiperidino, 4-carbamoylpiperidino, 4-methylpiperazino, or morpholino,
R^{4A} represents hydrogen atom, and
Q^{A} represents phenyl group that is optionally substituted by one or two groups selected from the group consisting of methyl and methoxy,
(T-4) benzylpyrimidine derivatives in which
the group 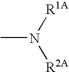
represents 1-pyrrolidinyl, piperidino, morpholino, or 1-pyrrolyl,
R^{3A} represents methyl or methoxymethyl,
R^{4A} represents chloro, and
Q^{A} represents phenyl or 1-naphthyl,
(T-5) benzylpyrimidine derivatives in which
the group 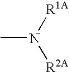
represents 1-azilidinyl, piperidino, or morpholino,
R^{3A} represents methylthio,
R^{4A} represents chloro, and
Q^{A} represents phenyl group substituted by methoxy, ethoxy, n-propoxy, iso-propoxy, n-butoxy, iso-butoxy, or allyloxy, and
(T-6) benzylpyrimidine derivatives in which
the group 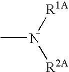
represents 1-azilidinyl,
R^{3A} represents hydrogen atom or amino,
R^{4A} represents chloro, and
Q^{A} represents phenyl group substituted by methoxy, ethoxy, or allyloxy.

11. A compound according to claim 10 wherein
R^{1A} and R^{2A}, together with the nitrogen atom to which they are bonded, form a heterocyclic group that is a monovalent group derived from a heterocycle selected from the group consisting of aziridine, azetidine, pyrrolidine, 3-pyrroline, piperidine, perhydroazepine, perhydroazocine, perhydro-1,2-diazepine, perhydro-1,2,5-oxadiazepine, 2-pyrazoline, thiazolidine, perhydroindole, 1,2,3,3a,4,7,7a-heptahydroisoindole, 1,2,3,6-tetrahydropyridine, perhydroquinoline, perhydroisoquinoline, 1,4,5,6-tetrahydropyridazine, morpholine, thiomorpholine, thiomorpholine-1,1-dioxide, piperazine, pyrrole, pyrazole, imidazole, 1,2,3-triazole, 1,2,4-triazole, tetrazole and 1H-indazole and may be optionally substituted by one to three groups selected from the group consisting of fluoro, bromo, C₁₋₄alkyl, C₁₋₄haloalkyl, C₁₋₄alkoxy, C₁₋₄alkylthio, benzylthio, hydroxyl-C₁₋₄alkyl, C₁₋₄alkoxy-C₁₋₄alkyl, aniline-C₁₋₄alkyl, C₁₋₄haloalkylene, C₁₋₄alkoxy-carbonyl, benzyloxycarbonyl, C₁₋₄alkyl-carbonyl, C₁₋₇haloalkyl-carbonyl, phenyl, benzyl, pyridyl, hydroxy, oxo, cyano, carboxy, carbamoyl, C₁₋₄alkoxy-carbonyl-C₁₋₄alkyl, C₁₋₄alkyl-carbonylamino, and C₁₋₄haloalkyl-carbonylamino,
R^{3A} represents hydrogen, chloro, cyano, hydroxy, amino, azido, C₁₋₆alkyl, C₁₋₆haloalkyl, C₁₋₆alkoxyC₁₋₆alkyl, C₃₋₇cycloalkyl, C₂₋₇alkenyl, C₂₋₇alkynyl, C₁₋₆alkoxy, C₁₋₆haloalkoxy, C₂₋₇alkenyloxy, C₂₋₇haloalkenyloxy, C₁₋₆alkylthio, C₂₋₇alkenylthio, C₂₋₇haloalkenylthio, C₁₋₆alkylsulfinyl, C₁₋₆alkylsulfonyl, phenoxy, benzyloxy, phenyl that is optionally substituted by one or two groups selected from the group consisting of chloro, C₁₋₆alkyl, C₁₋₆alkoxy, and C₁₋₆haloalkyl, phenyl-C₁₋₄alkyl that is optionally chloro-substituted, or phenoxyC₁₋₄alkyl that is optionally chloro-substituted; represents a heterocyclic group that is a monovalent group derived from a heterocycle selected from the group consisting of pyrrolidine, piperidine, morpholine, thiomorpholine, piperazine, thiophene, thiazole, pyridine, quinoline, isoquinoline, pyrazine, pyridazine, pyrimidine, imidazole, pyrazole, tetrazole, 1,2,4-triazole, and 2,3-dihydroindole and is optionally substituted by a group selected from the group consisting of chloro, bromo, C₁₋₆alkyl, and C₁₋₆haloalkyl; or represents a moiety selected from the group consisting of formulas A to H and J to M 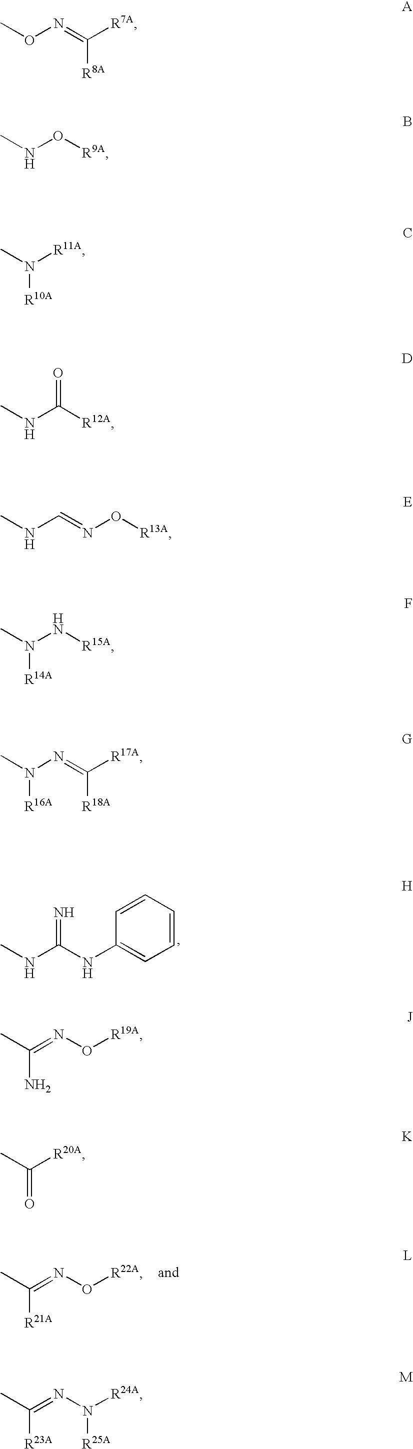
in which
R^{7A} represents hydrogen atom, C₁₋₆alkyl, or C₁₋₆haloalkyl,
R^{8A} represents C₁₋₆alkyl, phenyl, C₁₋₆alkoxy, or cyano,
R^{7A} and R^{8A}, together with the carbon atom to which they are bonded, form a C₅₋₈cycloalkylidene,
R^{9A} represents C₁₋₆alkyl, C₂₋₇haloalkenyl, or benzyl,
R^{10A} represents hydrogen atom or C₁₋₆alkyl,
R^{11A} represents C₁₋₆alkyl, C₁₋₆alkoxy-C₁₋₆alkyl, di(C₁₋₆alkyl)amino-C₁₋₆alkyl, phenyl, benzyl, or cyano,
R^{12A} represents C₁₋₆alkyl or phenyl,
R^{13A} represents C₁₋₆alkyl or benzyl,
R^{14A} represents hydrogen atom or C₁₋₆alkyl,
R^{15A} represents hydrogen atom, C₁₋₆haloalkyl, or phenyl,
R^{16A} represents hydrogen atom or C₁₋₆alkyl,
R^{17A} represents hydrogen atom, C₁₋₆alkyl, or C₁₋₆haloalkyl,
R^{18A} represents C₁₋₆alkyl or phenyl,
R^{19A} represents hydrogen atom or C₁₋₆alkyl,
R^{20A} represents C₁₋₆alkyl,
R^{21A} represents C₁₋₆alkyl,
R^{22A} represents C₁₋₆alkyl, C₂₋₇alkenyl, C₂₋₇haloalkenyl, C₁₋₆alkoxy-C₁₋₆alkyl, phenoxy-C₁₋₆alkyl, or C₁₋₆alkoxycarbonyl-C₁₋₆alkyl,
R^{23A} represents C₁₋₆alkyl, and
R^{24A} represents hydrogen atom or C₁₋₆alkyl, and
R^{25A} represents C₁₋₆alkyl or phenyl, or
R^{24A} and R^{25A}, together with the nitrogen atom to which they are bonded, form a saturated-monoheterocyclic group that is a monovalent group derived from a monoheterocycle selected from the group consisting of pyrrolidine, piperidine, morpholine, and piperazine and is optionally substituted with C₁₋₄alkyl,
R^{4A} represents hydrogen atom, fluoro, chloro, cyano, C₁₋₆alkyl, C₁₋₆haloalkyl, C₂₋₇alkenyl, C₂₋₇alkynyl, C₁₋₆alkoxy, C₁₋₆haloalkoxy, C₁₋₆alkylthio, C₁₋₆haloalkylthio, C₁₋₆alkylsulfinyl, C₁₋₆alkylsulfonyl, or pyrazolyl that is optionally C₁₋₆alkyl-substituted or C₁₋₆haloalkyl-substituted,
R^{5A} and R^{6A} each independently represents hydrogen atom, fluoro, C₁₋₄alkyl, C₁₋₄haloalkyl, or phenyl, and
Q^{A} represents naphthyl, phenyl that is optionally substituted, pyridyl that is optionally substituted, thienyl that is optionally substituted, or furyl that is optionally substituted, wherein substituents to phenyl, pyridyl, thienyl, and furyl are one to five groups selected from the group consisting of fluoro, chloro, C₁₋₄alkyl, C₁₋₄haloalkyl, C₁₋₄alkoxy, C₁₋₄haloalkoxy, cyano, nitro, amino, and phenyl,
with the proviso that the following cases (T-1) to (T-6) are excluded:
(T-1) benzylpyrimidine derivatives in which
the group 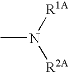
represents 1-pyrrolyl, 1-imidazolyl, 3-oxopiperidino, or 4-oxopiperidino,
R^{3A} represents hydrogen atom,
R^{4A} represents hydrogen atom, and
Q^{A} represents 1-naphthyl or phenyl group that is optionally substituted by one or two groups selected from the group consisting of chloro, methyl, ethyl, and trifluoromethyl,
(T-2) benzylpyrimidine derivatives in which
the group 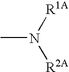
represents 3-oxopiperidino, 4-oxopiperidino, 4-hydroxypiperidino, 4-carbamoylpiperidino, 4-methylpiperazino, 4-ethylpiperazino, 4-(2-hydroxyethyl)piperazino, or morpholino,
R^{3A} represents amino,
R^{4A} represents hydrogen atom, and
Q^{A} represents 3-pyridyl or phenyl group that is optionally substituted by one to three groups selected from the group consisting of fluoro, chloro, methyl, ethyl, isopropyl, trifluoromethyl, and methoxy,
(T-3) benzylpyrimidine derivatives in which
the group 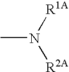
represents piperidino, 4-hydroxypiperidino, 4-methylpiperazino, or morpholino,
R^{3A} represents chloro, dimethylamino, anilino, piperidino, 4-methylpiperazino, or morpholino,
R^{4A} represents hydrogen atom, and
Q^{A} represents phenyl group that is optionally substituted by one or two groups selected from the group consisting of methyl and methoxy,
(T-4) benzylpyrimidine derivatives in which
the group 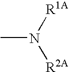
represents 1-pyrrolidinyl, piperidino, morpholino, or 1-pyrrolyl,
R^{3A} represents methyl or methoxymethyl,
R^{4A} represents chloro, and
Q^{A} represents phenyl or 1-naphthyl,
(T-5) benzylpyrimidine derivatives in which
the group 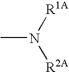
represents 1-azilidinyl, piperidino, or morpholino,
R^{3A} represents methylthio,
R^{4A} represents chloro, and
Q^{A} represents phenyl group substituted by methoxy, ethoxy, n-propoxy, iso-propoxy, n-butoxy, or iso-butoxy, and
(T-6) benzylpyrimidine derivatives in which
the group 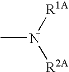
represents 1-azilidinyl,
R^{3A} represents hydrogen atom or amino,
R^{4A} represents chloro, and
Q^{A} represents phenyl group substituted by methoxy or ethoxy.

12. A compound according to claim 10 wherein
R^{1A} and R^{2A}, together with the nitrogen atom to which they are bonded, form a heterocyclic group that is a monovalent group derived from a heterocycle selected from the group consisting of aziridine, azetidine, pyrrolidine, 3-pyrroline, piperidine, perhydroazepine, perhydroazocine, perhydro-1,2-diazepine, perhydro-1,2,5-oxadiazepine, 2-pyrazoline, thiazolidine, perhydroindole, 1,2,3,3a,4,7,7a-heptahydroisoindole, 1,2,3,6-tetrahydropyridine, perhydroquinoline, perhydroisoquinoline, 1,4,5,6-tetrahydropyridazine, morpholine, thiomorpholine, thiomorpholine-1,1-dioxide, piperazine, pyrrole, pyrazole, imidazole, 1,2,3-triazole, 1,2,4-triazole, tetrazole, and 1H-indazole and is optionally substituted with 1 to 3 groups selected from the group consisting of fluoro, bromo, methyl, ethyl, n-propyl, fluoromethyl, trifluoromethyl, 2,2,2-trifluoroethyl, methoxy, methylthio, benzylthio, hydroxymethyl, 2-hydroxyethyl, methoxymethyl, anilinomethyl, difluoromethylene, dichloromethylene, methoxycarbonyl, ethoxycarbonyl, benzyloxycarbonyl, acetyl, trifluoromethylcarbonyl, trichloromethylcarbonyl, 1,1,2,2-tetrafluoroethylcarbonyl, perfluoroethylcarbonyl, perfluoroheptylcarbonyl, phenyl, benzyl, 2-pyridyl, hydroxy, oxo, cyano, carboxy, carbamoyl, ethoxycarbonylmethyl, methylcarbonylamino, and trifluoromethylcarbonylamino,
R^{3A} represents hydrogen, chloro, cyano, hydroxy, amino, azido, methyl, ethyl, iso-propyl, tert-butyl, trifluoromethyl, methoxymethyl, cyclopropyl, allyl, ethynyl, 1-propynyl, methoxy, ethoxy, n-propyloxy, n-butyloxy, 2,2,2-trifluoroethyloxy, allyloxy, 2-methyl-4-pentenyloxy, 3-chloro-4,4,4-trifluoro-2-butenyloxy, methylthio, ethylthio, n- or iso-propylthio, n-, sec- or tert-butylthio, allylthio, 3,3-dichloroallylthio, methylsulfinyl, methylsulfonyl, phenoxy, benzyloxy, phenyl that is optionally substituted with 1 to 2 groups selected from the group consisting of chloro, methyl, methoxy, and trifluoromethyl, benzyl that is optionally chloro-substituted, or phenoxymethyl that is optionally chloro-substituted; represents a heterocyclic group that is a monovalent group derived from a heterocycle selected from the group consisting of pyrrolidine, piperidine, morpholine, thiomorpholine, piperazine, thiophene, thiazole, pyridine, quinoline, isoquinoline, pyrazine, pyridazine, pyrimidine, imidazole, pyrazole, tetrazole, 1,2,4-triazole, and 2,3-dihydroindole and is optionally substituted by a group selected from the group consisting of chloro, bromo, methyl, and trifluoromethyl; or represents a moiety selected from the group consisting of formulas A to H and J to M 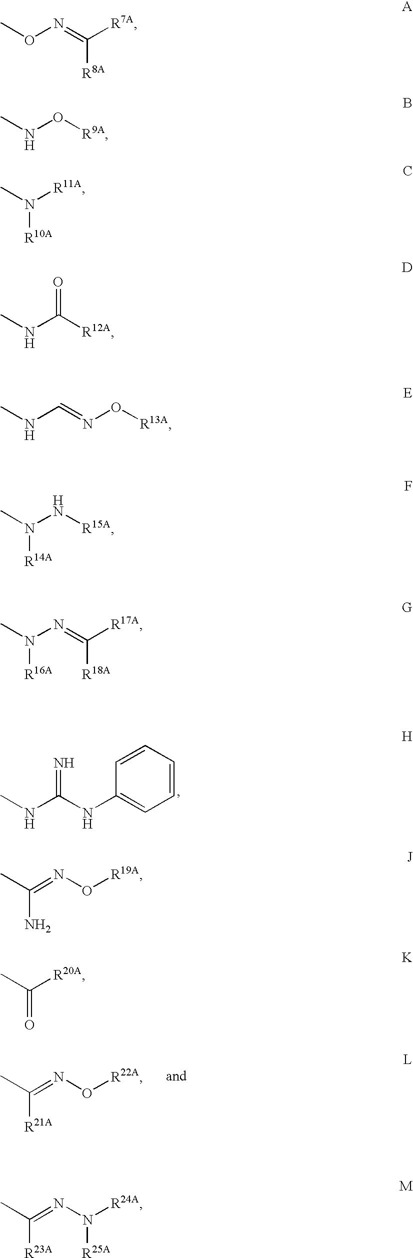
in which
R^{7A} represents hydrogen atom, methyl, or trifluoromethyl, and
R^{8A} represents methyl, iso- or tert-butyl, neo-pentyl, phenyl, ethoxy, or cyano, or
R^{7A} and R^{8A}, together with the carbon atom to which they are bonded, form a cyclopentylidene or cyclohexylidene,
R^{9A} represents methyl, 3,3-dichloroallyl, or benzyl,
R^{10A} represents hydrogen atom, methyl, or ethyl,
R^{11A} represents methyl, ethyl, iso-propyl, methoxyethyl, dimethylaminoethyl, phenyl, benzyl, or cyano,
R^{12A} represents methyl or phenyl,
R^{13A} represents methyl or benzyl,
R^{14A} represents hydrogen atom or methyl,
R^{15A} represents hydrogen atom, 2,2,2-trifluoroethyl, or phenyl,
R^{16A} represents hydrogen atom or methyl,
R^{17A} represents hydrogen atom, methyl, or trifluoromethyl,
R^{18A} represents methyl or phenyl,
R^{19A} represents hydrogen atom or methyl,
R^{20A} represents methyl, ethyl, or n- or iso-propyl,
R^{21A} represents methyl or ethyl,
R^{22A} represents methyl, ethyl, n-propyl, n- or tert-butyl, allyl, 2-chloro-2-propenyl, 3-chloro-2-propenyl, 3,3-dichloro-2-propenyl, 2-methoxyethyl, 2-phenoxypropyl, or tert-butoxycarbonylmethyl,
R^{23A} represents methyl, and
R^{24A} represents hydrogen atom or methyl, and
R^{25A} represents iso-propyl or phenyl, or
R^{24A} and R^{25A}, together with the nitrogen atom to which they are bonded, form a saturated monoheterocyclic group that is a monovalent group derived from a monoheterocycle selected from the group consisting of pyrrolidine, piperidine, morpholine, and piperazine and is optionally substituted by methyl,
R^{4A} represents hydrogen atom, chloro, cyano, methyl, trifluoromethyl, allyl, ethynyl, 1-propynyl, methoxy, 2,2,2-trifluoroethoxy, methylthio, C₁₋₆haloalkylthio, methylsulfinyl, methylsulfonyl, or pyrazolyl that is optionally methyl-substituted or trifluoromethyl-substituted,
R^{5A} and R^{6A} each independently represents hydrogen atom, fluoro, methyl, ethyl, iso-propyl, trifluoromethyl, or phenyl, and
Q^{A} represents naphthyl, phenyl that is optionally substituted, pyridyl that is optionally substituted, thienyl that is optionally substituted, or furyl that is optionally substituted, wherein substituents to phenyl, pyridyl, thienyl, and furyl are 1 to 5 groups selected from the group consisting of fluoro, chloro, methyl, tert-butyl, trifluoromethyl, methoxy, trifluoromethoxy, cyano, nitro, amino, and phenyl,
with the proviso that the following cases (T-1) to (T-6) are excluded:
(T-1) benzylpyrimidine derivatives in which
the group 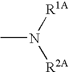
represents 1-pyrrolyl, 1-imidazolyl, 3-oxopiperidino, or 4-oxopiperidino,
R^{3A} represents hydrogen atom,
R^{4A} represents hydrogen atom, and
Q^{A} represents 1-naphthyl or phenyl group that is optionally substituted with 1 to 2 groups selected from the group consisting of chloro, methyl, and trifluoromethyl,
(T-2) benzylpyrimidine derivatives in which
the group 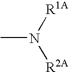
represents 3-oxopiperidino, 4-oxopiperidino, 4-hydroxypiperidino, 4-carbamoylpiperidino, 4-methylpiperazino, 4-ethylpiperazino, 4-(2-hydroxyethyl)piperazino, or morpholino,
R^{3A} represents amino,
R^{4A} represents hydrogen atom, and
Q^{A} represents 3-pyridyl or phenyl group that is optionally substituted by one to three groups selected from the group consisting of fluoro, chloro, methyl, trifluoromethyl, and methoxy,
(T-3) benzylpyrimidine derivatives in which
the group 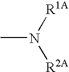
represents piperidino, 4-hydroxypiperidino, 4-methylpiperazino, or morpholino,
R^{3A} represents chloro, dimethylamino, anilino, piperidino, 4-methylpiperazino, or morpholino,
R^{4A} represents hydrogen atom, and
Q^{A} represents phenyl group that is optionally substituted by one or two groups selected from the group consisting of methyl and methoxy,
(T-4) benzylpyrimidine derivatives in which
the group 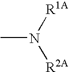
represents 1-pyrrolidinyl, piperidino, morpholino, or 1-pyrrolyl,
R^{3A} represents methyl or methoxymethyl,
R^{4A} represents chloro, and
Q^{A} represents phenyl or 1-naphthyl,
(T-5) benzylpyrimidine derivatives in which
the group 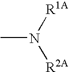
represents 1-azilidinyl, piperidino, or morpholino,
R^{3A} represents methylthio,
R^{4A} represents chloro, and
Q^{A} represents phenyl group substituted by methoxy, and
(T-6) benzylpyrimidine derivatives in which
the group 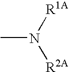
represents 1-azilidinyl,
R^{3A} represents hydrogen atom or amino,
R^{4A} represents chloro, and
Q^{A} represents phenyl group substituted by methoxy.

13. A process for the preparation of compounds of formula (IA) 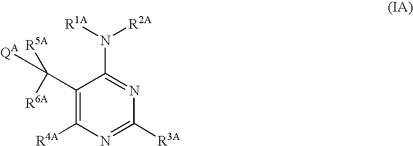
wherein
R^{1A} and R^{2A}, together with the nitrogen atom to which they are bonded, form a 3- to 10-membered heterocyclic group that is optionally substituted and optionally contains further one to three hetero atoms selected from the group consisting of nitrogen atom, oxygen atom, and S(O)ₘ in addition to the nitrogen atom to which R^{1A} and R^{2A} are bonded,
m represents 0, 1, or 2,
R^{3A} represents hydrogen, halogen, cyano, hydroxy, amino, azido, alkyl, haloalkyl, alkoxyalkyl, cycloalkyl, alkenyl, alkynyl, alkoxy, haloalkoxy, alkenyloxy, haloalkenyloxy, alkylthio, alkenylthio, haloalkenylthio, alkylsulfinyl, alkylsulfonyl, phenoxy that is optionally substituted, benzyloxy that is optionally substituted, phenyl that is optionally substituted, phenylalkyl that is optionally substituted, phenoxyalkyl that is optionally substituted, or 5- to 10-membered heterocyclic group that contains one to four hetero atoms selected from the group consisting of nitrogen atom, oxygen atom, and sulfur atom and is optionally substituted with a group selected from the group consisting of halogen, alkyl, and haloalkyl; or represents a moiety selected from the group consisting of formulas A to H and J to M 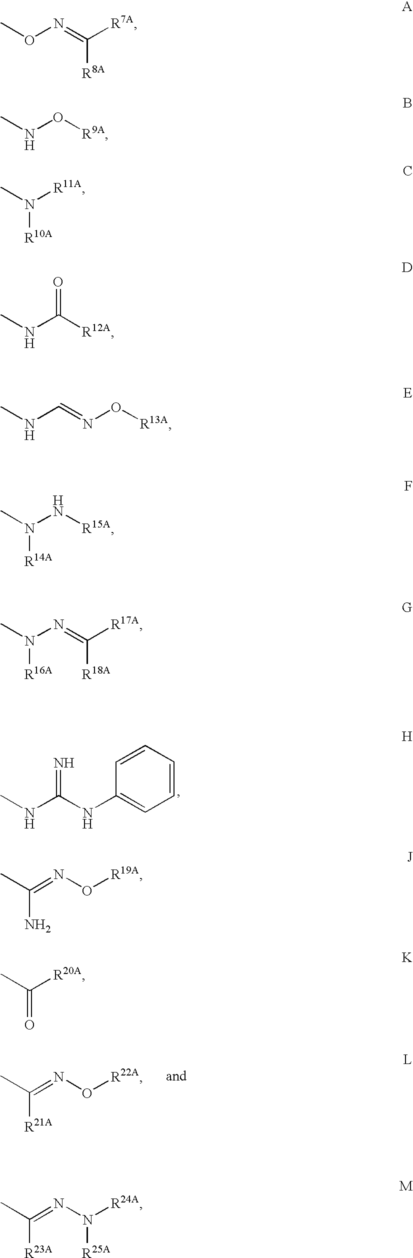
in which
R^{7A} represents hydrogen atom, alkyl, or haloalkyl, and
R^{8A} represents alkyl, phenyl, alkoxy or cyano, or
R^{7A} and R^{8A}, together with the carbon atom to which they are bonded, form a cycloalkylidene or cyclohexylidene,
R^{9A} represents alkyl, haloalkenyl, or benzyl,
R^{10A} represents hydrogen atom or alkyl,
R^{11A} represents alkyl, alkoxyalkyl, dialkylaminoalkyl, phenyl, benzyl, or cyano,
R^{12A} represents alkyl or phenyl,
R^{13A} represents alkyl or benzyl,
R^{14A} represents hydrogen atom or alkyl,
R^{15A} represents hydrogen atom, haloalkyl, or phenyl,
R^{16A} represents hydrogen atom or alkyl,
R^{17A} represents hydrogen atom, alkyl, or haloalkyl,
R^{18A} represents alkyl or phenyl,
R^{19A} represents hydrogen atom or alkyl,
R^{20A} represents alkyl,
R^{21A} represents alkyl,
R^{22A} represents alkyl, alkenyl, haloalkenyl, alkoxyalkyl, phenoxyalkyl, or alkoxycarbonylalkyl,
R^{23A} represents alkyl, and
R^{24A} represents hydrogen atom or alkyl, and
R^{25A} represents alkyl or phenyl, or
R^{24A} and R^{25A}, together with the nitrogen atom to which they are bonded, form a 5- to 8-membered saturated-monoheterocyclic group that is optionally substituted and optionally contains further one or two hetero atoms selected from the group consisting of nitrogen atom, oxygen atom, and S(O)ₙ in addition to the nitrogen atom to which R^{24A} and R^{25A} are bonded,
R^{4A} represents hydrogen atom, halogen, cyano, alkyl, haloalkyl, alkenyl, alkynyl, alkoxy, haloalkoxy, alkylthio, haloalkylthio, alkylsulfinyl, alkylsulfonyl, or a group 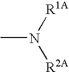
where R^{1A} and R^{2A} are as defined above,
R^{5A} and R^{6A} each independently represents hydrogen atom, halogen, alkyl, haloalkyl, or phenyl that is optionally substituted, and
Q^{A} represents aryl that is optionally substituted or a 5- or 6-membered heterocyclic group that contains one hetero atom selected from the group consisting of nitrogen atom, oxygen atom, and sulfur atom and is optionally substituted,
with the proviso that the following cases (T-1) to (T-6) are excluded:
(T-1) benzylpyrimidine derivatives in which
the group 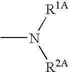
represents 1-indolyl, 1-pyrrolyl, 1-imidazolyl, 3-oxopiperidino, or 4-oxopiperidino,
R^{3A} represents hydrogen atom,
R^{4A} represents hydrogen atom, and
Q^{A} represents 1-naphthyl or phenyl group that is optionally substituted by one or two groups selected from the group consisting of chloro, bromo, methyl, ethyl, and trifluoromethyl,
(T-2) benzylpyrimidine derivatives in which
the group 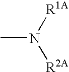
represents 3-oxopiperidino, 4-oxopiperidino, 4-hydroxypiperidino, 4-carbamoylpiperidino, 4-methylpiperazino, 4-ethylpiperazino, 4-(2-hydroxyethyl)piperazino, or morpholino,
R^{3A} represents amino,
R^{4A} represents hydrogen atom, and
Q^{A} represents 3-pyridyl or phenyl group that is optionally substituted by one to three groups selected from the group consisting of fluoro, chloro, bromo, methyl, ethyl, isopropyl, trifluoromethyl, hydroxy, methoxy, and 4-chlorobenzyloxy,
(T-3) benzylpyrimidine derivatives in which
the group 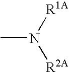
represents piperidino, 4-hydroxypiperidino, 4-methylpiperazino, morpholino, 6,7-dimethoxy-1,2,3,4-tetrahydroisoquinolin-2-yl, or 6,7-dimethoxy-1-(3,4-dimethoxybenzyl)-1,2,3,4-tetrahydroisoquinolin-2-yl,
R^{3A} represents chloro, dimethylamino, anilino, 2-(2-hydroxyethoxy)ethylamino, piperidino, 4-hydroxypiperidino, 4-carbamoylpiperidino, 4-methylpiperazino, or morpholino,
R^{4A} represents hydrogen atom, and
Q^{A} represents phenyl group that is optionally substituted by one or two groups selected from the group consisting of methyl and methoxy,
(T-4) benzylpyrimidine derivatives in which
the group 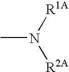
represents 1-pyrrolidinyl, piperidino, morpholino, or 1-pyrrolyl,
R^{3A} represents methyl or methoxymethyl,
R^{4A} represents chloro, and
Q^{A} represents phenyl or 1-naphthyl,
(T-5) benzylpyrimidine derivatives in which
the group 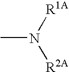
represents 1-azilidinyl, piperidino, or morpholino,
R^{3A} represents methylthio,
R^{4A} represents chloro, and
Q^{A} represents phenyl group substituted by methoxy, ethoxy, n-propoxy, iso-propoxy, n-butoxy, iso-butoxy, or allyloxy, and
(T-6) benzylpyrimidine derivatives in which
the group 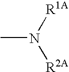
represents 1-azilidinyl,
R^{3A} represents hydrogen atom or amino,
R^{4A} represents chloro, and
Q^{A} represents phenyl group substituted by methoxy, ethoxy, or allyloxy, comprising
(a) for compounds of formula (IA) in which
R^{3A} represents hydrogen, alkyl, haloalkyl, alkoxyalkyl, cycloalkyl, alkenyl, alkylthio, alkenylthio, haloalkenylthio, phenyl that is optionally substituted, phenylalkyl that is optionally substituted, phenoxyalkyl that is optionally substituted, or 5- to 10-membered heterocyclic group that contains one to four hetero atoms selected from the group consisting of nitrogen atom, oxygen atom, and sulfur atom and is optionally substituted by a group selected from the group consisting of halogen, alkyl, and haloalkyl, and
R^{4A} represents hydrogen atom, halogen, alkyl, haloalkyl, or alkenyl,
reacting a compound of formula (II) 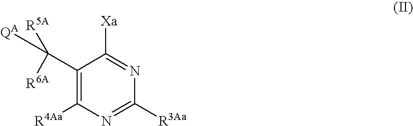
wherein
Xa represents halogen,
R^{3Aa} represents hydrogen, alkyl, haloalkyl, alkoxyalkyl, cycloalkyl, alkenyl, alkylthio, alkenylthio, haloalkenylthio, phenyl that is optionally substituted, phenylalkyl that is optionally substituted, phenoxyalkyl that is optionally substituted, or 5- to 10-membered heterocyclic group that contains one to four hetero atoms selected from the group consisting of nitrogen atom, oxygen atom, and sulfur atom and is optionally substituted by a group selected from the group consisting of halogen, alkyl, and haloalkyl,
R^{4Aa} represents hydrogen atom, halogen, alkyl, haloalkyl or alkenyl, and
R^{5A}, R^{6A}, and Q^{A} have the same definitions as for formula (IA),
with a compound of formula (III) 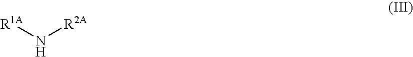
wherein R^{1A} and R^{2A} have the same definitions as for formula (IA), in the presence of an inert solvent and optionally in the presence of an acid binder,
or
(b) for compounds of formula (IA) in which
(i)
R^{3A} represents alkylsulfinyl or alkylsulfonyl, and
R^{4A} represents hydrogen atom, halogen, cyano, alkyl, haloalkyl, alkenyl, alkynyl, alkoxy, haloalkoxy or a group 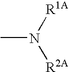
where R^{1A} and R^{2A} are as defined above,
reacting a compound of formula (IAb) 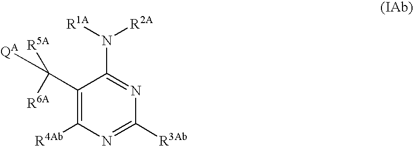
wherein
R^{3Ab} represents alkylthio, and
R^{4Ab} represents hydrogen atom, halogen, cyano, alkyl, haloalkyl, alkenyl, alkynyl, alkoxy, haloalkoxy, or a group 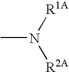
where R^{1A} and R^{2A} are as defined above, and
R^{1A}, R^{2A}, R^{5A}, R^{6A}, and Q^{A} have the same definitions as for formula (IA),
with an oxidizing agent in the presence of an inert solvent,
or
(ii)
R^{3A} represents hydrogen, alkyl, haloalkyl, alkoxyalkyl, cycloalkyl, alkenyl, phenyl that is optionally substituted, phenylalkyl that is optionally substituted, phenoxyalkyl that is optionally substituted, or 5- to 10-membered heterocyclic group that contains one to four hetero atoms selected from the group consisting of nitrogen atom, oxygen atom, and sulfur atom and is optionally substituted by a group selected from the group consisting of halogen, alkyl, and haloalkyl, and
R^{4A} represents alkylsulfinyl or alkylsulfonyl,
reacting a compound of formula (IAb) 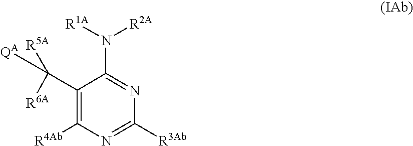
wherein
R^{3Ab} represents hydrogen, alkyl, haloalkyl, alkoxyalkyl, cycloalkyl, alkenyl, phenyl that is optionally substituted, phenylalkyl that is optionally substituted, phenoxyalkyl that is optionally substituted, or 5- to 10-membered heterocyclic group that contains one to four hetero atoms selected from the group consisting of nitrogen atom, oxygen atom, and sulfur atom and is optionally substituted by a group selected from the group consisting of halogen, alkyl, and haloalkyl, and
R^{4Ab} represents alkylthio, and
R^{1A}, R^{2A}, R^{5A}, R^{6A}, and Q^{A} have the same definitions as for formula (IA),
with an oxidizing agent in the presence of an inert solvent,
or
(c) for compounds of formula (IA) in which
R^{3A} represents cyano, hydroxy, azido, alkynyl, alkoxy, haloalkoxy, alkenyloxy, haloalkenyloxy, alkylthio, alkenylthio, haloalkenylthio, phenoxy that is optionally substituted, benzyloxy that is optionally substituted, or 5- to 10-membered heterocyclic group that contains one to four hetero atoms selected from the group consisting of nitrogen atom, oxygen atom, and sulfur atom and is optionally substituted by a group selected from the group consisting of halogen, alkyl, and haloalkyl; or represents a group A, group B, group C, group F, group G, or group H as defined for formula (IA), and
R^{4A} represents hydrogen atom, halogen, alkyl, haloalkyl, alkenyl, alkynyl, alkoxy, haloalkoxy, cyano, or a group 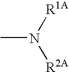
where R^{1A} and R^{2A} are as defined above,
reacting a compound of formula (IAc) 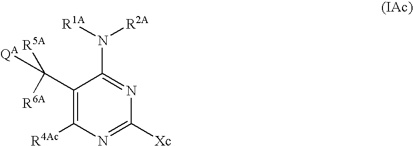
wherein
Xc represents halogen or methylsulfonyl,
R^{4Ac} represents hydrogen atom, halogen, alkyl, haloalkyl, alkenyl, alkynyl, alkoxy, haloalkoxy, cyano or a group 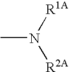
where R^{1A} and R^{2A} are as defined above, and
R^{1A}, R^{2A}, R^{5A}, R^{6A}, and Q^{A} have the same definitions as for formula (IA),
with a compound of formula (IV)
Y—R^{3Ac} (IV)
wherein
Y represents hydrogen, sodium, potassium, copper, trimethylsilyl, or tetraalkylammonium,
R^{3Ac} represents cyano, hydroxy, azido, alkynyl, alkoxy, haloalkoxy, alkenyloxy, haloalkenyloxy, alkylthio, alkenylthio, haloalkenylthio, phenoxy that is optionally substituted, benzyloxy that is optionally substituted, or 5- to 10-membered heterocyclic group that contains one to four hetero atoms selected from the group consisting of nitrogen atom, oxygen atom, and sulfur atom and is optionally substituted by a group selected from the group consisting of halogen, alkyl, and haloalkyl; or represents a group A, group B, group C, group F, group G, or group H as defined for R^{3A} of formula (IA),
in the presence of an inert solvent, optionally in the presence of an acid binder, and optionally in the presence of a catalyst,
or
(d) for compounds of formula (IA) in which
R^{3A} represents hydrogen, alkyl, haloalkyl, alkoxyalkyl, cycloalkyl, alkenyl, alkylthio, alkenylthio, haloalkenylthio, phenyl that is optionally substituted, phenylalkyl that is optionally substituted, phenoxyalkyl that is optionally substituted, or 5- to 10-membered heterocyclic group that contains one to four hetero atoms selected from the group consisting of nitrogen atom, oxygen atom, and sulfur atom and is optionally substituted by a group selected from the group consisting of halogen, alkyl, and haloalkyl, and
R^{4A} represents cyano, alkynyl, alkoxy, haloalkoxy, alkylthio, haloalkylthio or a group 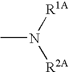
where R^{1A} and R^{2A} are as defined above,
reacting a compound of formula (IAd) 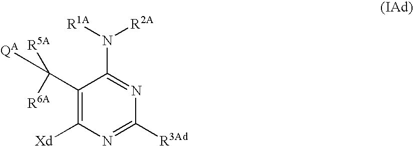
wherein
Xd represents halogen or methylsulfonyl,
R^{3Ad} represents hydrogen, alkyl, haloalkyl, alkoxyalkyl, cycloalkyl, alkenyl, alkylthio, alkenylthio, haloalkenylthio, phenyl that is optionally substituted, phenylalkyl that is optionally substituted, phenoxyalkyl that is optionally substituted, or 5- to 10-membered heterocyclic group that contains one to four hetero atoms selected from the group consisting of nitrogen atom, oxygen atom, and sulfur atom and is optionally substituted by a group selected from the group consisting of halogen, alkyl, and haloalkyl, and
R^{1A}, R^{2A}, R^{5A}, R^{6A}, and Q^{A} have the same definitions as for formula (IA),
with a compound of formula (V)
Y—R^{4Ad} (V)
wherein
Y represents hydrogen, sodium, potassium, copper, trimethylsilyl, or tetraalkylammonium, and
R^{4Ad} represents cyano, alkynyl, alkoxy, haloalkoxy, alkylthio, haloalkylthio, or a group 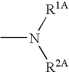
where R^{1A} and R^{2A} are as defined above,
in the presence of an inert solvent, optionally in the presence of an acid binder, and optionally in the presence of a catalyst,
or
(e) for compounds of formula (IA) in which
R^{3A} represents hydrogen, alkyl, haloalkyl, alkoxyalkyl, cycloalkyl, alkylthio, phenyl that is optionally substituted, phenylalkyl that is optionally substituted, phenoxyalkyl that is optionally substituted, or 5- to 10-membered heterocyclic group that contains one to four hetero atoms selected from the group consisting of nitrogen atom, oxygen atom, and sulfur atom and is optionally substituted by a group selected from the group consisting of halogen, alkyl, and haloalkyl, and
R^{4A} represents hydrogen,
hydrogenating a compound of formula (IAe) 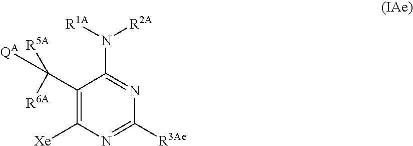
wherein
Xe represents halogen,
R^{3Ae} represents hydrogen, alkyl, haloalkyl, alkoxyalkyl, cycloalkyl, alkylthio, phenyl that is optionally substituted, phenylalkyl that is optionally substituted, phenoxyalkyl that is optionally substituted, or 5- to 10-membered heterocyclic group that contains one to four hetero atoms selected from the group consisting of nitrogen atom, oxygen atom, and sulfur atom and is optionally substituted by a group selected from the group consisting of halogen, alkyl, and haloalkyl, and
R^{1A}, R^{2A}, R^{5A}, R^{6A}, and Q^{A} have the same definitions as for formula (IA),
in the presence of an inert solvent, optionally in the presence of a catalyst, and optionally in the presence of an acid binder,
or
(f) for compounds of formula (IA) in which
R^{3A} represents hydrogen, halogen, cyano, hydroxy, amino, azido, alkyl, haloalkyl, alkoxyalkyl, cycloalkyl, alkynyl, alkoxy, haloalkoxy, alkylthio, haloalkylthio, alkylsulfinyl, alkylsulfonyl, phenoxy that is optionally substituted, benzyloxy that is optionally substituted, phenyl that is optionally substituted, phenylalkyl that is optionally substituted, phenoxyalkyl that is optionally substituted, or 5- to 10-membered heterocyclic group that contains one to four hetero atoms selected from the group consisting of nitrogen atom, oxygen atom, and sulfur atom and is optionally substituted by a group selected from the group consisting of halogen, alkyl, and haloalkyl; or represents a group A, group B, group C, group F, group G, or group H as defined for formula (IA),
R^{4A} represents hydrogen atom, halogen, cyano, alkyl, haloalkyl, alkynyl, alkoxy, haloalkoxy, alkylthio, haloalkylthio, alkylsulfinyl, alkylsulfonyl, or a group 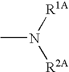
where R^{1A} and R^{2A} are as defined above,
reacting a compound of formula (IAf) 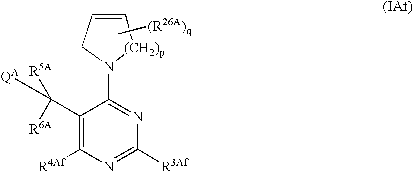
wherein
R^{3Af} represents hydrogen, halogen, cyano, hydroxy, amino, azido, alkyl, haloalkyl, alkoxyalkyl, cycloalkyl, alkynyl, alkoxy, haloalkoxy, alkylthio, haloalkylthio, alkylsulfinyl, alkylsulfonyl, phenoxy that is optionally substituted, benzyloxy that is optionally substituted, phenyl that is optionally substituted, phenylalkyl that is optionally substituted, phenoxyalkyl that is optionally substituted, or 5- to 10-membered heterocyclic group that contains one to four hetero atoms selected from the group consisting of nitrogen atom, oxygen atom, and sulfur atom and is optionally substituted by a group selected from the group consisting of halogen, alkyl, and haloalkyl; or represents a group A, group B, group C, group F, group G, or group H as defined for R^{3A} of formula (IA),
R^{4Af} represents hydrogen atom, halogen, cyano, alkyl, haloalkyl, alkynyl, alkoxy, haloalkoxy, alkylthio, haloalkylthio, alkylsulfinyl, alkylsulfonyl, or a group 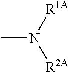
where R^{1A} and R^{2A} are as defined above,
R^{5A}, R^{6A}, and Q^{A} have the same definitions as for formula (IA),
R^{26A} represents alkyl,
p represents 1 or 2, and
q represents 0, 1, or 2,
with difluorocarbene derived from sodium chlorodifluoroacetate or with dichlorocarbene derived from chloroform, in the presence of an inert solvent and optionally in the presence of a phasetransfer catalyst,
or
(g) for compounds of formula (IA) in which R^{3A} represents amino,
hydrogenating or reacting with metal hydride a compound of formula (IAg) 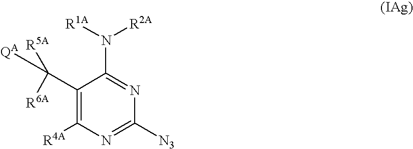
wherein R^{1A}, R^{2A}, R^{4A}, R^{5A}, R^{6A}, and Q^{A} have the same definitions as for formula (IA),
in the presence of an inert solvent and optionally in the presence of a catalyst,
or
(h) for compounds of formula (IA) in which R^{3A} represents halogen,
(1) in a first step, reacting a compound of formula (IAh) 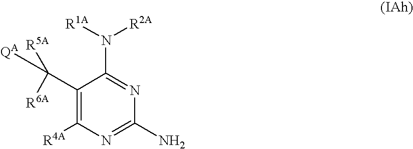
wherein R^{1A}, R^{2A}, R^{4A}, R^{5A}, R^{6A}, and Q^{A} have the same definitions as for formula (IA),
with a nitrite ester or nitrous acid in the presence of an inert solvent and optionally in the presence of acid catalyst to form a diazonium salt, and
(2) in a second step, reacting the diazonium salt obtained in the first step according to Sandmeyer process or Gattermann process in the presence of copper halide, potassium halide, or copper powder, in the presence an inert solvent and optionally in the presence of acid catalyst,
or
(i) for compounds of formula (IA) in which R^{3A} represents group E,
(1) in a first step, reacting a compound of formula (IAh) with dimethylformamide dimethylacetal in the presence of an inert solvent, to form a compound of formula (VI) 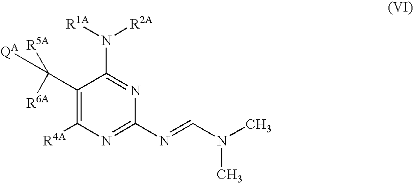
wherein R^{1A}, R^{2A}, R^{4A}, R^{5A}, R^{6A} and Q^{A} have the same definitions as for formula (IA), and
(2) in a second step, reacting the compound of formula (VI) obtained in the first step with a compound of formula (VII) 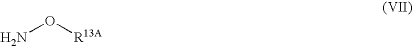
wherein R^{13A} has the same definition as for formula (IA),
in the presence of an inert solvent, optionally in the presence of an acid binder, and optionally in the presence of an acid catalyst,
or
(j) for compounds of formula (IA) in which R^{3A} represents the group D,
reacting a compound of formula (IAh) with a compound of formula (VIII) 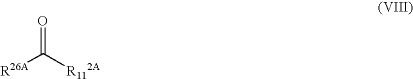
wherein R^{26A} represents chloro or a group 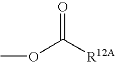
wherein R^{12A} has the same definition as for formula (IA),
in the presence of an inert solvent and optionally in the presence of an acid binder,
or
(k) for compounds of formula (IA) in which
R^{3A} represents the group K, and
R^{4A} represents hydrogen atom, halogen, alkyl, haloalkyl, alkenyl, alkynyl, alkoxy, haloalkoxy, alkylthio, haloalkylthio, alkylsulfinyl, alkylsulfonyl, or a group 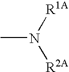
where R^{1A} and R^{2A} are as defined above,
reacting a compound of formula (IAk) 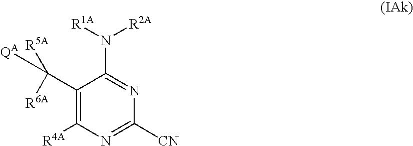
wherein
R^{4A} represents hydrogen atom, halogen, alkyl, haloalkyl, alkenyl, alkynyl, alkoxy, haloalkoxy, alkylthio, haloalkylthio, alkylsulfinyl, alkylsulfonyl, or a group 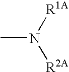
where R^{1A} and R^{2A} are as defined above, and
R^{1A}, R^{2A}, R^{5A}, R^{6A}, and Q^{A} have the same definitions as for formula (IA),
with a compound of formula (IX)
R^{20A}—Mg—Xk (IX)
wherein
Xk represents halogen, and
R^{20A} has the same definition as for formula (IA),
in the presence of an inert solvent,
or
(l) for compounds of formula (IA) in which
R^{3A} represents group L or group M, and
R^{4A} represents hydrogen atom, halogen, alkyl, haloalkyl, alkenyl, alkynyl, alkoxy, haloalkoxy, alkylthio, haloalkylthio, alkylsulfinyl, alkylsulfonyl, or a group 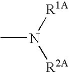
where R^{1A} and R^{2A} are as defined above,
reacting a compound of formula (IAl) 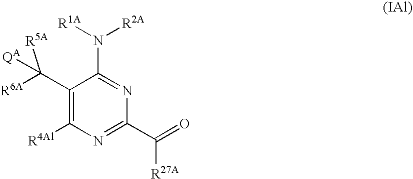
wherein
R^{27A} represents alkyl,
R^{4Al} represents hydrogen atom, halogen, alkyl, haloalkyl, alkenyl, alkynyl, alkoxy, haloalkoxy, alkylthio, haloalkylthio, alkylsulfinyl, alkylsulfonyl, or a group 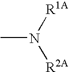
where R^{1A} and R^{2A} are as defined above, and
R^{1A}, R^{2A}, R^{5A}, R^{6A}, and Q^{A} have the same definitions as for formula (IA),
with a compound of formula (X)
H₂N—R^{28A} (X)
wherein R^{28A} represents a group —O—R^{22A} or a group 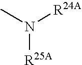
wherein R^{22A}, R^{24A}, and R^{25A} have the same definitions as for formula (IA),
in the presence of an inert solvent, optionally in the presence of acid binder, and optionally in the presence of acid catalyst,
or
(m) for compounds of formula (IA) in which R^{3A} represents group J,
reacting a compound of formula (IAk) with a compound of formula (XI)
H₂NO—R^{19A} (XI)
wherein R^{19A} has the same definition as for formula (IA),
in the presence of AN inert solvent, optionally in the presence of acid binder, and optionally in the presence of acid catalyst.

14. An agrohorticultural fungicide comprising a benzylpyrimidine derivative of formula (I) 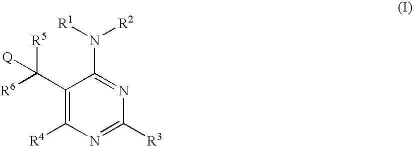
wherein
R¹ and R², together with the nitrogen atom to which they are bonded, form a 3- to 10-membered heterocyclic group that is optionally substituted and optionally contains one to three further hetero atoms selected from the group consisting of nitrogen atom, oxygen atom, and S(O)ₙ in addition to the nitrogen atom to which R¹ and R² are bonded,
n represents 0, 1, or 2,
R³ represents hydrogen, halogen, cyano, hydroxy, amino, azido, alkyl, haloalkyl, alkoxyalkyl, cycloalkyl, alkenyl, alkynyl, alkoxy, haloalkoxy, alkenyloxy, haloalkenyloxy, alkylthio, alkenylthio, haloalkenylthio, alkylsulfinyl, alkylsulfonyl, phenoxy that is optionally substituted, benzyloxy that is optionally substituted, phenyl that is optionally substituted, phenylalkyl that is optionally substituted, phenoxyalkyl that is optionally substituted, or 5- to 10-membered heterocyclic group that contains one to four hetero atoms selected from the group consisting of nitrogen atom, oxygen atom, and sulfur atom and is optionally substituted with a group selected from the group consisting of halogen, alkyl, and haloalkyl; or represents a moiety selected from the group consisting of formulas A to H and J to M 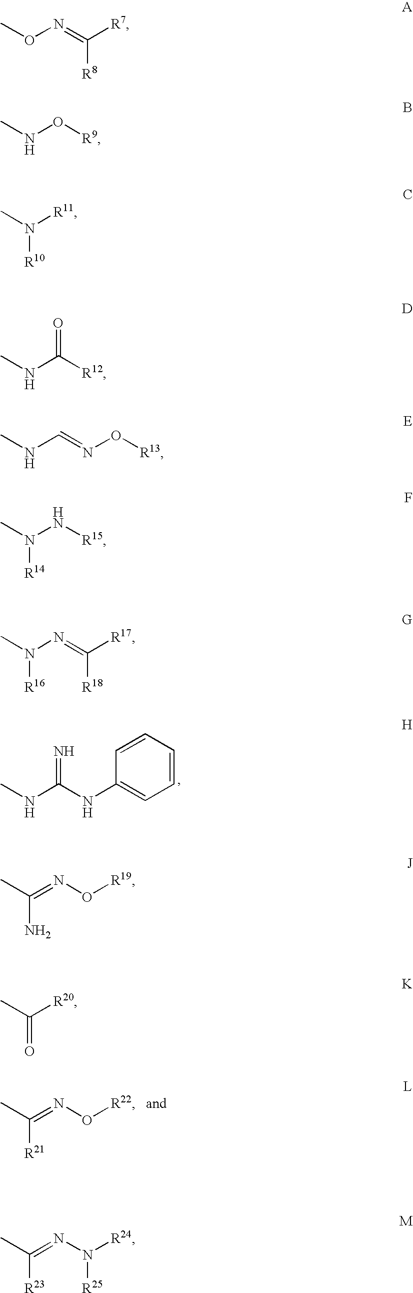
in which
R⁷ represents hydrogen atom, alkyl, or haloalkyl, and
R⁸ represents alkyl, phenyl, alkoxy or cyano, or
R⁷ and R⁸, together with the carbon atom to which they are bonded, form a cycloalkylidene,
R⁹ represents alkyl, haloalkenyl, or benzyl,
R¹⁰ represents hydrogen atom or alkyl,
R¹¹ represents alkyl, alkoxyalkyl, dialkylaminoalkyl, phenyl, benzyl, or cyano,
R¹² represents alkyl or phenyl,
R¹³ represents alkyl or benzyl,
R¹⁴ represents hydrogen atom or alkyl,
R¹⁵ represents hydrogen atom, haloalkyl, or phenyl,
R¹⁶ represents hydrogen atom or alkyl,
R¹⁷ represents hydrogen atom, alkyl, or haloalkyl,
R¹⁸ represents alkyl or phenyl,
R¹⁹ represents hydrogen atom or alkyl,
R²⁰ represents alkyl,
R²¹ represents alkyl,
R²² represents alkyl, alkenyl, haloalkenyl, alkoxyalkyl, phenoxyalkyl, or alkoxycarbonylalkyl,
R²³ represents alkyl, and
R²⁴ represents hydrogen atom or alkyl, and
R²⁵ represents alkyl or phenyl, or
R²⁴ and R²⁵, together with the nitrogen atom to which they are bonded, form a 5- to 8-membered saturated monoheterocyclic group that is optionally substituted and optionally contains one or two further hetero atoms selected from the group consisting of nitrogen atom, oxygen atom, and S(O)ₙ in addition to the nitrogen atom to which R²⁴ and R²⁵ are bonded,
R⁴ represents hydrogen atom, halogen, cyano, alkyl, haloalkyl, alkenyl, alkynyl, alkoxy, haloalkoxy, alkylthio, haloalkylthio, alkylsulfinyl, alkylsulfonyl, or a group 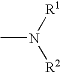
where R¹ and R² are as defined above,
R⁵ and R⁶ each independently represents hydrogen atom, halogen, alkyl, haloalkyl, or phenyl that is optionally substituted, and
Q represents aryl that is optionally substituted or a 5- or 6-membered heterocyclic group that contains one hetero atom selected from the group consisting of nitrogen atom, oxygen atom, and sulfur atom and is optionally substituted,
and optionally one or more extenders and/or carriers and/or surfactants and/or further formulation auxiliaries.

15. A process for the preparation of a microbicidal composition comprising mixing a benzylpyrimidine derivative of formula (I) according to claim 14 with one or more extenders and/or surface active agents.
